(19) United States
(12) Patent Application Publication
(10) Pub. No.: US 2019/0022107 A1
(43) Pub. Date: Jan. 24, 2019

(54) VAGINAL INSERTED ESTRADIOL PHARMACEUTICAL COMPOSITIONS AND METHODS

(71) Applicant: TherapeuticsMD, Inc., Boca Raton, FL (US)

(72) Inventors: Sebastian Mirkin, Boca Raton, FL (US); Julia M. Amadio, Boca Raton, FL (US); Brian A. Bernick, Boca Raton, FL (US)

(21) Appl. No.: 16/004,338

(22) Filed: Jun. 8, 2018

Related U.S. Application Data
(60) Provisional application No. 62/517,151, filed on Jun. 8, 2017.

(51) Int. Cl. A61K 31/565; A61K 9/48; A61K 9/02; A61K 9/00; A61K 47/14; A61K 47/10; A61P 15/02

(52) U.S. Cl. CPC A61K 31/565; A61K 9/4875; A61K 9/4825; A61P 15/02; A61K 9/0034; A61K 47/14; A61K 47/10; A61K 9/02

(57) ABSTRACT

The invention described herein relates to pharmaceutical compositions that are capable of delivering active pharmaceutical ingredients, such as estrogens, to the vagina and vagina-associated tissues and related methods of making and using such compositions (e.g., in the treatment of disease). Compositions of the invention are capable of being absorbed by the vagina or vagina-associated tissues such that subjects receiving treatment with the compositions may resume ambulatory activity immediately after receiving the treatment, the frequency or amount of discharge associated with the composition is low or negligible, there is little or no systemic absorption associated with the administration of the active pharmaceutical composition, or results in a combination of any or all thereof.

### CROSS-REFERENCES TO RELATED APPLICATIONS

This application claims priority to U.S. Provisional Patent Application No. 62/517,151, filed on Jun. 8, 2017, which application is incorporated herein by reference in its entirety.

### FIELD OF THE INVENTION

This application is directed to pharmaceutical compositions, methods, and devices related to hormone replacement therapy.

### BACKGROUND OF THE INVENTION

Postmenopausal women frequently suffer from atrophic vaginitis or vulvar and vaginal atrophy (hereinafter “vulvovaginal atrophy” or “VVA”) with symptoms including, for example, vaginal dryness, vaginal odor, vaginal or vulvar irritation or itching, dysuria (pain, burning, or stinging when urinating), dyspareunia (vaginal pain associated with sexual activity), or vaginal bleeding associated with sexual activity. Other symptoms include soreness; urinary frequency and urgency; urinary discomfort and incontinence also occurring (“estrogen-deficient urinary state(s)”). One symptom of vaginal atrophy is an increased vaginal pH, which creates an environment more susceptible to infections. The mucosal epithelium of the VVA patients also reported to show signs of severe atrophy and upon cytological examination accompanied by an increased number of the parabasal cells and a reduced number of superficial cells.

Each of these VVA-related states manifest symptoms associated with decreased estrogenization of the vulvovaginal tissue and can even occur in women treated with oral administration of an estrogen-based pharmaceutical drug product. Although VVA is most common with menopausal women, it can occur at any time in a woman's life cycle. VVA symptoms also interfere with sexual activity and satisfaction. Women with female sexual dysfunction (FSD) are almost 4 times more likely to have VVA than those without FSD.

Estrogen treatment has proven to be very successful in controlling menopausal symptoms, including VVA and FSD. Several studies have shown that the symptoms connected with vaginal atrophy are often relieved by estrogen treatment given either systemically or topically. The existing treatments have numerous problems including, for example compliance issues with patients not completing or continuing treatment due to the problems associated with the form of treatment.

Accordingly, there remains a need in the art for treatments for VVA and FSD that overcome these limitations.

### BRIEF SUMMARY OF THE INVENTION

Disclosed herein is, among other things, new soft gel vaginal pharmaceutical compositions and dosage forms containing one or more active pharmaceutical ingredients such as solubilized estradiol for the treatment of VVA and other conditions treatable via intravaginal delivery of an active pharmaceutical ingredient.

In one aspect, the present invention provides a method for treating moderate to severe dyspareunia in a subject, the method comprising: intravaginally administering a pharmaceutical composition comprising estradiol to the subject, wherein the pharmaceutical composition is administered to the lower third of the vagina closest to the vaginal opening, and wherein the estradiol is not transported to the uterus of the subject. The pharmaceutical composition is preferably a liquid pharmaceutical composition comprising 4 μg to 25 μg of estradiol, and wherein the liquid pharmaceutical composition is contained in a capsule. In one embodiment, the administering is carried out by digitally inserting the capsule into the lower third of the vagina closest to the vaginal opening, or by digitally inserting the capsule about two inches into the vagina closest to the vaginal opening.

Importantly, using this method, transport of estradiol to the uterus of the subject is avoided (it is minimal or de minims, or it is in an amount that is not sufficient to cause endometrial hyperplasia after 12 weeks of treatment), i.e., the estradiol that is administered is not transported to the uterus. Importantly, this method for treating moderate to severe dyspareunia in a subject does not cause endometrial hyperplasia.

In an embodiments of the above method, the capsule is a bioadhesive capsule. In another embodiment, the capsule is a gelatin capsule, such as a soft gelatin capsule.

In one embodiment of the above method, intravaginally administering the liquid pharmaceutical composition comprises digitally inserting the capsule into the lower third of the vagina of the subject or two inches into the vagina closest to the vaginal opening. In one embodiment of the above method, the capsule adheres to the vaginal tissue of the subject and dissolves, ruptures, or disintegrates, thereby releasing the liquid pharmaceutical composition. In one embodiment, the liquid pharmaceutical composition spreads over a surface area selected from the group consisting of the vagina, the vulva, the labia, and combinations thereof. In preferred embodiments of this method, the soft gelatin capsule and the liquid pharmaceutical composition are fully absorbed by the vaginal tissue of the subject. Importantly, using the above methods, transport of estradiol to the uterus of the subject is avoided (i.e., minimal or de minims or in an amount that is not sufficient to cause endometrial hyperplasia after 12 weeks of treatment), i.e., the estradiol that is administered is not transported to the uterus.

Some advantages of the above methods and the other methods disclosed herein include, but are not limited to the following: (i) vaginal secretions from the subject are not required for the capsule to dissolve, rupture, or disintegrate, thereby releasing the liquid pharmaceutical composition; (ii) the only discharge that occurs after intravaginally administering the liquid pharmaceutical composition is a natural discharge; (iii) the subject can be ambulatory immediately after intravaginally administering the liquid pharmaceutical composition, or the subject can be ambulatory for a period of time beginning 5 minutes to 120 minutes after intravaginally administering the liquid pharmaceutical composition; and (iv) importantly estradiol is not transported to the uterus, i.e., transport of estradiol to the uterus is avoided, with the methods of the invention.

In the above method, the liquid pharmaceutical compositions further comprise a solubilizing agent. In preferred embodiments, the solubilizing agent is an oil. In preferred embodiments, the oil comprises at least one C6-C12 fatty acid or a glycol, monoglyceride, diglyceride, or triglyceride ester thereof. In preferred embodiments, the liquid pharmaceutical composition further comprises a thickener or a surfactant. In certain embodiments, the liquid pharmaceutical compositions do not include a hydrophilic gel-forming bioadhesive agent. In preferred embodiments, estradiol is the only active hormone in the liquid pharmaceutical compositions.

In some embodiments, the liquid pharmaceutical compositions include 4 μg estradiol. In other embodiments, the liquid pharmaceutical compositions include 10 μg estradiol. In yet other embodiments, the liquid pharmaceutical compositions include 25 μg estradiol.

In the above methods, the intravaginal administration is preferably conducted daily for two weeks, and twice weekly thereafter. The intravaginal administration is preferably conducted at any time of day, but is conducted at about the same time each day.

The above methods are surprisingly effective within two weeks of the first administration. Importantly, the above method for treating moderate to severe dyspareunia in a subject provides: (i) increasing the level of vaginal secretions in a subject, as assessed by visual examination; (ii) increasing the number of vaginal rugae in the subject, as assessed by visual examination; (iii) decreasing vaginal bleeding or petechiae in the subject, as assessed by visual examination; and (iv) changing the color of the vaginal mucosa in the subject from transparent to pink, or from pale pink to pink, as assessed by visual examination. Importantly, the above method (i) decreases the severity of vaginal dryness within two weeks; (ii) decreases the severity of vulvar or vaginal itching within two weeks; and (iii) decreases the severity of decreases the severity of dyspareunia within two weeks. Importantly, the method provides these benefits while avoiding transport of the estradiol to the uterus.

In another aspect, the invention provides a method for avoiding transport of estradiol to the uterus of a subject in need of estradiol, the method comprising: intravaginally administering a pharmaceutical composition comprising estradiol to the subject, wherein the pharmaceutical composition is administered to the lower third of the vagina closest to the vaginal opening. In one embodiment, the pharmaceutical composition is a liquid pharmaceutical composition comprising 4 μg to 25 μg of estradiol, and wherein the liquid pharmaceutical composition is contained in a capsule. In another embodiment, the administering is carried out by digitally inserting the capsule into the lower third of the vagina closest to the vaginal opening, or by digitally inserting the capsule about two inches into the vagina closest to the vaginal opening. Importantly, this method of delivering estradiol to a subject in need thereof does not cause endometrial hyperplasia.

The compositions of the invention comprise a formulation that permits a therapeutically effective amount of the product to be administered while the subject (typically a female human) is upright and that does not require the subject to assume a supine position after administration. According to embodiments, compositions of the invention can be formulated such that the subject may immediately resume ambulatory activity after administration. According to embodiments, methods of the invention include the provision of instructions regarding the possibility of upright administration, resumption of ambulatory activity, or both, such as by healthcare provider instruction, product labeling, or a combination thereof.

According to embodiments, the composition of the invention comprises a delivery vehicle, such as a softgel capsule, which comprises a formulation comprising the API, and which disintegrates in the vagina releasing the formulation. According to embodiments, such complete disintegration occurs (or the capsule cannot be detected in the subject) at least about 95%, such as at least about 98% of the time in subjects, such as has been demonstrated in clinical trials of such products. According to embodiments, administration of such products results in little or no composition-associated discharge, at least in the substantial majority of cases (e.g., at least about 90%, at least about 95%, or at least about 99% of the time). According to certain embodiments, the softgel vaginal composition is administered digitally, without an applicator. According to certain embodiments, the softgel is inserted into the lower half of the vagina, for example approximately in the lower 2 inches of the vagina.

The compositions of the invention comprise one or more pharmaceutical ingredients that are effective in treating one or more conditions when administered to the vagina, such as estradiol or a non-estradiol estrogen.

Compositions of the invention comprise a solubilizing agent. In the case of delivery vehicle compositions, the solubilizing agent will typically be contained in the enclosed or associated formulation comprising the API. According to embodiments, at least about 20% of the composition or formulation comprises a medium chain oil or is otherwise composed of medium chain fatty acids. According to embodiments the composition or formulation further comprises a surfactant, and a thickening agent. According to various aspects and embodiments of this disclosure, the surfactant and thickening agent of the formulation originate from the same excipient or pre-established mixture of excipients. According to embodiments, the surfactant and thickener comprise a first polyethylene glycol (PEG) compound comprising 2-10 PEG units and a second PEG compound comprising 20-40 PEG units, such as would be present in a TEFOSE-type of surfactant, such as TEFOSE 63. In some embodiments, at least half of the formulation is comprised of the solubilizing agent and no more than 25% of the formulation is comprised of the surfactant and the thickening agent.

According to embodiments a formulation comprising a solubilizing agent, which is substantially composed of a medium chain oil, a thickening agent, and a surfactant, wherein the thickening agent and surfactant can added as a single composition of one or more chemical species, is contained in a softgel capsule. According to some embodiments, the softgel capsule, while in contact with the vaginal mucosa, ruptures and releases the contained formulation. In some embodiments, the softgel disintegrates within one day of administration without detectable discharge. According to embodiments the API is at least about 80%, such as at least about 95%, or at least about 99%, or even completely (within the limits of detection) solubilized in the solubilization agent. According to some embodiments, the formulation does not contain any bioadhesive gel or gel-forming agent other than the gelatin or hydrolyzed gelatin component of the softgel. According to some embodiments, the composition or formulation does not contain carboxyvinylic acids, gelatin, hydroxypropylcellulose, carboxymethylcellulose, xanthane gum, guar gum, aluminum silicate, colloidal silica, and mixtures thereof.

In some embodiments, the thickening agent provides the ability to modify the viscosity of the formulation such that without the thickening agent, the formulation is not retained within the vagina over the course of 24 hours after administration and formulation leaking is experience; but wherein inclusion of the thickening agent allows the formulation to be retained in the vagina over the course of 24 hours after administration without the subject experiencing non-normal discharge.

In some embodiments, the subject may administer the formulation at any time of day, may be optionally administered while the subject is upright, and allows the subject to remain upright and resume ambulation immediately following administration of the formulation.

According to certain embodiments, the surfactant and thickening agent formulation allows for the formulation to be retained such that the subject does not experience non-normal discharge however allows for spreading of the formulation such that, for example in the case of the API estradiol, the vagina, vulva, and labia are re-estrogenized. According to embodiments, the formulation, API, or both, will detectably spread about 50 cm² to about 120 cm² following administration (e.g., on average in an individual or in a population of individuals, such as those populations described herein in connection with this and other properties of the compositions of the invention). According to embodiments the API is delivered with low or no systemic absorption of the API over baseline, placebo, or both. According to embodiments, the medium chain oil or the entire solubilizing agent has a viscosity of between about 5 centipoise (“cps”) and about 50 cps at 25 degrees C. and the formulation, after addition of a thickening agent and surfactant, has a viscosity of between about 70 cps and about 120 cps at 25 degrees C. According to embodiments, the ratio of the solubilizing agent to the combination of the surfactant and the thickener in the composition is between about 4:1 and about 12:1, such as between about 7:1 and about 11:1 or between about 8:1 and about 10:1. According to embodiments, the surfactant (or surfactant and thickener if provided as a combined agent or a combined mixture of compounds) is a non-ionic surfactant having an HLB of between 7 and about 15 (e.g., between about 8 and 14, such as between about 9 and 13).

Compositions of the invention include combinations of inventive features that allow the compositions and methods provided herein to overcome or mitigate limitations found with other vaginal delivery systems. The soft gel vaginal pharmaceutical composition of embodiments of the invention can ease vaginal administration, provides improved safety of insertion, minimizes vaginal discharge following administration, and provides a more effective dosage form having improved efficacy, safety, patient compliance, and user experiences. According to embodiments, the size of a delivery vehicle of the invention, such as a softgel capsule, will not exceed about 0.75 inches in any dimension. According to embodiments, the amount of formulation contained in the delivery vehicle, such as a softgel capsule, will not exceed 1000 mg and in more particular embodiments will be between about 50 mg and about 500 mg (e.g., about 100-500 mg, about 100-400 mg, about 150-375 mg, or about 200-400 mg). According to embodiments, the delivery vehicle, such as a softgel capsule, comprises a first end having a maximum width that is less than about 75%, less than about 66%, less than about 50%, or less than about 33% (such as less than about 25%, 20%, or even 10%) of the maximum width of the second end, and in such cases methods of the invention can optionally include the step of instructing the subject to administer the gel cap by inserting the narrower first end of the capsule first. The inclusion of such features in combination with the properties of the formulations provided by this invention can increase the user experience associated with such products and thereby increase compliance in use, as demonstrated in connection with exemplary products provided herein.

The compositions of the invention can be used for the treatment of a number of conditions, depending on the APIs delivered by the composition. In an exemplary aspect, the API is an estrogen, such as estradiol, and the use of the composition provides a treatment for women suffering with moderate to severe symptoms of VVA. In another aspect, the invention provides a method for treating a state of deficiency of an endogenous vaginal tissue-associated molecule, such as estradiol. Thus, for example, the invention can provide a method of treating an estrogen-deficient state in a subject, such as a human female.

In some embodiments of the methods provided herein, treatment includes reducing the severity of one or more symptoms selected from the group consisting of: vaginal dryness, dyspareunia, vaginal or vulvar irritation, vaginal or vulvar burning, vaginal or vulvar itching, dysuria, and vaginal bleeding associated with sexual activity.

In some embodiments of the methods provided herein treatment includes reducing the vaginal pH of the patient. For example, treatment includes reducing the vaginal pH of the patient to a pH of less than about 5.0.

In some embodiments of the methods provided herein treatment includes a change in cell composition of the patient. For example, the change in cell composition includes reducing the number of parabasal vaginal cells or increasing the number of superficial vaginal cells. In some embodiments, the number of parabasal vaginal cells in the patient are reduced by at least about 35% (e.g., at least about 50%). In some embodiments, the number of superficial vaginal cells are increased by at least about 5% (e.g., at least about 35%).

Further provided herein is a method for reducing vaginal discharge following administration of a suppository, the method comprising administering to a patient in need thereof, a suppository provided herein, wherein the vaginal discharge following administration of the suppository is compared to the vaginal discharge following administration of a reference drug.

Also provided herein is a method for treating female sexual dysfunction in a female subject in need thereof. The method includes administering to the subject a vaginal suppository as described herein. In some embodiments, treating female sexual dysfunction includes increasing the subject's desire, arousal, lubrication, satisfaction, and/or orgasms.

### BRIEF DESCRIPTION OF THE DRAWINGS

The above-mentioned features and objects of this disclosure will become more apparent with reference to the following description taken in conjunction with the accompanying drawings wherein like reference numerals denote like elements and in which:

FIG. 1 is a flow diagram illustrating a process in accordance with various embodiments of the invention;

FIG. 2 illustrates a suppository in accordance with various embodiments of the invention;

FIG. 3 is a linear plot of mean plasma estradiol-baseline adjusted concentrations versus time (N=34);

FIG. 4 is a semi-logarithmic plot of mean plasma estradiol-baseline adjusted concentrations versus time (N=34);

FIG. 5 is a linear plot of mean plasma estrone-baseline adjusted concentrations versus time (N=33);

FIG. 6 is a semi-logarithmic plot of mean plasma estrone-baseline adjusted concentrations versus time (N=33);\

FIG. 7 is a linear plot of mean plasma estrone sulfate-baseline adjusted concentrations versus time (N=24); and

FIG. 8 is a semi-logarithmic plot of mean plasma estrone sulfate-baseline adjusted concentrations versus time (N=24).

FIG. 9 is a study schematic diagram.

FIG. 10 shows the percentage change in superficial cells at 12 weeks compared to placebo.

FIG. 11 shows the percentage change in superficial cells at week 2, week 6, week 8, and week 12 compared to placebo.

FIG. 12 shows percentage change in superficial cells per dose for each of week 2, week 6, week 8, and week 12 compared to placebo.

FIG. 13 shows the percentage change in parabasal cells at 12 weeks compared to placebo.

FIG. 14 shows the percentage change in parabasal cells at week 2, week 6, week 8, and week 12 compared to placebo.

FIG. 15 shows the percentage change in parabasal cells per dose for each of week 2, week 6, week 8, and week 12 compared to placebo

FIG. 16 shows the percentage change in pH at 12 weeks compared to placebo.

FIG. 17 shows the percentage change in pH at week 2, week 6, week 8, and week 12 compared to placebo.

FIG. 18 shows the percentage change in pH per dose for each of week 2, week 6, week 8, and week 12 compared to placebo.

FIG. 19A shows the change in visual assessments from baseline to week 12 in vaginal color in a modified intent to treat (MITT) population.

FIG. 19B shows the change in visual assessments from baseline to week 12 in vaginal epithelial integrity in a modified intent to treat (MITT) population.

FIG. 19C shows the change in visual assessments from baseline to week 12 in vaginal epithelial thickness a modified intent to treat (MITT) population.

FIG. 19D shows the change in visual assessments from baseline to week 12 in vaginal secretions in a modified intent to treat (MITT) population.

FIG. 20A shows the correlation between the total sum of four visual assessments and dyspareunia at week 12 in an intent to treat (ITT) population.

FIG. 20B shows the correlation between the total sum of four visual assessments and vaginal dryness at week 12 in an intent to treat (ITT) population.

FIG. 21 shows baseline adjusted estradiol serum concentration (pg/mL) assessed on Day 1 (squares) and Week 12 (diamonds) for four treatment arms.

FIG. 22 shows baseline adjusted estradiol serum concentration (pg/mL) assessed on Day 14 (squares) and Week 12 (diamonds) for four treatment arms.

FIG. 23 shows estradiol plasma levels measured in subjects following a supine period after administration of the estradiol formulation, compared with plasma levels measured in subjects following an ambulatory period after administration of the estradiol formulation.

FIG. 24 shows mean change from baseline in Total FSFI score at Week 12.

FIG. 25A shows the mean change from baseline to week 12 in the individual FSFI lubrication score.

FIG. 25B shows the mean change from baseline to week 12 in the individual FSFI arousal score.

FIG. 25C shows the mean change from baseline to week 12 in the individual FSFI satisfaction score.

FIG. 25D shows the mean change from baseline to week 12 in the individual FSFI desire score.

FIG. 25E shows the mean change from baseline to week 12 in the individual FSFI orgasm score.

FIG. 26A shows an estradiol softgel capsule held with the larger end between the fingers.

FIG. 26B shows insertion of an estradiol softgel capsule in a reclining position. The softgel is inserted into the lower third of the vagina with the smaller end up.

FIG. 26C shows insertion of an estradiol softgel capsule in a standing position. The softgel is inserted into the lower third of the vagina with the smaller end up.

### DETAILED DESCRIPTION OF THE INVENTION

In the following detailed description of embodiments of this disclosure, reference is made to the accompanying drawings in which like references indicate similar elements, and in which is shown by way of illustration specific embodiments in which this disclosure may be practiced. These embodiments are described in sufficient detail to enable those skilled in the art to practice this disclosure, and it is to be understood that other embodiments may be utilized and that other changes may be made without departing from the scope of the this disclosure. The following detailed description is, therefore, not to be taken in a limiting sense, and the scope of this disclosure is defined only by the appended claims. As used in this disclosure, the term “or” shall be understood to be defined as a logical disjunction (i.e., and/or) and shall not indicate an exclusive disjunction unless expressly indicated as such with the terms “either,” “unless,” “alternatively,” and words of similar effect.

### I. DEFINITIONS

The term “active pharmaceutical ingredient” (“API”) as used herein, means the active compound(s) used in formulating a drug product.

The term “co-administered” as used herein, means that two or more drug products (or APIs) are administered simultaneously or sequentially on the same or different days.

The term “drug product” as used herein means at least one active pharmaceutical ingredient in combination with at least one excipient and provided in unit dosage form.

The term “area under the curve” (“AUC”) refers to the area under the curve defined by changes in the blood concentration of an active pharmaceutical ingredient (e.g., estradiol or progesterone), or a metabolite of the active pharmaceutical ingredient, over time following the administration of a dose of the active pharmaceutical ingredient. “AUC_{0-∞}” is the area under the concentration-time curve extrapolated to infinity following the administration of a dose. “AUC₀₋ₜ” is the area under the concentration-time curve from time zero to time t following the administration of a dose, wherein t is the last time point with a measurable concentration.

The term “Cₘₐₓ” refers to the maximum value of blood concentration shown on the curve that represents changes in blood concentrations of an active pharmaceutical ingredient (e.g., progesterone or estradiol), or a metabolite of the active pharmaceutical ingredient, over time.

The term “Tₘₐₓ” refers to the time that it takes for the blood concentration of an active pharmaceutical ingredient (e.g., estradiol or progesterone), or a metabolite of the active pharmaceutical ingredient, to reach the maximum value.

The term “bioavailability,” which has the meaning defined in 21 C.F.R. § 320.1(a), refers to the rate and extent to which an API or active ingredient or active moiety is absorbed from a drug product and becomes available at the site of action. For example, bioavailability can be measured as the amount of API in the blood (serum or plasma) as a function of time. Pharmacokinetic (PK) parameters such as AUC, Cₘₐₓ, or Tₘₐₓ may be used to measure and assess bioavailability. For drug products that are not intended to be absorbed into the bloodstream, bioavailability may be assessed by measurements intended to reflect the rate and extent to which the API or active ingredient or active moiety becomes available at the site of action.

The term “bioequivalent,” which has the meaning defined in 21 C.F.R. § 320.1(e), refers to the absence of a significant difference in the rate and extent to which the API or active ingredient or active moiety in pharmaceutical equivalents or pharmaceutical alternatives becomes available at the site of drug action when administered at the same molar dose under similar conditions in an appropriately designed study. Where there is an intentional difference in rate (e.g., in certain extended release dosage forms), certain pharmaceutical equivalents or alternatives may be considered bioequivalent if there is no significant difference in the extent to which the active ingredient or moiety from each product becomes available at the site of drug action. This applies only if the difference in the rate at which the active ingredient or moiety becomes available at the site of drug action is intentional and is reflected in the proposed labeling, is not essential to the attainment of effective body drug concentrations on chronic use, and is considered medically insignificant for the drug. In practice, two products are considered bioequivalent if the 90% confidence interval of the AUC, Cₘₐₓ, or optionally Tₘₐₓ is within 80.00% to 125.00%.

The term “bio-identical,” “body-identical,” or “natural” used in conjunction with the hormones disclosed herein, means hormones that match the chemical structure and effect of those that occur naturally or endogenously in the human body. An exemplary body-identical or natural estrogen is estradiol.

The term “bio-identical hormone” or “body-identical hormone” refers to an active pharmaceutical ingredient that is structurally identical to a hormone naturally or endogenously found in the human body (e.g., estradiol and progesterone).

The term “estradiol” refers to (17β)-estra-1,3,5(10)-triene-3,17-diol. Estradiol is also interchangeably called 17β-estradiol, oestradiol, or E2, and is found endogenously in the human body. As used herein, estradiol refers to the bio-identical or body-identical form of estradiol found in the human body having the structure:

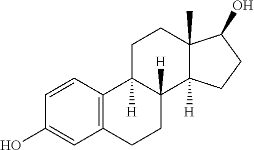

Estradiol is supplied in an anhydrous or hemi-hydrate form. For the purposes of this disclosure, the anhydrous form or the hemihydrate form can be substituted for the other by accounting for the water or lack of water according to well-known and understood techniques.

The term “solubilized estradiol” means that the estradiol or a portion thereof is solubilized or dissolved in the solubilizing agent(s) or the formulations disclosed herein. Solubilized estradiol may include estradiol that is about 80% solubilized, about 85% solubilized, about 90% solubilized, about 95% solubilized, about 96% solubilized, about 97% solubilized, about 98% solubilized, about 99% solubilized or about 100% solubilized. In some embodiments, the estradiol is “fully solubilized” with all or substantially all of the estradiol being solubilized or dissolved in the solubilizing agent. Fully solubilized estradiol may include estradiol that is about 97% solubilized, about 98% solubilized, about 99% solubilized or about 100% solubilized. Solubility can be expressed as a mass fraction (% w/w, which is also referred to as wt %).

The term “progesterone” refers to pregn-4-ene-3,20-dione. Progesterone is also interchangeably called P4 and is found endogenously in the human body. As used herein, progesterone refers to the bio-identical or body-identical form of progesterone found in the human body having the structure:

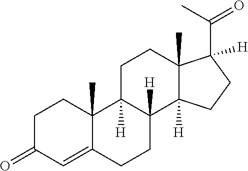

The term “solubilized progesterone” means that the progesterone or a portion thereof is solubilized or dissolved in the solubilizing agent(s) or the formulations disclosed herein. In some embodiments, the progesterone is “partially solubilized” with a portion of the progesterone being solubilized or dissolved in the solubilizing agent and a portion of the progesterone being suspended in the solubilizing agent. Partially solubilized progesterone may include progesterone that is about 1% solubilized, about 5% solubilized, about 10% solubilized, about 15% solubilized, about 20% solubilized, about 30% solubilized, about 40% solubilized, about 50% solubilized, about 60% solubilized, about 70% solubilized, about 80% solubilized, about 85% solubilized, about 90% solubilized or about 95% solubilized. In other embodiments, the progesterone is “fully solubilized” with all or substantially all of the progesterone being solubilized or dissolved in the solubilizing agent. Fully solubilized progesterone may include progesterone that is about 97% solubilized, about 98% solubilized, about 99% solubilized or about 100% solubilized. Solubility can be expressed as a mass fraction (% w/w, which is also referred to as wt %).

The terms “micronized progesterone” and “micronized estradiol,” as used herein, include micronized progesterone and micronized estradiol having an X50 particle size value below about 15 microns or having an X90 particle size value below about 25 microns. The term “X50” means that one-half of the particles in a sample are smaller in diameter than a given number. For example, micronized progesterone having an X50 of 5 microns means that, for a given sample of micronized progesterone, one-half of the particles have a diameter of less than 5 microns. Similarly, the term “X90” means that ninety percent (90%) of the particles in a sample are smaller in diameter than a given number.

The term “glyceride” is an ester of glycerol (1,2,3-propanetriol) with acyl radicals of fatty acids and is also known as an acylglycerol. If only one position of the glycerol molecule is esterified with a fatty acid, a “monoglyceride” or “monoacylglycerol” is produced; if two positions are esterified, a “diglyceride” or “diacylglycerol” is produced; and if all three positions of the glycerol are esterified with fatty acids, a “triglyceride” or “triacylglycerol” is produced. A glyceride is “simple” if all esterified positions contain the same fatty acid; whereas a glyceride is “mixed” if the esterified positions contained different fatty acids. The carbons of the glycerol backbone are designated sn-1, sn-2 and sn-3, with sn-2 being in the middle carbon and sn-1 and sn-3 being the end carbons of the glycerol backbone.

The term “solubilizing agent” refers to an agent or combination of agents that solubilize an active pharmaceutical ingredient (e.g., estradiol or progesterone). For example and without limitation, suitable solubilizing agents include medium chain oils and other solvents and co-solvents that solubilize or dissolve an active pharmaceutical ingredient to a desirable extent. Solubilizing agents suitable for use in the formulations disclosed herein are pharmaceutical grade solubilizing agents (e.g., pharmaceutical grade medium chain oils). It will be understood by those of skill in the art that other excipients or components can be added to or mixed with the solubilizing agent to enhance the properties or performance of the solubilizing agent or resulting formulation. Examples of such excipients include, but are not limited to, surfactants, emulsifiers, thickeners, colorants, flavoring agents, etc. In some embodiments, the solubilizing agent is a medium chain oil and, in some other embodiments, the medium chain oil is combined with a co-solvent(s) or other excipient(s).

The term “medium chain” is used to describe the aliphatic chain length of fatty acid containing molecules. “Medium chain” specifically refers to fatty acids, fatty acid esters, or fatty acid derivatives that contain fatty acid aliphatic tails or carbon chains that contain 6 (C6) to 14 (C14) carbon atoms, 8 (C8) to 12 (C12) carbon atoms, or 8 (C8) to 10 (C10) carbon atoms.

The terms “medium chain fatty acid” and “medium chain fatty acid derivative” are used to describe fatty acids or fatty acid derivatives with aliphatic tails (i.e., carbon chains) having 6 to 14 carbon atoms. Fatty acids consist of an unbranched or branched aliphatic tail attached to a carboxylic acid functional group. Fatty acid derivatives include, for example, fatty acid esters and fatty acid containing molecules, including, without limitation, mono-, di- and triglycerides that include components derived from fatty acids. Fatty acid derivatives also include fatty acid esters of ethylene or propylene glycol. The aliphatic tails can be saturated or unsaturated (i.e., having one or more double bonds between carbon atoms). In some embodiments, the aliphatic tails are saturated (i.e., no double bonds between carbon atoms). Medium chain fatty acids or medium chain fatty acid derivatives include those with aliphatic tails having 6-14 carbons, including those that are C6-C14, C6-C12, C8-C14, C8-C12, C6-C10, C8-C10, or others. Examples of medium chain fatty acids include, without limitation, caproic acid, caprylic acid, capric acid, lauric acid, myristic acid, and derivatives thereof.

The term “oil,” as used herein, refers to any pharmaceutically acceptable oil, especially medium chain oils, and specifically excluding peanut oil, that can suspend or solubilize bioidentical progesterone or estradiol, including starting materials or precursors thereof, including micronized progesterone or micronized estradiol as described herein.

The term “medium chain oil” refers to an oil wherein the composition of the fatty acid fraction of the oil is substantially medium chain (i.e., C6 to C14) fatty acids, i.e., the composition profile of fatty acids in the oil is substantially medium chain. With respect to medium chain oils, the term “substantially” means that between 20% and 100% (inclusive of the upper and lower limits) of the fatty acid fraction of the oil is made up of medium chain fatty acids, i.e., fatty acids with aliphatic tails (i.e., carbon chains) having 6 to 14 carbons. Elsewhere in this disclosure, the term “substantially” is used to describe an amount of at least 80% (for example, about 80%, about 85%, at least about 90%, at least about 95%, at least about 97%, at least about 99%, at least about 99.5% or about 100%. In some embodiments, about 25%, about 30%, about 35%, about 40%, about 45%, about 50%, about 55%, about 60%, about 65%, about 70%, about 75%, about 85%, about 90% or about 95% of the fatty acid fraction of the oil is made up of medium chain fatty acids. As used herein, “predominantly” means that greater than or equal to 50% of the fatty acid fraction of the oil is made up of medium-chain fatty acids, i.e., fatty acids with aliphatic carbon chains having 6 to 14 carbon atoms. Those of skill in the art that will readily appreciate that the terms “alkyl content” or “alkyl distribution” of an oil can be used in place of the term “fatty acid fraction” of an oil in characterizing a given oil or solubilizing agent, and these terms are used interchangeable herein. As such, medium chain oils suitable for use in the formulations disclosed herein include medium chain oils wherein the fatty acid fraction of the oil is substantially medium chain fatty acids, or medium chain oils wherein the alkyl content or alkyl distribution of the oil is substantially medium chain alkyls (C6-C12 alkyls). It will be understood by those of skill in the art that the medium chain oils suitable for use in the formulations disclosed herein are pharmaceutical grade (e.g., pharmaceutical grade medium chain oils). Examples of medium chain oils include, for example and without limitation, medium chain fatty acids, medium chain fatty acid esters of glycerol (e.g., for example, mono-, di-, and triglycerides), medium chain fatty acid esters of propylene glycol, medium chain fatty acid derivatives of polyethylene glycol, and combinations thereof.

The term “ECN” or “equivalent carbon number” means the sum of the number of carbon atoms in the fatty acid chains of an oil, and can be used to characterize an oil as, for example, a medium chain oil or a long-chain oil. For example, tripalmitin (tripalmitic glycerol), which is a simple triglyceride containing three fatty acid chains of 16 carbon atoms, has an ECN of 3×16=48. Conversely, a triglyceride with an ECN=40 may have “mixed” fatty acid chain lengths of 8, 16 and 16; 10, 14 and 16; 8, 14 and 18; etc. Naturally occurring oils are frequently “mixed” with respect to specific fatty acids, but tend not to contain both long chain fatty acids and medium chain fatty acids in the same glycerol backbone. Thus, triglycerides with ECN's of 21-42 typically contain predominantly medium chain fatty acids; while triglycerides with ECN's of greater than 43 typically contain predominantly long chain fatty acids. For example, the ECN of corn oil triglyceride in the USP would be in the range of 51-54. Medium chain diglycerides with ECN's of 12-28 will often contain predominantly medium chain fatty chains, while diglycerides with ECN's of 32 or greater will typically contain predominantly long chain fatty acid tails. Monoglycerides will have an ECN that matches the chain length of the sole fatty acid chain. Thus, monoglyceride ECN's in the range of 6-14 contain mainly medium chain fatty acids, and monoglycerides with ECN's 16 or greater will contain mainly long chain fatty acids.

The average ECN of a medium chain triglyceride oil is typically 21-42. For example, as listed in the US Pharmacopeia (USP), medium chain triglycerides have the following composition as the exemplary oil set forth in the table below:

| Fatty-acid Tail Length | % of oil | Exemplary Oil |
|---|---|---|
| 6 | ≤2.0 | 2.0 |
| 8 | 50.0-80.0 | 70.0 |
| 10 | 20.0-50.0 | 25.0 |
| 12 | ≤3.0 | 2.0 |
| 14 | ≤1.0 | 1.0 |

and would have an average ECN of 3*[(6*0.02)+(8*0.70)+(10*0.25)+(12*0.02)+(14*0.01)]=25.8. The ECN of the exemplary medium chain triglycerides oil can also be expressed as a range (per the ranges set forth in the USP) of 24.9-27.0. For oils that have mixed mono-, di-, and triglycerides, or single and double fatty acid glycols, the ECN of the entire oil can be determined by calculating the ECN of each individual component (e.g., C8 monoglycerides, C8 diglycerides, C10 monoglycerides, and C10 monoglycerides) and taking the sum of the relative percentage of the component multiplied by the ECN normalized to a monoglyceride for each component. For example, the oil having C8 and C10 mono- and diglycerides shown in the table below has an ECN of 8.3, and is thus a medium chain oil.

| | | ECN as % of oil | ECN as % of oil |
|---|---|---|---|
| Fatty-acid Chain | | (chain length) × | normalized to |
| Length | % of oil | (% in oil) | monoglyceride |
| C8 monoglyceride | 7 | 8 × 0.47 = 3.76 | 3.76 |
| C10 monoglyceride | 8 | 10 × 0.08 = 0.8 | 0.8 |
| C8 diglyceride | 8 | 2 × (8 × 0.38) = 6.08 | 6.08/2 = 3.04 |
| C10 diglyceride | 7 | 2 × (10 × 0.07) = 1.4 | 1.4/2 = 0.7 |
| OIL ECN | | | 8.3 |
| (normalized to | | | |
| monoglycerides) | | | |

Expressed differently, ECN can be calculated as each chain length in the composition multiplied by its relative percentage in the oil: (8*0.85)+(10*0.15)=8.3.

The term “excipients,” as used herein, refers to non-API ingredients such as solubilizing agents, anti-oxidants, oils, lubricants, and others used in formulating pharmaceutical products.

The term “patient” or “subject” refers to an individual to whom the pharmaceutical composition is administered, such as a human, especially a woman.

The term “pharmaceutical composition” refers to a pharmaceutical composition comprising at least a solubilizing agent and estradiol. As used herein, pharmaceutical compositions are delivered, for example via suppository (i.e., vaginal suppository), or absorbed vaginally.

The term “progestin” means any natural or man-made substance that has pharmacological properties similar to progesterone.

The terms “treat,” “treating,” and “treatment” refer to achievement of an objective success in the treatment or amelioration of an injury, disease, or condition, including any objective diminishing of symptoms or making the injury, disease, or condition more tolerable to the patient or slowing in the rate of degeneration or decline. The treatment or amelioration of symptoms also or alternatively can be based on collection of certain medically or clinically accepted subjective information from patients, such as use of a pain scale diagnostic, or from experts, such as a visual assessment or medical examination scale or standard.

The terms “atrophic vaginitis,” “vulvovaginal atrophy,” “vaginal atrophy,” and “VVA” are used herein interchangeably. VVA is known in the art and aspects and symptoms of VVA are described above. The molecular morphology of VVA is well known in the medical field.

As used herein, “sexual dysfunction” refers to a condition having one or more symptoms of difficulty during any one or more stages of sexual activity. The dysfunction can prevent an individual from enjoying sexual activity. Non-limiting examples of symptoms of sexual dysfunction include: reduced sexual desire, reduced sexual pleasure, reduced sexual arousal and excitement, aversion to and avoidance of genital sexual contact, inability to attain or maintain arousal, and persistent or recurrent delay of, or absence of orgasm.

As used herein, “sexual desire” refers to the frequency of wanting to engage in sexual activity and/or the frequency of engaging in sexual activity as perceived by the individual. Sexual desire can be expressed, for example, in one or more cognitive activities, including the frequency of sexual thoughts, the extent of enjoyment of movies, books, music, etc. having sexual content and/or the extent of enjoyment or pleasure of thinking and fantasizing about sex as perceived by the individual.

As used herein, “sexual arousal” refers to the frequency of becoming sexually aroused, how readily sexual arousal occurs and/or if arousal is maintained, as perceived by the individual. Psychologically, arousal can include factors such as increased desire for sexual activity and excitement related to sexual activity. Physiologically, arousal can include increased blood flow to the genitals, causing clitoral engorgement, as well as vaginal lubrication.

As used herein, “lubrication” refers to wetness in and around the vagina before, during, or after sexual activity. Increasing lubrication can include increasing the frequency of lubrication; decreasing the difficulty of becoming lubricated; and/or decreasing the difficulty in maintaining lubrication.

As used herein, “satisfaction” refers to one or more positive emotions (e.g., contentment, fulfillment, gratification, and the like) related to a sexual activity or sexual relationship. Satisfaction can include, for example, satisfaction with occurrence of sexual arousal or orgasm, satisfaction with the amount of closeness with a partner, and satisfaction with overall sex life.

As used herein, “orgasm” refers to the highest point of sexual excitement characterized by a subjective experience of intense pleasure marked normally by vaginal contractions in females. Increasing orgasm can include increasing the frequency, duration, and/or intensity of orgasms in a subject. Increasing orgasm can also include decreasing the difficulty of reaching orgasm.

### II. INTRODUCTION

Provided herein are pharmaceutical compositions comprising a therapeutically effective amount of an estrogen, such as solubilized estradiol, designed to be absorbed vaginally. In some embodiments, the pharmaceutical composition contains a therapeutically effective amount of an active ingredient other than an estrogen designed to be absorbed vaginally for which a vaginal or vaginal-associated abnormality, disease, or condition is targeted (e.g., for treatment, prevention, or diagnosis). The pharmaceutical compositions disclosed herein are designed to be absorbed and have their therapeutic effect locally, e.g., in vaginal and/or surrounding tissue. Further disclosed herein are data demonstrating efficacy of the pharmaceutical compositions disclosed herein, as well as methods relating to using the pharmaceutical compositions. Generally, the pharmaceutical compositions disclosed herein are useful for treating aspects of VVA, including dyspareunia, and other conditions or indications caused by decrease or lack of estrogen in patients. In alternative embodiments, the pharmaceutical compositions are useful for treating or diagnosing abnormal conditions of the vagina or related genitalia, e.g. the labia or vulva, especially those for which absorption of the active in the composition is preferred. If the active is unable to be absorbed, e.g. due to size, conformation, or other chemical or physical characteristic of either the active or the vaginal environment, or is specifically designed to be vaginally non-absorbable, such an active may in some cases still be used with the methods, compositions and formulations described herein due to the spreading and physical distribution effects and characteristics of the formulation providing useful features for the delivery of such active pharmaceutical ingredients or other agents. In some embodiments, active pharmaceutical ingredients other than an estrogen are provided in reduced doses as compared to doses in conventional or previously used formulations, especially those representing the current leading treatment or state of the art with respect to such active pharmaceutical ingredients, for example doses of active pharmaceutical ingredients that are about 75%, 60%, 50%, 45%, 30%, 25%, 10%, 5% lower than the dosage of the API in existing formulations, while providing the same or superior level of efficacy, are provided by embodiments of the invention.

Additional aspects and embodiments of this disclosure include: providing increased patient ease of use with respect to compositions taught in the prior art while potentially minimizing certain side effects from inappropriate insertion, minimizing incidence of vulvovaginal mycotic infection compared to incidence of vulvovaginal mycotic infection due to usage of other vaginally applied estradiol products; and, providing an improved side effect profile (e.g., with respect to pruritus) compared to estrogen compositions taught in the prior art, for example, Vagifem® (estradiol vaginal tablets, Novo Nordisk; Princeton, N.J.).

### III. PHARMACEUTICAL COMPOSITIONS

Functionality

According to embodiments, the pharmaceutical compositions disclosed herein are alcohol-free or substantially alcohol-free. The pharmaceutical compositions disclosed herein provide for improved patient compliance over prior offerings because of improvements over the prior offerings. According to embodiments, the pharmaceutical compositions disclosed herein are encapsulated in soft gelatin capsules, which improve comfort during use. According to embodiments, the pharmaceutical compositions are substantially liquid compositions having a viscosity and composition such that the compositions are more readily absorbed in the vaginal tissue and also are dispersed over a larger surface area of the vaginal and surrounding tissue, providing for improved user experiences.

Estradiol

In some embodiments, the suppository includes about 1 μg to about 25 μg of estradiol. For example, the suppository can include about 1 μg to about 10 μg of estradiol; and about 10 μg to about 25 μg of estradiol.

According to embodiments, the pharmaceutical compositions disclosed herein are for vaginal insertion in a single or multiple unit dosage form and comprise a therapeutically effective amount of estradiol as an active ingredient or are characterized in having estradiol as the only active pharmaceutical ingredient in the composition. According to embodiments, the estradiol in the pharmaceutical compositions is at least about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% solubilized. According to embodiments and where the estradiol is not 100% solubilized, the remaining estradiol is present in a micronized (crystalline) form that is absorbable by the body and retains biological functionality, either in its micronized form or in another form which the micronized form is converted to after administration.

According to embodiments, all or some of the estradiol is solubilized in a solubilizing agent during the manufacturing process. According to embodiments, all or some of the estradiol is solubilized following administration (e.g., the micronized portion where the estradiol is not 100% solubilized is solubilized in a body fluid after administration). According to embodiments, because the estradiol is solubilized, the solubilizing agents taught herein, with or without additional excipients other than the solubilizing agents, are liquid or semi-solid. To the extent the estradiol is not fully solubilized at the time of administration/insertion, the estradiol typically will be substantially solubilized at a body temperature (average of 37° C.) and, generally, at the pH of the vagina (ranges from 3.8 to 4.5 in healthy patients; or 4.6 to 6.5 in VVA patients) in methods of the invention.

According to embodiments, the estradiol can be added to the pharmaceutical compositions disclosed herein as estradiol, estradiol hemihydrate, or other grade estradiol forms used in pharmaceutical compositions or formulations. According to other embodiments, estradiol in the compositions, formulations, and methods of the invention can be substituted, in whole, or in part, with another estrogen. According to still other embodiments, compositions and methods of the invention can include one or more non-estrogen active agents, such as one or more non-estrogen active pharmaceutical ingredients.

According to embodiments, estradiol dosage strengths vary. Estradiol (or estradiol hemihydrate, for example, to the extent the water content of the estradiol hemihydrate is accounted for) dosage strength is from at least about 1 microgram (μg or μg) to at least about 50 μg. Specific dosage embodiments contain at least about: 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, or 50 μg estradiol. According to embodiments, the pharmaceutical compositions contain at least about 2.5 μg; 4 μg; 6.25 μg, 7.5 μg, 12.5 μg, or 18.75 μg of estradiol. According to embodiments, the pharmaceutical compositions contain from about 1 μg to about 10 μg, from 3 μg to 7 μg, from about 3 μg to about 12.5 μg, from about 3.5 μg to about 11.5 μg, from about 7.5 μg to 12.5 μg, from about 10 μg to about 25 μg, about 1 μg, about 2.5 μg, from about 23.5 μg to 27.5 μg, from about 7.5 μg to 22.5 μg, from 10 μg to 25 μg of estradiol. According to embodiments, the lowest clinically effective dose of estradiol is used for treatment of VVA and other indications set forth herein. In some embodiments, the estradiol dosage is about 4 μg. In one embodiment, the estradiol dosage is about 10 μg. In another embodiment, the estradiol dosage is about 25 μg.

Solvent System

According to embodiments, the methods or compositions of the invention include solvent systems that solubilize the estradiol and that comprise medium chain fatty acid-based solvents, together with other excipients. According to embodiments, the solvent systems include non-toxic, pharmaceutically acceptable solvents, co-solvents, surfactants, and/or other excipients suitable for vaginal delivery and/or absorption. According to embodiments, the solvent or solubilizer system must, in combination with any other excipient or active in the fill, be uptaken, distributed, or absorbed within 36 hours or less, for example within 32 hours, within 28 hours, or more specifically within 1 day or less of administration.

According to embodiments, oils having medium chain fatty acids as a majority component of the solvent system or even of the entire formulation are used as solubilizing agents to solubilize estradiol. According to embodiments, the solubilizing agents comprise medium chain fatty acid esters (e.g., esters of glycerol, ethylene glycol, or propylene glycol) or mixtures thereof. According to embodiments, the medium chain fatty acids comprise chain lengths from C6 to C14. According to embodiments the medium chain fatty acids comprise chain lengths from C6 to C12. According to embodiments the medium chain fatty acids substantially (or predominantly) comprise chain lengths from C8-C10. ECNs for medium chain oils typically will be in the range of 21-42 for triglycerides, 12-28 for diglycerides, and 6-14 for monoglycerides.

According to embodiments, the medium chain fatty acids are saturated. According to embodiments, the medium chain fatty acids are predominantly saturated, i.e., greater than about 50%, or greater than about 60% or greater than about 75% of the medium chain fatty acids are saturated.

According to embodiments, estradiol is soluble in the solubilizing agent at room temperature, although it may be desirable to warm certain solubilizing agents during manufacture to improve viscosity. According to embodiments, the solubilizing agent is liquid at between room temperature and about 50° C., at or below 50° C., at or below 40° C., or at or below 30° C.

According to embodiments, the amount of estradiol in the medium chain oil, medium chain fatty acid, or solubilizing agent (or oil/surfactant) is at least about 0.0005 wt %, 0.001 wt %, 0.005 wt %, 0.01 wt %, 0.02 wt %, 0.05 wt %, 0.06 wt %, 0.08 wt %, 0.1 wt %, 0.3 wt %, 0.3 wt %, 0.4 wt %, 0.5 wt %, 0.6 wt %, 0.7 wt %, 0.8 wt %, 0.9 wt %, 1.0 wt %, or higher.

According to embodiments, medium chain solubilizing agents include, for example and without limitation saturated medium chain fatty acids: caproic acid (C6), enanthic acid (C7), caprylic acid (C8), pelargonic acid (C9), capric acid (C10), undecylic acid (C11), lauric acid (C12), tridecylic acid (C13), or myristic acid (C14). According to embodiments, the solubilizing agent includes oils made of these free medium chain fatty acids; oils of medium chain fatty acid esters of glycerin, propylene glycol, or ethylene glycol; or combinations of any or all thereof. These examples comprise predominantly saturated medium chain fatty acids (i.e., greater than 50% of the fatty acids are medium chain saturated fatty acids). According to embodiments, the inclusion of predominantly C6 to C12 saturated fatty acids in the compositions and methods of the invention is contemplated. According to embodiments, the solubilizing agent is selected from at least one of a solvent or a co-solvent.

In some embodiments, the solubilizing agent includes at least one ester selected from the group consisting of: an ester of caproic fatty acid, an ester of caprylic fatty acid, an ester of capric fatty acid, and combinations thereof. For example, the solubilizing agent can include a caprylic/capric triglyceride.

According to embodiments, glycerin based solubilizing agents include: mono-, di-, or triglycerides and combinations and derivatives thereof. Exemplary glycerin based solubilizing agents include MIGLYOLs®, which are caprylic/capric triglycerides (SASOL Germany GMBH, Hamburg). MIGLYOLs includes MIGLYOL 810 (caprylic/capric triglyceride), MIGLYOL 812 (caprylic/capric triglyceride), MIGLYOL 816 (caprylic/capric triglyceride), and MIGLYOL 829 (caprylic/capric/succinic triglyceride). Exemplary glycerin-based solubilizing agents containing fatty acid compositions include but may not be limited to fatty acid content ranging from about <7% C6, about 35-80% C8, about 15-50% C10, about 1-10% C12, about <7% C14, and about 5-30% (e.g., about 2.5-25%, such as about 2.5-20%, about 1-25%, about 1-20%, or about 5-20%) succinic acid. In some embodiments, compositions may include a mix of any or all of the above fatty acids. Also or alternatively, compositions may include mixtures of fatty acids, alone or in combination, such as a mix of C6 and C8 fatty acids ranging from about 1% to about 90%, a mix of C6 and C10 ranging from about 1% to about 70%, a mix of C6 and C12 fatty acids ranging from about 1% to about 15%, a mix of C6 and C14 fatty acids from about 1% to about 10%, a mix of C8 and C10 fatty acids ranging from about 10% to about 99.99%, a mix of C8 and C12 ranging from about 1% to about 99.99%, a mix of C8 and C14 fatty acids from about 1% to about 85%, a mix of C10 and C12 fatty acids from about 1% to about 70%, a mix of C10 and C14 fatty acids from about 1% to about 60%, and a mix of C12 and C14 fatty acids from about 1% to about 20%. Such mixtures may or may not include succinic acid, which, if present, typically will be present in a concentration of less than about 40% succinic acid (e.g., less than about 30%, such as about 5-30%, 7.5-27.5%, 10-25%, 12.5-22.5%, or about 15-20%).

Thus, in one respect the compositions and methods of the invention comprise a mix of caprylic and capric triglycerides, optionally in combination with succinic triglyceride. Other caprylic/capric triglyceride solubilizing agents are likewise contemplated, including, for example: caproic/caprylic/capric/lauric triglycerides; caprylic/capric/linoleic triglycerides; caprylic/capric/succinic triglycerides. According to embodiments, CAPMUL MCM, medium chain mono- and di-glycerides, is the solubilizing agent. Other triglycerides of fractionated vegetable fatty acids, and combinations or derivatives thereof can be the solubilizing agent, according to embodiments. For example, the solubilizing agent can be 1,2,3-propanetriol (glycerol, glycerin, glycerine) esters of saturated coconut and palm kernel oil and derivatives thereof.

Ethylene and propylene glycol (which include polyethylene and polypropylene glycols) solubilizing agents include: glyceryl mono- and di-caprylates; propylene glycol monocaprylate (e.g., CAPMUL® PG-8 (the CAPMUL brands are owned by ABITEC, Columbus, Ohio)); propylene glycol monocaprate (e.g., CAPMUL PG-10); propylene glycol mono- and dicaprylates; propylene glycol mono- and dicaprate; diethylene glycol mono ester (e.g., TRANSCUTOL®, 2-(2-ethoxyethoxy)ethanol, GATTEFOSSÉ SAS); and diethylene glycol monoethyl ether. Other combinations of mono- and di-esters of propylene glycol or ethylene glycol are expressly contemplated as the solubilizing agent. Use of such propylene glycol mono- and dicaprylates and propylene glycol mono- and dicaprates, alone or in combination, may include higher concentrations of C8 fatty acids in the medium chain oil or solubilizing agent than in other embodiments such that more than about 80%, about 85% or more, about 90% or more, about 95% or more, about 99% or more, or even about 99.9% of the solubilizing agent or medium chain oil can consist of C8 fatty acids or related molecules. According to embodiments, the medium chain oil or solubilizing agent can also or alternatively comprise a relatively small amount of C10 fatty acids, such that, for example, the content of C10 fatty acids is about 10% or less, such as the content of C10 fatty acids or related compounds being less than or equal to 5%. According to embodiments the content of fatty acids C12 or higher also or alternatively is limited to about 5% or less, such as about 3% or less, such as about 2% or less, or about 1.5% or less (e.g., about 1% or less, or even about 0.25% or less or 0.1% or less). In certain embodiments, the content of propylene glycol monoesters in the solubilizing agent or the composition may be greater than or equal to about 80%. In some embodiments, the fill comprises a solubilizing agent wherein at least about 35% of constituent fatty acids are C8, C10, C12, or C14 fatty acids. According to embodiments, the solubilizing agent includes combinations of mono- and di-propylene and ethylene glycols and mono-, di-, and triglyceride combinations. According to embodiments, polyethylene glycol glyceride (GELUCIRE®, GATTEFOSSE SAS, Saint-Priest, France) can be used herein as the solubilizing agent or as a surfactant. For example, GELUCIRE 44/14 (PEG-32 glyceryl laurate EP), a medium chain fatty acid ester of polyethylene glycol, is a polyethylene glycol glyceride composed of mono-, di- and triglycerides and mono- and diesters of polyethylene glycol.

According to embodiments, commercially available fatty acid glycerol and glycol ester solubilizing agents are often prepared from natural oils and therefore may comprise components in addition to the fatty acid esters that predominantly comprise and characterize the solubilizing agent. Such other components may be, e.g., other fatty acid mono-, di-, and triglycerides; fatty acid mono- and diester ethylene or propylene glycols, free glycerols or glycols, or free fatty acids, for example. In some embodiments, when an oil/solubilizing agent is described herein as a saturated C8 fatty acid mono- or diester of glycerol, the predominant component of the oil, i.e., >50 wt % (e.g., >75 wt %, >85 wt % or >90 wt %) is caprylic monoglycerides and caprylic diglycerides. For example, the Technical Data Sheet by ABITEC for CAPMUL MCM C8 describes CAPMUL MCM C8 as being composed of mono and diglycerides of medium chain fatty acids (mainly caprylic) and describes the alkyl content as ≤1% C6, ≥95% C8, ≤5% C10, and ≤1.5% C12 and higher.

For example, MIGLYOL 812 is a solubilizing agent that is generally described as a C8-C10 triglyceride because the fatty acid composition is at least about 80% triglyceride esters of caprylic acid (C8) and capric acid (C10). However, it also includes small amounts of other fatty acids, e.g., less than about 5% of caproic acid (C6), lauric acid (C12), and myristic acid (C14). The product information sheet for various MIGLYOLs illustrate the various fatty acid components as follows:

| Tests | 810 | 812 | 818 | 829 | 840 |
|---|---|---|---|---|---|
| Caproic | Max 2.0 | Max. 2.0 | Max. 2 | Max. 2 | Max. 2 |
| acid | | | | | |
| (C6:0) | | | | | |
| Caprylic | 65.0-80.0 | 50.0-65.0 | 45-65 | 45-55 | 65-80 |
| acid | | | | | |
| (C8:0) | | | | | |
| Capric | 20.0-35.0 | 30.0-45.0 | 30-45 | 30-40 | 20-35 |
| acid | | | | | |
| (C10:0) | | | | | |
| Lauric | Max. 2 | Max. 2 | Max. 3 | Max. 3 | Max. 2 |
| acid | | | | | |
| (C12:0) | | | | | |
| Myristic | Max. 1.0 | Max. 1.0 | Max. 1 | Max. 1 | Max. 1 |
| acid | | | | | |
| (C14:0) | | | | | |
| Linoleic | — | — | 2-5 | — | — |
| acid | | | | | |
| (C18:2) | | | | | |
| Succinic | — | — | — | 15-20 | — |
| acid | | | | | |
| ECN | 25.5-26.4 | 26.1-27 | 26.52-28.56 | 26-27.6 | 25.5-26.4 |

According to embodiments, anionic or non-ionic surfactants may be used in pharmaceutical compositions containing solubilized estradiol. Ratios of solubilizing agent(s) to surfactant(s) vary depending upon the respective solubilizing agent(s) and the respective surfactant(s) and the desired physical characteristics of the resultant pharmaceutical composition. For example and without limitation, CAPMUL MCM and a non-ionic surfactant may be used at ratios including 65:35, 70:30, 75:25, 80:20, 85:15 and 90:10. Other non-limiting examples include: CAPMUL MCM and GELUCIRE 39/01 used in ratios including, for example and without limitation, 6:4, 7:3, and 8:2; CAPMUL MCM and GELUCIRE 43/01 used in ratios including, for example and without limitation, 7:3, and 8:2; CAPMUL MCM and GELUCIRE 50/13 used in ratios including, for example and without limitation, 7:3, and 8:2, and 9:1. More generally, the ratio of solubilizing agent(s) to surfactant(s) in the formulation, composition, or fill may be 5:1 to 12:1, 6:1 to 11:1, and 7:1 to 10:1, for example, 7.5:1 to 9.5:1, or 8.5:1 to 9.5:1. In some embodiments, a MIGLYOL may be used as an alternative to a CAPMUL. In some embodiments, TEFOSE 63 (GATTEFOSSE SAS, Saint-Priest, France) may be used as an alternative to GELUCIRE. In such formulations, ratios of solubilizing agent(s) to surfactant(s) may be 5:1 to 12:1, 6:1 to 11:1, and 7:1 to 10:1, for example 7.5:1 to 9.5:1, or 8.5:1 to 9.5:1.

According to some embodiments, the surfactant may also serve as a thickening agent having the ability to increase the viscosity of the formulation by up to about 10%, about 20%, about 50%, about 75%, about 100%, about 125%, about 150% about 160%, about 180%, 200%, 250%, 300%, 350% or up to about 400%.

### Other Excipients

According to embodiments, the pharmaceutical composition further includes a surfactant. The surfactant can be a nonionic surfactant, cationic surfactant, anionic surfactant, or mixtures thereof. Suitable surfactants include, for example, water-insoluble surfactants having a hydrophilic-lipophilic balance (HLB) value less than 12 and water-soluble surfactants having a HLB value greater than 12. Surfactants that have a high HLB and hydrophilicity, aid the formation of oil-water droplets. The surfactants are amphiphilic in nature and are capable of dissolving or solubilizing relatively high amounts of hydrophobic drug compounds.

Non-limiting examples of surfactants, include, Tween, Dimethylacetamide (DMA), Dimethyl sulfoxide (DMSO), Ethanol, Glycerin, N-methyl-2-pyrrolidone (NMP), PEG 300, PEG 400, Poloxamer 407, Propylene glycol, Phospholipids, Hydrogenated soy phosphatidylcholine (HSPC), Distearoylphosphatidylglycerol (DSPG), L-α-dimyristoylphosphatidylcholine (DMPC), L-α-dimyristoylphosphatidylglycerol (DMPG), Polyoxyl 35 castor oil (e.g., CREMOPHOR EL or CREMOPHOR ELP), Polyoxyl 40 hydrogenated castor oil (e.g., CREMOPHOR RH 40), Polyoxyl 60 hydrogenated castor oil (CREMOPHOR RH 60), Polysorbate 20 (TWEEN 20), Polysorbate 80 (TWEEN 80), d-α-tocopheryl polyethylene glycol 1000 succinate (TPGS), Solutol HS-15, Sorbitan monooleate (SPAN 20), PEG 300 caprylic/capric glycerides (SOFTIGEN 767), PEG 400 caprylic/capric glycerides (LABRASOL), PEG 300 oleic glycerides (LABRAFIL M-1944CS), Polyoxyl 35 Castor oil (ETOCAS 35), Glyceryl Caprylate (Mono- and Diglycerides) (IMWITOR), PEG 300 linoleic glycerides (LABRAFIL M-2125CS), Polyoxyl 8 stearate (PEG 400 monosterate), Polyoxyl 40 stearate (PEG 1750 monosterate), and combinations thereof. Additionally, suitable surfactants include, for example, polyoxyethylene derivative of sorbitan monolaurate such as polysorbate, caprylcaproyl macrogol glycerides, polyglycolyzed glycerides, and the like. Products previously sold under the CREMOPHOR trade name have more recently been sold under the trade name KOLLIPHOR (BASF, Lampertheim, Germany).

According to embodiments, the non-ionic surfactant is selected from one or more of glycerol and polyethylene glycol esters of long chain fatty acids, for example, lauroyl macrogol-32 glycerides or lauroyl polyoxyl-32 glycerides, commercially available as GELUCIRE, including, for example, GELUCIRE 39/01 (glycerol esters of saturated C12-C18 fatty acids), GELUCIRE 43/01 (hard fat NF/JPE) and GELUCIRE 50/13 (stearoyl macrogol-32 glycerides EP, stearoyl polyoxyl-32 glycerides NF, stearoyl polyoxylglycerides (USA FDA IIG)). These surfactants may be used at concentrations greater than about 0.01%, and typically in various amounts of about 0.01%-10.0%, 2%-20%, 2.5%-15%, 3%-15%, 5%-12.5%, 7.5%-15%, 7.5%-12.5%, 9%-11%, 10.1%-20%, and 20.1%-30%. In some embodiments, surfactants may be used at concentrations of about 1% to about 15%, such as about 1% to about 10% (e.g., about 1% to about 5%, about 2% to about 4%, about 3% to about 8%, about 4% to about 14%, about 3% to about 13%, about 5% to about 15%, about 2% to about 12%, or about 8% to about 11%).

According to embodiments, non-ionic surfactants include, for example and without limitation: one or more of oleic acid, linoleic acid, palmitic acid, and stearic acid. According to embodiments, non-ionic surfactants comprise polyethylene sorbitol esters, including polysorbate 80, which is commercially available under the trademark TWEEN® 80 (polysorbate 80) (Sigma Aldrich, St. Louis, Mo.). Polysorbate 80 includes approximately 60%-70% oleic acid with the remainder comprising primarily linoleic acids, palmitic acids, and stearic acids. Polysorbate 80 may be used in amounts ranging from about 5% to about 50%, and according to embodiments, can compose about 30% of the pharmaceutical composition's total mass.

According to embodiments, the non-ionic surfactant includes a mixture of both PEG-6 palmitostearate and ethylene glycol palmitostearate, which are combined in a commercially available surfactant currently sold as TEFOSE® 63 (GATTEFOSSE SAS, Saint-Priest, France) (which also includes PEG-32 stearate), which can be used with, for example, CAPMUL MCM having ratios of MCM to TEFOSE 63 of, for example, 8:2 or 9:1. According to embodiments, other solubilizing agents/non-ionic surfactants combinations include, for example, MIGLYOL 812:GELUCIRE 50/13 or MIGLYOL 812:TEFOSE 63. Alternative PEG-6 palmitostearate, PEG-32 stearate, and ethylene glycol palmitostearate formulations contemplated include DUB 1632 (STEARINERIE DUBOIS, Boulogne Billancourt, France). Alternatively, excipients similarly comprising a small PEG molecule component, i.e. PEG 2-PEG 10 compounds (e.g., PEG4-PEG8 stearates) and a medium PEG compound component, i.e. PEG 20-PEG 40 compounds (e.g., PEG 25-35 stearates) and, optionally, an ethylene glycol stearate are contemplated. In some embodiments, other known TEFOSE 63 alternatives may be used in the composition or fill in place of all or some of TEFOSE 63 in embodiments wherein TEFOSE63 is described herein. Examples of alternatives that have been used in place of TEFOSE63 in known compositions include sorbitan monostearates (e.g. Span; CRODA, Buenos Aires, Argentina), polyethylene glycol monostearates (e.g. Myrj; CRODA), d-alpha-tocopheryl polyethylene glycol 1000 succinate (e.g. TPGS), lauroyl polyoxyl-6 or macrogol-6 glycerides (e.g. Labrafil M 2130 CS, GATTEFOSSE), a mixture of triceteareth-4 phosphate (and) ethylene glycol palmitostearate EP/NF/JPE (and) diethylene glycol palmitostearate EP/NF/JPE (e.g. Sedefos 75, GATTEFOSSE), PEG-8 beeswax (e.g. Apifil, GATTEFOSSE), a mixture of Glycerol monostearate EP/NF (and) PEG-75 stearate NF/JPE/EP (e.g. Gelot 64, GATTEFOSSE), and a mixture of glycerol monostearate EP/NF (and) PEG-75 stearate NF/JPE/EP (e.g. Emulcire 61 WL 2659, GATTEFOSSE). According to some embodiments, alternative excipients would generally be selected from those demonstrating one or more of the following characteristics: non-ionic composition, oil-in-water emulsifier functionality, easily or readily forms emulsions or may be classified by those of ordinary skill in the art as a self-emulsifying base, having a hydrophilic-lipophilic balance (HLB) of 8-11 (for example 8.5-10.5, 9-10.5, or more specifically 9-10), and a satisfactory mucosal tolerance so as to be effective and/or highly tolerated in a mucosal environment. According to embodiments the composition or fill comprises a component that acts as a surfactant and a thickener, which in some embodiments is embodied in the same excipient composition, such as is the case with respect to TEFOSE 63. According to embodiments the inclusion of the thickener/surfactant results in improved spreading of the formulation in the vagina (and in some embodiments vaginal-associated tissue, such as the vulva), and/or retention of the compositions in the vagina, and may provide the benefit of immediate ambulation after administration without significant loss in efficacy.

According to certain embodiments, the non-ionic surfactant in the fill comprises PEG-6 stearate, glycol stearate and PEG-32 stearate wherein the PEG-6 stearate, glycol stearate and PEG-32 stearate are in a ratio of about 63:18.5:18.5, about 75:12.5:12.5, about 50:25:25, about 75:15:10 or ranges of such ratios. In embodiments, the fill may comprise PEG-6 stearate, glycol stearate and PEG-32 stearate in a combined amount of from about 1% to about 30%, from about 1% to about 20%, from about 3% to about 15%, from about 5% to about 10%, from about 7% to about 10%, about 9% or about 8%. In some embodiments, the fill may comprise PEG-6 stearate in an amount from about 1% to about 20% by weight, from about 1% to about 10% by weight, from about 4% to about 10% by weight or from about 4% to about 6% by weight. In some embodiments, the fill may comprise glycol stearate in an amount from about 0.1% to about 10%, from about 0.1% to about 8%, from about 0.5% to about 5%, from about 0.5% to about 3%, from about 0.5% to about 2%, or from about 0.8% to about 2%. In some embodiments, the fill may comprise PEG-32 stearate in an amount from about 0.1% to about 10%, from about 0.1% to about 8%, from about 0.5% to about 5%, from about 0.5%> to about 3%, from about 0.5%> to about 2%, or from about 0.8% to about 2%. In some embodiments, the fill may comprise PEG-6 may be present in an amount of about 5% w/w; glycol stearate may be present in an amount of about 1.5% w/w, PEG-32 stearate may be present in an amount of about 1.5% w/w.

Also provided herein is a suppository comprising: a) a therapeutically effective amount of estradiol; b) a caprylic/capric triglyceride; c) a non-ionic surfactant comprising PEG-6 palmitostearate and ethylene glycol palmitostearate; and d) a soft gelatin capsule.

According to embodiments, the surfactant can be an anionic surfactant, for example: ammonium lauryl sulfate, dioctyl sodium sulfosuccinate, perfluoro-octane sulfonic acid, potassium lauryl sulfate, or sodium stearate. Cationic surfactants are also contemplated.

According to embodiments, non-ionic or anionic surfactants can be used alone with at least one solubilizing agent or can be used in combination with other surfactants. Accordingly, such surfactants, or any other excipient as set forth herein, may be used to solubilize estradiol. The combination of solubilizing agent, surfactant, and other excipients should be designed whereby the estradiol is absorbed into the vaginal tissue. According to embodiments, the pharmaceutical composition will result in minimal vaginal discharge.

According to embodiments, the pharmaceutical composition further includes at least one thickening agent. Generally, a thickening agent is added when the viscosity of the pharmaceutical composition results in less than desirable absorption without the inclusion of the thickening agent. According to embodiments, the surfactant(s) disclosed herein may also provide thickening of the pharmaceutical composition that, upon release, will aid the estradiol in being absorbed by the vaginal mucosa while minimizing vaginal discharge. Examples of thickening agents include: hard fats; propylene glycol; a mixture of hard fat EP/NF/JPE, glyceryl ricinoleate, ethoxylated fatty alcohols (ceteth-20, steareth-20) EP/NF (available as OVUCIRE® 3460, GATTEFOSSE, Saint-Priest, France); a mixture of hard fat EP/NF/JPE, glycerol monooleate (type 40) EP/NF (OVUCIRE WL 3264; a mixture of hard fat EP/NF/JPE, glyceryl monooleate (type 40) EP/NF (OVUCIRE WL 2944); a non-ionic surfactant comprising PEG-6 stearate, ethylene glycol palmitostearate, and PEG-32 stearate; TEFOSE 63 or a similar product; and a mixture of various hard fats (WITEPSOL®, Sasol Germany GmbH, Hamburg, Germany). Other thickening agents such as the alginates, certain gums such as xanthan gums, agar-agar, iota carrageenans, kappa carrageenans, etc. Several other compounds can act as thickening agents like gelatin, and polymers like HPMC, PVC, and CMC. According to embodiments, the viscosity of pharmaceutical compositions in accordance with various embodiments may comprise from about 50 cps to about 1000 cps at 25° C., for example 25 cps to about 900 cps, 35 cps to about 600 cps, or for example about 90 cps to about 400 cps. Additional suitable viscosities are discussed later in this application. A person of ordinary skill in the art will readily understand and select from suitable thickening agents based on the properties of other agents in the composition and given the guidance provided herein with respect to the desired viscosity and functional properties of compositions of the invention.

According to embodiments, the thickening agent is a non-ionic surfactant. For example, polyethylene glycol saturated or unsaturated fatty acid ester or diester is the non-ionic surfactant thickening agent. In embodiments, the non-ionic surfactant includes a polyethylene glycol long chain (C16-C20) fatty acid ester and further includes an ethylene glycol long chain fatty acid ester, such as PEG-fatty acid esters or diesters of saturated or unsaturated C16-C18 fatty acids, e.g., oleic, lauric, palmitic, and stearic acids. In embodiments, the non-ionic surfactant includes a polyethylene glycol long chain saturated fatty acid ester and further includes an ethylene glycol long chain saturated fatty acid ester, such as PEG- and ethylene glycol-fatty acid esters of saturated C16-C18 fatty acids, e.g., palmitic and stearic acids. Such non-ionic surfactant can comprise PEG-6 stearate, ethylene glycol palmitostearate, and PEG-32 stearate, such as, but not limited to, TEFOSE 63 (GATTEFOSSE SAS, Saint-Priest, France).

According to embodiments, TEFOSE 63 is used to provide additional viscosity and/or spreadability in the vagina so as to retard flow of the composition out of the vagina. While the pharmaceutical composition remains liquid, the viscosity of such a pharmaceutical composition causes the liquid to remain in the API absorption area whereby the pharmaceutical composition is substantially absorbed by the tissue. Surprisingly, the addition of an excipient which increases the viscosity may also improve the spreadability and distribution of the pharmaceutical compositions herein, allowing the administration of a pharmaceutical composition that is liquid at body temperature but does not excessively discharge from the vagina when the patient is standing. Such a composition beneficially allows users or patients receiving the composition to be ambulatory soon or even immediately after administration of the pharmaceutical compositions. According to some embodiments, the API(s) of the formulation are detectable within the vaginal, labial, or vulvar tissue within 36 hours, for example within 24 hours, e.g. within 12 hours or for example within 6 hours or less of administration. In some embodiments, detectable effects of the API on the labia or vulva may be observed, measured, or experienced within 36 hours, for example within 24 hours, 12 hours, 6 hours or less of administration. Examples of such observations, measurements or experiences may include but not be limited to change in color (e.g. greater tissues pinkness), increased moisture, tissue, mucous/discharge or mucosal composition assays, or user experience such as improvements in itching, burning, or other bothersome symptoms for which treatment is being sought.

According to embodiments, the non-ionic surfactant used as a thickening agent is not hydrophilic and has good emulsion properties. An illustrative example of such surfactant is TEFOSE 63, which has a hydrophilic-lipophilic balance (HLB) value of about 9-10. In some embodiments, such a surfactant may have an HLB of about 7-14, for example about 8-11.

According to embodiments, the pharmaceutical composition further includes one or more mucoadherent agents to improve vaginal absorption of the estradiol by, for example, increasing the viscosity of the pharmaceutical composition whereby flow out of the vagina is retarded. According to other embodiments, alone or in addition to changes in viscosity, the mucoadhesive agent causes the pharmaceutical composition to adhere to the vaginal tissue chemically or mechanically. For example, a mucoadherent agent can be present to aid the pharmaceutical composition with adherence to the mucosa upon activation with water. According to embodiments, polycarbophil is the mucoadherent agent. According to embodiments, other mucoadherent agents include, for example and without limitation: poly (ethylene oxide) polymers having a molecular weight of from about 100,000 to about 900,000; chitosans; carbopols including polymers of acrylic acid crosslinked with allyl sucrose or allyl pentaerythritol; polymers of acrylic acid and C10-C30 alkyl acrylate crosslinked with allyl pentaerythritol; carbomer homopolymer or copolymer that contains a block copolymer of polyethylene glycol and a long chain alkyl acid ester; and the like. According to embodiments, various hydrophilic polymers and hydrogels may be used as the mucoadherent agent. According to certain embodiments, the polymers or hydrogels can swell in response to contact with vaginal tissue or secretions, enhancing moisturizing and mucoadherent effects. The selection and amount of hydrophilic polymer may be based on the selection and amount of solubilizing agent. In some embodiments, the pharmaceutical composition includes a hydrophilic polymer but optionally excludes a gelling agent. In embodiments having a hydrogel, from about 5% to about 10% of the total mass may comprise the hydrophilic polymer. In further embodiments, hydrogels may be employed. A hydrogel may comprise chitosan, which swell in response to contact with water. In various embodiments, a cream pharmaceutical composition may comprise PEG-90M. In some embodiments, a mucoadherent agent is present in the pharmaceutical formulation, in the soft gel capsule, or both.

According to embodiments, the pharmaceutical compositions include one or more thermoreversible gels, typically of the hydrophilic nature including for example and without limitation, hydrophilic sucrose and other saccharide-based monomers (U.S. Pat. No. 6,018,033, which is incorporated by reference).

According to embodiments, the pharmaceutical composition further includes a lubricant. In some embodiments, a lubricant can be present to aid in formulation of a dosage form. For example, a lubricant may be added to ensure that capsules or tablets do not stick to one another during processing or upon storage. Any suitable lubricant may be used. For example, in some embodiments, lecithin, which is a mixture of phospholipids, is the lubricant.

According to embodiments, the pharmaceutical composition further includes an antioxidant. Any suitable anti-oxidant may be used. For example, an antioxidant that can be included in the compositions of the invention can be selected from butylated hydroxytoluene, butylated hydroxyanisole, and Vitamin E TPGS.

According to embodiments, the pharmaceutical composition includes about 20% to about 80%, about 85%, about 90%, about 92.5%, or about 95% solubilizing agent by weight, about 1% to about 20%, such as about 2% to about 18%, about 3% to about 15%, about 4% to about 12%, or about 10% surfactant, if present, about 0.1% to about 5% lubricant by weight, if present, and about 0.01% to about 0.1% antioxidant by weight.

The choice of excipient will depend on factors such as, for example, the effect of the excipient on solubility and stability. Additional excipients used in various embodiments may include colorants and preservatives. Examples of colorants include FD&C colors (e.g., blue No. 1 and Red No. 40), D&C colors (e.g., Yellow No. 10), and opacifiers (e.g., Titanium dioxide). According to embodiments, colorants, comprise about 0.1% to about 2% of the pharmaceutical composition by weight. According to embodiments, preservatives in the pharmaceutical composition comprise methyl and propyl paraben, in a ratio of about 10:1, and at a proportion of about 0.005% and 0.05% by weight.

Generally, the solubilizing agents, excipients, other additives used in the pharmaceutical compositions described herein, are non-toxic, pharmaceutically acceptable, compatible with each other, and maintain stability of the pharmaceutical composition and the various components with respect to each other. Additionally, the combination of various components that comprise the pharmaceutical compositions will result in the desired therapeutic effect when administered to a subject.

### Solubility of Estradiol

According to embodiments, solubilizing agents comprising mixtures of medium chain fatty acid glycerides, e.g., C₆-C12, C₈-C12, or C₈-C10 fatty acid mono- and diglycerides or mono-, di-, and triglycerides dissolve estradiol. As illustrated in the Examples, good results were obtained with solubilizing agents that are predominantly a mixture of C8-C10 saturated fatty acid mono- and diglycerides, or medium chain triglycerides (e.g., MIGLYOL 810 or 812). Longer chain glycerides appear to be not as well suited for dissolution of estradiol.

A solubilizing agent comprising propylene glycol monocaprylate (e.g., CAPRYOL) and 2-(2-Ethoxyethoxy)ethanol (e.g., TRANSCUTOL) solubilized estradiol well.

### IV. Manufacture of the Pharmaceutical Composition

According to embodiments, the pharmaceutical composition is prepared via blending estradiol with a pharmaceutically acceptable solubilizing agent, including for example and without limitation, at least one medium chain fatty acid such as medium chain fatty acids consisting of at least one mono-, di-, or triglyceride, or derivatives thereof, or combinations thereof. According to embodiments, the pharmaceutical composition also includes at least one glycol or derivatives thereof or combinations thereof or combinations of at least one glyceride and glycol. The glycol(s) may be used as solubilizing agents or to adjust viscosity and, thus, may be considered thickening agents, as discussed further herein. Optionally added are other excipients including, for example and without limitation, anti-oxidants, lubricants, and the like. According to embodiments, the pharmaceutical composition includes sufficient solubilizing agent to fully solubilize the estradiol. It is expressly understood, however, that other volumes of solubilizing agent can be used depending on the level of estradiol solubilization desired. Persons of ordinary skill in the art will know and understand how to determine the volume of solubilizing agent and other excipients depending on the desired percent of estradiol to be solubilized in the pharmaceutical composition.

In illustrative embodiments, GELUCIRE 44/14 (lauroyl macrogol-32 glycerides EP, lauroyl polyoxyl-32 glycerides NF, lauroyl polyoxylglycerides (USA FDA IIG)) is heated to about 65° C. and CAPMUL MCM is heated to about 40° C. to facilitate mixing of the oil and non-ionic surfactant, although such heating is not necessary to dissolve the estradiol.

Specific Examples disclosed herein provide additional principles and embodiments illustrating the manufactures of the pharmaceutical compositions disclosed herein.

### V. DELIVERY VEHICLE

Generally, the pharmaceutical compositions described herein is delivered intravaginally inside of a delivery vehicle, for example a capsule. The soft gel vaginal pharmaceutical composition has been designed to mitigate common limitations found with other vaginal forms of estradiol. The soft gel vaginal pharmaceutical composition eases vaginal administration, provides improved safety of insertion, minimizes vaginal discharge following administration, and provides a more effective dosage form having improved efficacy, safety, patient compliance, and user experiences.

According to various aspects and embodiments of this disclosure, a soft gel vaginal pharmaceutical composition as a treatment for post-menopausal women suffering with moderate to severe symptoms of VVA is provided.

According to embodiments, the capsules are soft capsules made of materials well known in the pharmaceutical arts, for example, gelatin. According to embodiments, the capsule may for example contain gelatin which may provide limited bioadhesion. According to embodiments, no additional bioadhesive agent is included in either the vehicle or the fill contained within the vehicle. However, according to embodiments, the delivery vehicle alternatively is integral with the pharmaceutical composition (i.e., the pharmaceutical composition is the delivery vehicle). In such embodiments the pharmaceutical compositions is in the form of a gel, cream, ointment, tablet, or other preparation that is directly applied and absorbed vaginally.

According to embodiments, the capsule fill does not contain one or more of the following: a hydrophilic gel-forming bioadhesive agent, a lipophilic agent, a gelling agent for the lipophilic agent, and/or a hydrodispersible agent. According to embodiments, the capsules do not contain a hydrophilic gel-forming bioadhesive agent selected from: carboxyvinylic acid, hydroxypropylcellulose, carboxymethylcellulose, gelatin, xanthan gum, guar gum, aluminum silicate, and mixtures thereof. According to embodiments, the capsules do not contain a lipophilic agent selected from: a liquid triglyceride, a solid triglyceride (with a melting point of about 35° C.), carnauba wax, cocoa butter, and mixtures thereof. According to embodiments, the capsules do not contain a hydrophobic colloidal silica gelling agent. According to embodiments, the capsules do not contain a hydrodispersible agent selected from: polyoxyethylene glycol, polyoxyethylene glycol 7-glyceryl-cocoate, and mixtures thereof. In some embodiments, the estradiol is formulated as a liquid composition consisting of a therapeutically effective amount of estradiol; a caprylic/capric triglyceride; and a non-ionic surfactant comprising PEG-6 palmitostearate and ethylene glycol palmitostearate. In such embodiments, a hydrophilic gel-forming bioadhesive agent is in the liquid composition. In some such embodiments, the liquid composition is contained within a gelatin capsule as described herein. In some such embodiments, the capsule comprises gelatin and optionally one or more further components selected from the group consisting of gelatin, hydrolyzed gelatin, sorbitol-sorbitan solution, water, glycerin, titanium dioxide, FD&C Red #40, ethanol, ethyl acetate, propylene glycol, polyvinyl acetate phthalate, isopropyl alcohol, polyethylene glycol, and ammonium hydroxide.

According to embodiments, the delivery vehicle is designed for ease of insertion. According to embodiments, the delivery vehicle is sized whereby it can be comfortably inserted into the vagina. According to embodiments, the delivery vehicle is prepared in a variety of geometries. For example, the delivery vehicle is shaped as a tear drop, a cone with frustoconical end, a cylinder, a cylinder with larger “cap” portion, or other shapes suitable for and that ease insertion into the vagina. According to embodiments, the delivery vehicle is used in connection with an applicator. According to other embodiments, the delivery vehicle is inserted digitally. According to embodiments, the capsule is formulated as to contain a 50 to 1000 mg fill, for example the fill can be about 100 to about 800 mg, e.g. about 200 to about 600 mg, or about 250-400 mg, or more specifically about a 300 mg fill. Generally, the delivery vehicle, such as a soft gel capsule, used to deliver the active pharmaceutical ingredient in the fill, as described herein, is less than about 1 inch in length, for example less than about 0.75 inches in length, more specifically less than about 0.7 inches in length. In addition, the delivery vehicle described herein is generally less than 0.5 inches in width (e.g., diameter in the case of a vehicle comprising a circular or semi-circular portion), for example less than 0.4 inches, more specifically less than 0.35 inches in width. According to embodiments the delivery vehicle has a narrower end and a relatively wider, opposite end, so as to facilitate insertion to the vagina. According to some embodiments, the width of the delivery vehicle may vary from one end of the vehicle to the other, so as to form an egg, stretched oval, or tear drop-like shape. Such variation in a dimension of the delivery vehicle, such as width of the vehicle between one end and another, can be between, e.g., about 10%-about 99% (e.g., about 20%-95% or about 30%-95%) difference in width from the vehicle's narrower. end to the average of the vehicle's wider end, and can, in the case of a tapering section or the like include an area in which the width gradually decreases from a first, larger width to a second, smaller width. According to some embodiments, the ratio of the vehicle's widest width to its length is approximately 1:3, for example 1:2.5 or more specifically about 1:2.3, 1:2, 1:1.5, or 1:1.

According to embodiments, a method for the treatment of VVA or one or more symptoms or aspects thereof, including dyspareunia, vaginal dryness, and estrogen-deficient urinary states (including urinary tract infections), is provided wherein a composition for the treatment of VVA is digitally inserted approximately two inches or less into the vagina (e.g., about 1.75 inches or less, about 1.5 inches or less, about 1.25 inches or less, such as about 1 inch, such as about 0.5-2 inches, about 0.75-2 inches, about 1-2 inches, about 0.5-1.5 inches, about 0.75-1.75 inches, or about 0.75-1.5 inches) or in the approximately one third of the vagina closest to the opening of the vagina (e.g., the proximal ¼^{th} to ½ of the vagina) and results in at least one of: improved compliance compared to other products for the treatment of VVA; improved user experience compared to other products for the treatment of VVA; and statistically significantly improved symptoms of VVA, compared to placebo or baseline within one of two, four, six, eight, ten, or twelve or more weeks after initiation of administration. According to embodiments, a method for the treatment of VVA, including dyspareunia, vaginal dryness, and estrogen-deficient urinary states (including urinary tract infections), is provided wherein a delivery vehicle containing a composition for the treatment of VVA and a tear drop shape as disclosed herein is insert approximately two inches (e.g., about 1.5-about 3 inches) into the vagina or in the third of the vagina closest to the opening of the vagina and results in at least one of: improved compliance compared to other products for the treatment of VVA; improved user experience compared to other products for the treatment of VVA; and statistically significantly improved symptoms of VVA, compared to placebo or baseline within one of two, four, six, eight, ten, or twelve or more weeks after initiation of administration.

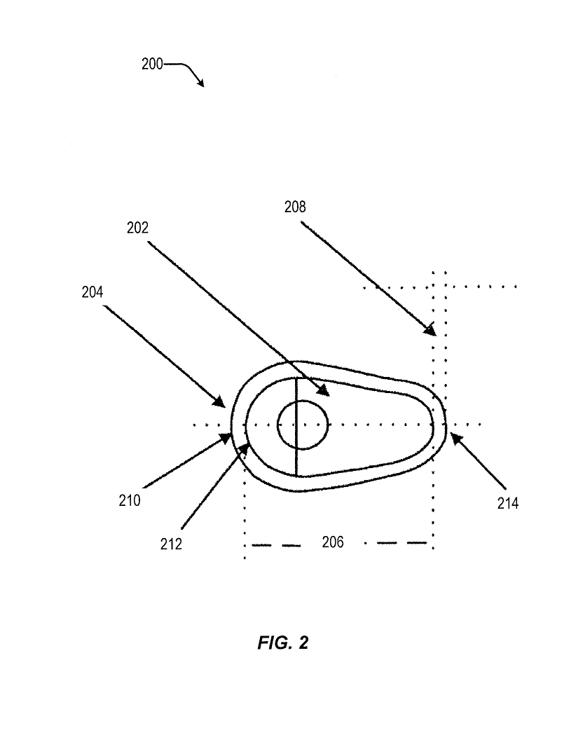

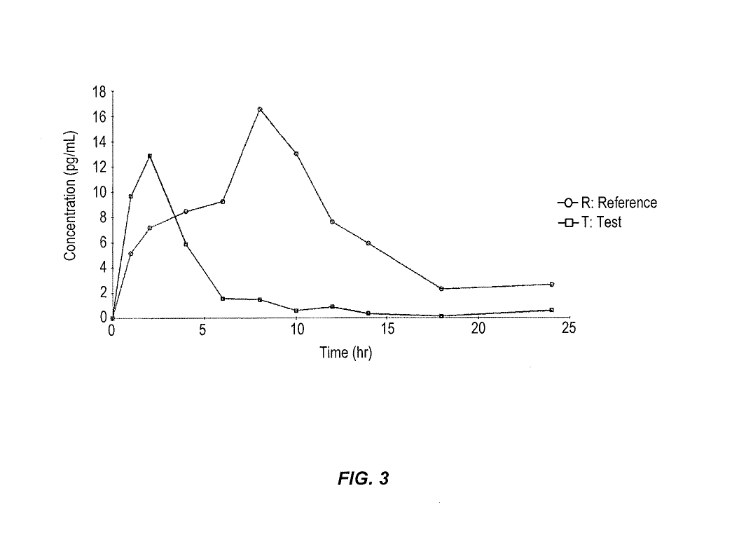

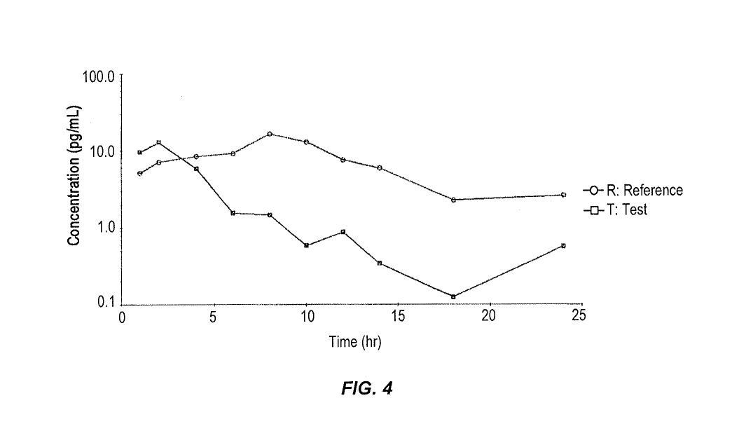

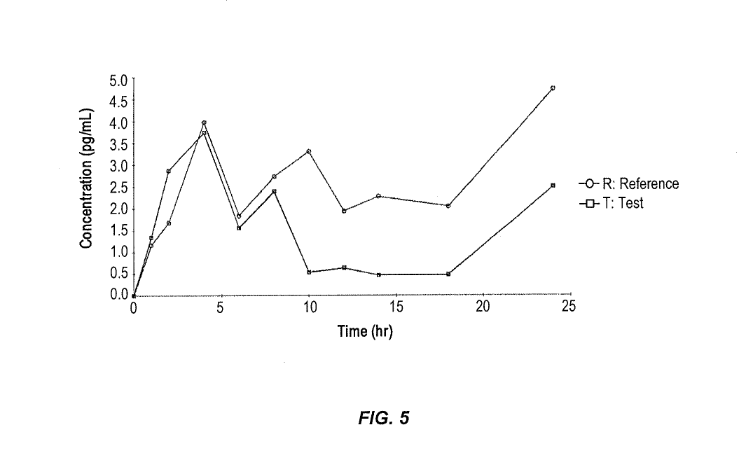

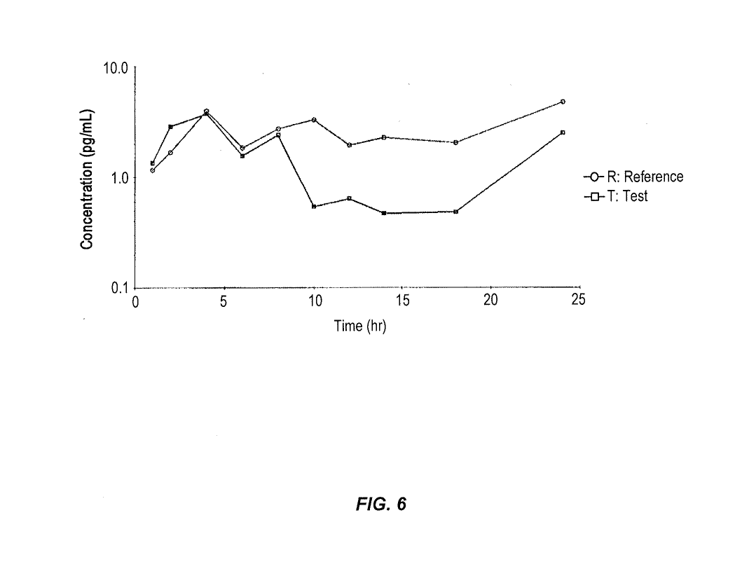

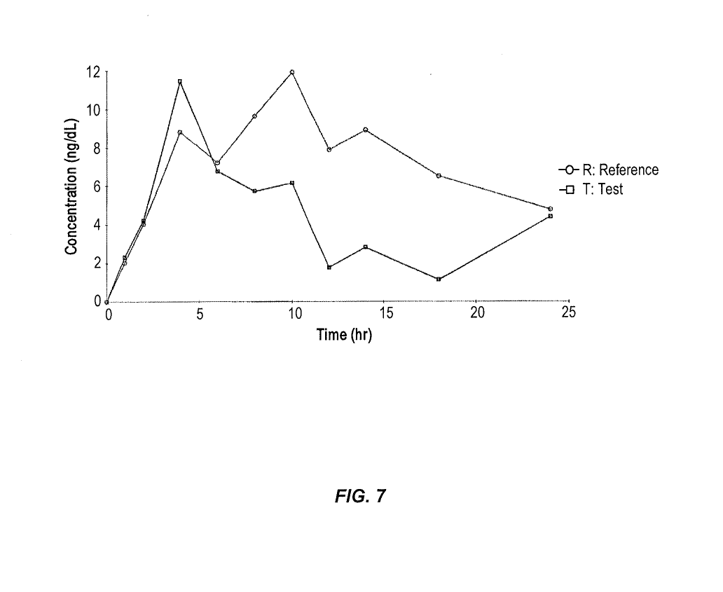

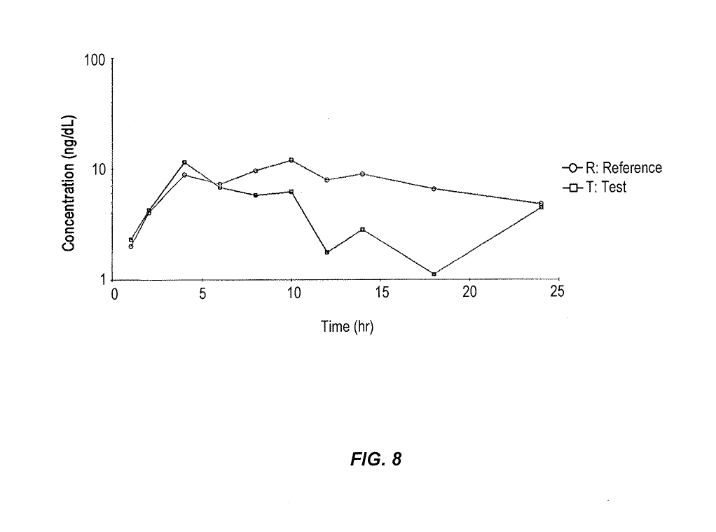

With reference to FIG. 2, delivery vehicle 200 includes pharmaceutical composition 202 and capsule 204. Width 208 represents the thickness of capsule 204, for example about 0.108 inches. The distance from one end of delivery vehicle 200 to another is represented by distance 206, for example about 0.690 inches. The size of delivery vehicle 200 may also be described by the arc swept by a radius of a given length. For example, arc 210, which is defined by the exterior of gelatin 204, is an arc swept by a radius of about 0.189 inches. Arc 212, which is defined by the interior of capsule 204, is an arc swept by a radius of about 0.0938 inches. Arc 214, which is defined by the exterior of gelatin 204 opposite arc 210, is an arc swept by a radius of about 0.108 inches. Suitable capsules of other dimensions may be provided. According to embodiments, capsule 204 has dimensions the same as or similar to the ratios as provided above relative to each other. In some embodiment, the gelatin capsule further comprises one or more components selected from the group consisting of hydrolyzed gelatin, sorbitol-sorbitan solution, water, glycerin, titanium dioxide, FD&C Red #40, ethanol, ethyl acetate, propylene glycol, polyvinyl acetate phthalate, isopropyl alcohol, polyethylene glycol, and ammonium hydroxide.

According to embodiments, the delivery vehicle is designed to remain in the vagina until the pharmaceutical compositions are released. According to embodiments, the delivery vehicle dissolves intravaginally and is absorbed into the vaginal tissue with the pharmaceutical composition, which minimizes vaginal discharge. In such embodiments, the delivery mechanism is made from constituents that are non-toxic, for example, gelatin.

Design Factors for Vaginally Inserted Pharmaceutical Compositions

According to embodiments, the pharmaceutical composition is designed to maximize favorable characteristics that lead to patient compliance (patients that discontinue treatment prior to completion of the prescribed course of therapy), without sacrificing efficacy. Favorable characteristics include, for example, lack of or reduction of irritation relative to other hormone replacement pessaries, lack of or reduction in vaginal discharge of the pharmaceutical composition and delivery vehicle relative to other hormone replacement pessaries, lack of or reduction of pharmaceutical composition or delivery vehicle residue inside the vagina, ease of administration compared to other hormone replacement pessaries, or improved efficacy of drug product relative to otherwise similar pharmaceutical compositions.

According to embodiments, the pharmaceutical composition is non-irritating or minimizes irritation. Patient irritation includes pain, pruritus (itching), soreness, excessive discharge, swelling, or other similar conditions. Patient irritation results in poor compliance. Non-irritating or reduced irritation pharmaceutical compositions can mean that the irritation measured relative to competing hormone pessaries, including tablets, creams, or other intravaginal estrogen delivery forms, is less with respect to the compositions of the invention.

According to embodiments, administration of the pharmaceutical compositions does not result in systemic exposure (e.g., blood circulation level of estradiol above baseline or placebo), which improves safety. According to other embodiments, the pharmaceutical compositions disclosed herein result in significantly reduced systemic exposure (e.g., blood circulation of estradiol) when compared to other vaginally administered drugs on the market for the treatment of VVA.

According to embodiments, the daily administration of a composition comprising 1-25 μg, such as about 4-25 μg of a composition of the invention (e.g., an estradiol gel cap of the invention) results in a mean estradiol Cₘₐₓ of less than about 40 pg/mL on day 1 and less than about 25 pg/mL on day 14. According to embodiments, administration of 4 μg of a composition of the invention every day for 14 days results in a mean estradiol Cₘₐₓ of less than about 10 pg/mL on day 1 and less than about 5 pg/mL on day 14.

According to embodiments, the daily administration of a composition comprising 1-25 μg, such as about 4-25 μg of a composition of the invention (e.g., an estradiol gel cap of the invention) results in a mean estradiol AUC of less than about 300 pg*hr/mL on day 1 and less than about 250 pg*hr/mL on day 14. According to embodiments, administration of 4 μg of a composition of the invention every day for 14 days results in a mean estradiol AUC of less than about 120 pg*hr/mL on day 1 and less than about 110 pg*hr/mL on day 14.

According to embodiments, the daily administration of a composition comprising 1-25 μg, such as about 4-25 μg of a composition of the invention (e.g., an estradiol gel cap of the invention) results in a mean estradiol C_{avg} of less than about 20 pg/mL on day 1 and less than about 15 pg/mL on day 14. According to embodiments, administration of 4 μg of a composition of the invention every day for 14 days results in a mean estradiol C_{avg} of less than about 6 pg/mL on day 1 and less than about 6 pg/mL on day 14.

According to embodiments, the daily administration of a composition comprising 1-25 μg, such as about 4-25 μg of a composition of the invention (e.g., an estradiol gel cap of the invention) results in a mean estradiol Tₘₐₓ of less than about 10 hr on day 1 and less than about 10 hr on day 14. According to embodiments, administration of 4 μg of a composition of the invention every day for 14 days results in a mean estradiol Tₘₐₓ of less than about 8 hr on day 1 and less than about 10 hr on day 14.

According to embodiments, the daily administration of a composition comprising 1-25 μg, such as about 4-25 μg of a composition of the invention (e.g., an estradiol gel cap of the invention) results in a mean estrone Cₘₐₓ of less than about 30 pg/mL on day 1 and less than about 30 pg/mL on day 14. According to embodiments, administration of 4 μg of a composition of the invention every day for 14 days results in a mean estrone Cₘₐₓ of less than about 20 pg/mL on day 1 and less than about 1 pg/mL on day 14.

According to embodiments, the daily administration of a composition comprising 1-25 μg, such as about 4-25 μg of a composition of the invention (e.g., an estradiol gel cap of the invention) results in a mean estrone AUC of less than about 600 pg*hr/mL on day 1 and less than about 600 pg*hr/mL on day 14. According to embodiments, administration of 4 μg of a composition of the invention every day for 14 days results in a mean estrone AUC of less than about 375 pg*hr/mL on day 1 and less than about 415 pg*hr/mL on day 14.

According to embodiments, the daily administration of a composition comprising 1-25 μg, such as about 4-25 μg of a composition of the invention (e.g., an estradiol gel cap of the invention) results in a mean estrone C_{avg} of less than about 30 pg/mL on day 1 and less than about 30 pg/mL on day 14. According to embodiments, administration of 4 μg of a composition of the invention every day for 14 days results in a mean estrone C_{avg} of less than about 20 pg/mL on day 1 and less than about 20 pg/mL on day 14.

According to embodiments, the daily administration of a composition comprising 1-25 μg, such as about 4-25 μg of a composition of the invention (e.g., an estradiol gel cap of the invention) results in a mean estrone Tₘₐₓ of less than about 15 hr on day 1 and less than about 15 hr on day 14. According to embodiments, administration of 4 μg of a composition of the invention every day for 14 days results in a mean estrone Tₘₐₓ of less than about 15 hr on day 1 and less than about 15 hr on day 14.

According to embodiments, the daily administration of a composition comprising 1-25 μg, such as about 4-25 μg of a composition of the invention (e.g., an estradiol gel cap of the invention) results in a mean estrone conjugates Cₘₐₓ of less than about 600 pg/mL on day 1 and less than about 650 pg/mL on day 14. According to embodiments, administration of 4 μg of a composition of the invention every day for 14 days results in a mean estrone conjugates Cₘₐₓ of less than about 1 pg/mL on day 1 and less than about 60 pg/mL on day 14.

According to embodiments, the daily administration of a composition comprising 1-25 μg, such as about 4-25 μg of a composition of the invention (e.g., an estradiol gel cap of the invention) results in a mean estrone conjugates AUC of less than about 8000 pg*hr/mL on day 1 and less than about 13000 pg*hr/mL on day 14. According to embodiments, administration of 4 of a composition of the invention every day for 14 days results in a mean estrone conjugates AUC of less than about 6500 pg*hr/mL on day 1 and less than about 7000 pg*hr/mL on day 14.

According to embodiments, the daily administration of a composition comprising 1-25 μg, such as about 4-25 μg of a composition of the invention (e.g., an estradiol gel cap of the invention) results in a mean estrone conjugates C_{avg} of less than about 550 pg/mL on day 1 and less than about 600 pg/mL on day 14. According to embodiments, administration of 4 μg of a composition of the invention every day for 14 days results in a mean estrone conjugates C_{avg} of less than about 350 pg/mL on day 1 and less than about 350 pg/mL on day 14.

According to embodiments, the daily administration of a composition comprising 1-25 μg, such as about 4-25 μg of a composition of the invention (e.g., an estradiol gel cap of the invention) results in a mean estrone conjugates Tₘₐₓ of less than about 15 hr on day 1 and less than about 15 hr on day 14. According to embodiments, administration of 4 μg of a composition of the invention every day for 14 days results in a mean estrone Tₘₐₓ of less than about 15 hr on day 1 and less than about 15 hr on day 14.

According to embodiments, composition of the invention having the above-referenced amount of estradiol can exhibit one, two, three, four, five, six, seven, eight, nine, or even more of any of the above-referenced pharmacokinetic results upon administration for the indicated times or such results in combination with any of the other results described herein in connection with the administration of such compositions

In certain embodiments, the administration of the pharmaceutical composition provides a mean concentration (Cₐᵥₑ) value below 20.6 pg/mL on Day 1 of the treatment, and/or a Cₐᵥₑ value below 19.4 pg/mL on Day 14 of the treatment, and/or a Cₐᵥₑ value below 11.5 pg/mL on Day 83 of the treatment. In certain embodiments, the administration of the pharmaceutical composition provides a mean concentration (Cₐᵥₑ) value below 10 pg/mL on Day 1 of the treatment, and/or a Cₐᵥₑ value below 7.3 pg/mL on Day 14 of the treatment, and/or a Cₐᵥₑ value below 5.5 pg/mL on Day 83 of the treatment.

According to embodiments, the pharmaceutical composition does not leave residue inside the vagina. Rather, the pharmaceutical composition and delivery vehicle are substantially absorbed or dispersed without resulting in unabsorbed residue or unpleasant sensations of non-absorbed or non-dispersed drug product. According to embodiments, at least 90%, for example about 95%, 97%, 99%, 99.5%, e.g. at least 99.99% of subjects receiving administration of a delivery vehicle according to the invention will report or at least 90%, for example about 95%, 97%, 99%, 99.5%, e.g. at least 99.99% of doctors examining subjects receiving the delivery vehicle of the invention will report that the delivery vehicle is undetectable at day 1 post administration. According to certain embodiments, at least 95% of subjects, for example at least 96%, 97%, 98%, 99% or 100% of subjects have no remaining delivery vehicle detectable at day 1 post administration, as determined by other subjective or objective measurements. According to embodiments the average amount of capsule material that is remaining in subjects 1 day after administration is about 5% or less, about 4% or less, about 3% or less, about 2% or less, about 1% or less, about 0.5% or less, about 0.1% or less, or about 0.01% or less or the average amount of material in the subjects will be 0.001% or less or below the level of detection. Measurement of lack of residue is relative to other vaginally inserted products or can be measured objectively with inspection of the vaginal tissues. For example, certain other vaginally inserted products contain starch which can result in greater discharge from the vagina following administration than compositions of the invention. In some embodiments, the pharmaceutical compositions provided herein provide a lower amount, duration, or frequency of discharge following administration compared to other vaginally inserted products (e.g., compressed tablets).

According to embodiments, the pharmaceutical composition improves vaginal discharge compared to other pessaries, including pessaries that deliver hormones. Ideally, vaginal discharge is eliminated, minimized, or improved compared to competing products.

According to embodiments, the pharmaceutical compositions disclosed herein are inserted digitally. According to embodiments, the pharmaceutical compositions are digitally inserted approximately two inches or other amount (e.g., about 0.5 inch to about 2.5 inches, such as about 0.75 inches to about 2.25 inches, or about 0.65 inches to about 2.25 inches) into the vagina without a need for an applicator. According to embodiments, the pharmaceutical compositions are designed to be also inserted with an applicator, if desired. According to some embodiments, because the site of VVA is in the proximal region of the vagina (towards the vaginal opening), the pharmaceutical compositions disclosed herein are designed to be inserted in the proximal portion (lower ½, lower ⅓^{rd}, or lower ¼^{th}) of the vagina. According to embodiments the delivery vehicle of the composition is not a tablet or is a delivery vehicle that is significantly softer than a tablet. According to embodiments, at least 50% of the formulation, such as at least 60% or at least 70% or more of the total formulation is in liquid form, at least in the body following administration.

Through extensive experimentation, various medium chain fatty acid esters of glycerol and propylene glycol demonstrated one or more favorable characteristics for development as a human drug product. According to embodiments, the solubilizing agent was selected from at least one of a solvent or co-solvent. Suitable solvents and co-solvents include any mono-, di- or triglyceride and glycols, and combinations thereof.

According to embodiments, the pharmaceutical composition is delivered via a gelatin capsule delivery vehicle. According to these embodiments, the pharmaceutical composition is a liquid pharmaceutical composition. According to embodiments, the delivery vehicle is a soft capsule, for example a soft gelatin capsule. Thus, the pharmaceutical composition of such embodiments is encapsulated in the soft gelatin capsule or other soft capsule.

According to embodiments, the pharmaceutical composition includes estradiol that is at least about 80% solubilized in a solubilizing agent comprising one or more C6 to C14 medium chain fatty acid mono-, di-, or triglycerides and, optionally, a thickening agent. According to embodiments, the pharmaceutical composition includes estradiol that is at least about 80% solubilized in one or more C6 to C12 medium chain fatty acid mono-, di-, or triglycerides, e.g., one or more C6 to C14 triglycerides, e.g., one or more C6 to C12 triglycerides, such as one or more C8-C10 triglycerides. These embodiments specifically contemplate the estradiol being at least 80% solubilized. These embodiments specifically contemplate the estradiol being at least 85% solubilized. These embodiments specifically contemplate the estradiol being at least 90% solubilized. These embodiments specifically contemplate the estradiol being at least 95% solubilized. These embodiments specifically contemplate the estradiol being fully solubilized.

As noted above, liquid pharmaceutical compositions are liquid at room temperature or at body temperature. For example, in some embodiments, a pharmaceutical composition provided herein is a liquid formulation contained within a soft gel capsule. Gels, hard fats, or other solid forms that are not liquid at room or body temperature are less desirable in embodiments of the pharmaceutical composition that are liquid.

The thickening agent serves to increase viscosity, e.g., up to about 10,000 cps (10,000 mPa-s), typically to no more than about 5000 cps, and more typically to between about 50 and 1000 cps. In embodiments, the non-ionic surfactant, e.g., GELUCIRE or TEFOSE, may be solid at room temperature and require melting to effectively mix with the solubilizing agent. However, in these embodiments, the resultant pharmaceutical composition remains liquid, albeit with greater viscosity, not solid. According to some embodiments, the formulation without the addition of the thickening agent has a viscosity too low to be retained, upon release from the softgel, within the vagina over the course of 24 hours. The existence of unsuitable viscosity can be determined by discharge above a desired frequency (e.g., detectable discharge in about 80% or more, about 85% or more, about 90% or more, about 95% or more or about 99% or more of subjects receiving the product), by too great of discharge in the cases where discharge occurs (e.g., an increase in overall discharge or API discharge of at least about 15%, at least about 25%, at least about 33%, or at least about 50%), or inability of the formulation or API to spread to a desired amount of tissue (e.g., an area of tissue disclosed elsewhere herein). The addition of the thickening agent serves to increase the viscosity of the formulation such that it may be retained in the vagina over the course of 24 hours and, according to embodiments, the increased viscosity further provides the formulation with the ability to spread to target tissues, e.g. the vulva or a portion thereof, such as the labia.

According to embodiments, the pharmaceutical composition includes estradiol, the medium chain solubilizing agent, and the thickening agent as the ingredients delivered via a soft capsule delivery vehicle. Other ingredients, e.g., colorants, antioxidants, preservatives, or other ingredients may be included as well. However, the addition of other ingredients should be in amounts that do not materially change the solubility of the estradiol, the pharmacokinetics of the pharmaceutical composition, or efficacy of the pharmaceutical composition. Other factors that should be considered when adjusting the ingredients of the pharmaceutical composition include the irritation, vaginal discharge, intravaginal residue, and other relevant factors, for example those that would lead to reduced patient compliance. Other contemplated ingredients include: oils or fatty acid esters, lecithin, mucoadherent agents, gelling agents, dispersing agents, or the like.

### VI. METHODS

This disclosure also provides a method of treating an estrogen-deficient state, the method comprising administering to a patient in need thereof, a suppository as provided herein. In some embodiments, a method of treating vulvovaginal atrophy is provided, the method comprising administering to a patient in need thereof, a suppository as provided herein.

According to embodiments, the pharmaceutical compositions disclosed herein can be used for the treatment of VVA, including the treatment of at least one VVA symptom including: vaginal dryness, vaginal or vulvar irritation or itching, dysuria, dyspareunia, and vaginal bleeding associated with sexual activity, among others. According to embodiments, the methods of treatment are generally applicable to females.

According to embodiments, the pharmaceutical compositions disclosed herein can be used for the treatment of estrogen-deficient urinary states. According to embodiments, the pharmaceutical compositions disclosed herein can be used for the treatment of dyspareunia, or vaginal bleeding associated with sexual activity.

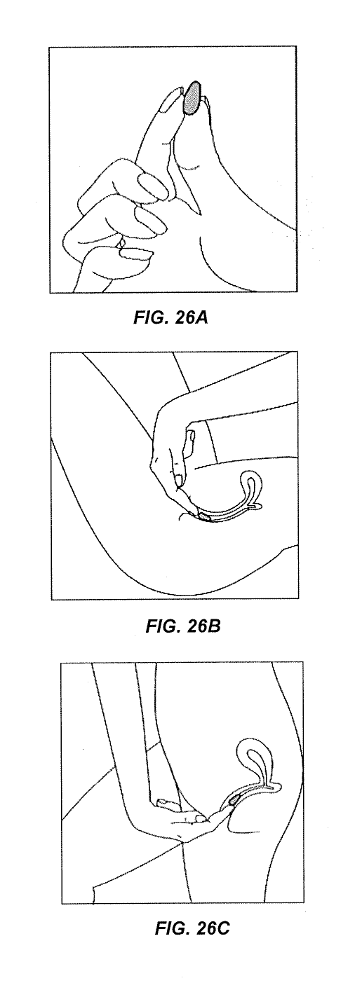

According to embodiments, treatment of VVA, estrogen-deficient urinary states, dyspareunia and vaginal bleeding associated with sexual activity occurs by administering the pharmaceutical compositions intravaginally. According to embodiments where the delivery vehicle is a capsule, the patient obtains the capsule and inserts the capsule into the vagina, where the capsule dissolves and the pharmaceutical composition is released into the vagina where it is absorbed into the vaginal tissue. In some embodiments, the pharmaceutical composition is completely absorbed into the vaginal tissue. In some embodiments, the pharmaceutical composition is substantially absorbed into the vaginal tissue (e.g., at least about 80% by weight, at least about 85% by weight, at least about 90% by weight, at least about 95% by weight, at least about 97% by weight, at least about 98% by weight, or at least about 99% by weight of the composition is absorbed). According to embodiments, the capsule is inserted about two inches into the vagina, however the depth of insertion is generally any depth that allows for adsorption of substantially all of the pharmaceutical composition. According to embodiments, the capsule can also be applied using an applicator that deposits the capsule at an appropriate vaginal depth as disclosed herein. According to embodiments, the capsule is insert into the lower third of the vagina (i.e., the third closest to the vaginal opening). According to embodiments, the softgel capsule can be held with the larger end between the fingers as shown in FIG. 26A.

The subject will select a position that is most comfortable (e.g., a reclining position as shown in FIG. 26B or a standing position as shown in FIG. 26C), and the subject will insert the softgel into the lower third of the vagina with the smaller end up. The softgel capsule will dissolve rapidly. The softgel can be inserted at any time of day and normal activities can be immediately resumed. According to embodiments, the same time of day for all insertions of the softgel is used.

According to embodiments where the pharmaceutical composition is a cream, gel, ointment, or other similar preparation, the pharmaceutical composition is applied digitally, as is well known and understood in the art.

Upon release of the pharmaceutical composition in the vagina, estradiol or other estrogen delivered by administration of the composition is locally absorbed. For example, following administration of the suppository to the proximal region of the vagina of a patient provides a therapeutically effective concentration of estradiol over 24 hours in the proximal region of the vagina.

According to embodiments, the timing of administration of the pharmaceutical composition of this disclosure may be conducted by any safe means as prescribed by an attending physician. According to embodiments, a patient will administer the pharmaceutical composition (e.g., a capsule) intravaginally each day for 14 days, then twice weekly thereafter. In some such embodiments, the doses administered during the twice weekly dosing period are administered approximately 3-4 days apart. Typically, doses administered during the twice weekly dosing period do not exceed more than twice in a seven-day period.

In one embodiment, the method comprises assessing whether the patient has one or more of the following indications: undiagnosed abnormal genital bleeding, known breast cancer, suspected breast cancer or history of breast cancer, a known estrogen-dependent neoplasia or a suspected estrogen dependent neoplasia, active deep vein thrombosis (DVT), active pulmonary embolism (PE), a history of DVT, a history of PE, active arterial thromboembolic disease, such as stroke and myocardial infarction (MI) or a history of stroke or MI. Also or alternatively, in certain embodiments, the method comprises assessing whether the patient has known anaphylactic reaction or angioedema with the treatments described herein. According to embodiments, subjects being identified with one or more of the preceding conditions are advised not to take the product, especially when the API is estradiol or another estrogen.

According to embodiments, the method comprises excluding subjects from treatment based on one or more of the following indications: undiagnosed abnormal genital bleeding, known breast cancer, suspected breast cancer or history of breast cancer, a known estrogen-dependent neoplasia or a suspected estrogen dependent neoplasia, active deep vein thrombosis (DVT), active pulmonary embolism (PE), a history of DVT, a history of PE, active arterial thromboembolic disease, such as stroke and myocardial infarction (MI), a history of stroke or MI, or a known anaphylactic reaction or angioedema with the treatments described herein. According to embodiments such a step is performed when the API of the composition is an estradiol or another estrogen.

According to embodiments, the method comprises providing a user with treatments described herein, such as treatment with a gel cap comprising an estradiol or estrogen, wherein the reported frequency of any adverse event from use of the prescribed treatment that is observed in greater than or equal to about 3% of patients being treated, or is numerically more common in patients receiving treatment, or both, is that of headaches, when use of the product is tested in a clinical trial, such as those described elsewhere herein in terms of number of patients or statistical significance. Also or alternatively, where headache is reported as an adverse event, the reporting of the adverse event of a headache does not occur in a frequency higher than about 25% above that reported by those treated by placebo when use of the product is tested in a clinical trial, such as those described elsewhere herein in terms of number of patients or statistical significance. According to embodiments, the pharmaceutical compositions are vaginally administered with co-administration of an orally administered estrogen-based (or progestin-based or progestin- and estrogen-based) pharmaceutical drug product, or patch, cream, gel, spray, transdermal delivery system or other parenterally-administered estrogen-based pharmaceutical drug product, each of which can include natural, bio-similar, or synthetic or other derived estrogens or progestins. According to embodiments, modulation of circulating estrogen levels provided via the administration of the pharmaceutical compositions disclosed herein, if any, are not intended to be additive to any co-administered estrogen product and its associated circulating blood levels. According to other embodiments, co-administrated estrogen products are intended to have an additive effect as would be determined by the patient physician.

According to certain embodiments, a patient receiving treatment with a product of the invention comprising an estradiol or an estrogen API is instructed to avoid concurrent intake of the treatments described herein and a P450 3A4 (CYP3A4) inducer. For example, a patient is instructed not to ingest any one of the following non-limiting examples: Hypericum perforatum (St. John's wort) preparations, phenobarbital, carbamazepine, and rifampin. Also or alternatively, according to certain embodiments, a patient is instructed to avoid concurrent intake of the treatments described herein and a P450 3A4 (CYP3A4) inhibitor. For example, a patient is instructed not to ingest any one of the following non-limiting examples: erythromycin, clarithromycin, ketoconazole, itraconazole, ritonavir or grapefruit juice.

According to embodiments, a method for estrogenizing vaginal tissue is provided. The method includes administration of a softgel vaginal estriol, estradiol, or other estrogen formulation (i.e., a suppository) or dosage as described herein. Estrogenized vaginal tissue is typically characterized by one or more of the following properties: the presence clear secretions on vaginal walls; rogation and elasticity of the vaginal walls; intact vaginal epithelium; and pink tissue color. In contrast, de-estrogenized vaginal tissue is characterized by decreased or absent secretions; smooth tissue with fewer or no rugae; bleeding of the vaginal surface; development of petechiae (i.e., pinpoint, round spots on the skin due to bleeding, appearing red, brown, or purple); and pale or transparent tissues. Accordingly, estrogenizing vaginal tissue according to the method disclosed herein can include, as a result of delivering an effective amount of an estrogen composition of the invention, increasing the level of vaginal secretions in a subject; increasing the number of vaginal rugae in the subject; and/or decreasing bleeding or petechiae in the subject. According to embodiments, a method for estrogenizing vaginal tissue is provided, the method including administering a suppository so as to provide an effective amount of an estrogen, such as an estradiol Cₘₐₓ or AUC as described herein. According to embodiments, a method for estrogenizing vaginal tissue is provided, the method including administering a suppository so as to provide an estrone Cₘₐₓ or AUC as described herein.

According to embodiments, a method for estrogenizing the labia majora and labia minora (collectively “labia”) is provided as described herein. Generally, the pharmaceutical composition is inserted digitally into the vagina approximately 0.5 inches to about 2.5 inches, such as about two inches or inserted into the third of the vagina closest to the vaginal opening as shown in FIGS. 26A, 26B, and 26C. The gelatin capsule containing the pharmaceutical composition dissolves, ruptures, or otherwise releases the pharmaceutical composition into the vagina, whereby the lower third of the vagina and labia are both reestrogenized. According to some embodiments, the pharmaceutical composition is a liquid that partially flows to the labia and directly reestrogenizes the labia.

According to embodiments, a method for estrogenizing the vulva is provided as described herein. Generally, the pharmaceutical composition is inserted digitally into the vagina approximately 0.5 inches to about 2.5 inches, such as about two inches or inserted into the third of the vagina closest to the vaginal opening as shown in FIGS. 26A, 26B, and 26C. The gelatin capsule containing the pharmaceutical composition dissolves, ruptures, or otherwise releases the pharmaceutical composition into the vagina, whereby the lower third of the vagina and vulva are both reestrogenized. According to some embodiments, the pharmaceutical composition is a liquid that partially flows to the vulval tissue and directly reestrogenizes the vulva.

According to embodiments, a method for treating vaginal dryness is provided. The method includes administration of a soft gel vaginal estradiol formulation (i.e., a suppository) or dosage as described herein. Treating vaginal dryness according to the method disclosed herein can include, decreasing the severity of vaginal dryness by 1%, 5%, 10%, 15%, 20%, 25%, 30%, 45%, 50%, 55%, 60%, 65%, 70%, 75%, 80%, 85%, 90%, 95%, or 99%. The decrease in severity can be obtained following 2 weeks of treatment, or 6 weeks of treatment, or 8 weeks of treatment, or 12 weeks of treatment. In some embodiments, vaginal dryness is assessed using a severity scale, ranging from 0 to 4 points wherein 0 indicates no dryness, 1 indicates mild dryness, 2 indicates moderate dryness, and 3 indicates severe dryness. According to certain embodiments, a decrease in vaginal dryness is experienced within 2 weeks of beginning treatment with the compositions described herein.

In some embodiments, the method for treating vaginal dryness includes reducing the dryness severity score from 3, prior to treatment of a subject, to 2, after 2 weeks of treatment of the subject. In some embodiments, the method for treating vaginal dryness includes reducing the dryness severity score from 2, prior to treatment of a subject, to 1, after 2 weeks of treatment of the subject. In some embodiments, the method for treating vaginal dryness includes reducing the dryness severity score from 1, prior to treatment of subject, to 0, after 2 weeks of treatment of the subject. In some embodiments administration of compositions of the invention result in such decreases in a statistically significant amount of VVA patients receiving a therapeutically effective amount of the composition for a course of treatment such as for at least about two weeks, at least about eight weeks, or at least about twelve weeks.

In some embodiments, the method for treating vaginal dryness includes reducing the dryness severity score from 3, prior to treatment of a subject, to 2, after 6 weeks of treatment of the subject. In some embodiments, the method for treating vaginal dryness includes reducing the dryness severity score from 2, prior to treatment of a subject, to 1, after 6 weeks of treatment of the subject. In some embodiments, the method for treating vaginal dryness includes reducing the dryness severity score from 1, prior to treatment of subject, to 0, after 6 weeks of treatment of the subject.

In some embodiments, the method for treating vaginal dryness includes reducing the dryness severity score from 3, prior to treatment of a subject, to 2, after 8 weeks of treatment of the subject. In some embodiments, the method for treating vaginal dryness includes reducing the dryness severity score from 2, prior to treatment of a subject, to 1, after 8 weeks of treatment of the subject. In some embodiments, the method for treating vaginal dryness includes reducing the dryness severity score from 1, prior to treatment of subject, to 0, after 8 weeks of treatment of the subject.

In some embodiments, the method for treating vaginal dryness includes reducing the dryness severity score from 3, prior to treatment of a subject, to 2, after 12 weeks of treatment of the subject. In some embodiments, the method for treating vaginal dryness includes reducing the dryness severity score from 2, prior to treatment of a subject, to 1, after 12 weeks of treatment of the subject. In some embodiments, the method for treating vaginal dryness includes reducing the dryness severity score from 1, prior to treatment of subject, to 0, after 12 weeks of treatment of the subject.

In some embodiments, the method for treating vaginal dryness includes decreasing the severity of dryness after two weeks of treatment, wherein the severity is assessed on a scale of 0-3 points, and the average decrease ranges from a 0.5-point decrease to a 1.25-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vaginal dryness includes decreasing the severity of dryness after six weeks of treatment, wherein the severity is assessed on a scale of 0-3 points, and the average decrease ranges from a 0.75-point decrease to a 1.5-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vaginal dryness includes decreasing the severity of dryness after eight weeks of treatment, wherein the severity is assessed on a scale of 0-3 points, and the average decrease ranges from a 0.9-point decrease to a 1.5-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vaginal dryness includes decreasing the severity of dryness after twelve weeks of treatment, wherein the severity is assessed on a scale of 0-3 points, and the average decrease ranges from a 0.9-point decrease to a 1.5-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vaginal dryness includes administering a suppository so as to provide an estradiol Cₘₐₓ or AUC as described herein. According to embodiments, a method for treating vaginal dryness is provided, the method including administering a suppository so as to provide an estrone Cₘₐₓ or AUC as described herein.

According to embodiments, a method for treating vulvar and/or vaginal itching or irritation is provided. The method includes administration of a soft gel vaginal estradiol formulation (i.e., a suppository) or dosage as described herein. Treating vulvar and/or vaginal itching or irritation according to the method disclosed herein can include, decreasing the severity of vulvar and/or vaginal itching or irritation by 1%, 5%, 10%, 15%, 20%, 25%, 30%, 45%, 50%, 55%, 60%, 65%, 70%, 75%, 80%, 85%, 90%, 95%, or 99%. The decrease in severity can be obtained following 2 weeks of treatment, or 6 weeks of treatment, or 8 weeks of treatment, or 12 weeks of treatment. In some embodiments, vulvar and/or vaginal itching or irritation is assessed using a severity scale, ranging from 0 to 4 points wherein 0 indicates no itching or irritation, 1 indicates mild itching or irritation, 2 indicates moderate itching or irritation, and 3 indicates severe itching or irritation.

In some embodiments, the method for treating vulvar and/or vaginal itching or irritation includes reducing the itching/irritation severity score from 3, prior to treatment of a subject, to 2, after 2 weeks of treatment of the subject. In some embodiments, the method for treating vulvar and/or vaginal itching or irritation includes reducing the itching/irritation severity score from 2, prior to treatment of a subject, to 1, after 2 weeks of treatment of the subject. In some embodiments, the method for treating vulvar and/or vaginal itching or irritation includes reducing the itching/irritation severity score from 1, prior to treatment of subject, to 0, after 2 weeks of treatment of the subject.

In some embodiments, the method for treating vulvar and/or vaginal itching or irritation includes reducing the itching/irritation severity score from 3, prior to treatment of a subject, to 2, after 6 weeks of treatment of the subject. In some embodiments, the method for treating vulvar and/or vaginal itching or irritation includes reducing the itching/irritation severity score from 2, prior to treatment of a subject, to 1, after 6 weeks of treatment of the subject. In some embodiments, the method for treating vulvar and/or vaginal itching or irritation includes reducing the itching/irritation severity score from 1, prior to treatment of subject, to 0, after 6 weeks of treatment of the subject.

In some embodiments, the method for treating vulvar and/or vaginal itching or irritation includes reducing the itching/irritation severity score from 3, prior to treatment of a subject, to 2, after 8 weeks of treatment of the subject. In some embodiments, the method for treating vulvar and/or vaginal itching or irritation includes reducing the itching/irritation severity score from 2, prior to treatment of a subject, to 1, after 8 weeks of treatment of the subject. In some embodiments, the method for treating vulvar and/or vaginal itching or irritation includes reducing the itching/irritation severity score from 1, prior to treatment of subject, to 0, after 8 weeks of treatment of the subject.

In some embodiments, the method for treating vulvar and/or vaginal itching or irritation includes reducing the itching/irritation severity score from 3, prior to treatment of a subject, to 2, after 12 weeks of treatment of the subject. In some embodiments, the method for treating vulvar and/or vaginal itching or irritation includes reducing the itching/irritation severity score from 2, prior to treatment of a subject, to 1, after 12 weeks of treatment of the subject. In some embodiments, the method for treating vulvar and/or vaginal itching or irritation includes reducing the itching/irritation severity score from 1, prior to treatment of subject, to 0, after 12 weeks of treatment of the subject.

In some embodiments, the method for treating vulvar and/or vaginal itching or irritation includes decreasing the severity of itching/irritation after two weeks of treatment, wherein the severity is assessed on a scale of 0-3 points, and the average decrease ranges from a 0.3-point decrease to a 0.6-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vulvar and/or vaginal itching or irritation includes decreasing the severity of itching/irritation after six weeks of treatment, wherein the severity is assessed on a scale of 0-3 points, and the average decrease ranges from a 0.5-point decrease to a 0.7-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vulvar and/or vaginal itching or irritation includes decreasing the severity of itching/irritation after eight weeks of treatment, wherein the severity is assessed on a scale of 0-3 points, and the average decrease ranges from a 0.5-point decrease to a 0.8-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vulvar and/or vaginal itching or irritation includes decreasing the severity of itching/irritation after twelve weeks of treatment, wherein the severity is assessed on a scale of 0-3 points, and the average decrease ranges from a 0.5-point decrease to a 1.0-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vulvar and/or vaginal itching or irritation includes administering a suppository so as to provide an estradiol Cₘₐₓ or AUC as described herein. According to embodiments, a method for treating vulvar and/or vaginal itching or irritation is provided, the method including administering a suppository so as to provide an estrone Cₘₐₓ or AUC as described herein.

According to embodiments, a method for treating dyspareunia is provided. The method includes administration of a suppository or dosage as described herein. Treating dyspareunia according to the method disclosed herein can include, decreasing the severity of dyspareunia by 1%, 5%, 10%, 15%, 20%, 25%, 30%, 45%, 50%, 55%, 60%, 65%, 70%, 75%, 80%, 85%, 90%, 95%, or 99%. The decrease in severity can be obtained following 2 weeks of treatment, or 6 weeks of treatment, or 8 weeks of treatment, or 12 weeks of treatment. In some embodiments, dyspareunia is assessed using a severity scale, ranging from 0 to 4 points wherein 0 indicates no pain associated with sexual activity (with vaginal penetration), 1 indicates mild pain associated with sexual activity (with vaginal penetration), 2 indicates moderate pain associated with sexual activity (with vaginal penetration), and 3 indicates severe pain associated with sexual activity (with vaginal penetration). According to certain embodiments, a decrease in severity of dyspareunia is seen within 12 weeks of beginning treatment with the compositions described herein.

In some embodiments, the method for treating dyspareunia includes reducing the dyspareunia severity score from 3, prior to treatment of a subject, to 2, after 2 weeks of treatment of the subject. In some embodiments, the method for treating dyspareunia includes reducing the dyspareunia severity score from 2, prior to treatment of a subject, to 1, after 2 weeks of treatment of the subject. In some embodiments, the method for treating dyspareunia includes reducing the dyspareunia severity score from 1, prior to treatment of subject, to 0, after 2 weeks of treatment of the subject.

In some embodiments, the method for treating dyspareunia includes reducing the dyspareunia severity score from 3, prior to treatment of a subject, to 2, after 6 weeks of treatment of the subject. In some embodiments, the method for treating dyspareunia includes reducing the dyspareunia severity score from 2, prior to treatment of a subject, to 1, after 6 weeks of treatment of the subject. In some embodiments, the method for treating dyspareunia includes reducing the dyspareunia severity score from 1, prior to treatment of subject, to 0, after 6 weeks of treatment of the subject.

In some embodiments, the method for treating dyspareunia includes reducing the dyspareunia severity score from 3, prior to treatment of a subject, to 2, after 8 weeks of treatment of the subject. In some embodiments, the method for treating dyspareunia includes reducing the dyspareunia severity score from 2, prior to treatment of a subject, to 1, after 8 weeks of treatment of the subject. In some embodiments, the method for treating dyspareunia includes reducing the dyspareunia severity score from 1, prior to treatment of subject, to 0, after 8 weeks of treatment of the subject.

In some embodiments, the method for treating dyspareunia includes reducing the dyspareunia severity score from 3, prior to treatment of a subject, to 2, after 12 weeks of treatment of the subject. In some embodiments, the method for treating dyspareunia includes reducing the dyspareunia severity score from 2, prior to treatment of a subject, to 1, after 12 weeks of treatment of the subject. In some embodiments, the method for treating dyspareunia includes reducing the dyspareunia severity score from 1, prior to treatment of subject, to 0, after 12 weeks of treatment of the subject.

In some embodiments, the method for treating dyspareunia includes decreasing the severity of dyspareunia after two weeks of treatment, wherein the severity is assessed on a scale of 0-3 points, and the average decrease ranges from a 0.9-point decrease to a 1.1-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating dyspareunia includes decreasing the severity of dyspareunia after six weeks of treatment, wherein the severity is assessed on a scale of 0-3 points, and the average decrease ranges from a 1.3-point decrease to a 1.5-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating dyspareunia includes decreasing the severity of dyspareunia after eight weeks of treatment, wherein the severity is assessed on a scale of 0-3 points, and the average decrease ranges from a 1.5-point decrease to a 1.8-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating dyspareunia includes decreasing the severity of dyspareunia after twelve weeks of treatment, wherein the severity is assessed on a scale of 0-3 points, and the average decrease ranges from a 1.5-point decrease to a 1.8-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating dyspareunia includes administering a suppository so as to provide an estradiol Cₘₐₓ or AUC as described herein. According to embodiments, a method for treating dyspareunia is provided, the method including administering a suppository so as to provide an estrone Cₘₐₓ or AUC as described herein.

According to embodiments, a method for treating urinary tract infections is provided. As used herein the term “urinary tract infection” refers to an infection of the kidneys, ureters, bladder and urethra by a microorganism such as Escherichia coli, Staphylococcus saprophyticus, Klebsiella sp., Enterobacter sp., or Proteus sp. The method for treating urinary tract infections generally includes administering a soft gel vaginal estradiol formulation (i.e., a suppository) as described herein. According to certain embodiments, the method further includes decreasing urethral discomfort, frequency or urination, hematuria, dysuria, and/or stress incontinence. According to certain embodiments, a method for treating urinary tract infections is provided, the method including administering a suppository as described herein and decreasing vaginal pH from above 4.5 to between 3.5 and 4.5 (inclusive). The method can be particularly effective for treating urinary tract infections in elderly subjects (e.g., subjects older than 65 years, or older than 75 years, or older than 85 years). According to embodiments, a method for treating urinary tract infections is provided, the method including administering a suppository so as to provide an estradiol Cmax or AUC as described herein. According to embodiments, a method for treating urinary tract infections is provided, the method including administering a suppository so as to provide an estrone Cmax or AUC as described herein. According to embodiments, a method for treating sexual dysfunction is provided. As used herein with respect to female subjects, the term “sexual dysfunction” generally refers to pain or discomfort during sexual intercourse, diminished vaginal lubrication, delayed vaginal engorgement, increased time for arousal, diminished ability to reach orgasm, diminished clitoral sensation, diminished sexual desire, and/or diminished arousal.

According to embodiments, a method for treating sexual dysfunction is provided, the method including administering a suppository so as to provide an estradiol Cmax or AUC as described herein. According to embodiments, a method for treating sexual dysfunction is provided, the method including administering a suppository so as to provide an estrone Cmax or AUC as described herein.

Sexual function and dysfunction can be assessed using the Female Sexual Function Index (FSFI) (see, Rosen R, Brown C, Heiman J, et al. “The Female Sexual Function Index (FSFI): A Multidimensional Self-Report Instrument for the Assessment of Female Sexual Function.” Journal of Sex & Marital Therapy 2000. 26: p. 191-208). The FSFI is useful for assessing various domains of sexual functioning (e.g. sexual desire, arousal, orgasm, satisfaction and pain). Accordingly, the method for treating sexual dysfunction as provided herein can include administering a vaginal soft gel formulation to a subject and increasing a subject's full-scale FSFI score, FSFI-desire score, FSFI-arousal score, FSFI-lubrication score and/or FSFI-orgasm score.

### Female Sexual Function Index (FSFI)

| Question | Answer Options |
|---|---|
| Q1: Over the past 4 weeks, how often did you feel | 5 = Almost always or always |
| sexual desire or interest? | 4 = Most times (more than half the time) |
| | 3 = Sometimes (about half the time) |
| | 2 = A few times (less than half the time) |
| | 1 = Almost never or never |
| Q2: Over the past 4 weeks, how would you rate your | 5 = Very high |
| level (degree) of sexual desire or interest? | 4 = High |
| | 3 = Moderate |
| | 2 = Low |
| | 1 = Very low or none at all |
| Q3. Over the past 4 weeks, how often did you feel | 0 = No sexual activity |
| sexually aroused (“turned on”) during sexual activity | 5 = Almost always or always |
| or intercourse? | 4 = Most times (more than half the time) |
| | 3 = Sometimes (about half the time) |
| | 2 = A few times (less than half the time) |
| | 1 = Almost never or never |
| Q4. Over the past 4 weeks, how would you rate your | 0 = No sexual activity |
| level of sexual arousal (“turn on”) during sexual | 5 = Very high |
| activity or intercourse? | 4 = High |
| | 3 = Moderate |
| | 2 = Low |
| | 1 = Very low or none at all |
| Q5. Over the past 4 weeks, how confident were you | 0 = No sexual activity |
| about becoming sexually aroused during sexual | 5 = Very high confidence |
| activity or intercourse? | 4 = High confidence |
| | 3 = Moderate confidence |
| | 2 = Low confidence |
| | 1 = Very low or no confidence |
| Q6. Over the past 4 weeks, how often have you been | 0 = No sexual activity |
| satisfied with your arousal (excitement) during sexual | 5 = Almost always or always |
| activity or intercourse? Response Options | 4 = Most times (more than half the time) |
| | 3 = Sometimes (about half the time) |
| | 2 = A few times (less than half the time) |
| | 1 = Almost never or never |
| Q7: Over the past 4 weeks, how often did you become | 0 = No sexual activity |
| lubricated (“wet”) during sexual activity or | 5 = Almost always or always |
| intercourse? | 4 = Most times (more than half the time) |
| | 3 = Sometimes (about half the time) |
| | 2 = A few times (less than half the time) |
| | 1 = Almost never or never |
| Q8. Over the past 4 weeks, how difficult was it to | 0 = No sexual activity |
| become lubricated (“wet”) during sexual activity or | 1 = Extremely difficult or impossible |
| intercourse? | 2 = Very difficult |
| | 3 = Difficult |
| | 4 = Slightly difficult |
| | 5 = Not difficult |
| Q9: Over the past 4 weeks, how often did you | 0 = No sexual activity |
| maintain your lubrication (“wetness”) until completion | 5 = Almost always or always |
| of sexual activity or intercourse? | 4 = Most times (more than half the time) |
| | 3 = Sometimes (about half the time) |
| | 2 = A few times (less than half the time) |
| | 1 = Almost never or never |
| Q10: Over the past 4 weeks, how difficult was it to | 0 = No sexual activity |
| maintain your lubrication (“wetness”) until completion | 1 = Extremely difficult or impossible |
| of sexual activity or intercourse? | 2 = Very difficult |
| | 3 = Difficult |
| | 4 = Slightly difficult |
| | 5 = Not difficult |
| Q11. Over the past 4 weeks, when you had sexual | 0 = No sexual activity |
| stimulation or intercourse, how often did you reach | 5 = Almost always or always |
| orgasm (climax)? | 4 = Most times (more than half the time) |
| | 3 = Sometimes (about half the time) |
| | 2 = A few times (less than half the time) |
| | 1 = Almost never or never |
| Q12: Over the past 4 weeks, when you had sexual | 0 = No sexual activity |
| stimulation or intercourse, how difficult was it for you | 1 = Extremely difficult or impossible |
| to reach orgasm (climax)? | 2 = Very difficult |
| | 3 = Difficult |
| | 4 = Slightly difficult |
| | 5 = Not difficult |
| Q13: Over the past 4 weeks, how satisfied were you | 0 = No sexual activity |
| with your ability to reach orgasm (climax) during | 5 = Very satisfied 4 |
| sexual activity or intercourse? | 4 = Moderately satisfied |
| | 3 = About equally satisfied and dissatisfied |
| | 2 = Moderately dissatisfied |
| | 1 = Very dissatisfied |
| Q14: Over the past 4 weeks, how satisfied have you | 0 = No sexual activity |
| been with the amount of emotional closeness during | 5 = Very satisfied |
| sexual activity between you and your partner? | 4 = Moderately satisfied |
| | 3 = About equally satisfied and dissatisfied |
| | 2 = Moderately dissatisfied |
| | 1 = Very dissatisfied |
| Q15: Over the past 4 weeks, how satisfied have you | 5 = Very satisfied |
| been with your sexual relationship with your partner? | 4 = Moderately satisfied |
| | 3 = About equally satisfied and dissatisfied |
| | 2 = Moderately dissatisfied |
| | 1 = Very dissatisfied |
| Q16: Over the past 4 weeks, how satisfied have you | 5 = Very satisfied |
| been with your overall sexual life? | 4 = Moderately satisfied |
| | 3 = About equally satisfied and dissatisfied |
| | 2 = Moderately dissatisfied |
| | 1 = Very dissatisfied |
| Q17: Over the past 4 weeks, how often did you | 0 = Did not attempt intercourse |
| experience discomfort or pain during vaginal | 1 = Almost always or always |
| penetration? | 2 = Most times (more than half the time) |
| | 3 = Sometimes (about half the time) |
| | 4 = A few times (less than half the time) |
| | 5 = Almost never or never |
| Q18: Over the past 4 weeks, how often did you | 0 = Did not attempt intercourse |
| experience discomfort or pain following vaginal | 1 = Almost always or always |
| penetration? | 2 = Most times (more than half the time) |
| | 3 = Sometimes (about half the time) |
| | 4 = A few times (less than half the time) |
| | 5 = Almost never or never |
| Q19. Over the past 4 weeks, how would you rate your | 0 = Did not attempt intercourse |
| level (degree) of discomfort or pain during or | 1 = Very high |
| following vaginal penetration? | 2 = High |
| | 3 = Moderate |
| | 4 = Low |
| | 5 = Very low or none at all |

### FSFI Scoring System

| Domain | Questions | Score Range | Factor | Minimum | Maximum |
|---|---|---|---|---|---|
| Desire | 1, 2 | 1-5 | 0.6 | 1.2 | 6.0 |
| Arousal | 3, 4, 5, 6 | 0-5 | 0.3 | 0 | 6.0 |
| Lubrication | 7, 8, 9, | 0-5 | 0.3 | 0 | 6.0 |
| | 10 | | | | |
| Orgasm | 11, 12, | 0-5 | 0.4 | 0 | 6.0 |
| | 13 | | | | |
| Satisfaction | 14, 15, | 0 (or 1)-5 | 0.4 | 0.8 | 6.0 |
| | 16 | | | | |
| Pain | 17, 18, | 0-5 | 0.4 | 0 | 6.0 |
| | 19 | | | | |

| | | |
|---|---|---|
| Full Scale Score Range: | 2.0 | 36.0 |

In some embodiments, the method for treating sexual dysfunction includes administering estradiol to the subject and increasing the FSFI-desire score by at least about 20%, or at least about 25%, or at least about 30% as compared to baseline.

In some embodiments, the method for treating sexual dysfunction includes administering estradiol to the subject and increasing the FSFI-arousal score by at least about 30%, or at least about 40%, or at least about 50% as compared to baseline.

In some embodiments, the method for treating sexual dysfunction includes administering estradiol to the subject and increasing the FSFI-lubrication score by at least about 85%, or at least about 95%, or at least about 115% as compared to baseline.

In some embodiments, the method for treating sexual dysfunction includes administering estradiol to the subject and increasing the FSFI-orgasm score by at least about 40%, or at least about 60% as compared to baseline.

In some embodiments, the method for treating sexual dysfunction includes administering estradiol to the subject and increasing the total FSFI score by at least about 50%, or at least about 55%, or at least about 70% as compared to baseline.

Examples of other metrics for assessment of sexual function include, but are not limited to, Changes in Sexual Function Questionnaire (“CSFQ”; Clayton et al., Psychopharmacol Bull. 33(4):731-45 (1997) and Clayton et al., Psychopharmacol. Bull. 33(4):747-53 (1997)); the Derogatis Interview for Sexual Functioning—Self-Report (“DISF-SR”; Derogatis, J Sex Marital Ther. 23:291-304 (1997)); the Golombok-Rust Inventory of Sexual Satisfaction (“GRISS”; Rust et al., Arch. Sex Behav. 15:157-165 (1986)); the Sexual Function Questionnaire (“SFQ”; Quirk et al., J Womens Health Gend Based Med. 11:277-289 (2002)); and the Arizona Sexual Experience Scale (“ASEX”; McGahuey et al., J Sex Marital Ther. 26:25-40 (2000)), the entire disclosures of which are incorporated herein by reference. For assessment using a questionnaire, a measure of sexual dysfunction function is increased when the score in the appropriate domain, subscale or subtest is indicative of sexual dysfunction, as established for that questionnaire. For instance, a female's sexual interest is considered reduced, when assessed using the CSFQ, if the subscale for sexual interest score is less than or equal to 9. Conversely, sexual dysfunction is considered improved when the score in the appropriate domain, subscale or subtest is indicative of higher (e.g., normal or desired) sexual function. For a clinician's assessment, sexual dysfunction may be assessed in comparison to a previous point in time for the patient and/or in comparison to a patient's peers with respect to age, gender, sexual experience, and health, or may also be determined via a validated questionnaire administered by the clinician.

According to embodiments, the efficacy and safety of the pharmaceutical compositions described herein in the treatment of the symptoms of VVA may be determined. According to embodiments, the size, effect, cytology, histology, and variability of the VVA may be determined using various endpoints to determine efficacy and safety of the pharmaceutical compositions described herein or as otherwise accepted in the art, at present or as further developed. One source of endpoints is with the US Food and Drug Administration's (FDA) published guidelines for treatment of VVA with estradiol.

According to embodiments, a method of treating VVA, including dyspareunia, vaginal dryness, and estrogen-deficient urinary states (including urinary tract infections), is provided that allows a subject to be ambulatory immediately or within minutes after a gelatin capsule containing the pharmaceutical compositions disclosed herein are administered. According to embodiments, a gelatin capsule containing a pharmaceutical composition as disclosed herein is administered by digitally inserting the gelatin capsule containing the pharmaceutical composition into the vagina approximately two inches or inserting into the third of the vagina closest to the vaginal opening as shown in FIGS. 26A, 26B, and 26C. According to embodiments, the capsule remains within the vagina and dissolves, ruptures, or otherwise disintegrates soon after being inserted into the vagina, regardless of user/patient mobility, thereby releasing the pharmaceutical composition. The pharmaceutical composition spreads onto the vaginal tissue and is rapidly absorbed. According to embodiments, the capsule is also fully absorbed by the vaginal tissue. According to some embodiments, a viscosity enhancer such as TEFOSE 63 provides increased viscosity to ensure the pharmaceutical composition stays within the desired absorption area, thereby estrogenizing the vagina, labia, and/or vulva. The combination of high viscosity, controlled spread, and rapid absorption prevents the need for subjects to remain supine after administration to allow the tissue to absorb the estradiol, thereby allowing subjects to be ambulatory immediately or almost immediately after administration.

According to embodiments the invention provides compositions and methods for delivering one or more active pharmaceutical ingredients (“APIs”) or other pharmaceutical agents, such as one or more estrogens, to the vagina, whereby the pharmaceuticals and the associated pharmaceutical composition, including any formulation in which the API is contained, adheres to or is absorbed in vaginal tissue and/or surrounding vagina-associated tissue (e.g., the vulva) with either low discharge of the composition (about 10% or less, about 5% or less, about 4% or less, about 3% or less, about 2% or less, or about 1% or less of subjects reporting discharge in a population of at least about 50 subjects, at least about 75 subjects, at least about 100 subjects, at least about 250 subjects, at least about 500 subjects, at least about 1,000 subjects) or no discharge and wherein the subject is instructed that the subject may be ambulatory immediately following administration of the composition (e.g., by a physician, a physician's assistant, or other healthcare provider and/or by provision of written instructions such as a label for the product, preferably a label approved by a regulatory authority responsible for approval of pharmaceutical products such as US FDA). According to embodiments, the composition is digitally inserted by the subject. According to embodiments, the composition is inserted in the lower portion (e.g., the lower ½, ⅓rd, or ¼th) of the vagina. According to embodiments, the composition is inserted in the first about 1 to about 2.5 inches of the vagina. According to embodiments, a detectable amount of the API is delivered locally to some or all of the vulva in addition to the vagina by spreading of the composition. According to embodiments, a detectable amount of the API is delivered locally to the labia by spreading of the composition. According to embodiments detection is determined by observation of one or more physiological changes in some or all of the vulva (e.g., the labia) that is associated with the presence of increased amounts of the API, such as the treatment of one or more conditions associated with delivery of a therapeutically effective amount of the API to such tissue(s).

According to embodiments, the amount of formulation (which may also be referred to as the “fill” in the case of formulations contained in a delivery vehicle, such as a soft gelcap) delivered by the method is between about 100 and about 1000 mg, such as between about 150 mg and about 900 mg, or between the following ranges: about 150 mg and about 750 mg, such as about 175 mg and about 875 mg or about 175 mg and about 700 mg, about 150 mg and about 600 mg or about 150 mg and 450 mg, about 200 mg and about 500 mg, about 200 mg and about 400 mg, or about 250 mg and about 350 mg, such as about 300 mg. According to embodiments, the fill is contained in a soft gel cap of between about 50 mg and about 300 mg, such as between about 50 mg and about 250 mg, e.g., between about 75 mg and about 225 mg or about 75 mg-about 150 mg, about 60 mg-about 140 mg, about 70 mg-about 130 mg, about 80 mg-about 120 mg, or about 100 mg.

In general, the teachings provided herein in connection with estradiol can be applied to other estrogens. Descriptions of methods or compositions provided herein applied to estradiol can also be considered to provide a description of a corresponding method applied to other estrogens.

According to other embodiments, certain teachings provided herein may also be applied to other non-estrogen APIs. For example, it can be expected that other estrogens and other non-estrogen actives will typically have different physical, chemical and/or pharmaceutical properties than the estradiol/estrogens of the present invention. As such, while the description of the physical effects relating to the delivery of estradiol/estrogen products herein can apply to products comprising other APIs and should be considered similarly disclosed (e.g. in connection with the spreading of the composition and/or lack of discharge of the composition), the medical effects associated with estradiol/estrogen products of the invention (e.g., reestrogenization of the vagina, labia, or vulva) will be typically understood as not being associated with such other API compositions. Other APIs will typically have known medical properties and such properties will be understood to be imparted by the use of a therapeutically effective amount of such APIs in the compositions of the invention.

According to embodiments, the pharmaceutical is an estrogen, such as an estrogen other than estradiol. According to some embodiments, the estrogen may be a natural estrogen such as estradiol, for example 17-beta-estradiol or estrone. In alternative embodiments the natural estrogen may be estriol. According to yet additional alternative embodiments, the active ingredient is a derivative of estradiol, estrone or estriol or other estrogen derivative. Such estradiol derivatives may include but may not be limited to 2- and 4-hydroxyestradiol, 4-methoxyestradiol, 16-alpha IE2 and LE2, cloxestradiol and cloxestradiol acetate, estradiol sulfate, and 17-alpha substituted estradiol derivatives. Additional estradiol derivatives include nitrogen mustard-coupled alkylating antineoplastic estradiol derivatives and 17-beta aminoestrogens. Contemplated estrone derivatives include 1- and 4-hydroxyestrone, 4-methoxyestrone, 16-alpha hydroxyestrone, estrone sulfate, and nitrogen mustard-coupled alkylating antineoplastic estrone derivatives. Contemplated estriol derivatives include estetrol, quinestradol, and 17-alpha-substituted estriol derivatives. Other estrogen derivatives may include 17-alpha estradiol, 16-beta epiestriol and myatrienediol. Those familiar with the field will recognize that potential natural estrogen derivatives are not limited to this list and therefore this list is not intended to be exclusive.

According to embodiments, the pharmaceutical comprises a minor estrogen such as and not limited to 27-hydroxycholesterol, dehydroepiandrosterone (DHEA), 7-oxo-DHEA, 7-alpha-hydroxy-DHEA, 16-alpha-hydroxy-DHEA, 7-beta-hydroxyepiandrosterone, androstenedione, and androstenediol. Those familiar with the field will recognized that potential minor estrogens are not limited to this list and therefore this list is not intended to be exclusive. According to alternative embodiments, the pharmaceutical comprises estrogen metabolites with estrogenic activity, such as but not limited to 2-hydroxyestradiol, 2-hydroxyestrone, 4-hydroxyestradiol, 4-hydroxyestrone, and 16-alpha-hydroxyestrone.

According to certain embodiments, the pharmaceutical comprises an equine estrogen. Such an equine estrogen may include but not be limited to equilin, equilenin, 17-alpha dihydroequilin, 17-beta dihydroequilin, 17-alpha dihydroequilenin, 17-beta dihydroequilenin, 8,9-dehydroestrone, 8,9-dehydroestradiol, and hippulin.

Other estrogen actives contemplated as components of the pharmaceutical include conjugated estrogens, esterified estrogens, and synthetic estrogens. Non-exclusive examples include estrone sulfate, alone or in combination with sodium estrone sulfate, sodium equilin sulfate, sodium 17-alpha dihydroequilin sulfate, estradiol acetate, and ethinyl estradiol. In some embodiments, the pharmaceutical comprises an estrogenic molecule, that is a non-estrogen with estrogenic activity, such as isoflavones, phytoestrogens, diethylstilbestrol, triphenylethylene derivative of tamoxifen without the antiestrogenic side chain, stilbene DES, substituted triphenylethylene trianisyl-chlorethylene, 4OHT, 4OHTPP, adenosine, Chinese Yam, and arbutin. Such a list is provided as an example and should not be interpreted as an exclusive list of possible actives with estrogenic activity.

According to embodiments, the pharmaceutical comprises at least one API that is not an estrogen. Such non-estrogen actives may yield efficacy through absorption into vaginal, labial or vulvar tissue, through topical contact vis-à-vis the spreading mechanism of the disclosed formulation(s), or some combination of both. Such non-estrogen actives may include but should not be considered limited to alpha-lipoic acid, antibiotics, antivirals and/or other microbicidal drugs, antibodies, proteins including peptides, RNA- or DNA-based molecules, vaccines, adrenocorticotropic hormone, angiotensin, beta-endorphin, blood factors, bombesin, calcitonin, calcitonin gene regulating polypeptide, cholecystokinin-8, colony stimulating factors, desmopressin, endothelin, enkephalin, erythropoietins, gastrins, glugagon, human atrial natriuretic polypeptide, interferons, insulin, growth factors, growth hormones, interleukins, luteinizing hormone release hormone, melanocyte stimulating hormone, muramyl-dipeptide, neurotensin, oxytocin, parathyroid hormone, peptide T, secretin, somatomedins, somatostatin, thyroid stimulating hormone, thyrotropin releasing hormone, thyrotropin stimulating hormone, vasoactive intestinal polypeptide, vasopressin, and any analogue or derivative of a compound or chemical listed as an example herein. According to some embodiments, the non-estrogen API may be systemically absorbed. According to alternative embodiments, the non-estrogen API may lack systemic absorption such that there is no statistically significant amount of API detected above that measured in comparison to a placebo.

According to certain embodiments, the pharmaceutical composition or vehicle delivery system is a diagnostic or comprises a diagnostic agent. For example, in addition to or as an alternative to an estrogen or non-estrogen API, the composition contains a coloring agent, for example an ink or dye which may be observed visually or via other imaging techniques known to those skilled in the art of imaging techniques, e.g. a radiopaque agent. Other diagnostic elements could comprise a marker, sensor, or other type of indicator or active capable of providing diagnostic assistance either through a visual change or by changes to the vaginal environment measured by follow-on diagnostic steps (e.g. assay). Other types of diagnostic applications contemplated include the ability to quantify natural discharge (e.g. moisture content of a post-menopause vagina), cancer indicators or cancerous cell identification, vaginal tissue composition (i.e. composition of parabasal vaginal cells, intermediate cells, superficial vaginal cells), measures of epithelial integrity, infection markers (e.g. diagnostics which react to and indicate the presence of yeast or bacterial presence), indicators of clinical or sub-clinical inflammation and the ability to discern healthy, glycogen-rich vaginal tissue from tissue less glycogen-rich, commonly seen in menopausal and post-menopausal women. Such examples of diagnostics are not intended to be exclusive. Those skilled in the art will recognize that the application of the present invention may be directed to a multitude of diagnostics.

According to embodiments, the composition or the fill will have a viscosity of about 50-175, about 60-150, about 65-140, about 70-130, about 75-125, about 80-110 might be some ranges with the reference to about 90 at about 25 degrees C. According to embodiments, the composition or fill comprises a thickener, in some embodiments a thickener surfactant, such as a thickening non-ionic surfactant, which imparts such a viscosity to the fill or composition. According to embodiments, the thickener (e.g., the thickener/surfactant) increases the viscosity of the remainder of the composition, formulation, or fill, such as at least about 0.5×, at least about 1× (100%), at least about 1.5×, at least about 2× (200%), at least about 2.5×, at least about 3×, at least about 3.5×, or at least about 4×. According to embodiments, the thickener is present in a ratio of about 1:6 to about 1:12, such as about 1:8 to about 1:10, such as about 1:9 with respect to the solubilizing agent of the fill, formulation, or composition. According to embodiments, the lack of discharge, the spreading of the composition, or both, is at least about 10%, at least about 20%, at least about 25%, at least about 30%, at least about 50%, at least about 100%, at least about 150%, at least about 200%, or at least about 250% greater due to the presence of the thickening agent. Spreading of compositions of the invention can be determined by directly assessing the presence of the relative active agent, such as estradiol, in the tissue, or can be assessed by evaluating physiological changes known to be causally linked with the presence of the active in the tissue (e.g., labia moisture, color, cellular composition, or lack of dyspareunia is associated with spreading of estradiol).

According to embodiments, a method for treating VVA or one or more symptoms or aspects of VVA, including dyspareunia, vaginal dryness, and estrogen-deficient urinary states (including urinary tract infections), without causing non-natural discharge (e.g., discharge of a pharmaceutical composition or a component thereof) is provided. According to the method, a soft gelatin capsule is administered containing a liquid pharmaceutical composition that is able to be fully absorbed by the vaginal tissue. According to embodiments, the pharmaceutical composition itself is fully absorbed by the vaginal tissue. According to embodiments, the pharmaceutical composition and gelatin capsule are administered in a volume and size, respectively, that allows a subject's vaginal tissue to fully absorb the pharmaceutical composition. According to embodiments, such absorption will occur contemporaneously with the subject being ambulatory. According to the method, the gelatin capsule and liquid pharmaceutical composition are fully absorbed by the vaginal tissue, wherein the only discharge that occurs after estrogenizing the vagina is natural discharge that a woman would have experienced prior to menopause. “Natural” vaginal discharge refers to a small amount of fluid that flows out of the vagina each day, carrying out old cells that have lined the vagina. Natural discharge is usually clear or milky. Non-natural discharge can refer to discharge that is higher in volume than natural discharge, different in color than natural discharge, or different in consistency than natural discharge. Non-natural discharge can also refer to the discharge (e.g., leaking) of a pharmaceutical composition from the vagina. According to embodiments, less than all of the estradiol in the formulation appears in any discharge; that is, any increase of active in the discharge post-administration as compared to pre-treatment would be less than the total amount of active in the formulation. According to embodiments, the amount of estradiol in any discharge does not increase more than about 25%, more than about 15%, more than about 10%, more than about 5%, more than about 2.5%, or more than about 1% in the discharge following administration during some or all of the course of treatment. According to embodiments the total amount of discharge does not increase more than about 25%, more than about 20%, more than about 12.5%, more than about 10%, or more than about 5% following the administration of the product over a period of treatment (e.g., about 2, 4, 6, 8, or 12 weeks) (e.g., as compared to an average amount of discharge before treatment in an individual or in a population of individuals, such as a population used for a statistically significant clinical study, examples of which are provided elsewhere herein). Although compositions of the invention, in embodiments, can spread to external vaginal-associated tissues, such as the labia or other portions of the vulva, that such spreading will not be considered “discharge” with respect to embodiments characterized by a low amount or rate of formulation/API/composition-related discharge or the lack of such treatment-related discharge.

According to embodiments, a method of treating VVA, including dyspareunia, vaginal dryness, and estrogen-deficient urinary states (including urinary tract infections), using a liquid pharmaceutical composition is provided. According to the method, a soft gelatin capsule containing a liquid composition for treating VVA is provided to a subject. The subject inserts the soft gelatin capsule containing the liquid composition for treating VVA into their vagina either digitally or with an applicator, wherein the soft gelatin capsule, when in contact with the vaginal mucosa, dissolves, ruptures, or disintegrates and the liquid composition is released into the vagina. According to embodiments, the liquid composition for treating VVA is a pharmaceutical composition disclosed herein. According to embodiments, the subject inserts the gelatin capsule about two inches into the vagina, or in the third of the vagina closest to the vaginal opening. According to embodiments, the subject is ambulatory immediately after or soon after administration.

According to embodiments, a method is provided for avoiding transport of estradiol to the uterus comprising administration of an estradiol containing composition into the lower third of the vagina closest to the vaginal opening as shown in FIGS. 26A, 26B, and 26C. In this method, the estradiol containing composition releases the estradiol in the lower third of the vagina, which substantially eliminates transport of the estradiol to the uterus, where unopposed estradiol can cause endometrial hyperplasia, which could potentially lead to uterine cancer. In certain embodiments, the pharmaceutical compositions disclosed herein are administered to the lower third of the vagina to prevent transport of the estradiol to the uterus in a manner similar to that disclosed in FIGS. 26A-26C.

According to embodiments, a method is disclosed herein for avoiding transport of estradiol to the uterus of a subject in need of estradiol, the method comprising: administering a pharmaceutical composition comprising estradiol to the subject, wherein the pharmaceutical composition is administered to the lower third of the vagina closest to the vaginal opening. In some embodiments, the pharmaceutical composition is a liquid pharmaceutical composition comprising 4 μg to 25 μg of estradiol, and the liquid pharmaceutical composition is contained in a capsule. In some embodiments, the administering is carried out by digitally inserting the capsule into the lower third of the vagina closest to the vaginal opening. It has been found that delivery of the pharmaceutical compositions disclosed herein to the lower third of the vagina advantageously prevents (i.e., substantially minimized/reduces) transport of the estradiol to the uterus. In certain embodiments, the pharmaceutical compositions further comprising a solubilizing agent. The solubilizing agent can be any oil that is capable of solubilizing estradiol such that the estradiol is at least 80% solubilized, at least 85% solubilized, at least 90% solubilized, at least 95% solubilized, or at least 100% solubilized. In certain embodiments, the oil comprises at least one C6-C12 fatty acid or a glycol, monoglyceride, diglyceride, or triglyceride ester thereof, and preferably predominantly C6-C12 fatty acids or glycol, monoglyceride, diglyceride, or triglyceride esters thereof. In certain embodiments, the oil further comprises a thickener or a surfactant, such as, for example, Tefose63 or Gelucire 44/14.

According to embodiments, a method is disclosed herein for treating VVA, including dyspareunia, vaginal dryness, and estrogen-deficient urinary states (including urinary tract infections), comprising improving the symptoms of VVA, compared to placebo or baseline, within two weeks by vaginally administering a composition for the treatment of VVA. According to embodiments, the composition for the treatment of VVA is a liquid pharmaceutical composition as disclosed herein. According to embodiments, the composition for the treatment of VVA is a liquid containing from 1 μg to 25 μg of estradiol. According to embodiments, the method of administration is a method disclosed herein, including the insertion method shown in FIGS. 26A, 26B, and 26C. According to embodiments, at the two week point of measurement, the estradiol is not detected systemically when measured using standard pharmaceutical pharmacokinetic parameters, such as AUC and Cₘₐₓ.

According to embodiments, a method is disclosed herein for treating VVA, including dyspareunia, vaginal dryness, and estrogen-deficient urinary states (including urinary tract infections), comprising improving the symptoms of VVA, compared to placebo or baseline, within four weeks by vaginally administering a composition for the treatment of VVA. One of skill in the art will understand that the improvements can be assessed statistically as described herein, and that any improvement can be a statistically significant improvement. According to embodiments, the composition for the treatment of VVA is a liquid pharmaceutical composition as disclosed herein. According to embodiments, the composition for the treatment of VVA is a liquid containing from 1 μg to 25 μg of estradiol. According to embodiments, the method of administration is a method disclosed herein, including the insertion method shown in FIGS. 26A, 26B, and 26C. According to embodiments, at the two week point of measurement and/or the four week point of measurement, the estradiol is not detected systemically when measured using standard pharmaceutical pharmacokinetic parameters, such as AUC and Cₘₐₓ.

According to embodiments, a method is disclosed herein for treating VVA, including dyspareunia, vaginal dryness, and estrogen-deficient urinary states (including urinary tract infections), comprising improving the symptoms of VVA, compared to placebo or baseline, within eight weeks by vaginally administering a composition for the treatment of VVA. One of skill in the art will understand that the improvements can be assessed statistically as described herein, and that any improvement can be a statistically significant improvement. According to embodiments, the composition for the treatment of VVA is a liquid pharmaceutical composition as disclosed herein. According to embodiments, the composition for the treatment of VVA is a liquid containing from 1 μg to 25 μg of estradiol. According to embodiments, the method of administration is a method disclosed herein, including the insertion method shown in FIGS. 26A, 26B, and 26C. According to embodiments, at the two week point of measurement and/or the eight week point of measurement, the estradiol is not detected systemically when measured using standard pharmaceutical pharmacokinetic parameters, such as AUC and Cₘₐₓ.

According to embodiments, a method is disclosed herein for treating VVA, including dyspareunia, vaginal dryness, and estrogen-deficient urinary states (including urinary tract infections), comprising improving the symptoms of VVA, compared to placebo or baseline, within ten weeks by vaginally administering a composition for the treatment of VVA. One of skill in the art will understand that the improvements can be assessed statistically as described herein, and that any improvement can be a statistically significant improvement. According to embodiments, the composition for the treatment of VVA is a liquid pharmaceutical composition as disclosed herein. According to embodiments, the composition for the treatment of VVA is a liquid containing from 1 μg to 25 μg of estradiol. According to embodiments, the method of administration is a method disclosed herein, including the insertion method shown in FIGS. 26A, 26B, and 26C. According to embodiments, at the two week point of measurement and/or the ten week point of measurement, the estradiol is not detected systemically when measured using standard pharmaceutical pharmacokinetic parameters, such as AUC and Cₘₐₓ.

According to embodiments, a method for treating VVA, including dyspareunia, vaginal dryness, and estrogen-deficient urinary states (including urinary tract infections), comprising administering a composition containing estradiol for the treatment of VVA is provided, wherein the method improves the symptoms of VVA, compared with baseline or placebo, in at least one of two weeks, four weeks, six weeks, eight weeks, or twelve weeks, wherein the estradiol is not detected systemically using standard pharmaceutical pharmacokinetic parameters, such as AUC and Cₘₐₓ. One of skill in the art will understand that the improvements can be assessed statistically as described herein, and that any improvement can be a statistically significant improvement. According to embodiments, the composition containing estradiol is a liquid composition as disclosed herein. According to embodiments, the composition contains 1 μg to 25 μg of estradiol.

According to embodiments, a method for reestrogenizing the vagina, labia, or vulva is provided, wherein the method comprises administering a composition containing estradiol for the treatment of VVA, wherein the composition is a liquid containing estradiol or a synthetic estrogen, and wherein the liquid spreads over a surface area of the vagina, labia, or vulva which is larger than the area covered by a solid composition. For example, the liquid can spread over a surface area ranging from about 50 cm² to about 120 cm² (e.g., from about 50 cm² to about 60 cm²; or from about 60 cm² to about 70 cm²; or from about 70 cm² to about 80 cm²; or from about 80 cm² to about 90 cm²; or from about 90 cm² to about 100 cm²; or from about 100 cm² to about 110 cm²; or from about 110 cm² to about 120 cm²; or from about 65 cm² to about 110 cm²). According to embodiments, the subject inserts a liquid composition into her vagina in a capsule, such as a hard or soft gelatin capsule, that then dissolves, ruptures, disintegrates, or otherwise releases the liquid in the vagina. According to embodiments, the liquid contains at least one of a bio-adhesive or viscosity enhancer to prevent the liquid from discharging from the vagina before the estradiol or synthetic estrogen can be absorbed into the vaginal tissue in a dose sufficient to effect reestrongenization of the vagina. According to embodiments, a statistically significantly number of subjects receiving a composition of the invention comprising estradiol, synthetic estrogen or other estrogen will have a reestrogenized vagina within two weeks of administration compared to baseline or placebo levels. According to embodiments, a statistically significant number of subjects will experience a reestrogenized vagina reestrogenized within four weeks of administration compared to baseline or placebo levels.

According to embodiments, a method for administering an active agent, such as an active pharmaceutical ingredient, to the vagina, for treating one or more conditions in the vagina, labia, or vulva, wherein the method comprises administering a composition containing a non-estradiol estrogen or non-estrogen for the treatment of disease or a bothersome symptom or for use as a diagnostic, wherein the composition is a liquid containing the active, and wherein the liquid spreads over a surface area of the vagina, labia, or vulva, such that the area of tissue that has come into contact with the composition, formulation, or fill after a period of time (e.g., at least about 3 hours, at least about 6 hours, at least about 12 hours, at least about 18 hours, or at least about 24 hours) is detectably larger (e.g., at least about 20%, at least about 50%, at least about 100% (1×), at least about 2×, at least about 2.5×, at least about 3×, at least about 4×, at least about 5×, or greater than the area initially or previously in contact with the formulation (i.e., before the second period of time). The spread of a non-estradiol estrogen composition of the invention or a non-estrogen API composition of this invention may be similar to that described herein for estradiol. For example, the liquid of such a composition can spread over a surface area ranging from about 50 cm² to about 120 cm² (e.g., from about 50 cm² to about 60 cm²; or from about 60 cm² to about 70 cm²; or from about 70 cm² to about 80 cm²; or from about 80 cm² to about 90 cm²; or from about 90 cm² to about 100 cm²; or from about 100 cm² to about 110 cm²; or from about 110 cm² to about 120 cm²; or from about 65 cm² to about 110 cm²). The spreading of an alternative API could be more or less than the spreading of an estrogen, such as estradiol, the amount of spreading of the relevant API being dependent on the size of the molecule or compound, and how the active interacts with the constituents and anatomy of the vaginal or vaginal-associated tissue environment. According to embodiments, the subject inserts a liquid composition into her vagina in a capsule, such as a hard or soft gelatin capsule, that then dissolves, ruptures, disintegrates, or otherwise releases the liquid in the vagina. According to embodiments, the liquid contains at least one of a bio-adhesive or viscosity enhancer to prevent the liquid from discharging from the vagina before the active can be absorbed into or spread across the vaginal tissue in a dose sufficient to effect treatment of the vagina, labia or vulva.

According to embodiments, a statistically significantly number of subjects will experience a reestrogenized vagina within six weeks of administration compared to baseline or placebo levels. According to embodiments, a statistically significantly number of subjects will experience a reestrogenized vagina within eight weeks of administration compared to baseline or placebo levels. According to embodiments, a statistically significantly number of subjects will experience a reestrogenized vagina within ten weeks of administration compared to baseline or placebo levels. According to embodiments, a statistically significantly number of subjects will experience a reestrogenized vagina within twelve or more weeks of administration compared to baseline or placebo levels.

### VII. MEASUREMENT OF EFFICACY

According to embodiments, administration of the pharmaceutical compositions described herein results in treatment of VVA. Patients with VVA experience shrinking of the vaginal canal in both length and diameter and the vaginal canal has fewer glycogen-rich vaginal cells to maintain moisture and suppleness. In addition, the vaginal wall can become thin, pale, dry, or sometimes inflamed (atrophic vaginitis). These changes can manifest as a variety of symptoms collectively referred to as VVA. Such symptoms include, without limitations, an increase in vaginal pH; reduction of vaginal epithelial integrity, vaginal secretions, or epithelial surface thickness; pruritus; vaginal dryness; dyspareunia (pain or bleeding during sexual intercourse); urinary tract infections; or a change in vaginal color. According to embodiments, efficacy of the method or composition of the invention is measured as a reduction of vulvar and vaginal atrophy in a patient back to premenopausal conditions. According to embodiments, the change is measured as a reduction in the severity of one or more atrophic effects measured at baseline (screening, Day 1) and compared to a measurement taken at Day 15 (end of treatment). Severity of the atrophic effect may be measured using a scale of 0 to 3 where, for example, none=0, mild=1, moderate=2, or severe=3. Such scoring is implemented to evaluate the pre-treatment condition of patients; to determine the appropriate course of a treatment regime; such as dosage, dosing frequency, and duration, among others; and post-treatment outcomes.

One of the symptoms of VVA is increased vaginal pH. In further aspects of this disclosure, treatment with the pharmaceutical compositions described herein resulted in a decrease in vaginal pH. A decrease in vaginal pH is measured as a decrease from the vaginal pH at baseline (screening) to the vaginal pH at Day 15, according to embodiments. In some embodiments, a pH of 5 or greater may be associated with VVA. In some embodiments, pH is measured using a pH indicator strip placed against the vaginal wall. In some embodiments, a change in vaginal pH is a change in a patient's vaginal pH to a pH of less than about pH 5.0. In some embodiments, a subject's vaginal pH may be less than about pH 4.9, pH 4.8, pH 4.7, pH 4.6, pH 4.5, pH 4.4, pH 4.3, pH 4.2, pH 4.1, pH 4.0, pH 3.9, pH 3.8, pH 3.7, pH 3.6, or pH 3.5. According to certain embodiments, a decrease in vaginal pH is seen within 12 weeks of beginning treatment with the compositions described herein.

According to embodiments, treatment with the pharmaceutical compositions described herein results in improvements in the vaginal Maturation Index. The Maturation Index is measured as a change in cell composition. According to embodiments and as related to VVA, a change in cell composition is measured as the change in percent of composition or amount of parabasal vaginal cells, intermediate cells, and superficial vaginal cells, such as a change in the composition or amount of parabasal vaginal cells compared with or, relative to, a change in superficial vaginal cells. A subject having VVA symptoms often has an increased number of parabasal cells and a reduced number of superficial cells (e.g., less than about 5%) compared with women who do not suffer from VVA. Conversely, a subject having decreasing VVA symptoms, or as otherwise responding to treatment, may demonstrate an improvement in the Maturation Index, specifically a decrease in the amount of parabasal cells or an increase in the amount of superficial cells compared to baseline (screening). In embodiments, a decrease in parabasal cells is measured as a reduction in the percent of parabasal cells; the percent reduction may be at least about an 85%, 80%, 75%, 70%, 65%, 60%, 55%, 50%, 45%, 40%, 35%, 30%, 25%, 20%, 15% or 10% reduction in the number of parabasal cells. In embodiments, a percent reduction may be at least about a 54% reduction in the number of parabasal cells. In embodiments, an increase in superficial cells is measured as an increase in the percent of superficial cells; the percent increase in superficial cells may be at least about 5%, 10%, 15%, 20%, 25%, 30%, 35%, 40%, 45%, or 50% increase in the number of superficial cells. In further embodiments, a percent increase may be at least about a 35% increase in the number of superficial cells. According to certain embodiments, an increase in superficial cell populations are seen within 12 weeks of beginning treatment with the compositions described herein. According to certain embodiments, a decrease in parabasal cell populations are seen within 12 weeks of beginning treatment with the compositions described herein.

In some embodiments, an improvement in the Maturation Index is assessed as a change over time. For example, as a change in cell composition measured at a baseline (screening) at Day 1 compared to the cell composition measured at Day 15. The change in cell composition may also be assessed as a change in the amount of parabasal cells over time, optionally in addition to measuring changes in parabasal cells and superficial cells as described above. Such cells may be obtained from the vaginal mucosal epithelium through routine gynecological examination and examined by means of a vaginal smear.

In various further aspects of this disclosure, treatment with the pharmaceutical compositions described herein resulted in any of: an increase in superficial cells; a decrease in parabasal cells; and an increase in intermediate cells.

In further aspects of this disclosure, samples may be collected to determine hormone levels, in particular, estradiol levels. In some embodiments, blood samples may be taken from a subject and the level of estradiol measured (pg/mL). In some embodiments, estradiol levels may be measured at 0 hours (for example, at time of first treatment), at 1 hour (for example, post first treatment), at 3 hours, and at 6 hours. In some embodiments, samples may be taken at day 8 (for example, post first treatment) and at day 15 (for example, one day post the last treatment on day 14). In some embodiments, descriptive statistics of plasma estradiol concentrations at each sampling time and observed Cₘₐₓ and Tₘₐₓ values may be measured and the AUC calculated.

In some embodiments, a suppository can comprise about 25 μg of estradiol. In such cases, administration of the suppository to a patient can provide, in a plasma sample from the patient, parameters including one or more parameters selected from: 1) a corrected geometric mean peak plasma concentration (Cₘₐₓ) of estradiol of about 19 pg*hr/mL to about 29 pg*hr/mL (e.g., 19.55 pg*hr/mL to about 28.75 pg*hr/mL); or 2) a corrected geometric mean area under the curve (AUC)₀₋₂₄ of estradiol of about 75 pg*hr/mL to about 112 pg*hr/mL (e.g., 75.82 pg*hr/mL to about 111.50). In some embodiments, administration of the suppository to a patient provides, in a plasma sample from the patient, one or more parameters selected from: 1) a corrected geometric mean peak plasma concentration (Cₘₐₓ) of estrone of about 9 pg*hr/mL to about 14 pg*hr/mL (e.g., 9.17 pg*hr/mL to about 13.49 pg*hr/mL); and 2) a corrected geometric mean area under the curve (AUC)₀₋₂₄ of estrone of about 43 pg*hr/mL to about 65 pg*hr/mL (e.g., 43.56 pg*hr/mL to about 64.06 pg*hr/mL). In some embodiments, administration of the suppository to a patient provides, in a plasma sample from the patient, provides one or more parameters selected from: 1) a corrected geometric mean peak plasma concentration (Cₘₐₓ) of estrone sulfate of about 416 pg*hr/mL to about 613 pg*hr/mL (e.g., 416.53 pg*hr/mL to about 612.55 pg*hr/mL); and 2) a corrected geometric mean area under the curve (AUC)₀₋₂₄ of estrone sulfate of about 3598 pg*hr/mL to about 5291 pg*hr/mL (e.g., 3598.04 pg*hr/mL to about 5291.24 pg*hr/mL).

In some embodiments, a suppository includes about 25 μg of estradiol. In some such embodiments, administration of the suppository to a patient can provide, in a plasma sample from the patient, parameters including one or more parameters selected from: 1) a corrected geometric mean peak plasma concentration (Cₘₐₓ) of estradiol ranging from about 20.9 pg/mL to about 32.8 pg/mL (e.g., 20.96 pg/mL to about 32.75 pg/mL); 2) a corrected geometric mean area under the curve (AUC)₀₋₂₄ of estradiol ranging from about 104.3 pg*hr/mL to about 163.1 pg*hr/mL (e.g., 104.32 pg*hr/mL to about 163.0 pg*hr/mL); and 3) an average concentration (C_{avg}) of estradiol ranging from about 4.3 pg/mL to about 6.8 pg/mL (e.g., 4.32 pg/mL to about 6.75 pg/mL), as assessed at day 1.

In some embodiments, administration of a suppository comprising about 25 μg of estradiol to a patient can provide, in a plasma sample from the patient, parameters including one or more parameters selected from: 1) a corrected geometric mean peak plasma concentration (Cₘₐₓ) of estradiol of about 26.2 pg/mL; 2) a corrected geometric mean area under the curve (AUC)₀₋₂₄ of estradiol of about 130 pg*hr/mL; and 3) an average concentration (C_{avg}) of estradiol of about 5.4 pg/mL, as assessed at day 1.

In some embodiments, administration of a suppository comprising about 25 μg of estradiol to a patient can provide, in a plasma sample from the patient, parameters including one or more parameters selected from: 1) a corrected geometric mean peak plasma concentration (Cₘₐₓ) of estradiol ranging from about 9.5 pg/mL to about 15.1 pg/mL (e.g., 9.60 pg*hr/mL to about 15.00 pg/mL); 2) a corrected geometric mean area under the curve (AUC)₀₋₂₄ of estradiol ranging from about 67.6 pg*hr/mL to about 105.8 pg*hr/mL (e.g., 67.68 pg*hr/mL to about 105.75 pg*hr/mL); and 3) an average concentration (C_{avg}) of estradiol ranging from about 2.7 pg/mL to about 4.4 pg/mL (e.g., 2.80 pg/mL to about 4.38 pg/mL) of estradiol as assessed at day 14.

In some embodiments, administration of a suppository comprising about 25 μg of estradiol to a patient can provide, in a plasma sample from the patient, parameters including one or more parameters selected from: 1) a corrected geometric mean peak plasma concentration (Cₘₐₓ) of estradiol of about 12.0 pg/mL; 2) a corrected geometric mean area under the curve (AUC)₀₋₂₄ of estradiol of about 84.6 pg*hr/mL; and 3) an average concentration (C_{avg}) of estradiol of about 3.5 pg/mL, as assessed at day 14.

In some embodiments, administration of a suppository comprising about 25 μg of estradiol to a patient provides, in a plasma sample from the patient, one or more parameters selected from: 1) a corrected geometric mean peak plasma concentration (Cₘₐₓ) of estrone conjugates ranging from about 158.8 pg/mL to about 248.3 pg/mL (e.g., 158.88 hr/mL to about 248.25 pg*hr/mL); and 2) a corrected geometric mean area under the curve (AUC)₀₋₂₄ of estrone conjugates ranging from about 1963.1 pg*hr/mL to about 3067.6 pg*hr/mL (e.g., 1963.20 pg*hr/mL to about 3067.50 pg*hr/mL) as assessed at day 1.

In some embodiments, administration of a suppository comprising about 25 μg of estradiol to a patient provides, in a plasma sample from the patient, one or more parameters selected from: 1) a corrected geometric mean peak plasma concentration (Cₘₐₓ) of estrone conjugates of about 198.6 pg/mL; and 2) a corrected geometric mean area under the curve (AUC)₀₋₂₄ of estrone conjugates of about 2454 pg*hr/mL as assessed at day 1.

In some embodiments, administration of a suppository comprising about 25 μg of estradiol to a patient provides, in a plasma sample from the patient, one or more parameters selected from: 1) an unadjusted arithmetic mean area under the curve (AUC)₀₋₂₄ of estradiol ranging from about 173.5 pg*hr/mL to about 271.3 pg*hr/mL (e.g., from 173.60 pg*hr/mL to about 271.25 pg*hr/mL; or about 217 pg*hr/mL), as assessed at day 1; 2) a corrected arithmetic mean peak plasma concentration (C_{avg[0-24]}) of estradiol ranging from about 7.2 pg/mL to about 11.4 pg/mL (e.g., from 7.25 pg/mL to about 11.33 pg/mL; or about 9.06 pg/mL), as assessed at day 1; 3) an unadjusted arithmetic mean area under the curve (AUC)₀₋₂₄ of estradiol ranging from about 137.5 pg*hr/mL to about 215.1 pg*hr/mL (e.g., from 137.60 pg*hr/mL to about 215.00 pg*hr/mL; or about 172 pg*hr/mL), as assessed at day 14; and 4) a corrected arithmetic mean peak plasma concentration (C_{avg[0-24]}) of estradiol ranging from about 5.7 pg/mL to about 9.0 pg/mL (e.g., from 5.72 pg/mL to about 8.94 pg/mL; or about 7.15 pg/mL), as assessed at day 14.

In some embodiments, administration of a suppository comprising about 25 μg of estradiol to a patient provides, in a plasma sample from the patient, one or more parameters selected from: 1) an unadjusted arithmetic mean area under the curve (AUC)₀₋₂₄ of estrone ranging from about 335.1 pg*hr/mL to about 523.8 pg*hr/mL (e.g., from 335.20 pg*hr/mL to about 523.75 pg*hr/mL; or about 419 pg*hr/mL), as assessed at day 1; 2) a corrected arithmetic mean peak plasma concentration (C_{avg[0-24]}) of estrone ranging from about 13.9 pg/mL to about 21.9 pg/mL (e.g., from 14.00 pg/mL to about 21.88 pg/mL; or about 17.5 pg/mL), as assessed at day 1; 3) an unadjusted arithmetic mean area under the curve (AUC)₀₋₂₄ of estrone ranging from about 343.1 pg*hr/mL to about 536.2 pg*hr/mL (e.g., from 343.20 pg*hr/mL to about 536.25 pg*hr/mL; or about 429 pg*hr/mL), as assessed at day 14; and 4) a corrected arithmetic mean peak plasma concentration (C_{avg[0-24]}) of estrone ranging from about 14.3 pg/mL to about 22.4 pg/mL (e.g., from 14.32 pg/mL to about 22.38 pg/mL; or about 17.9 pg/mL), as assessed at day 14.

In some embodiments, administration of a suppository comprising about 25 μg of estradiol to a patient provides, in a plasma sample from the patient, one or more parameters selected from: 1) an unadjusted arithmetic mean area under the curve (AUC)₀₋₂₄ of estrone conjugates ranging from about 7,300.7 pg*hr/mL to about 11,407.6 pg*hr/mL (e.g., from 7,300.80 pg*hr/mL to about 11,407.50 pg*hr/mL; or about 9,126 pg*hr/mL), as assessed at day 1; 2) a corrected arithmetic mean peak plasma concentration (C_{avg[0-24]}) of estrone conjugates ranging from about 303.9 pg/mL to about 475.1 pg/mL (e.g., from 304.00 pg/mL to about 475.00 pg/mL; or about 380 pg/mL), as assessed at day 1; 3) an unadjusted arithmetic mean area under the curve (AUC)₀₋₂₄ of estrone conjugates ranging from about 7,943.9 pg*hr/mL to about 12,412.6 pg*hr/mL (e.g., from 7,944.00 pg*hr/mL to about 12,412.50 pg*hr/mL; or about 9,930 pg*hr/mL), as assessed at day 14; and 4) a corrected arithmetic mean peak plasma concentration (C_{avg[0-24]}) of estrone conjugates ranging from about 331.1 pg/mL to about 517.4 pg/mL (e.g., from 331.20 pg/mL to about 517.50 pg/mL; or about 414 pg/mL), as assessed at day 14.

In some embodiments, a suppository can comprise about 10 μg of estradiol. In such cases, administration of the suppository to a patient can provide, in a plasma sample from the patient, one or more parameters selected from: 1) a corrected geometric mean peak plasma concentration (Cₘₐₓ) of estradiol of about 12 pg*hr/mL to about 18 pg*hr/mL (e.g., 12.22 pg*hr/mL to about 17.98 pg*hr/mL); 2) a corrected geometric mean area under the curve (AUC)₀₋₂₄ of estradiol of about 42 pg*hr/mL to about 63 pg*hr/mL (e.g., 42.18 pg*hr/mL to about 62.02 pg*hr/mL); and 3) a corrected geometric mean time to peak plasma concentration (Tₘₐₓ) of estradiol of about 1 hrs to about 3 hrs (e.g., 1.49 hrs to about 2.19 hrs). In some embodiments, administration of the suppository to a patient provides, in a plasma sample from the patient, one or more parameters selected from: 1) a corrected geometric mean peak plasma concentration (Cₘₐₓ) of estrone of about 4 pg*hr/mL to about 7 pg*hr/mL (e.g., 4.38 pg*hr/mL to about 6.44 pg*hr/mL); 2) a corrected geometric mean area under the curve (AUC)₀₋₂₄ of estrone of about 20 pg*hr/mL to about 31 pg*hr/mL (e.g., 20.60 pg*hr/mL to about 30.30 pg*hr/mL); and 3) a corrected geometric mean time to peak plasma concentration (Tₘₐₓ) of estrone of about 4 hrs to about 8 hrs (e.g., 4.99 hrs to about 7.34 hrs). In some embodiments, administration of the suppository to a patient provides, in a plasma sample from the patient, one or more parameters selected from: 1) a corrected geometric mean peak plasma concentration (Cₘₐₓ) of estrone sulfate of about 10 pg*hr/mL to about 16 pg*hr/mL (e.g., 10.34 pg*hr/mL to about 15.20 pg*hr/mL); 2) a corrected geometric mean area under the curve (AUC)₀₋₂₄ of estrone sulfate of about 56 pg*hr/mL to about 84 pg*hr/mL (e.g., 56.61 pg*hr/mL to about 83.25 pg*hr/mL); and 3) a corrected geometric mean time to peak plasma concentration (Tₘₐₓ) of estrone sulfate of about 4 hrs to about 7 hrs (e.g., 4.67 hrs to about 6.86 hrs).

In some embodiments, a suppository includes about 10 μg of estradiol. In some such embodiments, administration of the suppository to a patient can provide, in a plasma sample from the patient, a corrected geometric mean peak plasma concentration (Cₘₐₓ) of estradiol ranging from about 4.7 pg/mL to about 7.6 pg/mL (e.g., 4.80 pg*hr/mL to about 7.50 pg*hr/mL), as assessed at day 1. In some embodiments, administration of a suppository comprising about 10 μg of estradiol to a patient can provide, in a plasma sample from the patient, a corrected geometric mean peak plasma concentration (Cₘₐₓ) of estradiol ranging from about 2.3 pg*hr/mL to about 3.8 pg*hr/mL (e.g., 2.40 pg*hr/mL to about 3.75 pg*hr/mL) of estradiol as assessed at day 14.

In some embodiments, administration of a suppository comprising about 10 μg of estradiol to a patient provides, in a plasma sample from the patient, a corrected geometric mean peak plasma concentration (Cₘₐₓ) of estradiol of about 6.0 pg/mL, as assessed at day 1. In some embodiments, administration of a suppository comprising about 10 μg of estradiol to a patient can provide, in a plasma sample from the patient, a corrected geometric mean peak plasma concentration (Cₘₐₓ) of estradiol of about 3.0 pg/mL, as assessed at day 14.

In some embodiments, administration of a suppository comprising about 10 μg of estradiol to a patient provides, in a plasma sample from the patient, a corrected geometric mean area under the curve (AUC)₀₋₂₄ of estradiol ranging from about 17.5 pg/mL to about 27.4 pg/mL (e.g., 17.52 pg*hr/mL to about 27.37 pg*hr/mL), as assessed at day 1. In some embodiments, administration of a suppository comprising about 10 μg of estradiol to a patient can provide, in a plasma sample from the patient, a corrected geometric mean area under the curve (AUC)₀₋₂₄ of estradiol ranging from about 10.9 pg*hr/mL to about 17.2 pg*hr/mL (e.g., 10.96 pg*hr/mL to about 17.13 pg*hr/mL) of estradiol as assessed at day 14.

In some embodiments, administration of a suppository comprising about 10 μg of estradiol to a patient provides, in a plasma sample from the patient, a corrected geometric mean area under the curve (AUC)₀₋₂₄ of estradiol of about 21.9 pg*hr/mL, as assessed at day 1. In some embodiments, administration of a suppository comprising about 10 μg of estradiol to a patient can provide, in a plasma sample from the patient, a corrected geometric mean area under the curve (AUC)₀₋₂₄ of estradiol of about 13.7 pg*hr/mL, as assessed at day 14.

In some embodiments, administration of a suppository comprising about 10 μg of estradiol to a patient provides, in a plasma sample from the patient, an average concentration (C_{avg}) of estradiol ranging from about 0.6 pg/mL to about 1.1 pg/mL (e.g., 0.64 pg/mL to about 1.0 pg/mL), as assessed at day 1. In some embodiments, administration of a suppository comprising about 10 μg of estradiol to a patient can provide, in a plasma sample from the patient, an average concentration (C_{avg}) of estradiol ranging from about 0.1 pg/mL to about 0.3 pg/mL (e.g., 0.16 pg/mL to about 0.25 pg/mL) of estradiol as assessed at day 14.

In some embodiments, administration of a suppository comprising about 10 μg of estradiol to a patient provides, in a plasma sample from the patient, an average concentration (C_{avg}) of estradiol of about 0.8 pg/mL, as assessed at day 1. In some embodiments, administration of a suppository comprising about 10 μg of estradiol to a patient can provide, in a plasma sample from the patient, an average concentration (C_{avg}) of estradiol of about 0.2 pg/mL, as assessed at day 14.

In some embodiments, administration of a suppository comprising about 10 μg of estradiol to a patient provides, in a plasma sample from the patient, one or more parameters selected from: 1) a corrected geometric mean peak plasma concentration (Cₘₐₓ) of estrone conjugates ranging from about 72.1 pg/mL to about 112.8 pg/mL (e.g., 72.16 pg/mL to about 112.75 pg/mL); and 2) an average concentration (C_{avg}) of estrone conjugates ranging from about 6.3 pg/mL to about 10.1 pg/mL (e.g., 6.40 pg/mL to about 10.00 pg/mL) as assessed at day 1.

In some embodiments, administration of a suppository comprising about 10 μg of estradiol to a patient provides, in a plasma sample from the patient, one or more parameters selected from: 1) a corrected geometric mean peak plasma concentration (Cₘₐₓ) of estrone conjugates of about 90.2 pg/mL; and 2) an average concentration (C_{avg}) of estrone conjugates of about 8.0 pg/mL, as assessed at day 1.

In some embodiments, administration of a suppository comprising about 10 μg of estradiol to a patient provides, in a plasma sample from the patient, one or more parameters selected from: 1) an unadjusted arithmetic mean area under the curve (AUC)₀₋₂₄ of estradiol ranging from about 110.3 pg*hr/mL to about 172.6 pg*hr/mL (e.g., from 110.40 pg*hr/mL to about 172.50 pg*hr/mL; or about 138 pg*hr/mL), as assessed at day 1; 2) a corrected arithmetic mean peak plasma concentration (C_{avg[0-24]}) of estradiol ranging from about 4.6 pg/mL to about 7.8 pg/mL (e.g., from 4.61 pg/mL to about 7.20 pg/mL; or about 5.76 pg/mL), as assessed at day 1; 3) an unadjusted arithmetic mean area under the curve (AUC)₀₋₂₄ of estradiol ranging from about 87.9 pg*hr/mL to about 137.4 pg*hr/mL (e.g., from 88.00 pg*hr/mL to about 137.50 pg*hr/mL; or about 110 pg*hr/mL), as assessed at day 14; and 4) a corrected arithmetic mean peak plasma concentration (C_{avg[0-24]}) of estradiol ranging from about 3.6 pg/mL to about 5.8 pg/mL (e.g., from 3.67 pg/mL to about 5.74 pg/mL; or about 4.59 pg/mL), as assessed at day 14.

In some embodiments, administration of a suppository comprising about 10 μg of estradiol to a patient provides, in a plasma sample from the patient, one or more parameters selected from: 1) an unadjusted arithmetic mean area under the curve (AUC)₀₋₂₄ of estrone ranging from about 370.3 pg*hr/mL to about 578.8 pg*hr/mL (e.g., from 370.40 pg*hr/mL to about 578.75 pg*hr/mL; or about 463 pg*hr/mL), as assessed at day 1; 2) a corrected arithmetic mean peak plasma concentration (C_{avg[0-24]}) of estrone ranging from about 15.4 pg/mL to about 24.2 pg/mL (e.g., from 15.44 pg/mL to about 24.13 pg/mL; or about 19.3 pg/mL), as assessed at day 1; 3) an unadjusted arithmetic mean area under the curve (AUC)₀₋₂₄ of estrone ranging from about 371.1 pg*hr/mL to about 580.1 pg*hr/mL (e.g., from 371.20 pg*hr/mL to about 580.00 pg*hr/mL; or about 464 pg*hr/mL), as assessed at day 14; and 4) a corrected arithmetic mean peak plasma concentration (C_{avg[0-24]}) of estrone ranging from about 15.4 pg/mL to about 24.2 pg/mL (e.g., from 15.44 pg/mL to about 24.13 pg/mL; or about 19.3 pg/mL), as assessed at day 14.

In some embodiments, administration of a suppository comprising about 10 μg of estradiol to a patient provides, in a plasma sample from the patient, one or more parameters selected from: 1) an unadjusted arithmetic mean area under the curve (AUC)₀₋₂₄ of estrone conjugates ranging from about 4,745.5 pg*hr/mL to about 7,414.9 pg*hr/mL (e.g., from 4,745.60 pg*hr/mL to about 7,415.00 pg*hr/mL; or about 5,932 pg*hr/mL), as assessed at day 1; 2) a corrected arithmetic mean peak plasma concentration (C_{avg[0-24]}) of estrone conjugates ranging from about 197.5 pg/mL to about 308.8 pg/mL (e.g., from 197.60 pg/mL to about 308.75 pg/mL; or about 247 pg/mL), as assessed at day 1; 3) an unadjusted arithmetic mean area under the curve (AUC)₀₋₂₄ of estrone conjugates ranging from about 7,182.3 pg*hr/mL to about 11,222.6 pg*hr/mL (e.g., from 7,182.40 pg*hr/mL to about 11,222.50 pg*hr/mL; or about 8,978 pg*hr/mL), as assessed at day 14; and 4) a corrected arithmetic mean peak plasma concentration (C_{avg[0-24]}) of estrone conjugates ranging from about 299.1 pg/mL to about 467.6 pg/mL (e.g., from 299.20 pg/mL to about 467.50 pg/mL; or about 374 pg/mL), as assessed at day 14.

In some embodiments, a suppository can comprise about 4 μg of estradiol. In such cases, administration of the suppository to a patient can provide, in a plasma sample from the patient, one or more parameters selected from: 1) a corrected geometric mean peak plasma concentration (Cₘₐₓ) of estradiol of about 4 pg*hr/mL to about 8 pg*hr/mL; 2) a corrected geometric mean area under the curve (AUC)₀₋₂₄ of estradiol of about 16 pg*hr/mL to about 26 pg*hr/mL; and 3) a corrected geometric mean time to peak plasma concentration (Tₘₐₓ) of estradiol of about 0.25 hrs to about 2 hrs. In some embodiments, administration of the suppository to a patient provides, in a plasma sample from the patient, one or more parameters selected from: 1) a corrected geometric mean peak plasma concentration (Cₘₐₓ) of estrone of about 1 pg*hr/mL to about 3 pg*hr/mL; 2) a corrected geometric mean area under the curve (AUC)₀₋₂₄ of estrone of about 8 pg*hr/mL to about 13 pg*hr/mL; and 3) a corrected geometric mean time to peak plasma concentration (Tₘₐₓ) of estrone of about 1 hrs to about 4 hrs. In some embodiments, administration of the suppository to a patient provides, in a plasma sample from the patient, one or more parameters selected from: 1) a corrected geometric mean peak plasma concentration (Cₘₐₓ) of estrone sulfate of about 4 pg*hr/mL to about 7 pg*hr/mL; 2) a corrected geometric mean area under the curve (AUC)₀₋₂₄ of estrone sulfate of about 22 pg*hr/mL to about 34 pg*hr/mL; and 3) a corrected geometric mean time to peak plasma concentration (Tₘₐₓ) of estrone sulfate of about 1 hrs to about 3 hrs.

In some embodiments, a suppository includes about 4 μg of estradiol. In some such embodiments, administration of the suppository to a patient can provide, in a plasma sample from the patient, one or more parameters selected from: 1) a corrected geometric mean peak plasma concentration (Cₘₐₓ) of estradiol ranging from about 2.0 pg/mL to about 3.3 pg/mL (e.g., 2.08 pg*hr/mL to about 3.25 pg*hr/mL); and 2) a corrected geometric mean area under the curve (AUC)₀₋₂₄ of estradiol ranging from about 9.5 pg*hr/mL to about 15.1 pg*hr/mL (e.g., 9.60 pg*hr/mL to about 15.0 pg*hr/mL), as assessed at day 1. In some embodiments, administration of a suppository comprising about 4 μg of estradiol to a patient can provide, in a plasma sample from the patient, one or more parameters selected from: 1) a corrected geometric mean peak plasma concentration (Cₘₐₓ) of estradiol ranging from about 1.0 pg*hr/mL to about 1.7 pg*hr/mL (e.g., 1.04 pg*hr/mL to about 1.63 pg*hr/mL) of estradiol, and 2) a corrected geometric mean area under the curve (AUC)₀₋₂₄ of estradiol ranging from about 5.7 pg*hr/mL to about 9.1 pg*hr/mL (e.g., 5.76 pg*hr/mL to about 9.0 pg*hr/mL).

In some embodiments, administration of a suppository comprising about 4 μg of estradiol to a patient provides, in a plasma sample from the patient, one or more parameters selected from: 1) a corrected geometric mean peak plasma concentration (Cₘₐₓ) of estradiol of about 2.6 pg/mL; and 2) a corrected geometric mean area under the curve (AUC)₀₋₂₄ of estradiol of about 12 pg*hr/mL, as assessed at day 1. In some embodiments, administration of a suppository comprising about 10 μg of estradiol to a patient can provide, in a plasma sample from the patient, one or more parameters selected from: 1) a corrected geometric mean peak plasma concentration (Cₘₐₓ) of estradiol of about 1.3 pg/mL; 2) a corrected geometric mean area under the curve (AUC)₀₋₂₄ of estradiol of about 7.2 pg*hr/mL, as assessed at day 14.

In some embodiments, administration of a suppository comprising about 4 μg of estradiol to a patient provides, in a plasma sample from the patient, a corrected geometric mean peak plasma concentration (Cₘₐₓ) of estrone conjugates ranging from about 0.3 pg/mL to about 0.5 pg/mL (e.g., 0.32 pg/mL to about 0.5 pg/mL) as assessed at day 1.

In some embodiments, administration of a suppository comprising about 4 μg of estradiol to a patient provides, in a plasma sample from the patient, a corrected geometric mean peak plasma concentration (Cₘₐₓ) of estrone conjugates of about 0.4 pg/mL as assessed at day 1.

In some embodiments, administration of a suppository comprising about 4 μg of estradiol to a patient provides, in a plasma sample from the patient, one or more parameters selected from: 1) an unadjusted arithmetic mean area under the curve (AUC)₀₋₂₄ of estradiol ranging from about 73.3 pg*hr/mL to about 114.7 pg*hr/mL (e.g., from 73.36 pg*hr/mL to about 114.63 pg*hr/mL; or about 91.7 pg*hr/mL), as assessed at day 1; 2) a corrected arithmetic mean peak plasma concentration (C_{avg[0-24]}) of estradiol ranging from about 3.1 pg/mL to about 4.8 pg/mL (e.g., from 3.14 pg/mL to about 4.90 pg/mL; or about 3.92 pg/mL), as assessed at day 1; 3) an unadjusted arithmetic mean area under the curve (AUC)₀₋₂₄ of estradiol ranging from about 69.7 pg*hr/mL to about 108.9 pg*hr/mL (e.g., from 69.76 pg*hr/mL to about 109.00 pg*hr/mL; or about 87.2 pg*hr/mL), as assessed at day 14; and 4) a corrected arithmetic mean peak plasma concentration (C_{avg[0-24]}) of estradiol ranging from about 2.8 pg/mL to about 4.6 pg/mL (e.g., from 2.90 pg/mL to about 4.54 pg/mL; or about 3.63 pg/mL), as assessed at day 14.

In some embodiments, administration of a suppository comprising about 4 μg of estradiol to a patient provides, in a plasma sample from the patient, one or more parameters selected from: 1) an unadjusted arithmetic mean area under the curve (AUC)₀₋₂₄ of estrone ranging from about 231.9 pg*hr/mL to about 362.4 pg*hr/mL (e.g., from 232.00 pg*hr/mL to about 362.50 pg*hr/mL; or about 290 pg*hr/mL), as assessed at day 1; 2) a corrected arithmetic mean peak plasma concentration (C_{avg[0-24]}) of estrone ranging from about 10.3 pg/mL to about 16.3 pg/mL (e.g., from 10.40 pg/mL to about 16.25 pg/mL; or about 13 pg/mL), as assessed at day 1; 3) an unadjusted arithmetic mean area under the curve (AUC)₀₋₂₄ of estrone ranging from about 261.5 pg*hr/mL to about 408.8 pg*hr/mL (e.g., from 261.60 pg*hr/mL to about 408.75 pg*hr/mL; or about 327 pg*hr/mL), as assessed at day 14; and 4) a corrected arithmetic mean peak plasma concentration (C_{avg[0-24]}) of estrone ranging from about 10.8 pg/mL to about 17.1 pg/mL (e.g., from 10.88 pg/mL to about 17.00 pg/mL; or about 13.6 pg/mL), as assessed at day 14.

In some embodiments, administration of a suppository comprising about 4 μg of estradiol to a patient provides, in a plasma sample from the patient, one or more parameters selected from: 1) an unadjusted arithmetic mean area under the curve (AUC)₀₋₂₄ of estrone conjugates ranging from about 4,062.3 pg*hr/mL to about 6,347.6 pg*hr/mL (e.g., from 4,062.40 pg*hr/mL to about 6,347.50 pg*hr/mL; or about 5,078 pg*hr/mL), as assessed at day 1; 2) a corrected arithmetic mean peak plasma concentration (C_{avg[0-24]}) of estrone conjugates ranging from about 172.7 pg/mL to about 270.1 pg/mL (e.g., from 172.80 pg/mL to about 270.00 pg/mL; or about 216 pg/mL), as assessed at day 1; 3) an unadjusted arithmetic mean area under the curve (AUC)₀₋₂₄ of estrone conjugates ranging from about 4,138.3 pg*hr/mL to about 6,466.3 pg*hr/mL (e.g., from 4,138.40 pg*hr/mL to about 6,466.25 pg*hr/mL; or about 5173 pg*hr/mL), as assessed at day 14; and 4) a corrected arithmetic mean peak plasma concentration (C_{avg[0-24]}) of estrone conjugates ranging from about 172.7 pg/mL to about 270.1 pg/mL (e.g., from 172.80 pg/mL to about 270.00 pg/mL; or about 216 pg/mL), as assessed at day 14.

A pharmaceutical composition provided herein can result in substantially local delivery of estradiol. For example, plasma concentrations of estradiol, estrone, and estrone sulfate measured in the plasma of a patient following administration of a pharmaceutical composition as provided herein be statistically similar to those measured following administration of a placebo formulation (i.e., a similar formulation lacking the estradiol). Accordingly, in some embodiments, the plasma concentrations of estradiol, estrone, or estrone sulfate measured following administration of a pharmaceutical composition provided herein may be low compared to RLD formulations.

In some embodiments, a suppository can include about 1 μg to about 25 μg of estradiol. Upon administration the suppository to a patient, a plasma sample from the patient can provide a corrected geometric mean peak plasma concentration (Cₘₐₓ) of estradiol that is less than about 30 pg*hr/mL. For example, administration of the suppository to a patient provides a corrected geometric mean peak plasma concentration (Cₘₐₓ) of estradiol that is less than about 18 pg*hr/mL. In some embodiments, administration of the suppository to a patient provides a corrected geometric mean area under the curve (AUC)0-24 of estradiol that is less than about 112 pg*hr/mL. For example, administration of the suppository to a patient provides a corrected geometric mean area under the curve (AUC)0-24 of estradiol that is less than about 63 pg*hr/mL.

In some embodiments, administration of the suppository to a patient provides a corrected geometric mean peak plasma concentration (Cₘₐₓ) of estrone that is less than about 14 pg*hr/mL. For example, administration of the suppository to a patient provides a corrected geometric mean peak plasma concentration (Cₘₐₓ) of estrone that is less than about 7 pg*hr/mL. In some embodiments, administration of the suppository to a patient provides a corrected geometric mean area under the curve (AUC)₀₋₂₄ of estrone that is less than about 65 pg*hr/mL. For example, administration of the suppository to a patient provides a corrected geometric mean area under the curve (AUC)₀₋₂₄ of estrone that is less than about 31 pg*hr/mL.

In some embodiments, administration of the suppository to a patient provides a corrected geometric mean peak plasma concentration (Cₘₐₓ) of estrone sulfate that is less than about 613 pg*hr/mL. For example, administration of the suppository to a patient provides a corrected geometric mean peak plasma concentration (Cₘₐₓ) of estrone sulfate that is less than about 16 pg*hr/mL. In some embodiments, administration of the suppository to a patient provides a corrected geometric mean area under the curve (AUC)0-24 of estrone sulfate that is less than about 5291 pg*hr/mL. For example, administration of the suppository to a patient provides a corrected geometric mean area under the curve (AUC)0-24 of estrone sulfate that is less than about 84 pg*hr/mL.

In further aspects of this disclosure, capsule disintegration may be determined. In some embodiments, delivery vehicle disintegration or absorption (presence or absence of the delivery vehicle after administration) at day 1 of treatment (for example, at 6 hours post first treatment) and at day 15 (for example, one day post the last treatment on day 14).

The pharmaceutical compositions can be formulated as described herein to provide desirable pharmacokinetic parameters in a subject (e.g., a female subject) to whom the composition is administered. In some embodiments, a pharmaceutical composition as described herein produces desirable pharmacokinetic parameters for estradiol in the subject. In some embodiments, a pharmaceutical composition as described herein produces desirable pharmacokinetic parameters for one or more metabolites of estradiol in the subject, for example, estrone or total estrone.

Following the administration of a composition comprising estradiol to a subject, the concentration and metabolism of estradiol can be measured in a sample (e.g., a blood, serum, or plasma sample) from the subject. Estradiol is typically converted reversibly to estrone, and both estradiol and estrone can be converted to the metabolite estriol. In postmenopausal women, a significant proportion of circulating estrogens exist as sulfate conjugates, especially estrone sulfate. Thus, estrone can be measured with respect to “estrone” amounts (excluding conjugates such as estrone sulfate) and “total estrone” amounts (including both free, or unconjugated, estrone and conjugated estrone such as estrone sulfate).

The pharmaceutical compositions of this disclosure can be characterized for one or more pharmacokinetic parameters of estradiol or a metabolite thereof following administration of the composition to a subject or to a population of subjects. These pharmacokinetic parameters include AUC, Cₘₐₓ, C_{avg}, and Tₘₐₓ. AUC is a determination of the area under the curve (AUC) plotting the blood, serum, or plasma concentration of drug along the ordinate (Y-axis) against time along the abscissa (X-axis). AUCs are well understood, frequently used tools in the pharmaceutical arts and have been extensively described. Cₘₐₓ is well understood in the art as an abbreviation for the maximum drug concentration in blood, serum, or plasma of a subject. Tₘₐₓ is well understood in the art as an abbreviation for the time to maximum drug concentration in blood, serum, or plasma of a subject.

In some embodiments, one or more pharmacokinetic parameters, e.g., AUC, Cₘₐₓ, C_{avg}, or Tₘₐₓ, is measured for estradiol. In some embodiments, one or more pharmacokinetic parameters, e.g., AUC, Cₘₐₓ, C_{avg}, or Tₘₐₓ, is measured for estrone. In some embodiments, one or more pharmacokinetic parameters, e.g., AUC, Cₘₐₓ, C_{avg}, or Tₘₐₓ, is measured for total estrone. Any pharmacokinetic parameter can be a “corrected” parameter, wherein the parameter is determined as a change over a baseline level.

Any of a variety of methods can be used for measuring the levels of estradiol, estrone, or total estrone in a sample, including immunoassays, mass spectrometry (MS), high performance liquid chromatography (HPLC) with ultraviolet fluorescent detection, liquid chromatography in conjunction with mass spectrometry (LC-MS), tandem mass spectrometry (MS/MS), and liquid chromatography-tandem mass spectrometry (LC-MS/MS). In some embodiments, the levels of estradiol, estrone, or total estrone are measured using a validated LC-MS/MS method. Methods of measuring hormone levels are well described in the literature.

### Statistical Measurements

According to embodiments, pharmacokinetics of the pharmaceutical composition disclosed herein are measured using statistical analysis. According to embodiments, Analysis of Variance (“ANOVA”) or Analysis of CoVariance (“ANCOVA”) are used to evaluate differences between a patient receiving treatment with a pharmaceutical composition comprising an active pharmaceutical composition (for example, a pharmaceutical composition comprising estradiol) and a patient receiving treatment with a placebo (for example, the same pharmaceutical composition but without estradiol) or a reference drug. A person of ordinary skill in the art will understand how to perform statistical analysis of the data collected.

### VII-A. EXEMPLARY EMBODIMENTS

In one aspect, the present invention provides a method comprising administering a composition comprising a hydrophobic active pharmaceutical ingredient (“API”) in a formulation that comprises (a) a pharmaceutically acceptable solubilizing agent where at least about 90% of the API is solubilized in the solubilizing agent, at least 50% of the formulation being composed of the solubilizing agent, and the solubilizing agent having a first viscosity that is not sufficiently great enough to maintain a formulation of the solubilizing agent in the vagina over a period of twenty-four hours and (b) a pharmaceutically acceptable surfactant, and (c) a pharmaceutically acceptable thickening agent, the surfactant and thickening agent optionally originating from a single excipient or single excipient mixture, wherein no more than 25% of the formulation is comprised of the surfactant and the thickening agent, the thickening agent having a second viscosity such that the presence of the thickening agent results in the formulation being retained in the vagina following administration, the method comprising (x) administering a therapeutically effective amount of the composition to the vagina of a subject wherein the amount of the composition administered in any single act of administration is between about 100 mg and 1000 mg, (y) providing an instruction to the person administering the composition that the subject may be upright or supine when the composition is administered and not requiring the subject to be supine after administration, and (z) repeating steps (x) and (y) for a number of times over a period of time sufficient to provide the subject with a therapeutic effect associated with the administration of the API to the vagina.

In connection with the above, the method further comprises the subject administering the compound to the vagina and instructing the subject to insert the compound about two inches into the vagina, and the step of administration is performed without an applicator. The method also further comprises providing the subject with instruction that immediately resuming ambulatory activity after administration is acceptable. In the above method, the surfactant and thickener are from a mixture of excipients that is formulated as a unit prior to inclusion in the formulation. In one embodiment, the surfactant and thickener comprise a first polyethylene glycol (PEG) compound comprising 2-10 PEG units and a second PEG compound comprising 20-40 PEG units. In another embodiment, the surfactant and thickener is TEFOSE 63.

In some embodiments, the ratio of the solubilizing agent to the combination of the surfactant and the thickener in the composition is between about 4:1 and about 12:1. In other embodiments, the ratio of the solubilizing agent to the combination of the surfactant and the thickener in the composition is between about 7:1 and 11:1. In yet other embodiments, the ratio of the solubilizing agent to the combination of the surfactant and the thickener in the composition is between about 8:1 and 10:1. In embodiments, the surfactant is a non-ionic surfactant having an HLB of between 7 and about 15. In some embodiments, the surfactant comprises one or more PEG stearate compounds. In other embodiments, the surfactant comprises a mix of small PEG stearates (PEG2-PEG10 stearates) and medium PEG stearates (PEG20-PEG40 stearates). In the above method, the solubilizing agent is primarily composed of medium chain fatty acids.

In one embodiment, a detectable amount of API is absorbed in both the vagina and vulva, and, in another embodiment, the API is absorbed in the labia. In a further embodiment, the formulation is delivered in a softgel capsule that is retained in the vagina upon administration and disintegrates in the vagina within one day of administration without any detectable discharge of the composition in at least 98% of subjects. In another embodiment, the formulation is delivered in a softgel capsule that is retained in the vagina upon administration and disintegrates in the vagina releasing the formulation and the API within one day of administration without any detectable discharge of the composition in at least 98% of subjects. In yet another embodiment, the formulation is delivered in a softgel capsule that is retained in the vagina upon administration and disintegrates in the vagina releasing the formulation and the API within one day of administration without any detectable discharge of the composition in at least 98% of subjects.

In one embodiment, the method is carried out in a population of subjects, i.e., patients, such as female patients. In one such embodiment, wherein in 95% or more subjects in a statistically significant population of subjects there is no statistically significant amount of systemic absorption of the API. In another such embodiment, wherein in 95% or more subjects in a statistically significant population of subjects there is no statistically significant amount of systemic absorption of the API.

In a preferred embodiment, the API is endogenous to the subject and the lack of systemic absorption of the API is determined by comparison of API levels after treatment to baseline, placebo, or both. In one embodiment, the lack of systemic absorption of the API is measured by comparison to placebo. In another embodiment, the API is endogenous to the subject and the lack of systemic absorption of the API is determined by comparison of API levels after treatment to baseline, placebo, or both. In one embodiment, the lack of systemic absorption of the API is measured by comparison to placebo.

In one embodiment, the API is estradiol, but in other embodiments, the API is an estrogen other than estradiol.

In preferred embodiments, the solubilizing agent has a viscosity of between about 5 centipoise (“cps”) and about 50 cps at 25 degrees C. and the formulation has a viscosity of between about 70 cps and about 120 cps at 25 degrees C.

In connection with the above method, the instructions relating to carrying out the method are provided by a health care provider, in a product label, or a combination thereof.

In preferred embodiments, the composition is a liquid composition, and the composition spreads over an area of vaginal tissue or vaginal and vulvar tissue of about 50 cm2 to about 120 cm2 following administration.

In preferred embodiments, the liquid composition is encapsulated in a capsule, such as a bioadhesive capsule (e.g., a gelatin capsule, such as a soft gelatin capsule), wherein the longest dimension of the capsule does not exceed 0.75 inches and wherein the amount of formulation contained in the capsule does not exceed 1000 mg. In embodiments, the amount of formulation contained in the capsule is between 50 mg and 500 mg. In some embodiments, the capsule comprises a first end having a width that is less than 50% of the width of the second end, and wherein the method optionally includes the step of instructing the subject to administer the gel cap by inserting the first end of the capsule first.

In some embodiments of the above method, the composition lacks any gelatin or gel-forming bioadhesive other than gelatin or hydrolyzed gelatin. In some embodiments, the composition lacks any gelatin or gel-forming bioadhesive other than gelatin or hydrolyzed gelatin. In some embodiments, the formulation does not contain carboxyvinylic acids, gelatin, hydroxypropylcellulose, carboxymethylcellulose, xanthane gum, guar gum, aluminum silicate and mixtures thereof or colloidal silica.

In another aspect, the present invention provides a method for treating the symptoms of vulvo-vaginal atrophy (VVA) comprising: intravaginally administering a liquid pharmaceutical composition comprising 4 μg to 25 μg of estradiol to a subject having VVA, wherein the liquid pharmaceutical composition is contained in a capsule. In one embodiment, the symptoms of VVA comprise one or more symptoms selected from vaginal dryness, dyspareunia, vaginal or vulvar irritation, burning, itching, dysuria, urinary tract infection, and vaginal bleeding associated with sexual activity.

In yet another aspect, the present invention provides a method for reestrogenizing the vagina, labia, or vulva, the method comprising: intravaginally administering a liquid pharmaceutical composition comprising 4 μg to 25 μg of estradiol to a subject in need thereof, wherein the liquid pharmaceutical composition is contained in a capsule. Importantly, using this method, transport of estradiol to the uterus of the subject is avoided (it is minimal or de minims, or it is in an amount that is not sufficient to cause endometrial hyperplasia after 12 weeks of treatment), i.e., the estradiol that is administered is not transported to the uterus.

In still another aspect, the present invention provides a method for treat a urinary tract infection in a subject in need thereof, the method comprising: intravaginally administering a liquid pharmaceutical composition comprising 4 μg to 25 μg of estradiol to the subject in need thereof, wherein the liquid pharmaceutical composition is contained in a capsule. Importantly, using this method, transport of estradiol to the uterus of the subject is avoided (it is minimal or de minims, or it is in an amount that is not sufficient to cause endometrial hyperplasia after 12 weeks of treatment), i.e., the estradiol that is administered is not transported to the uterus.

In embodiments of the above methods, the capsule is a bioadhesive capsule. In another embodiments, the capsule is a gelatin capsule, such as a soft gelatin capsule.

In one embodiment of the above methods, intravaginally administering the liquid pharmaceutical composition comprises digitally inserting the capsule into the lower third of the vagina of the subject. In one embodiment of the above methods, the capsule adheres to the vaginal tissue of the subject and dissolves, ruptures, or disintegrates, thereby releasing the liquid pharmaceutical composition. In embodiments, the liquid pharmaceutical composition spreads over a surface area selected from the group consisting of the vagina, the vulva, the labia, and combinations thereof. In preferred embodiments of these methods, the soft gelatin capsule and the liquid pharmaceutical composition are fully absorbed by the vaginal tissue of the subject. Importantly, using the above methods, transport of estradiol to the uterus of the subject is avoided (i.e., minimal or de minims or in an amount that is not sufficient to cause endometrial hyperplasia after 12 weeks of treatment), i.e., the estradiol that is administered is not transported to the uterus.

Some advantages of the above methods and the other methods disclosed herein include, but are not limited to the following: (i) vaginal secretions from the subject are not required for the capsule to dissolve, rupture, or disintegrate, thereby releasing the liquid pharmaceutical composition; (ii) the only discharge that occurs after intravaginally administering the liquid pharmaceutical composition is a natural discharge; (iii) the subject can be ambulatory immediately after intravaginally administering the liquid pharmaceutical composition, or the subject can be ambulatory for a period of time beginning 5 minutes to 120 minutes after intravaginally administering the liquid pharmaceutical composition; and (iv) importantly estradiol is not transported to the uterus, i.e., transport of estradiol to the uterus is avoided, with the methods of the invention.

In the above methods, the liquid pharmaceutical compositions further comprise a solubilizing agent. In preferred embodiments, the solubilizing agent is an oil. In preferred embodiments, the oil comprises at least one C6-C12 fatty acid or a glycol, monoglyceride, diglyceride, or triglyceride ester thereof. In preferred embodiments, the liquid pharmaceutical composition further comprises a thickener or a surfactant. In certain embodiments, the liquid pharmaceutical compositions do not include a hydrophilic gel-forming bioadhesive agent. In preferred embodiments, estradiol is the only active hormone in the liquid pharmaceutical compositions.

In some embodiments, the liquid pharmaceutical compositions include 4 μg estradiol. In other embodiments, the liquid pharmaceutical compositions include 10 μg estradiol. In yet other embodiments, the liquid pharmaceutical compositions include 25 μg estradiol.

In the above methods, the intravaginal administration is preferably conducted daily for two weeks, and twice weekly thereafter. The intravaginal administration is preferably conducted at any time of day, but is conducted at about the same time each day.

The above methods are surprisingly effective within two weeks of the first administration. Importantly, the above method for treating VVA provides: (i) increasing the level of vaginal secretions in a subject, as assessed by visual examination; (ii) increasing the number of vaginal rugae in the subject, as assessed by visual examination; (iii) decreasing vaginal bleeding or petechiae in the subject, as assessed by visual examination; and (iv) changing the color of the vaginal mucosa in the subject from transparent to pink, or from pale pink to pink, as assessed by visual examination. Importantly, the above method (i) decreases the severity of vaginal dryness within two weeks; (ii) decreases the severity of vulvar or vaginal itching within two weeks; and (iii) decreases the severity of decreases the severity of dyspareunia within two weeks. Importantly, the method provides these benefits while avoiding transport of the estradiol to the uterus.

### VIII. EXAMPLES

The following examples are of pharmaceutical compositions, delivery vehicles, and combinations thereof. Methods of making are also disclosed. Data generated using the pharmaceutical compositions disclosed herein are also disclosed.

### Example 1: Pharmaceutical Composition

In embodiments, estradiol is procured and combined with one or more pharmaceutically acceptable solubilizing agents. The estradiol is purchased as a pharmaceutical grade ingredient, often as micronized estradiol, although other forms can also be used. In embodiments, the pharmaceutical composition includes estradiol in a dosage strength of from about 1 μg to about 50 μg. In embodiments, the pharmaceutical composition includes 10 μg of estradiol. In embodiments, the pharmaceutical composition includes 25 μg of estradiol.

In embodiments, the estradiol is combined with pharmaceutically acceptable solubilizing agents, and, optionally, other excipients, to form a pharmaceutical composition. In embodiments, the solubilizing agent is one or more of Capmul MCM, Miglyol 812, Gelucire 39/01, Gelucire 43/01, Gelucire 50/13, and Tefose 63.

Gelucire 39/01 and Gelucire 43/01 each have an HLB value of 1. Gelucire 50/13 has an HLB value of 13. Tefose 63 has an HLB value of between 9 and 10.

Various combinations of pharmaceutically acceptable solubilizing agents were combined with estradiol and examined as shown in Table 1.

TABLE 1
Capmul MCM (“MCM”), Gelucire 39/01 (“39/01”), Gelucire 43/01(“43/01”), Gelucire
50/13(“50/13”), and Tefose (“Tefose 63”)

| | | | Physical | Physical | | | |
|---|---|---|---|---|---|---|---|
| | | | state @ | state | | | Dispersion |
| | | | Room | @ 37° C. | | Melting | in |
| | Vehicle | | Tem- | after | Viscosity | Time @ | water |
| # | system | Ratio | perature | ~30 minutes | (cps) | 37° C. | 37° C. |
| 1 | MCM: | 8:2 | Solid | Clear liquid | 50 @ 37° C. | Start: 6 min | Small oil |
| | 39/01 | | | | | Finish: 12 | drops on top |
| | | | | | | min | |
| 2 | MCM: | 7:3 | Solid | Clear liquid | | Start: 9 min | |
| | 39/01 | | | | | Finish: 19 | |
| | | | | | | min | |
| 3 | MCM: | 6:4 | Solid | Clear liquid | | Start: 20 | |
| | 39/01 | | | | | min Finish: | |
| | | | | | | 32 min | |
| 4 | MCM: | 8:2 | Solid | Liquid with | | | |
| | 43/01 | | | solid particles | | | |
| 5 | MCM: | 7:3 | Solid | Liquid with | | | |
| | 43/01 | | | solid particles | | | |
| 6 | MCM: | 9:1 | Liquid/ | Liquid/ | 140@ 25° C. | Clear after | Uniformly |
| | 50/13 | | cloudy | cloudy | | 20 min | cloudy |
| | | | | | | | dispersion |
| 7 | MCM: | 8:2 | Liquid/ | Liquid/ | 190@ 25° C. | | Uniformly |
| | 50/13 | | cloudy | cloudy | | | cloudy |
| | | | | | | | dispersion |
| 8 | MCM: | 7:3 | Semisolid | Semisolid | | | |
| | 50/13 | | | | | | |
| 9 | MCM: | 9:1 | Semisolid | Liquid/ | 150@ 25° C. | Start: 1 min | Uniformly |
| | TEFOSE 63 | | | cloudy | | Finish: 5 | cloudy |
| | | | | | | min | dispersion |
| 10 | MCM: | 8:2 | Semisolid | Semisolid | 240@ 25° C. | Uniformly | |
| | TEFOSE 63 | | | | | | cloudy |
| | | | | | | | dispersion |
| 11 | MCM: | 7:3 | Semisolid | Semisolid | 380@ 25° C. | Semisolid | Uniformly |
| | TEFOSE 63 | | | | | after 30 min | cloudy |
| | | | | | | at 37° C., | dispersion |
| | | | | | | doesn't melt | |
| | | | | | | at 41° C. | |
| 12 | MIGLYOL 812: | 9:1 | Semisolid | Semisolid | 140@ 25° C. | | 2 phases, oil |
| | 50/13 | | | | | | on top |
| 13 | MIGLYOL 812: | 9:1 | Liquid/ | Liquid/ | 90@ 25° C. | Start: 1 min | 2 phases, oil |
| | TEFOSE 63 | | cloudy | cloudy | | Finish: 5 | on top |
| | | | | | | min | |

Pharmaceutical compositions in Table 1 that were liquid or semisolid at room temperature were tested using a Brookfield viscometer (Brookfield Engineering Laboratories, Middleboro, Mass.) at room temperature. Pharmaceutical compositions appearing in Table 1 that were solid at ambient temperature were tested using a Brookfield viscometer at 37° C.

Pharmaceutical compositions appearing in Table 1 that were solid at room temperature were assessed at 37° C. to determine their melting characteristics. The viscosity of the gels can be important during encapsulation of the formulation. For example, in some cases, it is necessary to warm the formulation prior to filing of the gelatin capsules. In addition, the melting characteristics of the composition can have important implications following administration of the formulation into the body. For example, in some embodiments, the formulation will melt at temperatures below about 37° C. Pharmaceutical Composition 11 (Capmul MCM/Tefose 63), for example, did not melt at 37° C. or 41° C.

A dispersion assessment of the pharmaceutical compositions appearing in Table 1 was performed. The dispersion assessment was performed by transferring 300 mg of each vehicle system in 100 mL of 37° C. water, without agitation, and observing for mixing characteristics. Results varied from formation of oil drops on the top to separation of phases to uniform, but cloudy dispersions. Generally speaking, it is believed that formulations able to readily disperse in aqueous solution will have better dispersion characteristics upon administration. It was surprisingly found, however, as shown below in Examples 7-9, that formulations that did not readily disperse in aqueous solution (e.g., Formulation 13) and instead formed two phases upon introduction to the aqueous solution were found to be the most effective when administered to the human body.

### Example 2: Delivery Vehicle

In embodiments, the pharmaceutical composition is delivered in a gelatin capsule delivery vehicle. The gelatin capsule delivery vehicle includes, for example, gelatin (e.g., Gelatin, NF (150 Bloom, Type B)), hydrolyzed collagen (e.g., GELITA®, GELITA AG, Eberbach, Germany), glycerin, sorbitol special, or other excipients in proportions that are well known and understood by persons of ordinary skill in the art. Sorbitol special may be obtained commercially and may tend to act as a plasticizer and humectant.

A variety of delivery vehicles were developed, as show in Table 2, Gels A through F. In Table 2, each delivery vehicle A through F differs in the proportion of one or more components.

TABLE 2
Gelatin Capsule Delivery Vehicles

| | A | B | C | D | E | F |
|---|---|---|---|---|---|---|
| Ingredient | % w/w | % w/w | % w/w | % w/w | % w/w | % w/w |
| Gelatin, NF (150 Bloom, Type B) | 41.0 | 41.0 | 41.0 | 41.0 | 43.0 | 43.0 |
| Glycerin 99.7%, USP | 6.0 | 6.0 | 6.0 | 6.0 | 18.0 | 18.0 |
| Sorbitol Special, USP | 15.0 | 15.0 | 15.0 | 15.0 | | |
| GELITA ® (hydrolyzed collagen) | 3 | | | | 3.0 | |
| Citric acid | | 0.1 | 0.5 | 1 | | 0.1 |
| Purified Water | 35.0 | 37.9 | 37.5 | 37.0 | 36.0 | 38.9 |
| Total | 100.0 | 100.0 | 100.0 | 100.0 | 100.0 | 100.0 |
| Dissolution gel strips, Avg of 3 | 48 min | 50 min | 75 min | 70 min | | |
| (500 mL DH2O, 50 rpm @ 37° C.) | (42, 45, 58) | (50, 51, 50) | (76, 75, 74) | (70, 71, 70) | | |
| Dissolution gel strips, Avg of 3 | 70 min | | | | 78 min | 82 min |
| (500 mL pH 4 buffer, 50 rpm @ | | | | | | |
| 37° C.) | | | | | | |

Each delivery vehicle A through F was prepared at a temperature range from about 45° C. to about 85° C. Each molten delivery vehicle A through F was cast into a film, dried, and cut into strips. The strips were cut into uniform pieces weighing about 0.5 g, with about 0.5 mm thickness. Strips were placed into a USP Type 2 dissolution vessel in either water or pH 4 buffer solution and the time for them to completely dissolve was recorded (see Table 2). Delivery vehicle A had the fastest dissolution in both water and pH 4 buffer solution.

### Example 3: Pharmaceutical Compositions and Delivery Vehicle

Various combinations of the pharmaceutical compositions from Table 1 and from Table 2 were prepared. The combinations are shown in Table 3.

TABLE 3

| | | | | Delivery |
|---|---|---|---|---|
| Trial | Pharmaceutical Composition | Ratio | Batch Size g | Vehicle |
| 1 | MCM:39/01 | 8:2 | 750 | A |
| 2 | MCM:50/13 | 8:2 | 750 | A |
| 3 | MCM:TEFOSE 63 | 8:2 | 750 | A |
| 4 | MCM:TEFOSE 63 | 8:2 | 750 | B |
| 5 | MIGLYOL 812:TEFOSE 63 | 9:1 | 750 | A |

Each aliquot of the pharmaceutical compositions of Table 3 about 300 mg to about 310 mg. Batch size was as listed in Table 3. To encapsulate the vehicle system, each 300 mg to about 310 mg pharmaceutical composition aliquot was encapsulated in about 200 mg of the gelatin capsule delivery vehicle. Thus, for example, in Trial 1, the pharmaceutical composition denoted by MCM:39/01 was encapsulated in gelatin capsule delivery vehicle A for a total encapsulated weight of about 500 mg to about 510 mg. The aliquot size is arbitrary depending on the concentration of the estradiol and the desired gelatin capsule delivery vehicle size. Artisans will readily understand how to adjust the amount of estradiol in the pharmaceutical composition to accommodate a given size of delivery vehicle, when the delivery vehicle encapsulates the pharmaceutical composition.

### Example 4: Estradiol Solubility

In various experiments, solubilizing agents were tested to determine whether they were able to solubilize 2 mg of estradiol for a total pharmaceutical composition weight of 100 mg. The solubilizing agents were considered suitable if estradiol solubility in the solubilizing agent was greater than or equal to about 20 mg/g. Initial solubility was measured by dissolving micronized estradiol into various solubilizing agents until the estradiol was saturated (the estradiol/solubilizing agent equilibrated for three days), filtering the undissolved estradiol, and analyzing the resulting pharmaceutical composition for estradiol concentration by HPLC.

TABLE 4
Solubility of Solubilizing Agents (*denotes literature reference)

| Ingredient | Solubility (mg/g) |
|---|---|
| PEG 400 | 105* |
| Propylene Glycol | 75* |
| Polysorbate 80 | 36* |
| TRANSCUTOL HP | 141 |
| CAPMUL PG8 | 31.2 |

### Example 5: Pharmaceutical Compositions

The following pharmaceutical compositions are contemplated.

### Gel Mass

| Ingredient | % w/w | Qty/Batch (kg) | |
|---|---|---|---|
| Gelatin 150 Bloom Limed Bone, NF | 41.00 | 82.00 | |
| Hydrolyzed Gelatin | 3.00 | 6.00 | |
| Glycerin 99.7% | 6.00 | 12.00 | |
| Sorbitol Special, NF | 15.00 | 30.00 | |
| Opatint White G-18006 | 1.20 | 2.40 | |
| Opatine Red DG-15001 | 0.06 | 0.12 | |
| Purified Water, USP | 33.74 | 67.48 | |
| Total | 100.00 | 200.00 | Kg |

### Pharmaceutical Composition 1: 10 μg Estradiol

| | Qty/Capsule | | | |
|---|---|---|---|---|
| Ingredients | (mg) | % w/w | Qty/Batch | |
| Estradiol hemihydrate micronized, | 0.010 | 0.003 | 0.10 | g |
| USP | | | | |
| CAPMUL ® MCM, NF (Glyceryl | 240.0 | 79.997 | 2.40 | kg |
| Caprylate/Caprate or Medium Chain | | | | |
| Mono- and Diglycerides) | | | | |
| GELUCIRE ® 50/13 (stearoyl | 60.0 | 20.0 | 600.0 | g |
| polyoxyl-32 glycerides NF) | | | | |
| Total | 300.0 | 100.0 | 3.0 | kg |

### Pharmaceutical Composition 2: 10 μg Estradiol

| | Qty/Capsule | | | |
|---|---|---|---|---|
| Ingredients | (mg) | % w/w | Qty/Batch | |
| Estradiol hemihydrate micronized, | 0.010 | 0.003 | 0.10 | g |
| USP | | | | |
| MIGLOYL ® 812 (medium chain | 270.0 | 89.997 | 2.70 | kg |
| triglyceride) | | | | |
| TEFOSE ® 63 (mixture of PEG-6 | 30.0 | 10.0 | 300.0 | g |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 3.00 | kg |

### Pharmaceutical Composition 3: 25 μg Estradiol

| | Qty/Capsule | | | |
|---|---|---|---|---|
| Ingredients | (mg) | % w/w | Qty/Batch | |
| Estradiol hemihydrate micronized, | 0.026* | 0.009 | 0.26 | g |
| USP | | | | |
| MIGLOYL ® 812 (medium chain | 270.0 | 89.991 | 2.70 | kg |
| triglyceride) | | | | |
| TEFOSE ® 63 (mixture of PEG-6 | 30.02 | 10.0 | 300.0 | g |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 3.0 | g |

*1.0 mg estradiol is equivalent to 1.03 mg estradiol hemihydrate

### Pharmaceutical Composition 4: 4 μg Estradiol

| | | | Qty/Batch | |
|---|---|---|---|---|
| | Qty/Capsule | | (alternate | |
| Ingredients | (mg) | % w/w | batch size) | |
| Estradiol hemihydrate micronized, | 0.0041* | 0.001 | 0.041 | g |
| USP | | | (0.615 | g) |
| MIGLOYL ® 812 (medium chain | 269.99 | 89.999 | 2700.0 | g |
| triglyceride) | | | (40.50 | kg) |
| TEFOSE ® 63 (mixture of PEG-6 | 30.0 | 10.0 | 300.0 | g |
| stearate or ethylene glycol | | | (4.50 | kg) |
| palmitostearate or PEG-32 | | | | |
| stearate; polyoxyl 6 and polyoxyl | | | | |
| 32 palmitostearate/glycol stearate) | | | | |
| Total | 300.0 | 100.0 | 3000.0 | g |
| | | | 45.0 | kg |

*1.0 mg estradiol is equivalent to 1.03 mg estradiol hemihydrate

### Pharmaceutical Composition 5: 10 μg Estradiol

| | Qty/Capsule | | | |
|---|---|---|---|---|
| Ingredients | (mg) | % w/w | Qty/Batch | |
| Estradiol hemihydrate micronized, | 0.0103* | 0.003 | 1.545 | g |
| USP | | | | |
| MIGLOYL ® 812 (medium chain | 269.99 | 89.997 | 40.5 | kg |
| triglyceride) | | | | |
| TEFOSE ® 63 (mixture of PEG-6 | 30.0 | 10.0 | 4.50 | kg |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 45.0 | g |

*1.0 mg estradiol is equivalent to 1.03 mg estradiol hemihydrate

### Pharmaceutical Composition 6: 25 μg Estradiol

| | Qty/Capsule | | | |
|---|---|---|---|---|
| Ingredients | (mg) | % w/w | Qty/Batch | |
| Estradiol hemihydrate micronized, | 0.026* | 0.009 | 3.90 | g |
| USP | | | | |
| MIGLOYL ® 812 (medium chain | 269.97 | 89.991 | 40.50 | kg |
| triglyceride) | | | | |
| TEFOSE ® 63 (mixture of PEG-6 | 30.0 | 10.0 | 4.50 | kg |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 45.0 | g |

*1.0 mg estradiol is equivalent to 1.03 mg estradiol hemihydrate

### Pharmaceutical Composition 7: Placebo

| | Qty/Capsule | | | |
|---|---|---|---|---|
| Ingredients | (mg) | % w/w | Qty/Batch | |
| Estradiol hemihydrate micronized, | 0.00 | 0.00 | 0.00 | g |
| USP | | | | |
| MIGLOYL ® 812 (medium chain | 270.0 | 90.0 | 40.5 | kg |
| triglyceride) | | | | |
| TEFOSE ® 63 (mixture of PEG-6 | 30.0 | 10.0 | 4.5 | kg |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 3000.0 | g |

In the Examples below, TX-004HR is Pharmaceutical Compositions 4, 5, and 6 (TX-004HR 4 μg, TX-004HR 10 μg, and TX-004HR 25 μg) compared to Pharmaceutical Composition 7.

### Example 6: Process

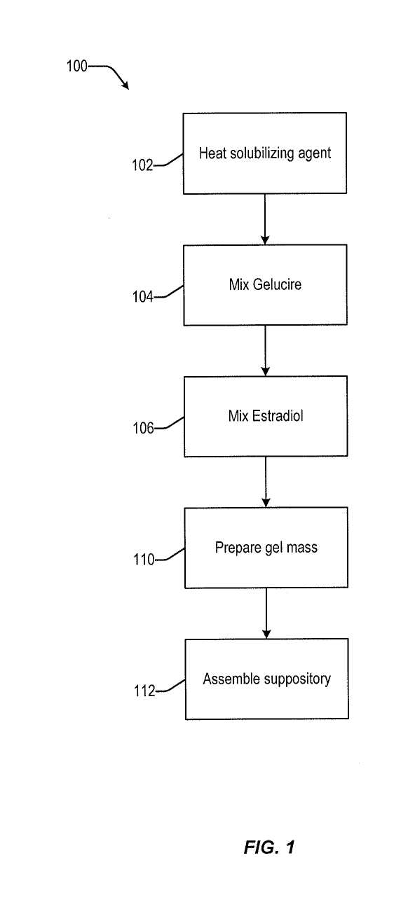

FIG. 1 illustrates an embodiment of a method making pharmaceutical composition comprising estradiol solubilized in CapmulMCM/Gelucire solubilizing agent encapsulated in a soft gelatin delivery vehicle 100. In operation 102, the CapmulMCM is heated to 40° C.±5° C. Heating may be accomplished through any suitable means. The heating may be performed in any suitable vessel, such as a stainless steel vessel. Other pharmaceutical compositions can be made using the same general method by substituting various excipients, including the solubilizing agent.

In operation 104, Gelucire is mixed with the CapmulMCM to form the finished solubilizing agent. As used herein, any form of Gelucire may be used in operation 104. For example, one or more of Gelucire 39/01, Gelucire 43/01, Gelucire 50/13 may be used in operation 104. Mixing is performed as would be known to persons of ordinary skill in the art, for example by impeller, agitator, stirrer, or other like devices used to mix pharmaceutical compositions. Operation 104 may be performed under an inert or relatively inert gas atmosphere, such as nitrogen gas. Mixing may be performed in any vessels that are known to persons of ordinary skill in the art, such as a stainless steel vessel or a steel tank.

In operation 106 estradiol is mixed into the solubilizing agent. In embodiments, the estradiol in micronized when mixed into the solubilizing agent. In other embodiments, the estradiol added is in a non-micronized form. Mixing may be facilitated by an impeller, agitator, stirrer, or other like devices used to mix pharmaceutical compositions. Operation 106 may be performed under an inert or relatively inert gas atmosphere, such as nitrogen gas.

In embodiments, however, the addition of estradiol may be performed prior to operation 104. In that regard, operations 104 and 106 are interchangeable with respect to timing or can be performed contemporaneously with each other.

In operation 110, the gelatin delivery vehicle is prepared. Any of the gelatin delivery vehicles described herein may be used in operation 110. In embodiments, gelatin, hydrolyzed collagen, glycerin, and other excipients are combined at a temperature range from about 45° C. to about 85° C. and prepared as a film. Mixing may occur in a steel tank or other container used for preparing gelatin delivery vehicles. Mixing may be facilitated by an impellor, agitator, stirrer, or other devices used to combine the contents of gelatin delivery vehicles. Operation 110 may be performed under an inert or relatively inert gas atmosphere, such as nitrogen gas. In embodiments, the gelatin delivery vehicle mixture is degassed prior to being used to encapsulate the pharmaceutical composition.

In operation 112, the gelatin delivery vehicle encapsulates the pharmaceutical composition, according to protocols well known to persons of ordinary skill in the art. In operation 112, a soft gelatin capsule delivery vehicle is prepared by combining the pharmaceutical composition made in operation 106 with the gelatin delivery vehicle made in operation 110. The gelatin may be wrapped around the material, partially or fully encapsulating it or the gelatin can also be injected or otherwise filled with the pharmaceutical composition made in operation 106.

In embodiments, operation 112 is completed in a suitable die to provide a desired shape. Vaginal soft gel capsules may be prepared in a variety of geometries. For example, vaginal soft gel capsules may be shaped as a tear drop, a cone with frustoconical end, a cylinder, a cylinder with larger “cap” portion as illustrated in FIG. 2, or other shapes suitable for insertion into the vagina. The resulting pharmaceutical composition encapsulated in the soft gelatin delivery vehicle may be inserted digitally or with an applicator.

### Example 7: Study of Estradiol Pharmaceutical Composition on the Improvement of Vulvovaginal Atrophy (VVA)

The objective of this study was designed to evaluate the efficacy and safety of a pharmaceutical composition comprising 10 μg estradiol (i.e., Pharmaceutical Composition 2) in treating moderate to severe symptoms of VVA associated with menopause after 14 days of treatment, and to estimate the effect size and variability of vulvovaginal atrophy endpoints. In addition, the systemic exposure to estradiol from single and multiple doses of the pharmaceutical composition was investigated.

This study was a phase 1, randomized, double-blind, placebo-controlled trial to evaluate safety and efficacy of the pharmaceutical composition in reducing moderate to severe symptoms of vaginal atrophy associated with menopause and to investigate the systemic exposure to estradiol following once daily intravaginal administrations of a pharmaceutical composition for 14 days.

Postmenopausal subjects who met the study entry criteria were randomized to one of two treatment groups (pharmaceutical composition or placebo). During the screening period subjects were asked to self-assess the symptoms of VVA, including vaginal dryness, vaginal or vulvar irritation or itching, dysuria, vaginal pain associated with sexual activity, and vaginal bleeding associated with sexual activity. Subjects with at least one self-assessed moderate to severe symptom of VVA identified by the subject as being most bothersome to her were eligible to participate in the study.

Clinical evaluations were performed at the following time points:

Screening Period (up to 28 days);

Visit 1—Randomization/Baseline (day 1);

Visit 2—Interim (day 8); and

Visit 3—End of the treatment (day 15).

Eligible subjects were randomized in a 1:1 ratio to receive either pharmaceutical composition comprising estradiol 10 μg or a matching placebo vaginal softgel capsule, and self-administered their first dose of study medication at the clinical facility under the supervision of the study personnel. Serial blood samples for monitoring of estradiol level were collected at 0.0, 1.0, 3.0, and 6.0 hours relative to first dose administration on day 1. Subjects remained at the clinical site until completion of the 6-hour blood draw and returned to clinical facility for additional single blood draws for measurement of estradiol concentration on day 8 (before the morning dose) and day 15. Subjects were provided with enough study medication until the next scheduled visit and were instructed to self-administer their assigned study treatment once a day intravaginally at approximately the same time (±1 hour) every morning. Each subject was provided with a diary in which she was required to daily record investigational drug dosing dates and times. Subjects returned to clinical facility on day 8 for interim visit and on day 15 for end of treatment assessments and post study examinations. Capsule disintegration state was assessed by the investigator at day 1 (6 hours post-dose) and day 15.

The study involved a screening period of up to 28 days before randomization and treatment period of 14 days. Selection of dosage strength (estradiol 10 μg) and treatment regimen (once daily for two weeks) was based on the FDA findings on safety and efficacy of the RLD.

### Number of Subjects (Planned and Analyzed)

Up to 50 (25 per treatment group) postmenopausal female subjects 40 to 75 years old with symptoms of moderate to severe VVA were randomized. 50 subjects were enrolled, 48 subjects completed the study, and 48 subjects were analyzed.

### Diagnosis and Main Criteria for Inclusion

Fifty female subjects were enrolled in the study. Post-menopausal female subjects 40 to 75 years of age, with a mean age was 62.3 years were enrolled. Subjects' mean weight (kg) was 71.2 kg with a range of 44.5-100 kg. Subjects' mean height (cm) was 162.6 cm with a range of 149.9-175.2 cm, and the mean BMI (kg/m2) was 26.8 kg/m2 with a range of 19-33 kg/m2. Criteria of inclusion in the study included: self-identification of at least one moderate to severe symptom of VVA, for example, vaginal dryness, dyspareunia, vaginal or vulvar irritation, burning, or itching, dysuria, vaginal bleeding associated with sexual activity, that was identified by the subject as being most bothersome to her; ≤5% superficial cells on vaginal smear cytology; vaginal pH>5.0; and estradiol level ≤50 pg/mL. Subject who were judged as being in otherwise generally good health on the basis of a pre-study physical examination, clinical laboratory tests, pelvic examination, and mammography were enrolled.

### Estradiol 10 μg or Placebo, Dose, and Mode of Administration

Subjects were randomly assigned (in 1:1 allocation) to self-administer one of the following treatments intravaginally once daily for 14 days:

Treatment A: The pharmaceutical composition of Example 5 (Pharmaceutical Composition 2: 10 μg estradiol); or

Treatment B: Placebo vaginal softgel capsule, containing the same formulation as Treatment A, except for the 10 μg of estradiol.

The estradiol formulation was a tear drop shaped light pink soft gel capsule. Treatment B had the same composition, appearance, and route of administration as the Treatment A, but contained no estradiol.

### Duration of Treatment

The study involved a screening period of up to 28 days before randomization and a treatment period of 14 days.

### Criteria for Evaluation

### Efficacy Endpoints:

Change from baseline (screening) to day 15 in the Maturation Index (percent of parabasal vaginal cells, superficial vaginal cells, and intermediate vaginal cells) of the vaginal smear: data for this endpoint are shown in Tables 6-8. Change from baseline (screening) to day 15 in vaginal pH: data for this endpoint are shown in Table 9. Change from baseline (randomization) to day 15 in severity of the most bothersome symptoms: (1) vaginal dryness; (2) vaginal or vulvar irritation, burning, or itching; (3) dysuria; (4) dyspareunia; (5) vaginal bleeding associated with sexual activity: data for this endpoint are shown in Tables 13 and 15. Change from baseline (randomization) to day 15 in investigator's assessment of the vaginal mucosa: data for this endpoint are shown in Tables 18-21.

Unless otherwise noted, the efficacy endpoints were measured as a change-from Visit 1—Randomization/Baseline (day 1) to Visit 3—End of the treatment (day 15), except for vaginal bleeding which was expressed as either treatment success or failure.

Other endpoints include:

Vital signs, weight, changes in physical exam, pelvic and breast exam, and adverse events were evaluated as part of the safety endpoints; concentration of estradiol at each sampling time; peak concentration of estradiol on day 1 and sampling time at which peak occurred; delivery vehicle disintegration to measure the amount of residual delivery vehicle remains in the vagina post treatment.

Results from the assessment of plasma concentrations of estradiol are presented in Table 5.

TABLE 5
Safety Results: The descriptive statistics for Day 1 plasma
estradiol Cₘₐₓ and Tₘₐₓ are provided below.

| | Estradiol 10 μg | | Placebo | |
|---|---|---|---|---|
| | Cₘₐₓ | Tₘₐₓ | Cₘₐₓ | Tₘₐₓ |
| N | 24 | 24 | 26 | 26 |
| Mean ± SD | 30.7 ± 7.47 | 2.12 ± 1.73 | 27.5 ± 17.26 | 4.00 ± 2.68 |
| Geometric | 29.9 | — | 24.7 | — |
| Mean | | | | |
| Median | 29.8 | 1.00 | 22.1 | 6.00 |
| Min, Max | 19.7, 52.3 | 1.00, 6.00 | 15.1, 90.0 | 0.00, 6.00 |
| CV % | 24.3% | 81.3% | 62.9% | 67.1% |

Maturation Index Results

Vaginal cytology data was collected as vaginal smears from the lateral vaginal walls according to standard procedures to evaluate vaginal cytology at screening and Visit 3—End of treatment (day 15). The change in the Maturation Index was assessed as a change in cell composition measured at Visit 1—Baseline (day 1) compared to the cell composition measured at Visit 3—End of treatment (day 15). The change in percentage of superficial, parabasal, and intermediate cells obtained from the vaginal mucosal epithelium from a vaginal smear was recorded. Results from these assessments are presented in Tables 6, 7, and 8.

TABLE 6
Primary Efficacy Analysis Results of Change from
Baseline (Screening) to Day 15 in the Maturation
Index of the Vaginal Smear (Percent Parabasal Cells)

| | | | | Difference | 90% | Estradiol |
|---|---|---|---|---|---|---|
| | | | | Between | CI for | 10 μg vs. |
| | Sta- | Estradiol | | Treatment | Differ- | Placebo |
| Population | tistics | 10 μg | Placebo | Means | ence | P-value |
| Intent-to- | N | 24 | 24 | — | — | — |
| Treat | | | | | | |
| | Least- | −54.4 | −4.80 | −49.6 | (−60.4, | <0.0001 |
| | Squares | | | | −38.8) | |
| | Mean | | | | | |
| | Mean ± | −53.8 ± | −5.4 ± | — | — | — |
| | SD | 39.7 | 22.3 | | | |
| | Median | −60.0 | −5.0 | — | — | — |
| | Min, | −100.0, | −60.0, | — | — | — |
| | Max | 0.0 | 60.0 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

TABLE 7
Primary Efficacy Analysis Results of Change from
Baseline (Screening) to Day 15 in the Maturation
Index of the Vaginal Smear (Superficial Cells)

| | | | | Difference | 90% | Estradiol |
|---|---|---|---|---|---|---|
| | | | | Between | CI for | 10 μg vs. |
| | Sta- | Estradiol | | Treatment | Differ- | Placebo |
| Population | tistics | 10 μg | Placebo | Means | ence | P-value |
| Intent-to- | N | 24 | 24 | — | — | — |
| Treat | | | | | | |
| | Least- | 35.2 | 8.75 | 26.5 | (15.4, | 0.0002 |
| | Squares | | | | 37.6) | |
| | Mean | | | | | |
| | Mean ± | 35.2 ± | 8.8 ± | — | — | — |
| | SD | 26.4 | 18.7 | | | |
| | Median | 40.0 | 0.0 | — | — | — |
| | Min, | 0.0, | 0.0, | — | — | — |
| | Max | 80.0 | 90.0 | | | |

¹Confidence interval for the estradiol 10 μg-Placebo from ANOVA with treatment as a fixed effect.
²P-value for treatment comparison from ANOVA with treatment as a fixed effect.

TABLE 8
Primary Efficacy Analysis Results of Change from
Baseline (Screening) to Day 15 in the Maturation
Index of the Vaginal Smear (Intermediate Cells)

| | | | | Difference | 90% | Estradiol |
|---|---|---|---|---|---|---|
| | | | | Between | CI for | 10 μg vs. |
| | Sta- | Estradiol | | Treatment | Differ- | Placebo |
| Population | tistics | 10 μg | Placebo | Means | ence | P-value² |
| Intent-to- | N | 24 | 24 | — | — | — |
| Treat | | | | | | |
| | Least- | 18.7 | −3.54 | 22.3 | (11.1, | 0.0017 |
| | Squares | | | | 33.5) | |
| | Mean | | | | | |
| | Mean ± | 18.5 ± | −3.3 ± | — | — | — |
| | SD | 42.7 | 21.6 | | | |
| | Median | 22.5 | −5.0 | — | — | — |
| | Min, | −60.0, | −60.0, | — | — | — |
| | Max | 100.0 | 20.0 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

### Change in pH Results

Vaginal pH was measured at Screening and Visit 3—End of treatment (day 15). The pH measurement was obtained by pressing a pH indicator strip against the vaginal wall. The subjects entering the study were required to have a vaginal pH value greater than 5.0 at screening. pH values were recorded on the subject's case report form. The subjects were advised not to have sexual activity and to refrain from using vaginal douching within 24 hours prior to the measurement. Results from these assessments are presented in Table 9.

TABLE 9
Primary Efficacy Analysis Results of Change from Baseline
(Screening) to Day 15 in Vaginal pH

| | | | | Differ- | | |
|---|---|---|---|---|---|---|
| | | | | ence | | |
| | | | | Between | 90% | Estradiol |
| | | | | Treat- | CI for | 10 μg vs. |
| Pop- | Sta- | Estradiol | | ment | Differ- | Placebo |
| ulation | tistics | 10 μg | Placebo | Means | ence¹ | P-value² |
| Intent- | N | 24 | 24 | — | — | — |
| to- | | | | | | |
| Treat | | | | | | |
| | Least- | −0.974 | −0.339 | −0.635 | (−0.900,- | 0.0002 |
| | Squares | | | | 0.368) | |
| | Mean | | | | | |
| | Mean ± | −0.917 ± | −0.396 ± | — | — | — |
| | SD | 0.686 | 0.659 | | | |
| | Median | −1.00 | −0.500 | — | — | — |
| | Min, | −2.00, | −1.50, | — | — | — |
| | Max | 0.500 | 0.500 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

### Most Bothersome Symptoms Data

Subjects were asked to specify the symptom that she identified as the “most bothersome symptom.” During the screening period all of the subjects were provided with a questionnaire to self-assess the symptoms of VVA: (1) vaginal dryness; (2) vaginal or vulvar irritation, burning, or itching; (3) dysuria; (4) dyspareunia; (5) vaginal bleeding associated with sexual activity. Each symptom, with the exception of vaginal bleeding associated with sexual activity, was measured on a scale of 0 to 3, where 0=none, 1=mild, 2=moderate, and 3=severe. Vaginal bleeding associated with sexual activity was measured in a binary scale: N=no bleeding; Y=bleeding. The subject's responses were recorded. All randomized subjects were also provided a questionnaire to self-assess the symptoms of VVA at Visit 1—Randomization/Baseline (day 1) and at Visit 3—End of the treatment (day 15). Subjects recorded their self-assessments daily in a diary and answers were collected on days 8 and 15 (end of treatment). Pre-dose evaluation results obtained at Visit 1 were considered as baseline data for the statistical analyses. Data from these assessments are presented in Tables 10 and 11.

TABLE 10
Baseline Characteristics for Vaginal Atrophy Symptoms
(ITT Population)

| | | | | Estradiol 10 μg vs. |
|---|---|---|---|---|
| | | | | Placebo |
| VVA Symptom | Statistics | Estradiol 10 μg | Placebo | P-value¹ |
| Vaginal dryness | N of Subjects | 24 | 24 | — |
| | Mean | 2.292 | 2.375 | 0.68231 |
| Vaginal or vulvar irritation/ | N of Subjects | 24 | 24 | — |
| burning/itching | Mean | 0.875 | 1.333 | 0.08721 |
| Pain, burning or stinging | N of Subjects | 24 | 24 | — |
| when urinating | Mean | 0.583 | 0.625 | 0.87681 |
| Vaginal pain associated | N of Subjects² | 12 | 12 | — |
| with sexual activity | Mean | 2.083 | 2.333 | 0.54281 |
| Vaginal bleeding | N of Subjects² | 12 | 12 | |
| associated with sexual | Percent³ | 25.00 | 33.33 | 0.31463 |
| activity | | | | |

¹P-value for treatment comparison from ANOVA/ANCOVA with treatment as a fixed effect and Baseline as a covariate when appropriate.
²N = number of subjects sexually active at baseline.
³Percent of subjects with bleeding, evaluated using Fisher's Exact Test.

TABLE 11
Additional Efficacy Analysis Results of Change from Baseline (Randomization) to
Day 15 in Severity of Vaginal Atrophy Symptoms

| | | | | Difference | | Estradiol |
|---|---|---|---|---|---|---|
| | | Least-Squares Mean | | Between | | 10 μg vs. |
| | Statistical | TEstradiol | | Treatment | 90% CI for | Placebo |
| Symptom | Method¹ | 10 μg | PPlacebo | Means | Difference² | P-value |
| Vaginal dryness | AANCOVA | −0.980 | −0.729 | −0.251 | (−0.706, 0.204) | 00.3597 |
| Vaginal or vulvar | AANCOVA | −0.694 | −0.514 | −0.180 | (−0.549, 0.189) | 00.4159 |
| Irritation/ | | | | | | |
| burning/itching | | | | | | |
| Pain/Burning/ | AANCOVA | −0.391 | −0.359 | −0.032 | (−0.263, 0.200) | 00.8185 |
| Stinging | | | | | | |
| (Urination) | | | | | | |
| Vaginal pain | AANOVA | −0.800 | −0.500 | −0.300 | (−1.033, 0.433) | 00.4872 |
| associated with | | | | | | |
| sexual activity | | | | | | |

¹ANOVA model contained a fixed effect for treatment. ANCOVA added baseline as a covariate to the model.
²Confidence interval for the difference between estradiol 10 μg and Placebo treatment least-squares means.

Changes to the most bothersome symptom from the baseline was scored according to the evaluation of VVA symptoms generally set forth above. Tables 13 and 14 show a comparison between the pharmaceutical composition 1 and placebo generally for most bothersome symptom and vaginal atrophy symptom. It is noteworthy to point out that these measurement demonstrated a trend of improvement, though not statistically significant, at day 15.

TABLE 13
Primary Efficacy Analysis Results of Change from Baseline (Randomization)
to Day 15 in Severity of the Most Bothersome VVA

| | | | | Difference | | Estradiol |
|---|---|---|---|---|---|---|
| | | | | Between | | 10 μg vs. |
| | | Estradiol | | Treatment | 90% CI for | Placebo |
| Population | Statistics | 10 μg | Placebo | Means | Difference¹ | P-value² |
| Intent-to- | N | 24 | 24 | — | — | — |
| Treat | | | | | | |
| | Least- | −1.043 | −1.042 | −0.002 | (−0.497, | 0.9951 |
| | Squares | | | | 0.493) | |
| | Mean | | | | | |
| | Mean ± | −1.043 ± | −1.042 ± | — | — | — |
| | SD | 0.928 | 1.08 | | | |
| | Median | −1.00 | −1.00 | — | — | — |
| | Min, Max | −3.00, | −3.00, | — | — | — |
| | | 0.00 | 0.00 | | | |

¹Confidence interval for the estradiol 10 μg-Placebo from ANOVA with treatment as a fixed effect.
²P-value for treatment comparison from ANOVA with treatment as a fixed effect.

TABLE 14
Additional Efficacy Analysis Results of Change from Baseline (Randomization) to
Day 15 in Severity of Vaginal Atrophy Symptoms

| | | | | Difference | | TX-12-004- |
|---|---|---|---|---|---|---|
| | | Least-Squares Mean | | Between | | HR vs. |
| | Statistical | TX-12-004- | | Treatment | 90% CI for | Placebo P- |
| Symptom | Method¹ | HR | Placebo | Means | Difference² | value |
| Dryness | ANCOVA | −0.980 | −0.729 | −0.251 | (−0.706, 0.204) | 0.3597 |
| Irritation | ANCOVA | −0.694 | −0.514 | −0.180 | (−0.549, 0.189) | 0.4159 |
| Pain (Sex) | ANOVA | −0.800 | −0.500 | −0.300 | (−1.033, 0.433) | 0.4872 |
| Pain/Burning/ | ANCOVA | −0.391 | −0.359 | −0.032 | (−0.263, 0.200) | 0.8185 |
| Stinging | | | | | | |
| (Urination) | | | | | | |

¹ANOVA model contained a fixed effect for treatment. ANCOVA added baseline as a covariate to the model.
²Confidence interval for the difference between TX-12-004-HR and Placebo treatment least-squares means.

With respect to the most bothersome symptoms data presented in Tables 13 and 14, the period over which the data was measured is generally considered insufficient to make meaningful conclusions. However, the trends observed as part of this study suggest that the data will show improvement of the most bothersome symptoms when data for a longer time period is collected.

The absence or presence of any vaginal bleeding associated with sexual activity was also measured as one of the most bothersome symptoms. The data for vaginal bleeding associated with sexual activity is reported in Table 15.

TABLE 15
Primary Efficacy Analysis Results of Change from Baseline
(Randomization) to Day 15 in Vaginal Bleeding Associated
with Sexual Activity

| | | Baseline (Randomization) and | | | |
|---|---|---|---|---|---|
| | | Day 15 Summary of Vaginal Bleeding | | | |
| | | Bleeding/ | | No | |
| | | No | Bleeding/ | Bleeding/ | No Bleeding/ |
| | | Bleeding | Bleeding | Bleeding | No Bleeding |
| Treatment | N* | (Success)² | (Failure) | (Failure) | (NC) |
| Estradiol 10 μg | 10 | 2 (100%) | 0 | 0 | 8 |
| Placebo | 10 | 1 (20%) | 3 | 1 | 5 |

| | | | | |
|---|---|---|---|---|
| P-Value for | 0.1429 | — | — | — |
| Estradiol 10 μg vs. | | | | |
| Placebo¹ | | | | |

*N = Total number of patients within each treatment group who were sexually active at both Baseline and Day 15 and provided a response at both visits.
NC = No Change - not considered in the statistical comparison.
¹P-value for treatment comparison from Fisher's Exact Test.
²Percent is based on the number of subjects classified as either a Success or a Failure (N = 2 for estradiol 10 μg; N = 5 for Placebo

### Estradiol Level/Pharmacokinetics Data

In this study, the systemic exposure to estradiol following once daily intravaginal administration of estradiol 10 μg for 14 days was investigated. Descriptive statistics of the plasma estradiol concentrations taken at each sampling time and the observed Cmax and Tmax values were recorded in Tables 16 and 17. No statistically significant difference in the systemic concentration of estradiol 10 μg versus the placebo group was observed, which suggests the estradiol is not carried into the blood stream where it will have a systemic effect. Rather, it remains in localized tissues; the effect of estradiol is therefore believed be local to the location of administration (i.e., the vagina). The lower limits of detection of the assays used to measure the pharmacokinetic data may have affected the measured the accuracy of the PK values presented. Additional PK studies were performed with more accurate assays in Examples 8 and 9.

For the purpose of monitoring the estradiol level during the study blood samples were collected at 0.0, 1.0, 3.0, and 6.0 hours relative to dosing on day 1; prior to dosing on day 8; and prior to dosing on day 15. Efforts were made to collect blood samples at their scheduled times. Sample collection and handling procedures for measurement of estradiol blood level was performed according to procedure approved by the sponsor and principal investigator. All baseline and post-treatment plasma estradiol concentrations were determined using a validated bioanalytical (UPLC-MS/MS) methods. These data are shown in Tables 16 and 17.

TABLE 16
Descriptive Statistics of Estradiol Concentrations (pg/mL) at Each Sampling Time

| | Sampling Time | | | | | |
|---|---|---|---|---|---|---|
| | | | | | Pre-dose | Pre-dose |
| Treatment | 0 Hour | 1 Hour | 3 Hours | 6 Hours | Day 8 | Day 15 |
| Estradiol 10 μg | | | | | | |
| N | 24 | 24 | 24 | 24 | 24 | 22 |
| Mean ± SD | 20.1 ± 5.74 | 28.7 ± 5.89 | 25.7 ± 5.71 | 23.4 ± 7.91 | 21.4 ± 9.28 | 23.4 ± 8.72 |
| Median | 20.2 | 28.9 | 24.7 | 22.3 | 20.7 | 20.7 |
| Min, Max | 2.63, 38.3 | 18.8, 43.9 | 19.3, 47.5 | 3.31, 52.3 | 2.09, 52.2 | 17.9, 54.7 |
| Placebo | | | | | | |
| N | 26 | 26 | 26 | 26 | 25 | 24 |
| Mean ± SD | 20.5 ± 4.29 | 21.0 ± 6.14 | 19.0 ± 5.92 | 26.9 ± 17.36 | 29.9 ± 22.51 | 28.1 ± 16.80 |
| Median | 20.8 | 20.8 | 20.9 | 21.7 | 21.6 | 21.1 |
| Min, Max | 4.03, 29.1 | 3.19, 41.2 | 3.15, 26.9 | 15.1, 90.0 | 15.0, 116.2 | 14.7, 81.3 |

TABLE 17
Descriptive Statistics of Estradiol Cₘₐₓ and Tₘₐₓ on Day 1

| | Estradiol 10 μg | | Placebo | |
|---|---|---|---|---|
| | Cₘₐₓ | Tₘₐₓ | Cₘₐₓ | Tₘₐₓ |
| N | 24 | 24 | 26 | 26 |
| Mean ± SD | 30.7 ± 7.47 | 2.12 ± 1.73 | 27.5 ± 17.26 | 4.00 ± 2.68 |
| Geometric | 29.9 | — | 24.7 | — |
| Mean | | | | |
| Median | 29.8 | 1.00 | 22.1 | 6.00 |
| Min, Max | 19.7, 52.3 | 1.00, 6.00 | 15.1, 90.0 | 0.00, 6.00 |
| CV % | 24.3% | 81.3% | 62.9% | 67.1% |

### Assessment of Vaginal Mucosa Data

The investigators rated the vaginal mucosal appearance at day 1 (pre-dose) and day 15. Vaginal color, vaginal epithelial integrity, vaginal epithelial surface thickness, and vaginal secretions were evaluated according to the following degrees of severity: none, mild, moderate, or severe using scales 0 to 3, where 0=none, 1=mild, 2=moderate, and 3=severe. Results from these investigators rated assessments are presented in Tables 18, 19, 20, and 21.

TABLE 18
Primary Efficacy Analysis Results of Change from Baseline
(Randomization) to Day 15 in Investigator's Assessment of
the Vaginal Mucosa (Assessment of Vaginal Color)

| | | | | Difference | | Estradiol |
|---|---|---|---|---|---|---|
| | | | | Between | 90% | 10 μg vs. |
| | | Estradiol | | Treatment | CI for | Placebo |
| Population | Statistics | 10 μg | Placebo | Means | Difference¹ | P-value² |
| Intent-to- | N | 24 | 24 | — | — | — |
| Treat | | | | | | |
| | Least- | −0.199 | −0.009 | −0.191 | 0.052) | 0.1945 |
| | squares | | | | | |
| | Mean | | | | | |
| | Mean ± | −0.333 ± | 0.125 ± | | | |
| | SD | 0.565 | 0.741 | | | |
| | Median | 0.00 | 0.00 | — | — | — |
| | Min, | −2.00, | −1.00, | — | — | — |
| | Max | 0.00 | 2.00 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

TABLE 19
Primary Efficacy Analysis Results of Change from Baseline
(Randomization) to Day 15 in Investigator's Assessment of the
Vaginal Mucosa (Assessment of Vaginal Epithelial Integrity)

| | | | | Difference | | Estradiol |
|---|---|---|---|---|---|---|
| | | | | Between | 90% | 10 μg vs. |
| | | Estradiol | | Treatment | CI for | Placebo |
| Population | Statistics | 10 μg | Placebo | Means | Difference¹ | P-value² |
| Intent-to- | N | 24 | 24 | — | — | — |
| Treat | | | | | | |
| | Least- | −0.342 | 0.176 | −0.518 | (−0.726, | 0.0001 |
| | squares | | | | −0.311) | |
| | Mean | | | | | |
| | Mean ± | −0.417 ± | 0.250 ± | | | |
| | SD | 0.584 | 0.442 | | | |
| | Median | 0.00 | 0.00 | — | — | — |
| | Min, | −1.00, | 0.00, | — | — | — |
| | Max | 1.00 | 1.00 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

TABLE 20
Primary Efficacy Analysis Results of Change from Baseline (Randomization)
to Day 15 in Investigator's Assessment of the Vaginal Mucosa
(Assessment of Vaginal Epithelial Surface Thickness)

| | | | | Difference | | Estradiol |
|---|---|---|---|---|---|---|
| | | | | Between | 90% | 10 μg vs. |
| | | Estradiol | | Treatment | CI for | Placebo |
| Population | Statistics | 10 μg | Placebo | Means | Difference¹ | P-value² |
| Intent-to- | N | 24 | 24 | — | — | — |
| Treat | | | | | | |
| | Least- | −0.034 | −0.133 | 0.099 | (−0.024, | 0.1820 |
| | squares | | | | 0.221) | |
| | Mean | | | | | |
| | Mean ± | −0.125 ± | −0.042 ± | — | — | — |
| | SD | 0.338 | 0.550 | | | |
| | Median | 0.00 | 0.00 | — | — | — |
| | Min, | −1.00, | −1.00, | — | — | — |
| | Max | 0.00 | 1.00 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

TABLE 21
Primary Efficacy Analysis Results of Change from Baseline
(Randomization) to Day 15 in Investigator's Assessment of the
Vaginal Mucosa (Assessment of Vaginal Secretions)

| | | | | Difference | | Estradiol |
|---|---|---|---|---|---|---|
| | | | | Between | 90% | 10 μg vs. |
| | | Estradiol | | Treatment | CI for | Placebo |
| Population | Statistics | 10 μg | Placebo | Means | Difference¹ | P-value² |
| Intent-to- | N | 24 | 24 | — | — | — |
| Treat | | | | | | |
| | Least- | −0.643 | −0.274 | −0.369 | (−0.661, | 0.0401 |
| | squares | | | | −0.076) | |
| | Mean | | | | | |
| | Mean ± | −0.792 ± | −0.125 ± | — | — | — |
| | SD | 0.779 | 0.741 | | | |
| | Median | −1.00 | 0.00 | — | — | — |
| | Min, | | | | | |
| | Max | −2.00, | −2.00, | — | — | — |
| | | 1.00 | 2.00 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

Delivery Vehicle Disintegration Data

Assessment of capsule disintegration in the vagina (presence or absence) at Day 1 (6 hours after dosing) and Day 15. Results of this assessment is presented in Table 22.

TABLE 22
Capsule Disintegration State in the Vagina on Day 1 and Day 15

| | Estradiol 10 μg | | Placebo | |
|---|---|---|---|---|
| | DDay 1 | DDay 15 | DDay 1 | DDay 15 |
| No evidence of | 223 (95.8%) | 224 (100.0%) | 226 (100.0%) | 224 (92.3%) |
| capsule present | | | | |
| Evidence of capsule | 0 (0.0%) | 0 (0.0%) | 0 (0.0%) | 0 (0.0%) |
| present | | | | |
| Assessment not | 1 (4.2%) | 0 (0.0%) | 0 (0.0%) | 2 (7.7%) |
| done | | | | |

Serum hormone level data was collected to measure the serum concentrations of estradiol. These data were used for screening inclusion and were determined using standard clinical chemistry methods.

### Appropriateness of Measurements

The selection of the efficacy measurements used in this study was based on FDA's recommendations for studies of estrogen and estrogen/progestin drug products for the treatment of moderate to severe vasomotor symptoms associated with the menopause and moderate to severe symptoms of vulvar and vaginal atrophy associated with the menopause (Food and Drug Administration, Guidance for Industry, Estrogen and Estrogen/Progestin Drug Products to Treat Vasomotor Symptoms and Vulvar and Vaginal Atrophy Symptoms—Recommendations for Clinical Evaluation. January 2003, hereby incorporated by reference).

Standard clinical, laboratory, and statistical procedures were utilized in the trial. All clinical laboratory procedures were generally accepted and met quality standards.

### Statistical Methods:

Efficacy:

Analysis of variance (ANOVA) was used to evaluate the change from baseline differences between the subjects receiving estradiol 10 μg and placebo capsules for all efficacy endpoints, except for vaginal bleeding, to estimate the effect size and variability of the effect. In some cases, for example, for some vaginal atrophy symptoms, the change from baseline (post dose response) was correlated with the baseline value (p<0.05), so baseline was included as a covariate to adjust for this correlation (Analysis of Covariance, ANCOVA). The 90% confidence intervals on the differences between estradiol 10 μg and placebo endpoint means were determined to evaluate the effect size. The change from baseline in vaginal bleeding associated with sexual activity was evaluated in terms of the proportion of subjects who had treatment success or failure. Any subject reporting bleeding at baseline who did not report bleeding at Day 15 was considered to have been successfully treated. Any subject reporting bleeding at day 15 was considered a treatment failure, regardless of whether they reported baseline bleeding or not. Subjects reporting no bleeding at both baseline and day 15 were classified as no-change and were excluded from the statistical evaluation. The difference in the proportion of subjects with success between the two treatment groups was statistically evaluated using Fisher's Exact Test. Results of this difference in proportion are presented in Table 10.

### Measurements of Treatment Compliance

Subjects were required to complete a diary in order to record treatment compliance. Diaries were reviewed for treatment compliance at day 8 and day 15 visits. A total of 45 subjects (21 subjects in the estradiol 10 μg group and 24 subjects in the placebo group) were 100% compliant with the treatment regimen.

Due to the investigative nature of the study, no adjustments were made for multiplicity of endpoints.

Safety:

The frequency and severity of all adverse events were summarized descriptively by treatment group.

Results: All forty eight (48) subjects who completed the study were included in the primary efficacy analyses. The results of efficacy analyses are presented throughout Tables 5, 6, and 7.

### Conclusions

Efficacy:

The two-week treatment with pharmaceutical composition 10 μg led to a statistically significant greater mean decrease in percent of parabasal cells than did placebo treatment (54% vs. 5%, p<0.0001), as illustrated in Table 6. At the same time, a significantly greater mean increase in the percent of superficial cells was observed with the pharmaceutical composition (35%) than with the placebo capsules (9%), with the difference being highly statistically significant (p=0.0002), as illustrated in Table 7. The difference in pH reduction between the pharmaceutical composition (0.97 units) compared to that for the placebo (0.34 units) was only slightly greater than 0.5 units, but the difference was detected as statistically significant (p=0.0002), as illustrated in Table 9.

While the decrease in severity of the most bothersome symptom was essentially the same (˜1 unit) for both pharmaceutical composition and placebo, the reductions in the severity of the individual symptoms of vaginal dryness, irritation and pain during sexual activity were all marginally better for the active treatment than for the placebo treatment. None of the differences between the two treatments, all of which were ≤0.3 units, were detected as statistically significant. There was no difference between the two treatments in regard to reduction of pain/burning/stinging during urination (˜0.4 unit reduction). The length of the study was not long enough to show a separation between the most bothersome symptoms in the pharmaceutical composition and placebo. However, the trends of most bothersome symptoms suggest that with a suitable period of time, significantly significant differences between the two treatments would be observed.

The two-week treatment with estradiol 10 μg capsules showed no statistically detectable difference in regard to reduction of severity from baseline according to the investigator's assessment of vaginal color or vaginal epithelial surface thickness. Pharmaceutical composition capsules did demonstrate a statistically significant greater reduction than did placebo in severity of atrophic effects on vaginal epithelial integrity (−0.34 vs. 0.18, p=0.0001) and vaginal secretions (−0.64 vs. −0.27, p=0.0401).

Descriptive statistical analyses (mean, median, geometric mean, standard deviation, CV, minimum and maximum, Cmax, and Tmax) were conducted on the estradiol concentrations at each sampling time, the peak concentration on day 1 and the time of peak concentration. Results from this assessment are presented in Tables 16 and 17.

A pharmaceutical composition comprising estradiol 10 μg outperformed placebo treatment in regard to improvement in the Maturation Index, reduction in vaginal pH, reduction in the atrophic effects on epithelial integrity and vaginal secretions. The lack of statistical significance between the two treatments in regard to reduction of severity for the most bothersome symptom, and the individual vaginal atrophy symptoms of dryness, irritation, pain associated with sexual activity, and pain/burning/stinging during urination, is not unexpected given the small number of subjects in the study and the short duration of therapy. Too few subjects in the study had vaginal bleeding associated with sexual activity to permit any meaningful evaluation of this vaginal atrophy symptom.

Of the 48 subjects enrolled in the study, 45 subjects were 100% compliant with the treatment regimen. Of the remaining three subjects, one removed herself from the study due to personal reasons and the other two subjects each missed one dose due to an adverse event.

Safety:

Although the Day 1 mean plasma estradiol peak concentration for the pharmaceutical composition was somewhat higher than that for the Placebo (ratio of geometric means=1.21: Test Product (estradiol 10 μg) 21%>Placebo), no statistically significant difference was determined. However, the assay methods were questionable, resulting in questionable PK data. Additional PK studies were performed in Examples 8 and 9.

There were no serious adverse events in the study.

Overall, the pharmaceutical composition comprising estradiol 10 μg was well tolerated when administered intravaginally in once daily regimen for 14 days.

### Example 8: PK Study (25 μg Formulation)

A PK study was undertaken to compare the 25 μg formulation disclosed herein (Pharmaceutical Composition 3) to the RLD. The results of the PK study for estradiol are summarized in Table 23. The p values for these data demonstrate statistical significance, as shown in Table 24.

TABLE 23
Statistical Summary of the Comparative Bioavailability Data
for Unscaled Average BE studies of Estradiol, Least Square
Geometric Means of Estradiol, Ratio of Means and 90%
Confidence Intervals, Fasting/Fed Bioequivalence Study
(Study No.: ESTR-1K-500-12), Dose 25 μg estradiol

| Parameter | Test | N | RLD | N | Ratio (%) | 90% C.I. |
|---|---|---|---|---|---|---|
| Cₘₐₓ | 23.0839 | 36 | 42.7024 | 36 | 54.06 | 44.18- |
| (pg/mL) | | | | | | 66.14 |
| AUC₀₋₂₄ | 89.2093 | 36 | 292.0606 | 36 | 30.54 | 23.72- |
| (pg · hr/mL) | | | | | | 39.34 |

TABLE 24
P-values for Table 23

P-Value

| Effect | Cₘₐₓ | AUC₀₋₂₄ |
|---|---|---|
| Treatment | <.0001 | <.0001 |
| Sequence | 0.4478 | 0.5124 |
| Period | 0.4104 | 0.7221 |

As illustrated in Table 23, baseline adjusted PK data illustrates that the formulations disclosed herein unexpectedly show a 54% decrease in Cmax and a 31% decrease in the AUC relative to the RLD. This result is desirable because the estradiol is intended only for local absorption. These data suggest a decrease in the circulating levels of estradiol relative to the RLD. Moreover, it is noteworthy to point out that the Cmax and AUC levels of estradiol relative to placebo are not statistically differentiable, which suggests that the formulations disclosed herein have a negligible systemic effect. As shown in Table 24, there was no significant difference between the test and reference products due to sequence and period effects. However, there was a significant difference due to treatment effect for both Cmax and AUC.

Pharmacokinetics for circulating total estrone, a metabolite of estradiol, is show in Table 25. These data show that the total circulating estrone for the formulations disclosed herein resulted in a 55% decrease in the Cmax for circulating estrone, and a 70% decrease in the AUC for circulating estrone.

TABLE 25
Statistical Summary of the Comparative Bioavailability
Data for Unscaled Average BE studies of Estrone,
Least Square Geometric Means, Ratio of Means
and 90% Confidence Intervals, Fasting/Fed Bioequivalence
Study (Study No.: ESTR-1K-500-12), Dose 25 μg estradiol

| Parameter | Test | N | RLD | N | Ratio (%) | 90% C.I. |
|---|---|---|---|---|---|---|
| Cₘₐₓ | 10.7928 | 36 | 23.5794 | 36 | 45.77 | 32.95 to |
| (pg/mL) | | | | | | 63.59 |
| AUC₀₋₂₄ | 51.2491 | 36 | 165.4664 | 36 | 30.97 | 19.8- |
| (pg · hr/mL) | | | | | | 48.45 |

TABLE 26
P-values for Table 25

P-Value

| Effect | Cₘₐₓ | AUC₀₋₂₄ |
|---|---|---|
| Treatment | 0.0002 | <.0001 |
| Sequence | 0.1524 | 0.0464 |
| Period | 0.0719 | 0.0118 |

There was a significant difference between test and reference products due to treatment effect whereas there was no significant difference due to sequence and period effects for Cmax. For AUC, there was a significant difference between test and reference products due to treatment, sequence, and period effects.

PK for circulating total estrone sulfate is shown in Table 27. These data show that the total circulating estrone sulfate for the pharmaceutical compositions disclosed herein resulted in a 33% decrease in the Cmax and a 42% decrease in the AUC for circulating estrone sulfate.

TABLE 27
Statistical Summary of the Comparative Bioavailability
Data for Unscaled Average BE studies of Estrone
Sulfate, Least Square Geometric Means of Estrone
Sulfate, Ratio of Means and 90% Confidence Intervals,
Fasting/Fed Bioequivalence Study
(Study No.: ESTR-IK-500-12), Dose 25 μg estradiol

| | | | | | Ratio | 90% |
|---|---|---|---|---|---|---|
| Parameter | Test | N | RLD | N | (%) | C.I. |
| Cₘₐₓ | 490.0449 | 36 | 730.5605 | 36 | 67.08 | 53.84- |
| (pg/mL) | | | | | | 83.57 |
| AUC₀₋₂₄ | 4232.9914 | 36 | 7323.0827 | 36 | 57.80 | 43.23- |
| (pg · hr/mL) | | | | | | 77.29 |

TABLE 28
P-values for Table 27

P-Value

| Effect | Cₘₐₓ | AUC₀₋₂₄ |
|---|---|---|
| Treatment | 0.0042 | 0.0031 |
| Sequence | 0.5035 | 0.9091 |
| Period | 0.1879 | 0.8804 |

There was a significant difference between test and reference products due to treatment effect whereas there was no significant difference due sequence and period effects for both Cmax and AUC.

### Example 9: PK Study (10 μs Formulation)

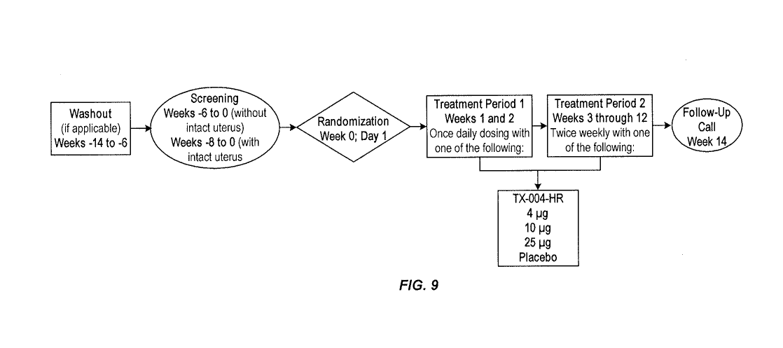

A PK study was undertaken to compare the 10 μg formulation disclosed herein (Pharmaceutical Composition 2) to the RLD. The results of the PK study for estradiol are summarized in Table 29-40, and FIGS. 9-14.

A PK study was undertaken to compare pharmaceutical compositions disclosed herein having 10 μg of estradiol to the RLD. The results of the PK study for estradiol are summarized in Tables 29-34, which demonstrate that the pharmaceutical compositions disclosed herein more effectively prevented systemic absorption of the estradiol. Table 35 shows that the pharmaceutical compositions disclosed herein had a 28% improvement over the RLD for systemic blood concentration Cmax and 72% AUC improvement over the RLD.

TABLE 29
Summary of Pharmacokinetic Parameters of Test product (T)
of Estradiol - Baseline adjusted (N = 34)

| | Arithmetic | | | | |
|---|---|---|---|---|---|
| | Mean ± | | | | |
| Pharmacokinetic | Standard | Coefficient | | | |
| Parameter | Deviation | of Variation | Median | Minimum | Maximum |
| Cₘₐₓ (pg/mL) | 15.7176 ± 7.9179 | 50.3761 | 13.9000 | 6.5000 | 49.6000 |
| AUC₀₋₂₄ (pg · hr/mL) | 53.0100 ± 19.5629 | 36.9041 | 49.9750 | 24.3000 | 95.1500 |
| tₘₐₓ (hr) | 1.98 ± 1.29 | 65.34 | 2.00 | 1.00 | 8.05 |

TABLE 30
Summary of Pharmacokinetic Parameters of Reference
product (R) of Estradiol - Baseline adjusted (N = 34)

| | Arithmetic | | | | |
|---|---|---|---|---|---|
| | Mean ± | | | | |
| Pharmacokinetic | Standard | Coefficient | | | |
| Parameter | Deviation | of Variation | Median | Minimum | Maximum |
| Cₘₐₓ (pg/mL) | 24.1882 ± 11.9218 | 49.2877 | 24.1500 | 1.0000 | 55.3000 |
| AUC₀₋₂₄ (pg · hr/mL) | 163.8586 ± 72.0913 | 43.9960 | 158.0375 | 2.0000 | 304.8500 |
| tₘₐₓ (hr) | 10.53 ± 5.58 | 52.94 | 8.06 | 2.00 | 24.00 |

TABLE 31
Geometric Mean of Test Product (T) and Reference product (R)
of Estradiol - Baseline adjusted (N = 34)

| | Geometric Mean | |
|---|---|---|
| Pharmacokinetic Parameter | Test Product (T) | Reference Product (R) |
| Cₘₐₓ (pg/mL) | 14.3774 | 20.3837 |
| AUC₀₋₂₄ (pg · hr/mL) | 49.6231 | 132.9218 |
| tₘₐₓ (hr) | 1.75 | 9.28 |

TABLE 32
Statistical Results of Test product (T) versus Reference
product (R) for Estradiol - Baseline adjusted (N = 34)

| | Geometric Least | | | | |
|---|---|---|---|---|---|
| | Square Mean | | | | |
| | Test | Reference | Intra | | 90% |
| Pharmacokinetic | Product | Product | Subject | T/R | Confidence |
| Parameter | (T) | (R) | CV % | Ratio % | Interval |
| Cₘₐₓ (pg/mL) | 14.4490 | 20.1980 | 60.68 | 71.54* | 56.82-90.08 |
| AUC₀₋₂₄ | 49.7310 | 131.0400 | 70.64 | 37.95* | 29.21-49.31 |
| (pg · hr/mL) | | | | | |

*Comparison was detected as statistically significant by ANOVA (α = 0.05).

The PK data for total estrone likewise demonstrated reduced systemic exposure when compared to the RLD. Table 33 shows the pharmaceutical compositions disclosed herein reduced systemic exposure by 25% for Cmax and 49% for AUC.

TABLE 33
Summary of Pharmacokinetic Parameters of Test product (T)
of Estrone - Baseline adjusted (N = 33)

| | Arithmetic | | | | |
|---|---|---|---|---|---|
| | Mean ± | | | | |
| Pharmacokinetic | Standard | Coefficient | | | |
| Parameter | Deviation | of Variation | Median | Minimum | Maximum |
| Cₘₐₓ (pg/mL) | 6.8485 ± 6.5824 | 96.1149 | 5.4000 | 1.3000 | 36.3000 |
| AUC₀₋₂₄ (pg · hr/mL) | 34.7051 ± 27.9541 | 80.5476 | 30.8500 | 3.3500 | 116.7500 |
| tₘₐₓ (hr) | 9.12 ± 8.83 | 96.80 | 4.00 | 1.00 | 24.00 |

TABLE 34
Summary of Pharmacokinetic Parameters of Reference
product (R) of Estrone - Baseline adjusted (N = 33)

| | Arithmetic | | | | |
|---|---|---|---|---|---|
| | Mean ± | | | | |
| Pharmacokinetic | Standard | Coefficient | | | |
| Parameter | Deviation | of Variation | Median | Minimum | Maximum |
| Cₘₐₓ (pg/mL) | 8.8333 ± 7.1469 | 80.9086 | 6.7000 | 2.7000 | 30.3000 |
| AUC₀₋₂₄ (pg · hr/mL) | 63.0042 ± 46.5484 | 73.8814 | 51.2800 | 8.8000 | 214.0000 |
| tₘₐₓ (hr) | 11.16 ± 7.24 | 64.95 | 10.00 | 4.00 | 24.00 |

TABLE 35
Geometric Mean of Test Product (T) and Reference product (R)
of Estrone - Baseline adjusted (N = 33)

| | Geometric Mean | |
|---|---|---|
| Pharmacokinetic Parameter | Test Product (T) | Reference Product (R) |
| Cₘₐₓ (pg/mL) | 5.1507 | 6.9773 |
| AUC₀₋₂₄ (pg · hr/mL) | 24.2426 | 48.2377 |
| tₘₐₓ (hr) | 5.87 | 9.07 |

TABLE 36
Statistical Results of Test product (T) versus Reference
product (R) for Estrone - Baseline adjusted (N = 33)

| | Geometric Least | | | | |
|---|---|---|---|---|---|
| | Square Mean | | | | |
| | Test | Reference | Intra | | 90% |
| Pharmacokinetic | Product | Product | Subject | T/R | Confidence |
| Parameter | (T) | (R) | CV % | Ratio % | Interval |
| Cₘₐₓ (pg/mL) | 5.1620 | 6.9280 | 47.59 | 74.50* | 61.69-89.97 |
| AUC₀₋₂₄ | 24.1960 | 47.9020 | 73.66 | 50.51* | 38.37-66.50 |
| (pg · hr/mL) | | | | | |

*Comparison was detected as statistically significant by ANOVA (α = 0.05).

The PK data for estrone sulfate likewise demonstrated reduced systemic exposure when compared to the RLD. Table 37 shows the pharmaceutical compositions disclosed herein reduced systemic exposure by 25% for Cmax and 42% for AUC.

TABLE 37
Summary of Pharmacokinetic Parameters of Test product (T)
of Estrone Sulfate - Baseline adjusted (N = 24)

| | Arithmetic | | | | |
|---|---|---|---|---|---|
| | Mean ± | | | | |
| Pharmacokinetic | Standard | Coefficient | | | |
| Parameter | Deviation | of Variation | Median | Minimum | Maximum |
| Cₘₐₓ (ng/mL) | 13.9042 ± 7.0402 | 50.6339 | 11.1500 | 1.3000 | 39.0000 |
| AUC₀₋₂₄ (ng · hr/mL) | 97.9953 ± 80.8861 | 82.5408 | 76.2750 | 5.1025 | 338.0000 |
| tₘₐₓ (hr) | 6.33 ± 4.56 | 71.93 | 4.00 | 4.00 | 24.00 |

TABLE 38
Summary of Pharmacokinetic Parameters of Reference
product (R) of Estrone Sulfate - Baseline adjusted (N = 24)

| | Arithmetic | | | | |
|---|---|---|---|---|---|
| | Mean ± | | | | |
| Pharmacokinetic | Standard | Coefficient | | | |
| Parameter | Deviation | of Variation | Median | Minimum | Maximum |
| Cₘₐₓ (ng/mL) | 19.2542 ± 11.3633 | 59.0173 | 15.2000 | 7.0000 | 53.7000 |
| AUC₀₋₂₄ (ng · hr/mL) | 177.6208 ± 166.2408 | 93.5931 | 124.0000 | 20.0000 | 683.0500 |
| tₘₐₓ (hr) | 10.33 ± 5.58 | 54.05 | 10.00 | 2.00 | 24.00 |

TABLE 39
Geometric Mean of Test Product (T) and Reference product (R)
of Estrone Sulfate - Baseline adjusted (N = 24)

| | Geometric Mean | |
|---|---|---|
| Pharmacokinetic Parameter | Test Product (T) | Reference Product (R) |
| Cₘₐₓ (ng/mL) | 12.1579 | 16.8587 |
| AUC₀₋₂₄ (ng · hr/mL) | 66.5996 | 121.5597 |
| tₘₐₓ (hr) | 5.49 | 8.83 |

TABLE 40
Statistical Results of Test product (T) versus Reference product (R)
for Estrone Sulfate - Baseline adjusted (N = 24)

| | Geometric Least | | | | |
|---|---|---|---|---|---|
| | Square Mean | | | | |
| | Test | Reference | Intra | | 90% |
| Pharmacokinetic | Product | Product | Subject | T/R | Confidence |
| Parameter | (T) | (R) | CV % | Ratio % | Interval |
| Cₘₐₓ (ng/mL) | 12.3350 | 16.5470 | 48.02 | 74.55* | 59.43-93.51 |
| AUC₀₋₂₄ | 68.5260 | 118.4170 | 73.87 | 57.87* | 41.68-80.35 |
| (ng · hr/mL) | | | | | |

*Comparison was detected as statistically significant by ANOVA (α = 0.05).

### Example 10: Randomized, Double-Blind, Placebo-Controlled Multicenter Study of Estradiol Vaginal Softgel Capsules for Treatment of VVA

### Investigational Plan

The study was a randomized, double-blind, placebo-controlled multicenter study design. Postmenopausal subjects who meet the study entry criteria will be randomized in a 1:1:1:1 ratio to receive Estradiol Vaginal Softgel Capsule 4 μg, Estradiol Vaginal Softgel Capsule 10 μg, Estradiol Vaginal Softgel Capsule 25 μg, or matching placebo. Subjects will be asked to self-assess the symptoms of vulvar or vaginal atrophy including vaginal pain associated with sexual activity, vaginal dryness, vulvar or vaginal itching or irritation by completing the VVA symptom self-assessment questionnaire and identification of her MBS at screening visit 1A to determine eligibility for the study. The VVA symptom Self-Assessment Questionnaire, vaginal cytology, vaginal pH, and vaginal mucosa will be assessed at screening visit 1B. These assessments will determine continued eligibility and will be used as the baseline assessments for the study. Randomized subjects will then complete the Questionnaire during visits 3, 4, 5, and 6.

The primary efficacy endpoints for the study included: (A) change from baseline to week 12 in the percentage of vaginal superficial cells (by vaginal cytologic smear) compared to placebo; (B) change from baseline to week 12 in the percentage of vaginal parabasal cells (by vaginal cytologic smear) compared to placebo; (C) change from baseline at week 12 in vaginal pH as compared to placebo; and (D) change from baseline to week 12 on the severity of the MBS of dyspareunia (vaginal pain associated with sexual activity) associated with VVA as compared to placebo.

The secondary efficacy endpoints for the study included: (E) change from baseline to weeks 2, 6, and 8 in the percentage of vaginal superficial cells (by vaginal cytologic smear) compared to placebo; (F) change from baseline to weeks 2, 6, and 8 in the percentage of vaginal parabasal cells (by vaginal cytologic smear) compared to placebo; (G) change from baseline to weeks 2, 6, and 8 in vaginal pH as compared to placebo; (H) change from baseline to weeks 2, 6, and 8 on the severity of the MBS of dyspareunia (vaginal pain associated with sexual activity) associated with VVA as compared to placebo; (I) change from baseline to weeks 2, 6, 8, and 12 on the severity of vaginal dryness and vulvar or vaginal itching or irritation associated with VVA as compared to placebo; (J) change in visual evaluation of the vaginal mucosa from baseline to weeks 2, 6, 8, and 12 compared to placebo; (K) assessment of standard PK parameters as defined in the SAP for serum estradiol, estrone, and estrone conjugates at Screening Visit 1A, days 1, 14, and 84 of treatment in a subset of subjects (PK substudy) utilizing baseline corrected and uncorrected values [as outlined in the Statistical Analysis Plan (SAP)]; and (L) change from baseline in the Female Sexual Function Index (FSFI) at week 12 compared to placebo.

The safety endpoints for the study included: (1) Adverse events; (2) Vital signs; (3) Physical examination findings; (4) Gynecological examination findings; (5) Clinical laboratory tests; (6) Pap smears; and (7) Endometrial biopsy.

Approximately 100 sites in the United States and Canada screened approximately 1500 subjects to randomize 747 subjects in this study (modified intent to treatment population, or all subjects who have taken at least one dose of the pharmaceutical compositions disclosed herein), with a target of 175 subjects randomized to each treatment group (175 in each active treatment group and 175 in the placebo group to complete 560 subjects). Actual subjects enrolled are 186 subjects in the 4 μg formulation group, 188 subjects in the 10 μg formulation group, 186 subjects in the 25 μg formulation group, and 187 subjects in the placebo group, for a total of 747 subjects in the study. Within each treatment group, 15 subjects also participated in a PK substudy. Subjects were assigned to one of four treatment groups: (1) 4 μg formulation; (2) 10 μg formulation; (3) 25 μg formulation; and (4) placebo.

Most subjects participated in the study for 20-22 weeks. This included a 6 to 8 week screening period (6 weeks for subjects without an intact uterus and 8 weeks for subjects with an intact uterus), 12 weeks on the investigational product, and a follow-up period of approximately 15 days after the last dose of investigational product. Some subjects' involvement lasted up to 30 weeks when an 8-week wash-out period was necessary. Subjects who withdrew from the study were not replaced regardless of the reason for withdrawal.

The study schematic diagram shown in FIG. 9. There were two treatment periods; once daily intravaginal administration of one of the listed investigational products for 2 weeks, followed by a twice weekly intravaginal administration for 10 weeks.

The subject inclusion criteria included: (1) postmenopausal female subjects between the ages of 40 and 75 years (at the time of randomization) with at least: 12 months of spontaneous amenorrhea (women <55 years of age with history of hysterectomy without bilateral oophorectomy prior to natural menopause must have follicle stimulating hormone (FSH) levels >40 mIU/mL); or 6 months of spontaneous amenorrhea with follicle stimulating hormone (FSH) levels >40 mlU/mL; or At least 6 weeks postsurgical bilateral oophorectomy.

The subject inclusion criteria also included: (2) ≤5% superficial cells on vaginal cytological smear; (3) Vaginal pH >5.0; (4) Moderate to severe symptom of vaginal pain associated with sexual activity considered the most bothersome vaginal symptom by the subject at screening visit 1A; (5) Moderate to severe symptom of vaginal pain associated with sexual activity at screening visit 1B; (6) Onset of moderate to severe dyspareunia in the postmenopausal years; (7) Subjects were sexually active (i.e., had sexual activity with vaginal penetration within approximately 1 month of screening visit 1A); and (8) Subjects anticipated having sexual activity (with vaginal penetration) during the conduct of the trial

For subjects with an intact uterus, the subject inclusion criteria also included: (9) subjects had an acceptable result from an evaluable screening endometrial biopsy. The endometrial biopsy reports by the two central pathologists at screening specified one of the following: proliferative endometrium; weakly proliferative endometrium; disordered proliferative pattern; secretory endometrium; endometrial tissue other (i.e., benign, inactive, or atrophic fragments of endometrial epithelium, glands, stroma, etc.); endometrial tissue insufficient for diagnosis; no endometrium identified; no tissue identified; endometrial hyperplasia; endometrial malignancy; or other findings (endometrial polyp not present, benign endometrial polyp, or other endometrial polyp). Identification of sufficient tissue to evaluate the biopsy by at least one pathologist was required.

For subjects with a Body Mass Index (BMI) less than or equal to 38 kg/m2, the subject inclusion criteria also included: (10) BMI values were rounded to the nearest integer (ex. 32.4 rounds down to 32, while 26.5 rounds up to 27).

In general, the inclusion criteria also included: (11) in the opinion of the investigator, the subject was believed likely to comply with the protocol and complete the study.

The exclusion criteria included: (1) use of oral estrogen-, progestin-, androgen-, or SERM-containing drug products within 8 weeks before screening visit 1A (entry of washout was permitted); use of transdermal hormone products within 4 weeks before screening visit 1A (entry of washout was permitted); use of vaginal hormone products (rings, creams, gels) within 4 weeks before screening visit 1A (entry of washout was permitted); use of intrauterine progestins within 8 weeks before screening visit 1A (entry of washout was permitted); use of progestin implants/injectables or estrogen pellets/injectables within 6 months before screening visit 1A (entry of washout was not permitted); or use of vaginal lubricants or moisturizers within 7 days before the screening visit 1B vaginal pH assessment.

The exclusion criteria also included: (2) a history or active presence of clinically important medical disease that might confound the study or be detrimental to the subject, including, for example: hypersensitivity to estrogens; endometrial hyperplasia; undiagnosed vaginal bleeding; a history of a chronic liver or kidney dysfunction/disorder (e.g., Hepatitis C or chronic renal failure); thrombophlebitis, thrombosis, or thromboembolic disorders; cerebrovascular accident, stroke, or transient ischemic attack; myocardial infarction or ischemic heart disease; malignancy or treatment for malignancy, within the previous 5 years, with the exception of basal cell carcinoma of the skin or squamous cell carcinoma of the skin (a history of estrogen dependent neoplasia, breast cancer, melanoma, or any gynecologic cancer, at any time, excluded the subject); and endocrine disease (except for controlled hypothyroidism or controlled non-insulin dependent diabetes mellitus).

The exclusion criteria also included: (3) recent history of known alcohol or drug abuse; (4) history of sexual abuse or spousal abuse that was likely to interfere with the subject's assessment of vaginal pain with sexual activity; (5) current history of heavy smoking (more than 15 cigarettes per day) or use of e-cigarettes; (6) use of an intrauterine device within 12 weeks before screening visit 1A; (7) use of an investigational drug within 60 days before screening visit 1A; (8) any clinically important abnormalities on screening physical exam, assessments, electrocardiogram (ECG), or laboratory tests; (9) known pregnancy or a positive urine pregnancy test; and (10) current use of marijuana.

In this study, if a subject discontinued or was withdrawn, the subject was not replaced. At the time of consent, each subject was given a unique subject number that identified their clinical site and sequential number. In addition to the assigned subject number, subject initials were used for identification. The clinical trial was performed in compliance with standard operating procedures as well as regulations set forth by FDA, ICH E6 (R1) guidelines, and other relevant regulatory authorities. Compliance was achieved through clinical trial-specific audits of clinical sites and database review.

### Statistical Methods:

Efficacy:

The primary objective of the trial was to assess the efficacy of estradiol vaginal softgel capsules (4 μg, 10 μg, and 25 μg) when compared to placebo on vaginal superficial cells, vaginal parabasal cells, vaginal pH, and on the symptom of moderate to severe dyspareunia (vaginal pain associated with sexual activity) as the MBS at week 12. To account for the multiple comparisons of testing placebo to each of the three doses of estradiol (4 μg, 10 μg, and 25 μg) and the multiple testing of the four co-primary endpoints, a closed procedure was performed (see, Edwards D, Madsen J. “Constructing multiple procedures for partially ordered hypothesis sets.” Stat Med 2007:26-5116-24, incorporated by reference herein).

Determination of Sample Size. The sample size needed per dose vs. placebo for each test of hypothesis in the modified intent-to-treat (MITT) population to achieve a given power was calculated using reference data from other studies (see, Bachman, G., et al. “Efficacy and safety of low-dose regimens of conjugated estrogens cream administered vaginally.” Menopause, 2009. 16(4): p. 719-27; Simon, J., et al. “Effective Treatment of Vaginal Atrophy With an Ultra-Low-Dose Estradiol Vaginal Tablet.” Obstetrics & Gynecology, 2008. 112(5):p. 1053-60; FDA Medical Officer's Review of Vagifem [NDA 20-908, Mar. 25, 1999, Table 6, p 12.], each incorporated by reference herein). Table 41 below provides the effect sizes, power, and sample size determinations for each of the primary endpoints. In general, subjects in the study met all inclusion/exclusion criteria and had moderate to severe dyspareunia as their most bothersome symptom of VVA. Based on the power analysis and the design considerations, approximately 175 subjects per treatment arm were enrolled.

TABLE 41
Power Analysis and Sample Size Determinations
Four Primary Endpoints in a Closed Procedure
Mean Change from Baseline to Week 12
Compared to Placebo (MMRM)
Power (One-way ANOVA, Alpha = 0.005, one-tailed)

| | | Power Based |
|---|---|---|
| | | Upon N = 140 |
| Primary Endpoint | Effect Size (%)* | per group per MITT |
| % Parabasal Cells | 150.3% | >0.999 |
| % Superficial Cells | 115.3% | >0.999 |
| Vaginal pH | 77.4% | >0.999 |
| Severity of Dyspareunia** | 30.0%, 41.2%, 70.5% | 0.50, 0.80, >0.999 |

*Range from 30% (Vagifem 10 μg; see, Simon 2008, supra), 41.2% (Vagifem 25 μg; see, FDA 1999, supra), 70.5% (Premarin cream 2/week; see, Bachman 2009, supra)
**Effect Size is calculated for all primary endpoints as 100% times difference (treated minus placebo) in mean changes at week 12 from baseline.

All subjects who were randomly assigned and had at least 1 dose of investigational product formed the intent-to-treat (ITT) population. The Modified intent-to-treat (MITT) population was defined as all ITT subjects with a baseline and at least one follow-up value for each of the primary endpoints, each subject having taken at least one dose of investigational product, and was the primary efficacy population. The efficacy-evaluable (EE) population was defined as all MITT subjects who completed the clinical trial, were at least 80% compliant with investigational product, had measurements for all primary efficacy endpoints, and were deemed to be protocol compliant, with no significant protocol violations. The safety population included all ITT subjects.

The primary efficacy analyses were conducted on the MITT subjects with supportive efficacy analyses conducted on the EE population. For analysis purposes, subjects were required to complete all visits, up to and including Visit 6 (week 12), to be considered as having completed the study.

Analysis of Efficacy Endpoints. For all numerically continuous efficacy endpoints, which included the four primary endpoints (mean change from baseline to week 12), active treatment group means were compared to placebo using an ANCOVA adjusting for the baseline level.

Primary and secondary efficacy endpoints were measured at baseline and at 2, 6, 8, and 12 weeks. The analysis examined change from baseline. Therefore, ANCOVAs were based on a repeated measures mixed effects model (MMRM) where the random effect was subject and the two fixed effects were treatment group and visit (2, 6, 8, and 12 weeks). Baseline measures and age were used as covariates. ANCOVAs were therefore not calculated independently for each study collection period. The analyses started with the full model but, interaction terms for visit (week 2, 6, 8, and 12) with treatment only remained where statistically significant (p<0.05).

The following three pair-wise comparisons were performed using the appropriate ANCOVA contrast for week 12 (primary) and weeks 2, 6, and 8 (secondary) changes from baseline: (1) active treatment, high dose group vs placebo; (2) active treatment, middle dose group vs placebo; and (3) active treatment, low dose group vs placebo.

Safety outcome measures. Adverse events, vital signs, physical examination findings, gynecological examination findings, clinical laboratory tests, pap smears, and endometrial biopsy were the safety parameters. Adverse events and SAEs were summarized for each treatment group and overall for all active treatment groups with the proportion of subjects reporting each event. Actual values and change from baseline in vital signs, and all laboratory test parameters were summarized for each treatment group and overall for all active treatment groups with descriptive statistics at each assessment obtained.

Endometrial Biopsy Assessment. Three independent pathologists with expertise in gynecologic pathology, blinded to treatment and to each other's readings, determined the diagnosis for endometrial biopsy slides during the conduct of the study. All visit 6, early termination, and on-treatment unscheduled endometrial biopsies were centrally read by three of the pathologists. Each pathologist's report was classified into one of the following three categories: category 1: not hyperplasia/not malignancy—includes proliferative endometrium, weakly proliferative endometrium, disordered proliferative pattern, secretory endometrium, endometrial tissue other (i.e., benign, inactive or atrophic fragments of endometrial epithelium, glands, stroma, etc.), endometrial tissue insufficient for diagnosis, no endometrium identified, no tissue identified, other; category 2: hyperplasia—includes simple hyperplasia with or without atypia and complex hyperplasia with or without atypia; category 3: malignancy—endometrial malignancy.

The final diagnosis was based on agreement of two of the three reads. Consensus was reached when two of the three pathologist readers agreed on any of the above categories. For example, any 2 subcategories of “not hyperplasia/not malignancy” were classified as “Category 1: not hyperplasia/not malignancy.” If all three readings were disparate (i.e., each fell into a different category—category 1, 2, or 3), the final diagnosis was based on the most severe of the three readings.

The analysis population for endometrium hyperplasia was the endometrial hyperplasia (EH) population. An EH subject at week 12 was one who was randomly assigned and took at least 1 dose of investigational product, with no exclusionary protocol violation (as detailed at the Statistical Analysis Plan), and had a pretreatment endometrial biopsy and a biopsy on therapy.

### Treatment of Subjects

The study used a double-blind design. Investigational product was supplied as 3 doses of Estradiol Vaginal Softgel Capsules (4 μg, 10 μg, and 25 μg) and matching placebo capsules. All subjects manually inserted one capsule into the vaginal cavity daily for 14 days (2 weeks) followed by twice weekly for 10 weeks according to one of the following treatment arms:

TABLE 42
Treatment Arms and Administration

| Regimen | Capsules | Capsules |
|---|---|---|
| Treatment 1 | 1 capsule daily of 4 μg vaginal | 1 capsule twice weekly |
| | softgel for 2 weeks | of 4 μg vaginal softgel |
| | | for 10 weeks |
| Treatment 2 | 1 capsule daily of 10 μg vaginal | 1 capsule twice weekly |
| | softgel for 2 weeks | of 10 μg vaginal softgel |
| | | for 10 weeks |
| Treatment 3 | 1 capsule daily of 25 μg vaginal | 1 capsule twice weekly |
| | softgel for 2 weeks | of 25 μg vaginal softgel |
| | | for 10 weeks |
| Treatment 4 | 1 capsule daily of placebo | 1 capsule twice weekly |
| | vaginal softgel for 2 weeks | of placebo vaginal |
| | | softgel for 10 weeks |

Investigational product was dispensed to all eligible subjects at visit 2. Each subject was provided a total of 30 soft gel capsules of investigational product in a labeled bottle, allowing for extra capsules for accidental loss or damage. A second bottle was dispensed at Visit 5. Each subject was trained by the clinical site to self-administer intravaginally one capsule daily at approximately the same hour for 2 weeks (14 days). The drug administration instructions included: “Remove vaginal capsule from the bottle; find a position most comfortable for you; insert the capsule with the smaller end up into vaginal canal for about 2 inches.” Starting on Day 15, each subject administered 1 capsule twice weekly for the remaining 10 weeks. Twice weekly dosing was approximately 3-4 days apart, and generally did not exceed more than twice in a seven day period. For example, if the Day 15 dose was inserted on Sunday, the next dose was inserted on Wednesday or Thursday. At randomization visit 2 (day 1), subjects received their first dose of investigational product at the clinical facility under the supervision of the study personnel.

The investigational estradiol vaginal softgel drug products used in the study are pear-shaped, opaque, light pink softgel capsules. The capsules contain the solubilized estradiol pharmaceutical compositions disclosed herein as Pharmaceutical Compositions 4-7. When the softgel capsules come in contact with the vaginal mucosa, the soft gelatin capsule releases the pharmaceutical composition, into the vagina. In embodiments, the soft gelatin capsule completely dissolves.

The placebo used in the study contained the excipients in the investigational estradiol vaginal softgel capsule without the estradiol (see, e.g., Pharmaceutical Composition 7). The packaging of the investigational products and placebo were identical to maintain adequate blinding of investigators. Neither the subject nor the investigator was able to identify the treatment from the packaging or label of the investigational products.

A subject was required to use at least 80% of the investigational product to be considered compliant with investigational medication administration. Capsule count and diary cards were be used to determine subject compliance at each study visit. Subjects were randomly assigned in a 1:1:1:1 ratio to receive Estradiol Vaginal Softgel Capsule 4 μg (Pharmaceutical Composition 4), Estradiol Vaginal Softgel Capsule 10 μg (Pharmaceutical Composition 5), Estradiol Vaginal Softgel Capsule 25 μg (Pharmaceutical Composition 6), or placebo (Pharmaceutical Composition 7).

Concomitant medications/treatments were used to treat chronic or intercurrent medical conditions at the discretion of the investigator. The following medications were prohibited for the duration of the study: investigational drugs other than the investigational Estradiol Vaginal Softgel Capsule; estrogen-, progestin-, androgen (i.e., DHEA) or SERM-containing medications other than the investigational product; medications, remedies, and supplements known to treat vulvar/vaginal atrophy; vaginal lubricants and moisturizers (e.g., Replens) be discontinued 7 days prior to Visit 1B vaginal pH assessment; and all medications excluded before the study.

### Efficacy Assessments

Vaginal cytological smears were collected from the lateral vaginal walls according to standard procedures and sent to a central laboratory to evaluate vaginal cytology. The percentage of superficial, parabasal, and intermediate cells was determined. All on-therapy/early termination vaginal cytology results were blinded to the Sponsor, Investigators, and subjects.

Vaginal pH was determined at screening Visit 1B and visits 3, 4, 5, and 6/end of treatment. Subjects were not allowed to use vaginal lubricants or moisturizers within 7 days of the screening vaginal pH assessment or at any time afterwards during the study. The subjects were advised not to have sexual intercourse and to refrain from using vaginal douching within 24 hours prior to the measurement for all scheduled vaginal pH assessments. After insertion of an unlubricated speculum, a pH indicator strip was applied to the lateral vaginal wall until it became wet, taking care to avoid cervical mucus, blood or semen that are known to affect vaginal pH. The color of the strip was compared immediately with a colorimetric scale and the measurement was recorded.

During the gynecological examinations, the investigator performed a visual evaluation of vaginal mucosa using a four-point scale (0=none, 1=mild, 2=moderate, and 3=severe) to assess parameters of vaginal secretions, vaginal epithelial integrity, vaginal epithelial surface thickness, and vaginal color according to the table below.

| Assessment | Severity | | | |
|---|---|---|---|---|
| Criteria | No atrophy | Mild | Moderate | Severe |
| Vaginal | normal clear | superficial coating of | scant not covering | none, inflamed, |
| secretions | secretions noted on | secretions, difficulty | the entire vaginal | ulceration noted, |
| | vaginal walls | with speculum | vault, may need | need lubrication with |
| | | insertion | lubrication with | speculum insertion to |
| | | | speculum insertion | prevent pain |
| | | | to prevent pain | |
| Vaginal | normal | vaginal surface | vaginal surface | vaginal surface has |
| epithelial | | bleeds with scraping | bleeds with light | petechiae before |
| integrity | | | contact | contact and bleeds |
| | | | | with light contact |
| Vaginal | rogation and | poor rogation with | smooth, some | smooth, no elasticity, |
| epithelial | elasticity of vault | some elasticity noted | elasticity of | constriction of the |
| surface | | of vaginal vault | vaginal vault | upper one third of |
| thickness | | | | vagina or loss of |
| | | | | vaginal tone |
| | | | | (cystocele and |
| | | | | rectocele) |
| Vaginal color | pink | lighter in color | pale in color | transparent, either no |
| | | | | color or inflamed |

The VVA symptom self-assessment questionnaire, shown below, is an instrument for subjects to self-assess their symptoms of vulvar or vaginal atrophy, including vaginal pain associated with sexual activity, vaginal dryness, vulvar or vaginal itching, or irritation. At screening visit 1A subjects were asked to complete the questionnaire and identify their most bothersome symptoms, and the results of the survey were used to determine initial eligibility for the study. At visit 1A, subjects were also asked to indicate which moderate or severe symptoms bothered them most. The questionnaire was administered again at screening visit 1B and used to determine continued eligibility for the study.

VVA SYMPTOMS SELF-ASSESSMENT

| Please Rate | | | | |
|---|---|---|---|---|
| your Vulvar | Severity Score | | | |
| and/or Vaginal | (Please select only ONE) | | | |
| Symptoms | 0 = None | 1 = Mild | 2 = Moderate | 3 = Severe |

| | |
|---|---|
| 1 | Pain associated |
| | with sexual |
| | activity (with |
| | vaginal |
| | penetration). |
| 2 | Vaginal |
| | dryness. |
| 3 | Vulvar and/or |
| | vaginal itching |
| | or irritation. |

Randomized subjects were asked to complete the VVA Symptom Self-Assessment Questionnaire at visits 3, 4, 5, and 6. Subjects were asked to indicate if no sexual activity with vaginal penetration was experience since the previous visit. Screening visit 1B evaluation results were considered as Baseline data for the statistical analyses.

The Female Sexual Function Index (FSFI) is a brief, multidimensional scale for assessing sexual function in women (see, Rosen, 2000, supra 26: p. 191-208, incorporated by reference herein). The scale consists of 19 items that assess sexual function over the past 4 weeks and yield domain scores in six areas: sexual desire, arousal, lubrication, orgasm, satisfaction, and pain. Further validation of the instrument was conducted to extend the validation to include dyspareunia/vaginismus (pain), and multiple sexual dysfunctions (see, Weigel, M., et al. “The Female Sexual Function Index (FSFI): Cross-Validation and Development of Clinical Cutoff Scores.” Journal of Sex & Marital Therapy, 2005. 31: p. 1-20, incorporated by reference herein). The FSFI was conducted at Visits 2 and 6. Subjects participating in the PK substudy were not assessed using FSFI.

### Safety Assessments

A complete medical history, including demographic data (age and race/ethnicity) gynecological, surgical, and psychiatric history and use of tobacco and alcohol was recorded at the washout/screening visit 1A prior to any washout period; this history included a review of all past and current diseases and their respective durations as well as any history of amenorrhea.

A complete physical examination was conducted at screening visit 1A and visit 6/end of treatment. The physical examination included, at a minimum, examination of the subject's general appearance, HEENT (head, eyes, ears, nose, and throat), heart, lungs, musculoskeletal system, gastrointestinal (GI) system, neurological system, lymph nodes, abdomen, and extremities. The subject's height was measured at washout/screening visit 1A only and body weight (while the subject is lightly clothed) was be measured at washout/screening visit 1A and end of treatment. BMI was calculated at washout/screening visit 1A. Vital signs (body temperature, heart rate [HR], respiration rate [RR], and sitting blood pressure [BP]) were measured at all visits after the subject had been sitting for ≥10 minutes. If the initial BP reading was above 140 mmHg systolic or 90 mmHg diastolic, the option for a single repeat assessment performed 15 minutes later was provided. A standard 12-lead ECG was obtained at screening visit 1A and visit 6 or early termination.

Subjects were required to have a pelvic examination and Pap smear performed during the screening visit 1B and visit 6 or early termination. The Pap smear was required for all subjects with or without an intact uterus and cervix. For subjects without an intact cervix the Pap smear was obtained by sampling the apex of the vaginal cuff. All subjects were required to have a Pap smear done during screening, regardless of any recent prior assessment. Subjects who discontinued the study after 2 weeks of investigational product were required to have an end of treatment Pap smear. Subjects had a breast examination performed during screening visit 1A and at visit 6 or early termination.

Endometrial biopsies were performed by a board-certified gynecologist at screening and at visit 6/end of treatment. Unscheduled endometrial biopsies were performed during the study, when indicated for medical reasons. The screening biopsy was performed at screening visit 1B, after the subject's initial screening visit assessments indicated that the subject was otherwise an eligible candidate for the study.

At screening, endometrial biopsies were read centrally by two pathologists. A candidate subject was excluded from the study if at least one pathologist assessed the endometrial biopsy as endometrial hyperplasia, endometrial cancer, proliferative endometrium, weakly proliferative endometrium, or disordered proliferative pattern, or if at least one pathologist identified an endometrial polyp with hyperplasia, glandular atypia of any degree (e.g., atypical nuclei), or cancer. Additionally, identification of sufficient tissue to evaluate the biopsy by at least one pathologist was required for study eligibility. The option for one repetition of the screening endometrial biopsy was made available when an initial endometrial biopsy was performed and both of the primary pathologists reported endometrial tissue insufficient for diagnosis, no endometrium was identified, or no tissue was identified, and if the subject had met all other protocol-specified eligibility criteria to date. The visit 6 (or early termination) endometrial biopsies and on treatment unscheduled biopsies were assessed by three pathologists.

During the study, at early termination, and at the end of the study, any subject with a diagnosis of endometrial hyperplasia was withdrawn and treated with 10 mg of Medroxyprogesterone acetate (MPA) for 6 months unless deemed otherwise by the PI. For unscheduled biopsies, the histological diagnosis of endometrial polyp did not force withdrawal unless atypical nuclei were present.

A urine pregnancy test was conducted at screening visit 1A unless the subject had a history of tubal ligation, bilateral oophorectomy, or was ≥55 years of age and had experienced cessation of menses for at least 1 year.

Blood samples for blood chemistry, hematology, coagulation tests, and hormone levels and urine samples for urine analysis were collected and sent to a central laboratory. Blood Chemistry (sodium, potassium, chloride, total cholesterol, blood urea nitrogen (BUN), iron, albumin, total protein, aspartate aminotransferase (AST), alanine aminotransferase (ALT), alkaline phosphatase, creatinine, calcium, phosphorous, uric acid, total bilirubin, glucose and triglycerides (must be fasting minimum of 8 hours). A fasting glucose of >125 mg/dL will require a HgA1C) was monitored. Hematology (complete blood count (CBC) including white blood cell count and differential, red blood cell count, hemoglobin, hematocrit, and platelet count) was monitored. Hormone Levels (follicle-stimulating hormone (FSH) (not required for subjects with ≥12 months of spontaneous amenorrhea or bilateral oophorectomy), estradiol, estrone, and estrone conjugates and SHBG for subjects in the PK substudy) were monitored. Urine Analysis (appearance, specific gravity, protein, and pH) was conducted.

### Pharmacokinetic Assessment

Seventy-two subjects were also enrolled in a pharmacokinetic (PK) substudy. In those subjects participating in the PK substudy, time 0 h serum blood samples were obtained at screening visit 1A, day 1, and day 14 prior to dosing for baseline. The baseline was characterized by the average of the two pre-treatment samples. Serum blood samples were then obtained on day 1 and day 14 at five post dose time points (2 h, 4 h, 6 h, 10 h, and 24 h). On study days 1 (visit 2) and 14 (visit 3) a baseline pretreatment blood sample (Time 0 h) was collected from each subject prior to insertion of the investigational product. After insertion of the product, blood samples were drawn at 2, 4, 6, 10, and 24 hours following insertion. The last PK sample (approximately day 84) was obtained 4 days following the last insertion of investigational product.

Blood samples were analyzed to characterize area under the curve (AUC), time of maximum concentration (tmax), minimum concentration (Cmin), and maximum concentration (Cmax). Blood samples were also analyzed to measure the levels of estradiol, estrone, and estrone conjugates. No fasting requirements were applied. Sex hormone binding globulin (SHBG) levels were obtained at pre-treatment baseline (day 1, visit 2), and day 14 at the 0 h and on the day 84 final hormone blood draw.

A symptoms/complaints and medications diary was dispensed at all visits and subjects were instructed on completion. The subjects used the diary to record symptoms/complaints (including stop and start dates and treatment received) and prior medications/treatments (including indication, stop, and start dates). A copy of the diary was made at each visit and re-dispensed to the subject. A dosing diary was dispensed at visit 2 and at visit 3 and subjects were be instructed on completion. Subjects recorded investigational product usage and sexual activity. The dosing diary dispensed at visit 3 was re-dispensed at visits 4 and 5. A copy of the diary was made at each visit prior to re-dispensing to the subject.

### Study Visits

Study visits were typically conducted so as to include the activities outlined in Table 43.

TABLE 43
Schedule of Assessments - Main Study

| | | | | Visit 2: | Visit 3: |
|---|---|---|---|---|---|
| | | | | Randomization/ | Interim |
| | Washout | Visit 1A | Visit 1B | Baseline | Week 2 |
| | Week −14 | Screening | Screening | Week 0 | Day 14 |
| Activity | to −6 | Week −6 to 0 | Week −4 to 0 | Day 1 | (±3 d) |
| Informed consent | X | X | | | |
| Demographics/Medical and | X | X | | | |
| Gynecological history and prior | | | | | |
| medications | | | | | |
| Weight | X | X | | | |
| Height and BMI calculation | X | X | | | |
| Vital signs | X | X | X | X | X |
| MBS | | X | | | |
| Subject VVA Self-Assessment | | X | X | | X |
| Questionnaire | | | | | |
| Physical examination including breast | | X | | | |
| exam | | | | | |
| Laboratory safety tests (Hematology, | | X | | | |
| Serum Chemistry, FSHP, Urinalysis) | | | | | |
| 12-Lead ECG | | X | | | |
| Pelvic exam | | | X | | |
| Vaginal pH | | | X | | X |
| Papanicolaou (Pap) smear | | | X | | |
| Investigator assessment of vaginal | | | X | | X |
| mucosa | | | | | |
| Vaginal cytological smear | | | X | | X |
| Mammogram | | X | | | |
| Endometrial biopsy | | | X | | |
| Diary Dispense | X | X | X | X | X |
| Diary Collection | | X | X | X | X |
| FSFI | | | | X | |
| Satisfaction Survey | | | | | |
| Urine pregnancy test | | X | | | |
| Randomization | | | | X | |
| Dispense Investigational Product bottle | | | | X | |
| Re-dispense Investigational Product | | | | | X |
| bottle | | | | | |
| Treatment administration instruction | | | | X | X |
| Collect unused investigational product | | | | | X |
| and used bottles; assess compliance | | | | | |
| Adverse event monitoring | | X | X | X | X |
| Concomitant medications | | X | X | X | X |

| | | | Visit 6: | |
|---|---|---|---|---|
| | | | End of | Telephone |
| | | | Treatment | Interview |
| | Visit 4: | Visit 5: | or Early | Week 14 |
| | Interim | Interim | Term | approximately |
| | Week 6 | Week 8 | Week 12 | 15 days after |
| | Day 42 | Day 56 | Day 84 | last dose |
| Activity | (± 3 d) | (± 3 d) | (±3 d) | of IP |
| Informed consent | | | | |
| Demographics/Medical | | | | |
| and Gynecological history | | | | |
| and prior medications | | | | |
| Weight | | | X | |
| Height and BMI | | | | |
| calculation | | | | |
| Vital signs | X | X | X | |
| MBS | | | | |
| Subject VVA Self- | X | X | X | |
| Assessment | | | | |
| Questionnaire | | | | |
| Physical examination | | | X | |
| including breast exam | | | | |
| Laboratory safety tests | | | X | |
| (Hematology, Serum | | | | |
| Chemistry, FSHP, | | | | |
| Urinalysis) | | | | |
| 12-Lead ECG | | | X | |
| Pelvic exam | | | X | |
| Vaginal pH | X | X | X | |
| Papanicolaou (Pap) smear | | | X | |
| Investigator assessment of | X | X | X | |
| vaginal mucosa | | | | |
| Vaginal cytological smear | X | X | X | |
| Mammogram | | | | |
| Endometrial biopsy | | | X | |
| Diary Dispense | X | X | | |
| Diary Collection | X | X | X | |
| FSFI | | | X | |
| Satisfaction Survey | | | X | |
| Urine pregnancy test | | | | |
| Randomization | | | | |
| Dispense Investigational | | X | | |
| Product bottle | | | | |
| Re-dispense | X | | | |
| Investigational Product | | | | |
| bottle | | | | |
| Treatment administration | X | X | | |
| instruction | | | | |
| Collect unused | X | X | X | |
| investigational product | | | | |
| and used bottles; assess | | | | |
| compliance | | | | |
| Adverse event monitoring | X | X | X | X |
| Concomitant medications | X | X | X | X |

Washout Period Visit (if applicable; Weeks −14 to −6). The purpose of this visit was to discuss the study with a potential subject and obtain informed consent that is signed and dated before any procedures, including washout are performed. Subjects who agreed to discontinue current treatment began washout after the consent form was signed. A symptoms/complaints and medication diary was dispensed at this visit and the subject was instructed in how to complete the diary. Once the washout period was completed, the subject will return to the site for visit 1A.

The activities and assessments conducted during the visit included: informed consent; demographics; medical/gynecological history; collection of prior and concomitant medication information; height, body weight measurement and BMI calculation; collection of vital signs (body temperature, HR, RR, and BP); dispensation of symptoms/complaints diary and instruction in how to complete the diary

Screening Period Visits (Visits 1A and 1B). Subjects not requiring washout begin screening procedures at visit 1A as described above for the washout period. With the exception of vital signs, procedures performed at washout will not be repeated at screening visit 1A. In general, screening visits 1A and 1B were completed within 6 weeks (42 days) for subjects without a uterus or within 8 weeks (56 days) for subjects with a uterus. All screening assessments were completed prior to randomization. The investigators reviewed the results from all screening procedures and determined if the subjects were eligible for enrollment into the study.

Visit 1A (approximately Week −6 to 0). Visit 1A was conducted after the wash-out period (if applicable) or after the subject provided informed consent. The subject was advised to fast for 8 hours prior to the visit for blood draws.

Procedures and evaluations conducted at the visit included: informed consent; demographics; medical/gynecological history; collection of the symptoms/complaints and medications diary from washout (if applicable) and review with the subject; recording of prior medication information; recording and assessment of adverse events (AEs) starting from the signing of informed consent; height, body weight measurement and BMI calculation; collection of vital signs (body temperature, HR, RR, and BP); physical examination; breast examination (including a mammogram conducted up to nine months prior to Visit 2); urine pregnancy test as required; blood and urine sample collection for blood chemistry (minimum fast of 8 hrs), hematology, and urinalysis; serum FSH as required; 12-Lead ECG.

At visit 1A, the VVA symptom self-assessment questionnaire was conducted and most bothersome symptoms were identified, with the subject self-identifying moderate or severe pain with sexual activity as her MBS to continue screening. The symptoms/complaints and medications diary was dispensed, and subjects were instructed in how to complete the diary. Subjects were instructed to refrain from use of vaginal lubricants for 7 days and sexual intercourse/vaginal douching for 24 hours prior to the vaginal pH assessment to be done at visit 1B.

Visit 1B (approximately Week −4 to Week 0). Visit 1B was conducted after the subject's initial screening visit and after the other screening results indicated that the subject was otherwise an eligible candidate for the study (preferably around the middle of the screening period).

Procedures and evaluations conducted at the visit included: VVA symptom self-assessment questionnaire, the subject having indicated moderate to severe pain with sexual activity with vaginal penetration in order to continue screening; collection of vital signs (body temperature, HR, RR, and BP); pelvic examination; investigator assessment of vaginal mucosa as described above; assessment of vaginal pH (sexual intercourse or vaginal douching within 24 hrs prior to the assessment being prohibited, and a subject's vaginal pH being >5.0 to continue screening); Pap smear; vaginal cytological smear (one repetition being permitted during screening if no results were obtained from the first smear); endometrial biopsy performed as described above; review of the symptoms/complaints and medications diary with the subject.

Visit 2 (Week 0; Randomization/Baseline). Subjects who met entry criteria were randomized to investigational product at this visit. Procedures and evaluations conducted at the visit included: self-administration of FSFI by subjects not participating in the PK substudy; review of the symptoms/complaints and medications diary with the subject; review of evaluations performed at screening visits and verification of present of all inclusion criteria and the absence of all exclusion criteria; collection of vital signs (body temperature, HR, RR, and BP); randomization, with subjects meeting all entry criteria being randomized and allocated a bottle number; dispensation of investigational product and instruction in how to insert the capsule vaginally, with subjects receiving their first dose of investigational product under supervision; dispensation of dosing diary and instruction on completion of the treatment diary, including recording investigational product usage and sexual activity.

Visit 3 (Week 2, Day 14±3 days). Procedures and evaluations conducted at the visit included: completion of the VVA symptom self-assessment questionnaire; review of the symptoms/complaints and medications diaries with the subject; collection of vital signs (body temperature, HR, RR, and BP); Assessment of vaginal mucosa; assessment of vaginal pH (with sexual intercourse or vaginal douching within 24 hrs prior to the assessment being prohibited); vaginal cytological smear; collection of unused investigational product and bottle for assessment of compliance/accountability; re-dispensation of investigational product and re-instruction in how to insert the capsule vaginally if necessary; review of the completed dosing diary with the subject.

Visit 4 (Week 6, Day 42±3 days). Procedures and evaluations conducted at the visit included: completion of the VVA symptom self-assessment questionnaire; review of the symptoms/complaints and medications diary with the subject; collection of vital signs (body temperature, HR, RR, and BP); assessment of vaginal mucosa as described above; vaginal cytological smear; assessment of vaginal pH (with sexual intercourse or vaginal douching within 24 hrs prior to the assessment being prohibited); collection of unused investigational product for assessment of compliance/accountability; re-dispensation of investigational product and re-instruction in how to insert the capsule vaginally if necessary; review of the completed dosing diary with the subject.

Visit 5 (Week 8, Day 56±3 days). Procedures and evaluations conducted at the visit included: completion of the VVA symptom self-assessment questionnaire; review of the symptoms/complaints and medications diary with the subject; collection of vital signs (body temperature, HR, RR, and BP); assessment of vaginal mucosa as described above; vaginal cytological smear; assessment of vaginal pH (with sexual intercourse or vaginal douching within 24 hrs prior to the assessment being prohibited); collection of unused investigational product for assessment of compliance/accountability; re-dispensation of investigational product and re-instruction in how to insert the capsule vaginally if necessary; review of the completed dosing diary with the subject.

Visit 6 (Week 12, Day 84±3 days or early termination). This visit was performed if a subject withdraws from the study before visit 6. Procedures performed at this visit included: completion of the VVA symptom self-assessment questionnaire; review of the subject the dosing diary, symptoms/complaints, and medications diaries with the subject; collection of blood and urine sample collection for blood chemistry (minimum fast of 8 hrs), hematology, and urinalysis; collection of vital signs (body temperature, HR, RR, and BP) and weight; performance of 12-lead-ECG; collection of unused investigational product and container for assessment of compliance/accountability; physical examination; breast exam; assessment of vaginal mucosa as described above; assessment of vaginal pH (with sexual intercourse or vaginal douching within 24 hrs prior to the assessment being prohibited); vaginal cytological smear; Pap smear; endometrial biopsy; self-administration of FSFI by subjects not participating in the PK substudy; self-administration of survey titled “Acceptability of product administration Survey” by subjects.

Follow-up Interview (approximately 15 days after the last dose of investigational product). Each subject who received investigational product received a follow-up phone call, regardless of the duration of therapy, approximately 15 days following the last dose of investigational product. The follow-up generally took place after receipt of all safety assessments (e.g., endometrial biopsy and mammography results). The follow-up included: review of ongoing adverse events and any new adverse events that occurred during the 15 days following the last dose of investigational product; review of ongoing concomitant medications and any new concomitant medications that occurred during the 15 days following the last dose of investigational product; and discussion of all end of study safety assessments and determination if further follow up or clinic visit is required.

### PK Substudy Visit Procedures and Schedule

Screening Visit 1A. In addition to the procedures listed described above, activities in the PK substudy also included: provision of informed consent by subject and agreement to participate in the PK substudy; collection of a serum blood sample during the visit for baseline assessment of estradiol, estrone, and estrone conjugates.

Visit 2 (Week 0, Day 1). In addition to the procedures listed described above, activities in the PK substudy also included collection of serum blood sample obtained prior to the administration of investigational product (timepoint 0 h) for baseline assessment of estradiol, estrone, estrone conjugates, and SHBG. The investigational product was self-administered by the subject after the pre-treatment blood sample has been taken. After investigational product administration, serum blood samples were obtained at 2 h, 4 h, 6 h, 10 h, and 24 h timepoints for estradiol, estrone, and estrone conjugates (serum samples were generally taken within +/−5 minutes at 2 h and 4 h, within +/−15 minutes at 6 h, and within +/−1 h at 10 h and 24 h). The subject was released from the site after the 10 hour sample and instructed to return to the site the next morning for the 24 hour blood draw. The subject was instructed not to self-administer the day 2 dose until instructed by the site personnel to dose at the clinical site. The subject was released from the clinical site following the 24 hour blood sample and administration of the day 2 dose.

Visit 3 (Week 2, Day 14). The visit must occurred on day 14 with no visit window allowed. In addition to the procedures listed above, the PK substudy included collection of a serum blood sample prior to the administration of day 14 dose (timepoint 0 h) for SHBG and PK assessments. The subject self-administered the day 14 dose at the clinical site, and serum blood samples were obtained at 2 h, 4 h, 6 h, 10 h, and 24 h timepoints for estradiol, estrone, and estrone conjugates. The subject was released from the site after the 10 hour sample and instructed to return to the site the next morning for the 24 hour blood draw. The subject was instructed not to self-administer the day 15 dose until instructed by the site personnel to dose at the clinical site. The subject was released from the clinical site following the 24 hour blood sample and administration of the day 15 dose. The subject was be instructed to administer the next dose of study drug on day 18 or day 19 and continue dosing on a bi-weekly basis at the same time of day for each dose.

Visit 6 (Week 12, Day 84±3 days, or at early termination). The visit took place 4 days after last IP dose or early termination. A serum sample for estradiol, estrone, and estrone conjugates and SHBG was drawn in addition to the procedures described above.

PK sub-study visits were typically conducted so as to include the activities outlined in Table 44.

TABLE 44
Schedule of Assessments for PK Sub-study

| | | | | Visit 2: | |
|---|---|---|---|---|---|
| | | | | Randomization/ | Visit 3: Interim |
| | Washout | Visit 1A | Visit 1B | Baseline | Week 2 |
| | Week −14 | Screening | Screening | Week 0 | Day 14 |
| Activity | to −6 | Week −6 to 0 | Week −4 to 0 | Day 1 | (no window) |
| PK sub-study Informed | X | X | | | |
| consent | | | | | |
| Demographics/Medical | X | X | | | |
| and Gynecological history | | | | | |
| and prior medications | | | | | |
| Weight | X | X | | | |
| Height and BMI | X | X | | | |
| calculation | | | | | |
| Vital signs | X | X | X | X | X |
| MBS | | X | | | |
| Subject VVA Self- | | X | X | | X |
| Assessment | | | | | |
| Questionnaire | | | | | |
| Physical examination | | X | | | |
| including breast exam | | | | | |
| Laboratory safety tests | | X | | | |
| (Hematology, Serum | | | | | |
| Chemistry, FSHP, | | | | | |
| Urinalysis) | | | | | |
| PK Serum Blood Samples | | X | | X | X |
| (Estradiol, Estrone, | | | | | |
| Estrone Conjugates) | | | | | |
| Serum blood samples for | | | | X | X |
| SHBG | | | | | |
| 12-Lead ECG | | X | | | |
| Pelvic exam | | | X | | |
| Vaginal pH | | | X | | X |
| Papanicolaou (Pap) smear | | | X | | |
| Investigator assessment of | | | X | | X |
| vaginal mucosa | | | | | |
| Vaginal cytological smear | | | X | | X |
| Mammogram | | X | | | |
| Endometrial biopsy | | | X | | |
| Diary Dispense | X | X | X | X | X |
| Diary Collection | | X | X | X | X |
| Satisfaction Survey | | | | | |
| Urine pregnancy test | | X | | | |
| Randomization | | | | X | |
| Dispense new | | | | X | |
| Investigational Product | | | | | |
| (IP) bottle | | | | | |
| Re-dispense | | | | | X |
| Investigational Product | | | | | |
| (IP) bottle | | | | | |
| IP administration | | | | X | X |
| instruction | | | | | |
| Collect unused IP and | | | | | X |
| used bottles; assess | | | | | |
| compliance | | | | | |
| Adverse event monitoring | | X | X | X | X |
| Concomitant medications | | X | X | X | X |

| | | | Visit 6: End | |
|---|---|---|---|---|
| | | | of treatment | |
| | | | or Early | Telephone |
| | Visit 4: | Visit 5: | Term | Interview |
| | Interim | Interim | Week 12 | Week 14 |
| | Week 6 | Week 8 | Day 84 (±3 d) | approximately |
| | Day 42 | Day 56 | (4 days after | 15 days after |
| Activity | (±3 d) | (±3 d) | last IP dose) | last dose of IP |
| PK sub-study Informed | | | | |
| consent | | | | |
| Demographics/Medical | | | | |
| and Gynecological | | | | |
| history and prior | | | | |
| medications | | | | |
| Weight | | | X | |
| Height and BMI | | | | |
| calculation | | | | |
| Vital signs | X | X | X | |
| MBS | | | | |
| Subject VVA Self- | X | X | X | |
| Assessment | | | | |
| Questionnaire | | | | |
| Physical examination | | | X | |
| including breast exam | | | | |
| Laboratory safety tests | | | X | |
| (Hematology, Serum | | | | |
| Chemistry, FSHP, | | | | |
| Urinalysis) | | | | |
| PK Serum Blood | | | X | |
| Samples (Estradiol, | | | | |
| Estrone, Estrone | | | | |
| Conjugates) | | | | |
| Serum blood samples | | | X | |
| for SHBG | | | | |
| 12-Lead ECG | | | X | |
| Pelvic exam | | | X | |
| Vaginal pH | X | X | X | |
| Papanicolaou (Pap) | | | X | |
| smear | | | | |
| Investigator assessment | X | X | X | |
| of vaginal mucosa | | | | |
| Vaginal cytological | X | X | X | |
| smear | | | | |
| Mammogram | | | | |
| Endometrial biopsy | | | X | |
| Diary Dispense | X | X | | |
| Diary Collection | X | X | X | |
| Satisfaction Survey | | | X | |
| Urine pregnancy test | | | | |
| Randomization | | | | |
| Dispense new | | X | | |
| Investigational Product | | | | |
| (IP) bottle | | | | |
| Re-dispense | X | | | |
| Investigational Product | | | | |
| (IP) bottle | | | | |
| IP administration | X | X | | |
| instruction | | | | |
| Collect unused IP and | X | X | X | |
| used bottles; assess | | | | |
| compliance | | | | |
| Adverse event | X | X | X | X |
| monitoring | | | | |
| Concomitant | X | X | X | X |
| medications | | | | |

An Adverse Event (AE) in the study was defined as the development of an undesirable medical condition or the deterioration of a pre-existing medical condition following or during exposure to a pharmaceutical product, whether or not considered casually related to the product. An AE could occur from overdose of investigational product. In this study, an AE can include an undesirable medical condition occurring at any time, including baseline or washout periods, even if no study treatment has been administered.

Relationship to Investigational Product

The investigators determined the relationship to the investigational product for each AE (Not Related, Possibly Related, or Probably Related). The degree of “relatedness” of the adverse event to the investigational product was described as follows: not related—no temporal association and other etiologies are likely the cause; possible—temporal association, but other etiologies are likely the cause. However, involvement of the investigational product cannot be excluded; probable—temporal association, other etiologies are possible but unlikely. The event may respond if the investigational product is discontinued.

### Example 11: Efficacy Results of Randomized, Double-Blind, Placebo-Controlled Multicenter Study

Each of the three doses showed statistical significance compared with placebo for the primary endpoints. Each of the three doses showed statistical significance compared with placebo for the secondary endpoints. Table 45 shows the statistical significance of the experimental data for each of the four co-primary endpoints. Each of the dosages met each of the four co-primary endpoints at a statistically significant level. The 25 mcg dose of TX-004HR demonstrated highly statistically significant results at the p≤0.0001 level compared to placebo across all four co-primary endpoints. The 10 mcg dose of TX-004HR demonstrated highly statistically significant results at the p≤0.0001 level compared to placebo across all four co-primary endpoints. The 4 mcg dose of TX-004HR also demonstrated highly statistically significant results at the p≤0.0001 level compared to placebo for the endpoints of superficial vaginal cells, parabasal vaginal cells, and vaginal pH; the change from baseline compared to placebo in the severity of dyspareunia was at the p=0.0255 level.

TABLE 45
Statistical Significance of Results for Co-Primary
Endpoints (Based on Mean Change from Baseline
to Week 12 Compared to Placebo)

| | 25 mcg | 10 mcg | 4 mcg |
|---|---|---|---|
| Superficial Cells | P < 0.0001 | P < 0.0001 | P < 0.0001 |
| Parabasal Cells | P < 0.0001 | P < 0.0001 | P < 0.0001 |
| Vaginal pH | P < 0.0001 | P < 0.0001 | P < 0.0001 |
| Severity of | P = 0.0001 | P = 0.0001 | P = 0.0255 |
| Dyspareunia | | | |

Statistical improvement over placebo was also observed for all three doses at the first assessment at week two and sustained through week 12. The pharmacokinetic data for all three doses demonstrated low systemic absorption, supporting the previous Phase 1 trial data. TX-004HR was well tolerated, and there were no clinically significant differences compared to placebo-treated women with respect to adverse events. There were no drug-related serious adverse events reported.

As shown in the data below, in the MITT population (n=747) at week 12, all TX-004HR doses compared with placebo significantly decreased the percentage of parabasal cells and vaginal pH, significantly increased the percentage of superficial cells, and significantly reduced the severity of dyspareunia (all p≤0.00001 except dyspareunia at 4 μg p=0.0149).

At weeks 2, 6, and 8, the percentage of parabasal cells and vaginal pH significantly decreased p<0.00001); the percentage of superficial cells significantly increased (p<0.00001); and the severity of dyspareunia significantly improved from baseline with all TX-004HR doses vs placebo (4 μg p<0.03; 10 μg and 25 μg p<0.02).

Moderate-to-severe vaginal dryness was reported by 93% at baseline and significantly improved (p<0.02) for all doses at weeks 2, 6, 8, and 12 (except 4 μg at week 2). Vulvar and/or vaginal itching or irritation significantly improved (p<0.05) for 10 μg at weeks 8 and 12, and for 25 μg at week 12.

TX-004HR was well tolerated, had high acceptability, and no treatment-related serious AEs were reported in the safety population (n=764). There were no clinically significant differences in any AEs or treatment-related SAEs between TX-004HR and placebo. Very low to negligible systemic levels of estradiol were observed.

All TX-004HR doses were safe and effective and resulted in very low to negligible systemic absorption of E2 in women with VVA and moderate-to-severe dyspareunia. Onset of effect was seen as early as 2 weeks and was maintained throughout the study and acceptability was very high. This novel product provides a promising new treatment option for women experiencing menopausal VVA.

### Cytology

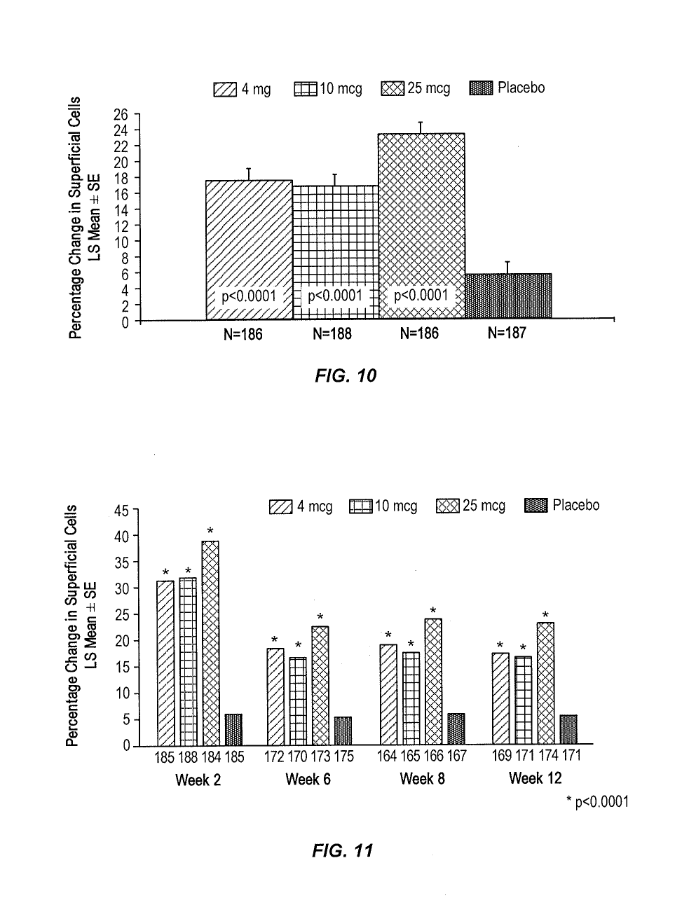

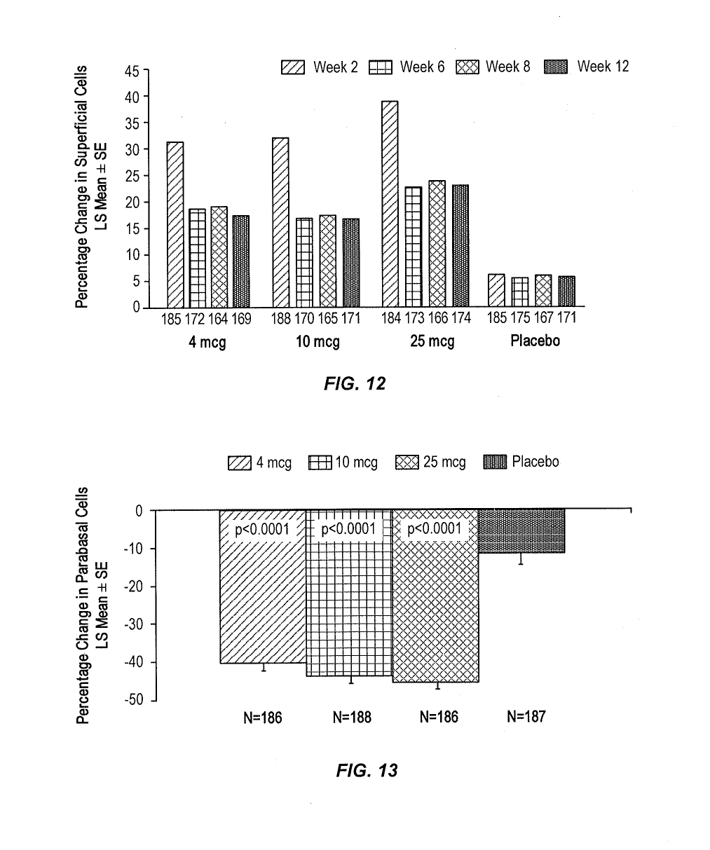

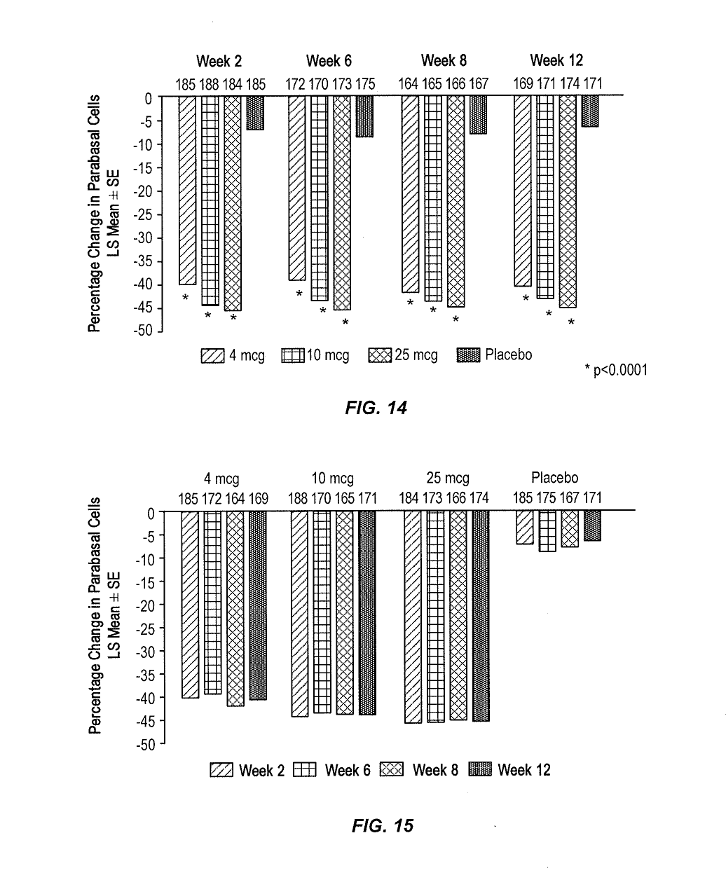

Vaginal cytology data was collected as vaginal smears from the lateral vaginal walls according to procedures presented above to evaluate vaginal cytology at screening and Visit 6—End of treatment (day 84). The change in the Maturation Index was assessed as a change in cell composition measured at Visit 1—Baseline (day 1) compared to the cell composition measured at Visit 3—End of treatment (day 84). The change in percentage of superficial, parabasal, and intermediate cells obtained from the vaginal mucosal epithelium from a vaginal smear was recorded. Results from these assessments for superficial cells are presented in Table 46 and Table 47, as well as FIG. 10, FIG. 11, and FIG. 12. Results from these assessments for parabasal cells are presented in Table 48 and Table 49, as well as FIG. 13, FIG. 14, and FIG. 15.

### Superficial Cells

TABLE 46
Superficial Cells P-values by Treatment Week

| | 4 μg | 10 μg | 25 μg |
|---|---|---|---|
| Week 2 | <0.0001 | <0.0001 | <0.0001 |
| Week 6 | <0.0001 | <0.0001 | <0.0001 |
| Week 8 | <0.0001 | <0.0001 | <0.0001 |
| Week 12 | <0.0001 | <0.0001 | <0.0001 |

TABLE 47
Superficial Cells Change in Severity from Baseline by Treatment Week
(change in percent of total vaginal cells)

| | 4 μg | 10 μg | 25 μg | Placebo |
|---|---|---|---|---|
| Week 2 | 31.35 (1.496) | 31.93 (1.488) | 38.85 (1.5) | 6.05 (1.498) |
| Week 6 | 18.41 (1.536) | 16.88 (1.543) | 22.65 (1.532) | 5.43 (1.525) |
| Week 8 | 19.04 (1.561) | 17.41 (1.558) | 23.88 (1.554) | 5.98 (1.551) |
| Week | 17.5 (1.542) | 16.72 (1.54) | 23.2 (1.529) | 5.63 (1.537) |
| 12 | | | | |

The study showed the formulations disclosed herein across all doses increased the percentage of superficial cells across all dosages in a statistically significant way.

### Parabasal Cells

TABLE 48
Parabasal Cells P-values by Treatment Week

| | 4 μg | 10 μg | 25 μg |
|---|---|---|---|
| Week 2 | <0.0001 | <0.0001 | <0.0001 |
| Week 6 | <0.0001 | <0.0001 | <0.0001 |
| Week 8 | <0.0001 | <0.0001 | <0.0001 |
| Week 12 | <0.0001 | <0.0001 | <0.0001 |

TABLE 49
Parabasal Cells Change in Severity from Baseline by
Treatment Week (change in percent of total vaginal cells)

| | 4 μg | 10 μg | 25 μg | Placebo |
|---|---|---|---|---|
| Week | −40.23 (1.719) | −44.42 (1.708) | −45.6 (1.723) | −7 (1.72) |
| 2 | | | | |
| Week | −39.36 (1.75) | −43.55 (1.752) | −45.61 (1.746) | −9.23 (1.741) |
| 6 | | | | |
| Week | −41.87 (1.768) | −43.78 (1.764) | −45.08 (1.762) | −7.86 (1.76) |
| 8 | | | | |
| Week | −40.63 (1.755) | −44.07 (1.751) | −45.55 (1.745) | −6.73 (1.75) |
| 12 | | | | |

The increase of superficial cells and decrease of parabasal cells showed statistical significance over placebo at week 2 and for every week thereafter, including at week 12. Administration of the pharmaceutical formulation resulted in rapid onset of action, as early as two weeks after the initial administration. Rapid onset of action may be coupled with the rapid absorption demonstrated in the pharmacokinetic data presented below.

pH

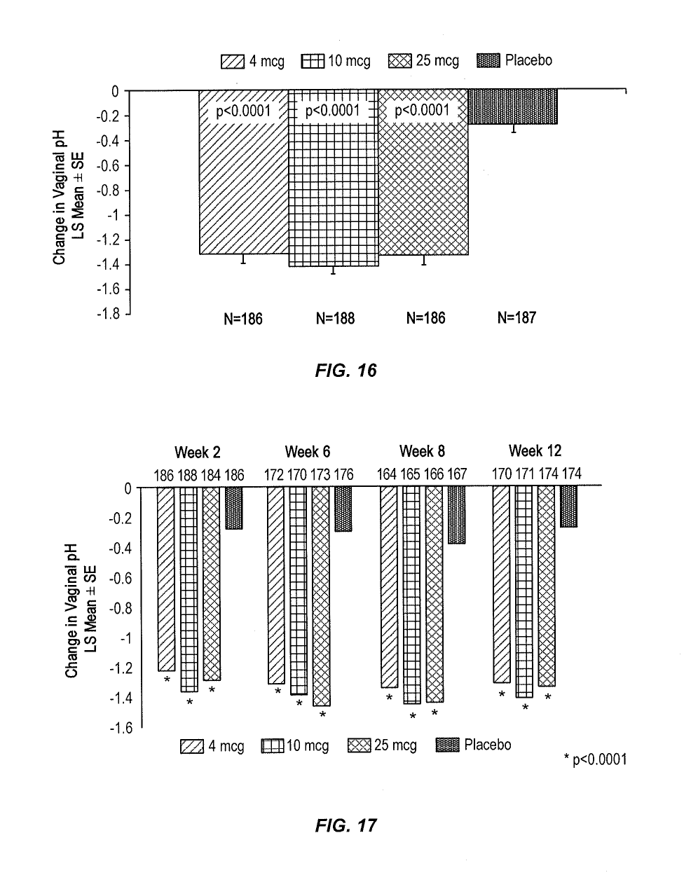

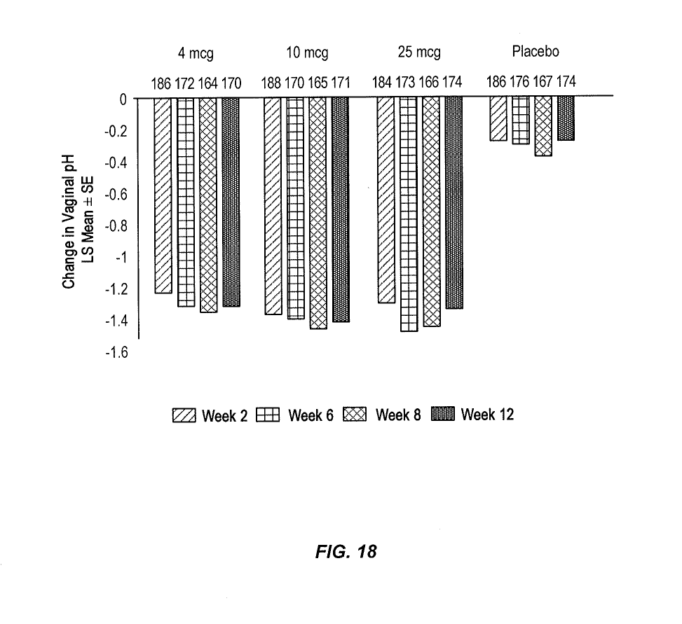

Vaginal pH was measured at Screening and Visit 6—End of treatment (day 84). The pH measurement was obtained as disclosed herein. Results from these assessments are presented in Table 50 and Table 51, and FIG. 16, FIG. 17, and FIG. 18.

TABLE 50
pH P-values by Treatment Week

| | 4 μg | 10 μg | 25 μg |
|---|---|---|---|
| Week 2 | <0.0001 | <0.0001 | <0.0001 |
| Week 6 | <0.0001 | <0.0001 | <0.0001 |
| Week 8 | <0.0001 | <0.0001 | <0.0001 |
| Week 12 | <0.0001 | <0.0001 | <0.0001 |

TABLE 51
pH Change in Severity from Baseline
by Treatment Week (change in pH)

| | 4 μg | 10 μg | 25 μg | Placebo |
|---|---|---|---|---|
| Week | −1.23 (0.064) | −1.37 (0.064) | −1.3 (0.065) | −0.28 (0.064) |
| 2 | | | | |
| Week | −1.32 (0.066) | −1.4 (0.066) | −1.48 (0.066) | −0.3 (0.065) |
| 6 | | | | |
| Week | −1.35 (0.067) | −1.46 (0.067) | −1.45 (0.066) | −0.38 (0.066) |
| 8 | | | | |
| Week | −1.32 (0.066) | −1.42 (0.066) | −1.34 (0.066) | −0.28 (0.066) |
| 12 | | | | |

The decrease in vaginal pH was observed at statistically significant levels at week 2 and through the end of the study. Surprisingly, the pH decreased in all three pharmaceutical formulations tested and at all three dosages of over a full pH unit for all three doses.

### Most Bothersome Symptoms

### Dyspareunia

Subjects were asked to specify the symptom that she identified as the “most bothersome symptom.” During the screening period all of the subjects were provided with a questionnaire to self-assess the symptoms of VVA: (1) dyspareunia; (2) vaginal dryness; and (3) vaginal or vulvar irritation, burning, or itching. Each symptom was measured on a scale of 0 to 3, where 0=none, 1=mild, 2=moderate, and 3=severe. Each subject was given a questionnaire at each visit and the responses were recorded. All randomized subjects were also provided a questionnaire to self-assess the symptoms of VVA at Visit 1 and on each subsequent visit through Visit 6—End of the treatment (day 84). Subjects recorded their self-assessments daily in a diary and answers were collected on visits 8 and 15 (end of treatment). Pre-dose evaluation results obtained at Visit 1 were considered as baseline data for the statistical analyses. Data from these assessments for dyspareunia are presented in Table 52 and Table 53. Data from these assessments for dryness are presented in Table 54 and Table 55.

TABLE 52
Dyspareunia P-values by Treatment Week

| | 4 μg | 10 μg | 25 μg |
|---|---|---|---|
| Week 2 | 0.026 | 0.0019 | 0.0105 |
| Week 6 | 0.0069 | 0.0009 | <0.0001 |
| Week 8 | 0.0003 | <0.0001 | <0.0001 |
| Week 12 | 0.0149 | <0.0001 | <0.0001 |

TABLE 53
Dyspareunia Change in Severity from Baseline by Treatment Week
(0 to 3 severity scale)

| | 4 μg | 10 μg | 25 μg | Placebo |
|---|---|---|---|---|
| Week | −0.99 (0.072) | −1.08 (0.072) | −1.02 (0.073) | −0.76 (0.072) |
| 2 | | | | |
| Week | −1.3 (0.072) | −1.37 (0.072) | −1.48 (0.072) | −1.03 (0.07) |
| 6 | | | | |
| Week | −1.52 (0.073) | −1.64 (0.074) | −1.62 (0.075) | −1.15 (0.072) |
| 8 | | | | |
| Week | −1.52 (0.071) | −1.69 (0.071) | −1.69 (0.071) | −1.28 (0.07) |
| 12 | | | | |

Each of the 4 μg, 10 μg, and 25 μg formulations tests demonstrated an early onset of action at week 2 for the most bothersome symptom of dyspareunia, evidenced by the statistically significant results (measured by p-value) in Table 52. After two weeks, each dose demonstrated separation from placebo in improvement in the most bothersome symptom of dyspareunia.

Coupled with the PK data presented below, these results show that the formulations disclosed herein provide a bolus of estradiol within two hours of administration, which resulted in a decrease in the severity of dyspareunia as early as two weeks later. Estradiol is rapidly absorbed at around two hours, which is significantly faster than the formulations of the prior art that sought an extended release profile. The rapid absorption of estradiol is believed to be a result of administration with a liquid formulation.

Surprisingly, the 4 μg formulation showed clinical effectiveness at two weeks along with the 25 μg and 10 μg dosage levels. These data demonstrate that 4 μg is an effective dose, and can be effective as early as two weeks after administration for the most bothersome symptom of dyspareunia.

### Dryness

TABLE 54
Dryness P-values by Treatment Week

| | 4 μg | 10 μg | 25 μg |
|---|---|---|---|
| Week 2 | 0.1269 | 0.0019 | 0.0082 |
| Week 6 | 0.0094 | 0.0001 | 0.0005 |
| Week 8 | 0.0128 | <0.0001 | 0.0008 |
| Week 12 | 0.0014 | <0.0001 | <0.0001 |

TABLE 55
Dryness Change in Severity from Baseline
by Treatment Week (0 to 3 severity scale)

| | 4 μg | 10 μg | 25 μg | Placebo |
|---|---|---|---|---|
| Week 2 | −0.86(0.066) | −1.01(0.065) | −0.96(0.066) | −0.72(0.066) |
| Week 6 | −1.14(0.067) | −1.27(0.068) | −1.23(0.067) | −0.9(0.067) |
| Week 8 | −1.25(0.069) | −1.44(0.068) | −1.34(0.068) | −1.01(0.068) |
| Week 12 | −1.27(0.068) | −1.47(0.067) | −1.47(0.067) | −0.97(0.067) |

Each of the 4 μg, 10 μg, and 25 μg formulations tests demonstrated an early onset of action at week 2 for the most bothersome symptom of dryness, evidenced by the statistically significant results (measured by p-value) in Table 54. After two weeks, each dose demonstrated separation from placebo in improvement in the most bothersome symptom of dryness.

### Irritation/Itching

TABLE 56
Irritation/Itching P-values by Treatment Week

| | 4 μg | 10 μg | 25 μg |
|---|---|---|---|
| Week 2 | 0.9616 | 0.2439 | 0.6518 |
| Week 6 | 0.7829 | 0.2328 | 0.4118 |
| Week 8 | 0.0639 | 0.0356 | 0.0914 |
| Week 12 | 0.0503 | 0.0055 | 0.0263 |

TABLE 57
Irritation/Itching Change in Severity from Baseline
by Treatment Week (0 to 3 severity scale)

| | 4 μg | 10 μg | 25 μg | Placebo |
|---|---|---|---|---|
| Week 2 | −0.47(0.054) | −0.56(0.053) | −0.51(0.054) | −0.47(0.054) |
| Week 6 | −0.57(0.055) | −0.64(0.055) | −0.61(0.055) | −0.55(0.055) |
| Week 8 | −0.74(0.056) | −0.76(0.056) | −0.73(0.056) | −0.59(0.056) |
| Week 12 | −0.75(0.055) | −0.81(0.055) | −0.77(0.055) | −0.6(0.055) |

Vulvar and/or vaginal itching or irritation significantly improved (p<0.05) for 10 μg at weeks 8 and 12, and for 25 μg at week 12. Moreover, the trend for 4 μg was an improvement in itching week over week to nearly being statistically significant at week 12.

Coupled with the PK data presented below, these results show that the formulations disclosed herein provide a bolus of estradiol within two hours of administration, which resulted in a decrease in the severity of dryness as early as two weeks later. Estradiol is rapidly absorbed at around two hours, which is significantly faster than the formulations of the prior art that sought an extended release profile. The rapid absorption of estradiol is believed to be a result of administration with a liquid formulation.

Surprisingly, the 4 μg formulation showed clinical effectiveness at two weeks along with the 25 μg and 10 μg dosage levels. These data demonstrate that 4 μg is an effective dose, and can be effective as early as two weeks after administration for the most bothersome symptom of dryness.

As described above, each dose was compared with placebo for change from baseline to week 12 in the percentages of vaginal superficial cells and parabasal cells, vaginal pH, and severity of dyspareunia (co-primary endpoints). The proportion of responders (defined as women with ≥2 of the following at week 12: vaginal superficial cells >5%, vaginal pH<5.0, ≥1 category improvement from baseline dyspareunia score) was compared in TX-004HR groups vs placebo. Pre-specified subgroup analyses of co-primary endpoints were analyzed by age (≤56 years, 57-61 years, and ≥62 years), BMI (≤24 kg/m2, 25-28 kg/m2, and ≥29 kg/m2), uterine status, parity, and vaginal births. Pharmacokinetic (PK) parameters were compared with placebo in a sub-analysis of the main study.

The proportion of responders was significantly higher for all TX-004HR dose groups vs placebo (p<0.0001 for all). All TX-004HR doses vs placebo significantly improved percentage of superficial and parabasal cells, vaginal pH, and severity of dyspareunia at 12 weeks. Subgroup analyses showed generally similar results for percentage of superficial and parabasal cells and vaginal pH irrespective of age, BMI, uterine status, parity, and vaginal births. Severity of dyspareunia was significantly reduced at 12 weeks with all TX-004HR doses vs placebo in most subgroups (Table 57A).

The PK sub-analysis (n=72), described in more detail below, found AUC and Cavg parameters for E2 and estrone (E1) with 4 μg and 10 μg TX-004HR to be similar to placebo. Increases occurred in E2 AUC and Cavg with 25 μg vs placebo but remained within the normal postmenopausal range. E2 levels at day 84 were similar between the TX-004HR groups and placebo, indicating no systemic drug accumulation.

All doses of TX-004HR were associated with robust efficacy and demonstrated a statistically significant difference vs placebo for increasing superficial cells, decreasing parabasal cells and vaginal pH, and reducing the severity of dyspareunia. Age, BMI, uterine status, parity and vaginal births generally did not affect TX-004HR efficacy. These results occurred with negligible systemic absorption of TX-004HR estradiol doses of 4 μg, 10 μg, and 25 pg.

TABLE 57A
Change from baseline to week 12 in the severity of dyspareunia
(LS mean change ± SE).

| | | | | TX-004HR | | TX-004HR | | TX-004HR | |
|---|---|---|---|---|---|---|---|---|---|
| | | Placebo | | 4 μg | | 10 μg | | 25 μg | |
| Key clinical factors | | (n = 187) | | (n = 186) | | (n = 188) | | (n = 186) | |
| Age, years | ≤56 | n = 52 | −1.25 ± 0.119 | n = 50 | −1.58 ± 0.122 | n = 61 | −1.77 ± 0.112^{†} | n = 65 | −1.86 ± 0.108^{‡} |
| | 57-61 | n = 53 | −1.39 ± 0.118 | n = 50 | −1.42 ± 0.121 | n = 49 | −1.63 ± 0.121 | n = 47 | −1.79 ± 0.125* |
| | ≥62 | n = 58 | −1.19 ± 0.122 | n = 51 | −1.52 ± 0.126 | n = 44 | −1.66 ± 0.138^{†} | n = 47 | −1.38 ± 0.135 |
| BMI, | ≤24 | n = 56 | −1.14 ± 0.115 | n = 58 | −1.48 ± 0.113* | n = 56 | −1.6 ± 0.117^{†} | n = 51 | −1.72 ± 0.123^{‡} |
| kg/m² | 25 to 28 | n = 57 | −1.48 ± 0.118 | n = 45 | −1.51 ± 0.131 | n = 52 | −1.78 ± 0.124 | n = 58 | −1.77 ± 0.117 |
| | ≥29 | n = 50 | −1.21 ± 0.125 | n = 48 | −1.56 ± 0.125 | n = 46 | −1.71 ± 0.129^{†} | n = 50 | −1.57 ± 0.124* |
| Uterine | Intact | n = 101 | −1.35 ± 0.086 | n = 82 | −1.66 ± 0.095* | n = 84 | −1.74 ± 0.095^{†} | n = 85 | −1.81 ± 0.094^{‡} |
| status | Non-intact | n = 62 | −1.15 ± 0.115 | n = 69 | −1.35 ± 0.108 | n = 70 | −1.63 ± 0.108^{†} | n = 74 | −1.55 ± 0.107* |
| Pregnancy | Pregnancy = 0 | n = 16 | −1.18 ± 0.220 | n = 17 | −1.28 ± 0.217 | n = 19 | −1.26 ± 0.209 | n = 13 | −1.64 ± 0.257 |
| status | Pregnancy ≥ 1 | n = 147 | −1.28 ± 0.073 | n = 134 | −1.55 ± 0.075* | n = 135 | −1.74 ± 0.076^{§} | n = 146 | −1.70 ± 0.073^{‡} |
| Vaginal | Vaginal birth = 0 | n = 26 | −1.19 ± 0.171 | n = 22 | −1.74 ± 0.189* | n = 29 | −1.68 ± 0.161* | n = 31 | −1.76 ± 0.160* |
| births | Vaginal birth ≥ 1 | n = 121 | −1.30 ± 0.080 | n = 112 | −1.51 ± 0.082 | n = 106 | −1.77 ± 0.085^{‡} | n = 115 | −1.69 ± 0.082^{‡} |

*p < 0.05;
^{†}p < 0.01;
^{‡}p < 0.001;
^{§}p < 0.0001 vs placebo.

Visual evaluation of the vaginal epithelium, a secondary endpoint of the trial, was performed during gynecological examinations at baseline and weeks 2, 6, 8, and 12. A four-point score (0=none, 1=mild, 2=moderate, 3=severe) was used to assess changes in vaginal color, vaginal epithelial integrity, vaginal epithelial surface thickness, and vaginal secretions. Change from baseline to each time point was compared with placebo using the mixed effect model repeat measurement (MMRM) analysis.

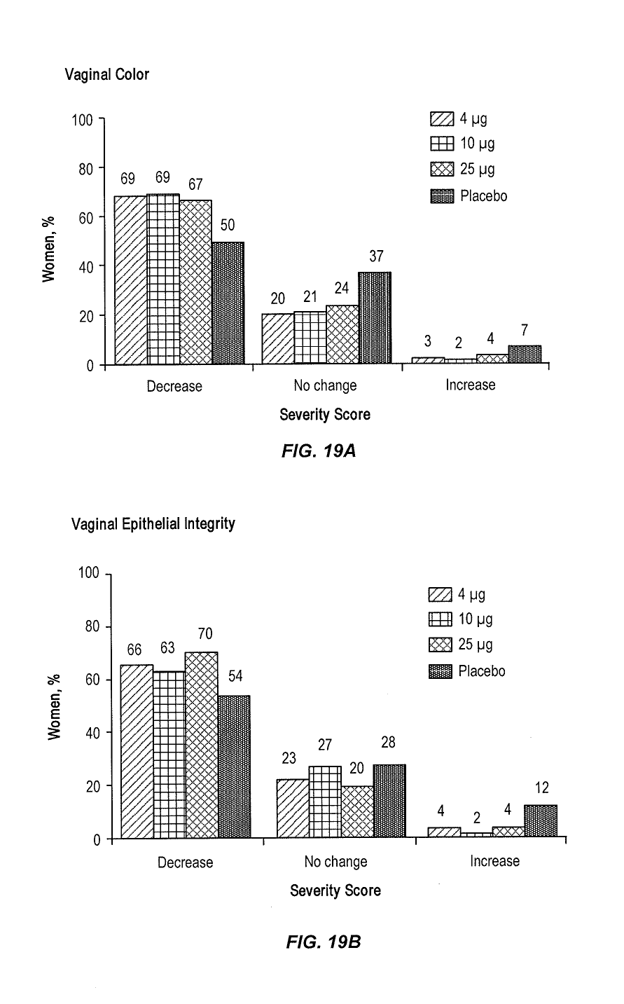

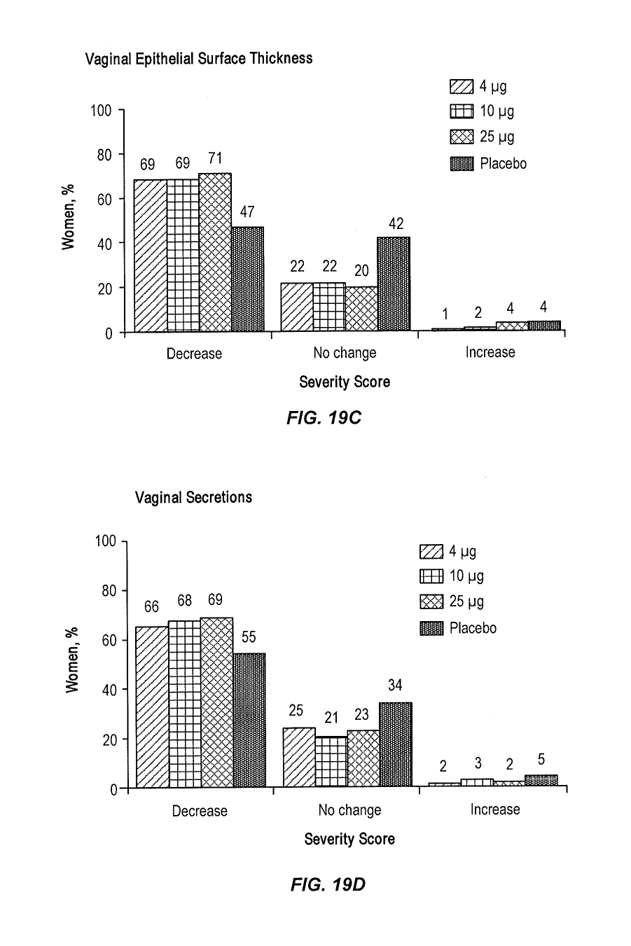

At baseline, women had mean scores of 1.8 for vaginal color, 1.5 for epithelial integrity, 1.9 for epithelial surface thickness, and 1.7 for secretions. These scores were consistent with VVA reflecting pallor, diminished vaginal wall integrity and thickness, and secretions. Significant improvements from baseline at weeks 2, 6, 8 and 12 (Table 57B; FIG. 19A-FIG. 19D) were observed for all 3 doses of TX-004HR compared with placebo in vaginal color (white to pink), epithelial integrity, epithelial surface thickness and secretions (p<0.001 for all). After 12 weeks, women in the active TX-004HR treatment groups had mean scores less than 1 in all four characterized categories. Vaginal visual examination of women in the 3 TX-004HR groups had greater reported improvements from baseline in all vaginal parameters examined than placebo subjects and at all time points. These improved vaginal visual scores reflect other observed measures of efficacy of TX-004HR (4 μg, 10 μg, and 25 μg) at treating moderate-to-severe VVA in postmenopausal women, with negligible to very low systemic E2 absorption.

TABLE 57B
Change from baseline at week 12 in vaginal parameters

| | | TX-004HR | | TX-004HR | | TX-004HR | | | |
|---|---|---|---|---|---|---|---|---|---|
| | | 4 μg | | 10 μg | | 25 μg | | Placebo | |
| Vaginal Parameters, mean (SD) | | (n = 171) | | (n = 173) | | (n = 175) | | (n = 175) | |
| Vaginal epithelial | Baseline | 1.8 | (0.61) | 1.7 | (0.59) | 1.8 | (0.60) | 1.7 | (0.64) |
| color | 12 weeks | 0.8 | (0.67) | 0.7 | (0.64) | 0.8 | (0.68) | 1.2 | (0.80) |
| | Change | −1.0 | (0.82) | −1.1 | (0.80) | −1.0 | (0.88) | −0.6 | (0.83) |
| | LS Mean (SE) | −0.97 | (0.05)* | −1.06 | (0.05)* | −0.96 | (0.05)* | −0.60 | (0.05) |
| Vaginal epithelial | Baseline | 1.6 | (0.84) | 1.4 | (0.83) | 1.5 | (0.77) | 1.5 | (0.84) |
| integrity | 12 weeks | 0.5 | (0.69) | 0.4 | (0.57) | 0.5 | (0.66) | 0.9 | (0.91) |
| | Change | −1.0 | (0.93) | −1.0 | (0.89) | −1.0 | (0.91) | −0.6 | (0.98) |
| | LS Mean (SE) | −0.97 | (0.05)* | −1.07 | (0.05)* | −1.01 | (0.05)* | −0.60 | (0.05) |
| Vaginal epithelial | Baseline | 1.9 | (0.67) | 1.8 | (0.63) | 1.9 | (0.59) | 1.9 | (0.65) |
| surface thickness | 12 weeks | 0.9 | (0.66) | 0.8 | (0.63) | 0.9 | (0.69) | 1.3 | (0.85) |
| | Change | −1.0 | (0.76) | −1.0 | (0.79) | −0.9 | (0.80) | −0.6 | (0.82) |
| | LS Mean (SE) | −0.98 | (0.05)* | −1.03 | (0.05)* | −0.94 | (0.05)* | −0.61 | (0.05) |
| Vaginal | Baseline | 1.8 | (0.68) | 1.7 | (0.66) | 1.7 | (0.63) | 1.8 | (0.63) |
| secretions | 12 weeks | 0.8 | (0.69) | 0.6 | (0.67) | 0.7 | (0.71) | 1.1 | (0.84) |
| | Change | −1.0 | (0.82) | −1.0 | (0.86) | −1.0 | (0.85) | −0.7 | (0.79) |
| | LS Mean (SE) | −1.01 | (0.05)* | −1.06 | (0.05)* | −1.04 | (0.05)* | −0.64 | (0.05) |

Data is mean (SD) unless otherwise noted;
*MMRM p < 0.0001 vs placebo.

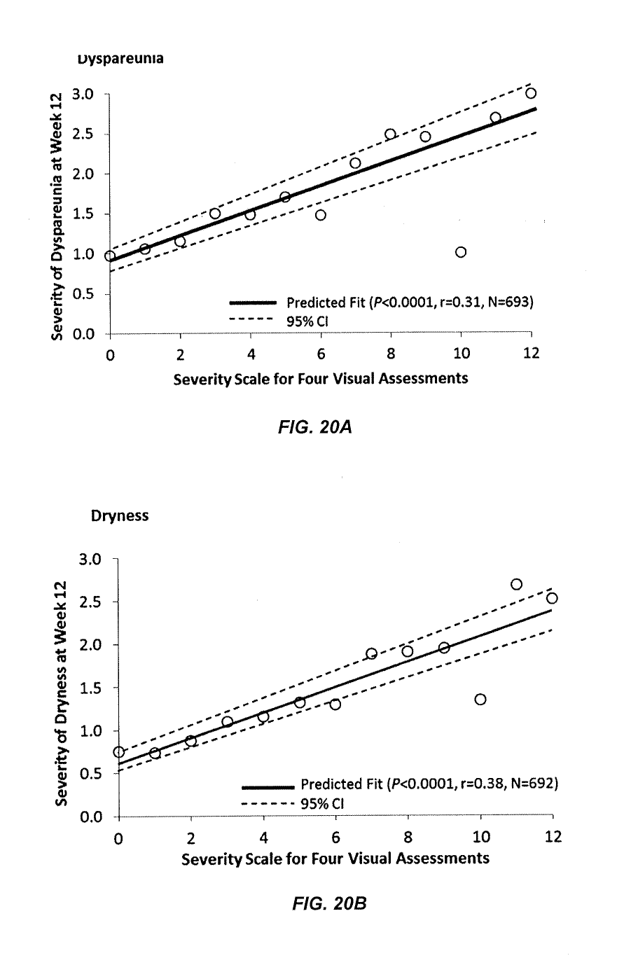

A direct correlation was observed between the total sum of the individual visual examination score and severity of dyspareunia (r=0.31; P<0.0001) as well as the severity of vaginal dryness (r=0.38; P<0.0001) at 12 weeks when all subjects were analyzed independent of treatment. See, FIG. 20A and FIG. 20B. Interestingly, women treated with placebo also showed some improvements in their scores at week 2, but while women treated with TX-004HR showed continued improvements through 12 weeks of treatment, such continued improvements were not observed to the same extent with the placebo. Three possible explanations for the improvements observed with the placebo include the potential lubricating effect of the excipient Miglyol, a fractionated coconut oil contained in all softgel capsules, improved appearance based on vaginal lubrication caused by increased sexual activity and/or bias on the part of the physicians performing the examinations as they may anticipate improvement. Nevertheless, TX-004HR still significantly improved evaluated signs and symptoms of VVA better than placebo.

Since visual inspection of the vagina with the 4-point assessment tool positively correlated with dyspareunia and vaginal dryness in this study, this tool may help healthcare professionals diagnose VVA and assess its treatment, and provide a vehicle for health care professionals to initiation discussion with their patients about a sensitive topic. Several large-scale studies have shown that it is difficult for patients to discuss vulvovaginal health openly with their health care professionals because they are either embarrassed, uninformed about VVA and its treatments, or believe that the topic is not appropriate for discussion. Therefore, of the 50% of postmenopausal women who have symptoms of VVA, far fewer seek treatment. Visual examination of the vagina may help practitioners identify women at risk of dyspareunia and vaginal dryness, and allow them to proactively engage women in conversations about VVA symptoms such as dyspareunia and dryness and discuss available treatment options.

### Example 12: Pharmacokinetics Results in Randomized, Double-Blind, Placebo-Controlled Multicenter Study

While some approved local estrogens effectively treat VVA, systemic estradiol may increase with local administration. TX-004HR is a new low-dose vaginal softgel capsule containing solubilized natural estradiol designed to provide excellent efficacy with negligible systemic absorption. Up to three times lower systemic estrogen levels were previously reported with TX-004HR vs an approved low-dose vaginal estradiol tablet. The present studies show that VVA efficacy can be achieved with negligible systemic absorption as measured by PK in postmenopausal women with moderate-to-severe dyspareunia.

The terms “minimal systemic effect,” “low systemic absorption,” and “negligible systemic absorption,” as used herein, mean that the disclosed formulations and methods result in low to minimal absorption of estradiol in women, especially women with VVA and/or dyspareunia. In fact, it has surprisingly been found that the disclosed formulations and methods result in negligible to very low systemic absorption of estradiol, which remains in the postmenopausal range. The finding is borne out by the examples provided herein that demonstrate that the Cmax and AUC levels of estradiol relative to placebo were not statistically differentiable, which indicates that the formulations disclosed herein have a negligible systemic effect. As such, the disclosed formulations and methods advantageously provide local benefits in patients with VVA and/or dyspareunia (i.e., the disclosed formulations are extremely effective in increasing the superficial cells, decreasing parabasal cells, and decreasing pH) without increasing systemic levels.

A PK substudy was part of a large, multicenter, double-blind, randomized, placebo-controlled phase 3 trial evaluating the efficacy and safety of TX-004HR (4 μg, 10 μg, and 25 μg) compared with placebo for treating postmenopausal moderate-to-severe dyspareunia. Women received TX-004HR or placebo once daily for 2 weeks then twice weekly for 10 wks.

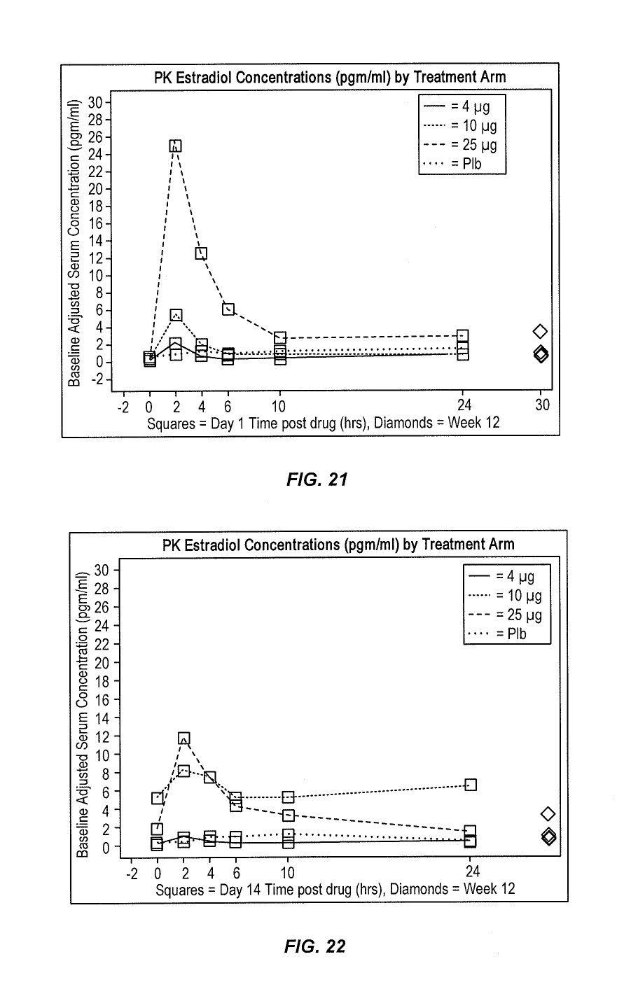

In this study, the systemic exposure to estradiol following once daily intravaginal administration of estradiol 25 μg, 10 μg, 4 μg, and placebo were investigated on days 1, 14, and 84 as described herein. Descriptive statistics of the plasma estradiol concentrations taken at each sampling time and the observed Cmax values were recorded, as shown in the tables below and FIG. 21 and FIG. 22, for estradiol, estrone, and estrone conjugates for all three doses. Serum estradiol, estrone, estrone conjugates, and sex hormone binding globulin were measured.

For PK, serum was sampled pre-dose and at 2, 4, 6, 10, and 24 h post-dose on days 1 and 14 for estradiol, estrone (E1), and estrone conjugates (E1Cs). Baseline-adjusted results are shown here; unadjusted data will be presented. Efficacy endpoints were change from baseline to week 12 for vaginal superficial cells (%), vaginal parabasal cells (%), vaginal pH, and severity of dyspareunia. Secondary endpoints were severity of dryness and itching/irritation. Blood chemistry was tested at week 12.

The substudy randomized 72 women (mean age 59 y) at 11 centers. Mean area under the concentration-time curve (AUC) and average concentration (Cavg) for estradiol were not significantly different vs placebo with 4 μg and 10 μg TX-004HR, but were significantly higher with 25 μg at day 1 (AUC 130 vs 13.8 h*pg/mL and Cavg 5.4 vs 0.4 pg/mL) and day 14 (AUC 84.6 vs 7.1 h*pg/mL and Cavg 3.5 vs −0.2 pg/mL).

Mean estradiol peak concentration (Cmax) was not significantly different with 4 μg (day 1: 2.6 pg/mL; day 14: 1.3 pg/mL) vs placebo (day 1: 2.1 pg/mL; day 14: 1.0 pg/mL), and although significant, was negligible with 10 μg (day 1: 6.0 pg/mL; day 14: 3.0 pg/mL) and very low for 25 μg (day 1: 26.2 pg/mL; day 14: 12.0 pg/mL).

E1 and E1Cs AUC, Cavg, Cmax, Cmin did not differ vs placebo, except for E1Cs on day 1 when AUC was significantly higher with 25 μg (2454 vs 83.0 h*pg/mL), Cmax with 10 μg and 25 μg (90.2 and 198.6 pg/mL, respectively vs 27.1 pg/mL), and Cavg with 10 μg (8.0 vs −33.7 pg/mL).

In the overall study TX004-HR showed robust efficacy for symptoms of dyspareunia, vaginal dryness and irritation at 12 weeks with all 3 doses compared with placebo.

Vaginal TX-004HR resulted in negligible to very low systemic absorption of estradiol, which remained in the postmenopausal range. TX-004HR improved the signs and symptoms of VVA. This study supports local benefits of estradiol without increasing systemic exposure.

The pharmacokinetic data for estradiol demonstrates the rapid absorption of the formulations disclosed herein for all three doses. Surprisingly, while the pharmacokinetic data was extremely low for all three doses, each dose was extremely effective in increasing the superficial cells, decreasing parabasal cells, and decreasing pH.

The pharmaceutical compositions disclosed herein provide an improved safety profile over other options for treating VVA. The combination of low systemic estradiol, while retaining efficacy was a surprising result for all three doses.

### Estradiol Concentration

TABLE 58
Pharmacokinetics Estradiol Baseline (pg/mL)

| | 4 μg | 10 μg | 25 μg | Placebo |
|---|---|---|---|---|
| Baseline | 4.7(4.41) | 5(3.52) | 3.6(1.86) | 4.6(2.56) |

TABLE 59
Pharmacokinetics Estradiol Day 1 (pg/mL)

| | 4 μg | 10 μg | 25 μg | Placebo |
|---|---|---|---|---|
| Predose | 3.1(1.56) | 4.9(3.47) | 3.6(1.81) | 4.1(2.45) |
| 2 hour | 6.1(2.3) | 10.4(4.89) | 28.7(17.91) | 4.8(3.33) |
| 4 hour | 4.3(1.68) | 6.7(3.59) | 16.1(14.75) | 5(3.59) |
| 6 hour | 3.7(1.96) | 5.7(3.16) | 9.7(6.86) | 4.8(3.53) |
| 10 hour | 3.7(1.47) | 5.5(2.92) | 6.2(2.37) | 5.2(3.61) |
| 24 hour | 4.2(2.02) | 5.4(4.44) | 6.2(8.43) | 5.1(4.42) |

TABLE 60
Pharmacokinetics Estradiol Day 14 (pg/mL)

| | 4 μg | 10 μg | 25 μg | Placebo |
|---|---|---|---|---|
| Predose | 3.5(1.63) | 3.8(2.56) | 5.2(2.89) | 4.2(3.07) |
| 2 hour | 4.3(2.01) | 6.3(2.29) | 15.3(7.72) | 4.2(2.44) |
| 4 hour | 4(1.7) | 5.9(2.55) | 11(4.86) | 4.7(3.2) |
| 6 hour | 3.9(1.92) | 5.1(2.32) | 7.9(3.35) | 4.7(2.97) |
| 10 hour | 3.8(2.12) | 5(3) | 6.8(3.76) | 5.1(3.53) |
| 24 hour | 3.6(1.89) | 3.7(2.05) | 4.9(4.35) | 3.9(2.43) |

TABLE 61
Pharmacokinetics Estradiol End of Study (pg/mL)

| | 4 μg | 10 μg | 25 μg | Placebo |
|---|---|---|---|---|
| Post Dosing | 4.3(2.69) | 4.8(2.57) | 6.7(11.51) | 4.4(2.6) |

### Estradiol Area Under the Curve (0-24 Hours)

TABLE 62
Estradiol Area Under the Curve (0-24 hours) (h*pg/mL)

| | 4 μg | 10 μg | 25 μg | Placebo |
|---|---|---|---|---|
| Day 1 | 91.7(37.86) | 138.2(75.22) | 217.4(99.02) | 116.6(77.3) |
| Day 14 | 87.2(42.77) | 110.1(54.57) | 171.6(80.13) | 104.2(66.39) |

TABLE 63
Estradiol Area Under the Curve (0-24
hours) (Baseline Adjusted) (h*pg/mL)

| | 4 μg | 10 μg | 25 μg | Placebo |
|---|---|---|---|---|
| Day 1 | 12(13.89) | 21.9(19.16) | 130.4(111.95) | 13.8(28.86) |
| Day 14 | 7.2(12.08) | 13.7(18.77) | 84.6(62.7) | 7.1(20.28) |

TABLE 64
Estradiol Area Under the Curve (0-24
hours) P-values Pairwise Test vs. 4 μg

| | 10 μg | 25 μg |
|---|---|---|
| Day 1 | 0.0242 | <0.0001 |
| Day 14 | 0.1777 | 0.0005 |

TABLE 65
Estradiol Area Under the Curve (0-24 hours)
P-values Pairwise Test vs. Placebo

| | 4 μg | 10 μg | 25 μg |
|---|---|---|---|
| Day 1 | 0.2292 | 0.4028 | 0.0021 |
| Day 14 | 0.3829 | 0.7724 | 0.0108 |

TABLE 66
Estradiol Area Under the Curve (0-24 hours) P-values
Pairwise Test vs. 4 μg (Baseline Adjusted)

| | 10 μg | 25 μg |
|---|---|---|
| Day 1 | 0.082 | 0.0001 |
| Day 14 | 0.2373 | <0.0001 |

TABLE 67
Estradiol Area Under the Curve (0-24 hours) P-values
Pairwise Test vs. Placebo (Baseline Adjusted)

| | 4 μg | 10 μg | 25 μg |
|---|---|---|---|
| Day 1 | 0.8134 | 0.3238 | 0.0002 |
| Day 14 | 0.979 | 0.3235 | <0.0001 |

TABLE 68
Estradiol Area Under the Curve (0-24 hours) Ratio (Day 14) of Day 14 to Day 1

| | 4 μg | 10 μg | 25 μg | Placebo |
|---|---|---|---|---|
| AUC Ratio of Day 14 | 0.971(0.2358) | 0.876(0.1937) | 0.955(0.6633) | 0.949(0.225) |
| to Day 1 | | | | |
| Pairwise test vs 4 ug | — | 0.2022 | 0.9246 | — |
| Pairwise test vs Placebo | 0.7859 | 0.3101 | 0.9748 | — |

### Estradiol Cₘₐₓ

TABLE 69
Cₘₐₓ (pg/mL)

| | 4 μg | 10 μg | 25 μg | Placebo |
|---|---|---|---|---|
| Day 1 | 6.5(2.13) | 10.9(5) | 29.8(17.51) | 6.6(4.85) |
| Day 14 | 4.8(2.31) | 7.3(2.36) | 15.7(7.61) | 5.5(3.43) |

TABLE 70
Cₘₐₓ (Baseline Adjusted) (pg/mL)

| | 4 μg | 10 μg | 25 μg | Placebo |
|---|---|---|---|---|
| Day 1 | 2.6(2.17) | 6(4.44) | 26.2(18.19) | 2.1(3.48) |
| Day 14 | 1.3(1.08) | 3(1.73) | 12(7.32) | 1(1.81) |

TABLE 71
Cₘₐₓ P-values Pairwise Test vs. 4 μg

| | 10 μg | 25 μg |
|---|---|---|
| Day 1 | 0.0013 | <0.0001 |
| Day 14 | 0.0033 | <0.0001 |

TABLE 72
Cₘₐₓ P-values Pairwise Test vs. Placebo

| | 4 μg | 10 μg | 25 μg |
|---|---|---|---|
| Day 1 | 0.9586 | 0.0116 | <0.0001 |
| Day 14 | 0.5174 | 0.0702 | <0.0001 |

TABLE 73
Cₘₐₓ P-values Pairwise Test vs. 4 μg (Baseline Adjusted)

| | 10 μg | 25 μg |
|---|---|---|
| Day 1 | 0.0055 | <0.0001 |
| Day 14 | 0.002 | <0.0001 |

TABLE 74
Cₘₐₓ P-values Pairwise Test vs. Placebo (Baseline Adjusted)

| | 4 μg | 10 μg | 25 μg |
|---|---|---|---|
| Day 1 | 0.6074 | 0.0059 | <0.0001 |
| Day 14 | 0.5088 | 0.0022 | <0.0001 |

TABLE 75
Cₘₐₓ Ratio (Day 14) of Day 14 to Day 1

| | 4 μg | 10 μg | 25 μg | Placebo |
|---|---|---|---|---|
| Cₘₐₓ Ratio of | 0.77(0.2633) | 0.804(0.3245) | 0.929(1.5011) | 0.933(0.2406) |
| Day 14 to Day 1 | | | | |
| Pairwise test vs | — | 0.7399 | 0.6702 | — |
| Pairwise test vs | 0.0702 | 0.1946 | 0.9931 | — |
| Placebo | | | | |

### Estradiol C_{avg}

TABLE 76
C_{avg} (pg/mL)

| | 4 μg | 10 μg | 25 μg | Placebo |
|---|---|---|---|---|
| Day 1 | 3.9(1.46) | 5.8(3.13) | 9.1(4.13) | 4.9(3.22) |
| Day 14 | 3.6(1.78) | 4.6(2.27) | 7.1(3.34) | 4.3(2.77) |

TABLE 77
C_{avg} (Baseline Adjusted) (pg/mL)

| | 4 μg | 10 μg | 25 μg | Placebo |
|---|---|---|---|---|
| Day 1 | 0(1.93) | 0.8(0.95) | 5.4(4.66) | 0.4(1.35) |
| Day 14 | 0.1(0.68) | 0.2(1.22) | 3.5(2.61) | −0.2(1.28) |

TABLE 78
C_{avg} P-values Pairwise Test vs. 4 μg

| | 10 μg | 25 μg |
|---|---|---|
| Day 1 | 0.0294 | <0.0001 |
| Day 14 | 0.1777 | 0.0005 |

TABLE 79
C_{avg} P-values Pairwise Test vs. Placebo

| | 4 μg | 10 μg | 25 μg |
|---|---|---|---|
| Day 1 | 0.267 | 0.4028 | 0.0021 |
| Day 14 | 0.3829 | 0.7724 | 0.0108 |

TABLE 80
C_{avg} P-values Pairwise Test vs. 4 μg (Baseline Adjusted)

| | 10 μg | 25 μg |
|---|---|---|
| Day 1 | 0.1076 | 0.0001 |
| Day 14 | 0.7759 | <0.0001 |

TABLE 81
C_{avg} P-values Pairwise Test vs. Placebo (Baseline Adjusted)

| | 4 μg | 10 μg | 25 μg |
|---|---|---|---|
| Day 1 | 0.5126 | 0.2564 | 0.0001 |
| Day 14 | 0.4098 | 0.3629 | <0.0001 |

TABLE 82
C_{avg} Ratio (Day 14) of Day 14 to Day 1

| | 4 μg | 10 μg | 25 μg | Placebo |
|---|---|---|---|---|
| C_{avg} Ratio of | 0.77(0.2633) | 0.804(0.3245) | 0.929(1.5011) | 0.933(0.2406) |
| Day 14 to Day 1 | | | | |
| Pairwise test vs | — | 0.7399 | 0.6702 | — |
| Pairwise test vs | 0.0702 | 0.1946 | 0.9931 | — |
| Placebo | | | | |

### Estradiol Tₘₐₓ

TABLE 83
Tₘₐₓ (h)

| | 4 μg | 10 μg | 25 μg | Placebo |
|---|---|---|---|---|
| Day 1 | 7(9.36) | 6.1(8.04) | 4.6(7.09) | 8.6(6.74) |
| Day 14 | 9.3(8.86) | 4(2.57) | 2.7(1.94) | 7.2(3) |

TABLE 84
Tₘₐₓ P-values Pairwise Test vs. 4 μg

| | 10 μg | 25 μg |
|---|---|---|
| Day 1 | 0.7566 | 0.3834 |
| Day 14 | 0.0206 | 0.004 |

TABLE 85
Tₘₐₓ P-values Pairwise Test vs. Placebo

| | 4 μg | 10 μg | 25 μg |
|---|---|---|---|
| Day 1 | 0.5705 | 0.3255 | 0.0943 |
| Day 14 | 0.3576 | 0.0019 | <0.0001 |

### Estrone Concentration

TABLE 86
Pharmacokinetics Estrone Baseline (pg/mL)

| | 4 μg | 10 μg | 25 μg | Placebo |
|---|---|---|---|---|
| Baseline | 15.9 (6.02) | 19.7 (9.18) | 16.3 (7.71) | 20.4 (9.67) |

TABLE 87
Pharmacokinetics Estrone Day 1 (pg/mL)

| | 4 μg | 10 μg | 25 μg | Placebo |
|---|---|---|---|---|
| Predose | 14.7 (4.44) | 21 (8.51) | 17.2 (8.5) | 18.3 (8.54) |
| 2 hour | 13.3 (4.52) | 20 (8.53) | 18.9 (6.7) | 18.9 (11.25) |
| 4 hour | 13 (4.68) | 19.3 (7.4) | 19.4 (7.06) | 19.9 (13.87) |
| 6 hour | 13.9 (6.04) | 19.6 (8.89) | 19.1 (8.1) | 19 (11.69) |
| 10 hour | 13.4 (4.94) | 19.7 (8.53) | 18.8 (7.18) | 19.3 (11.65) |
| 24 hour | 14.3 (5.92) | 21.2 (9.89) | 16.6 (6.06) | 22.9 (17.18) |

TABLE 88
Pharmacokinetics Estrone Day 14 (pg/mL)

| | 4 μg | 10 μg | 25 μg | Placebo |
|---|---|---|---|---|
| Predose | 15.8 (5.15) | 21.7 (14.25) | 18.6 (8.49) | 18.7 (9.38) |
| 2 hour | 13.6 (5.3) | 19.7 (10.2) | 19.8 (9.08) | 17.3 (7.99) |
| 4 hour | 14 (5.25) | 21 (13.46) | 19.9 (7.26) | 20.4 (11.41) |
| 6 hour | 14 (5.11) | 20.7 (10.4) | 19.3 (6.47) | 16.1 (7.54) |
| 10 hour | 14.2 (5.51) | 20.1 (11.93) | 19.3 (8.24) | 19 (8.17) |
| 24 hour | 14.5 (4.69) | 20.1 (9.34) | 16.7 (6.09) | 18.9 (8.24) |

TABLE 89
Pharmacokinetics Estrone End of Study (pg/mL)

| | 4 μg | 10 μg | 25 μg | Placebo |
|---|---|---|---|---|
| Post | 4.328 (2.7619) | 4.643 (2.5807) | 6.652 (11.508) | 4.363 (2.5982) |
| Dos- | | | | |
| ing | | | | |

### Estrone Area Under the Curve (0-24 Hours)

TABLE 90
Estrone Area Under the Curve (0-24 hours) (h * pg/mL)

| | 4 μg | 10 μg | 25 μg | Placebo |
|---|---|---|---|---|
| Day | 290.2 (123.67) | 462.7 (195.64) | 419.1 (147.85) | 467.9 (278.78) |
| 1 | | | | |
| Day | 326.6 (114.09) | 464.1 (243.92) | 428.7 (161.75) | 426.8 (180.67) |
| 14 | | | | |

TABLE 91
Estrone Area Under the Curve (0-24 hours)
(Baseline Adjusted) (h * pg/mL)

| | 4 μg | 10 μg | 25 μg | Placebo |
|---|---|---|---|---|
| Day 1 | 7.2 (20.91) | 10.9 (24.55) | 44.3 (54.27) | 43.5 (97.41) |
| Day 14 | 15 (41.53) | 43.2 (84.87) | 55.6 (78.06) | 17.4 (45.27) |

TABLE 92
Estrone Area Under the Curve (0-24 hours)
P-values Pairwise Test vs. 4 μg

| | 10 μg | 25 μg |
|---|---|---|
| Day 1 | 0.003 | 0.0076 |
| Day 14 | 0.042 | 0.0393 |

TABLE 93
Estrone Area Under the Curve (0-24 hours)
P-values Pairwise Test vs. Placebo

| | 4 μg | 10 μg | 25 μg |
|---|---|---|---|
| Day 1 | 0.0193 | 0.9487 | 0.519 |
| Day 14 | 0.0621 | 0.6117 | 0.9738 |

TABLE 94
Estrone Area Under the Curve (0-24 hours) P-values
Pairwise Test vs. 4 μg (Baseline Adjusted)

| | 10 μg | 25 μg |
|---|---|---|
| Day 1 | 0.6195 | 0.0104 |
| Day 14 | 0.2251 | 0.0658 |

TABLE 95
Estrone Area Under the Curve (0-24 hours) P-values
Pairwise Test vs. Placebo (Baseline Adjusted)

| | 4 μg | 10 μg | 25 μg |
|---|---|---|---|
| Day 1 | 0.1311 | 0.167 | 0.9761 |
| Day 14 | 0.8721 | 0.2746 | 0.0886 |

TABLE 96
Estrone Area Under the Curve (0-24 hours) Ratio (Day 14) of Day 14 to Day 1

| | 4 μg | 10 μg | 25 μg | Placebo |
|---|---|---|---|---|
| AUC Ratio of Day 14 | 1.234 (0.5824) | 1.023 (0.2675) | 1.039 (0.1941) | 1.006 (0.2316) |
| to Day 1 | | | | |
| Pairwise test vs | — | 0.1722 | 0.1866 | — |
| Pairwise test vs | 0.1432 | 0.848 | 0.6544 | — |
| Placebo | | | | |

### Estrone Cₘₐₓ

TABLE 97
Cₘₐₓ (pg/mL)

| | 4 μg | 10 μg | 25 μg | Placebo |
|---|---|---|---|---|
| Day 1 | 15.7 (6.07) | 23.5 (9.87) | 21.9 (7.73) | 25.7 (18.43) |
| Day 14 | 16 (5.5) | 23.9 (13.45) | 22.4 (8.95) | 22.8 (10.89) |

TABLE 98
Cₘₐₓ (Baseline Adjusted) (pg/mL)

| | 4 μg | 10 μg | 25 μg | Placebo |
|---|---|---|---|---|
| Day 1 | 0.4 (3.05) | 3.2 (2.99) | 5.1 (4.78) | 6.3 (12.81) |
| Day 14 | 0.6 (3.49) | 3.7 (8.79) | 5.6 (4.81) | 3.4 (5.69) |

TABLE 99
Cₘₐₓ P-values Pairwise Test vs. 4 μg

| | 10 μg | 25 μg |
|---|---|---|
| Day 1 | 0.007 | 0.0126 |
| Day 14 | 0.0301 | 0.0163 |

TABLE 100
Cₘₐₓ P-values Pairwise Test vs. Placebo

| | 4 μg | 10 μg | 25 μg |
|---|---|---|---|
| Day 1 | 0.0373 | 0.6567 | 0.4223 |
| Day 14 | 0.0275 | 0.7878 | 0.8979 |

TABLE 101
Cₘₐₓ P-values Pairwise Test vs. 4 μg (Baseline Adjusted)

| | 10 μg | 25 μg |
|---|---|---|
| Day 1 | 0.0087 | 0.0013 |
| Day 14 | 0.1975 | 0.0014 |

TABLE 102
Cₘₐₓ P-values Pairwise Test vs. Placebo (Baseline Adjusted)

| | 4 μg | 10 μg | 25 μg |
|---|---|---|---|
| Day 1 | 0.0659 | 0.3046 | 0.71 |
| Day 14 | 0.0938 | 0.933 | 0.2249 |

TABLE 103
Cₘₐₓ Ratio (Day 14) of Day 14 to Day 1

| | 4 μg | 10 μg | 25 μg | Placebo |
|---|---|---|---|---|
| Cₘₐₓ Ratio of | 1.029 (0.2346) | 1.042 | 1.041 | 1.039 |
| Day 14 to Day 1 | | (0.3436) | (0.2179) | (0.2916) |
| Pairwise test vs | — | 0.9035 | 0.8835 | — |
| Pairwise test vs | 0.9188 | 0.9788 | 0.982 | — |
| Placebo | | | | |

### Estrone C_{avg}

TABLE 104
C_{avg} (pg/mL)

| | 4 μg | 10 μg | 25 μg | Placebo |
|---|---|---|---|---|
| Day 1 | 13 (4.72) | 19.3 (8.15) | 17.5 (6.16) | 19.5 (11.62) |
| Day 14 | 13.6 (4.75) | 19.3 (10.16) | 17.9 (6.74) | 17.8 (7.53) |

TABLE 105
C_{avg} (Baseline Adjusted) (pg/mL)

| | 4 μg | 10 μg | 25 μg | Placebo |
|---|---|---|---|---|
| Day 1 | −2.3 (2.26) | −1.1 (2.66) | 0.7 (3.73) | 0.1 (5.03) |
| Day 14 | −1.7 (3.25) | −0.9 (5.91) | 1.1 (4.81) | −1.6 (3.8) |

TABLE 106
C_{avg} P-values Pairwise Test vs. 4 μg

| | 10 μg | 25 μg |
|---|---|---|
| Day 1 | 0.0075 | 0.0207 |
| Day 14 | 0.042 | 0.0393 |

TABLE 107
C_{avg} P-values Pairwise Test vs. Placebo

| | 4 μg | 10 μg | 25 μg |
|---|---|---|---|
| Day 1 | 0.0363 | 0.9487 | 0.519 |
| Day 14 | 0.0621 | 0.6117 | 0.9738 |

TABLE 108
C_{avg} P-values Pairwise Test vs. 4 μg (Baseline Adjusted)

| | 10 μg | 25 μg |
|---|---|---|
| Day 1 | 0.1345 | 0.0057 |
| Day 14 | 0.6351 | 0.0495 |

TABLE 109
C_{avg} P-values Pairwise Test vs. Placebo (Baseline Adjusted)

| | 4 μg | 10 μg | 25 μg |
|---|---|---|---|
| Day 1 | 0.0712 | 0.3751 | 0.691 |
| Day 14 | 0.912 | 0.7058 | 0.0742 |

TABLE 110
C_{avg} Ratio (Day 14) of Day 14 to Day 1

| | 4 μg | 10 μg | 25 μg | Placebo |
|---|---|---|---|---|
| C_{avg} Ratio of | 1.029 (0.2346) | 1.042 | 1.041 | 1.039 |
| Day 14 to Day 1 | | (0.3436) | (0.2179) | (0.2916) |
| Pairwise test vs | — | 0.9035 | 0.8835 | — |
| Pairwise test vs | 0.9188 | 0.9788 | 0.982 | — |
| Placebo | | | | |

### Estrone Tₘₐₓ

TABLE 111
Tₘₐₓ (h)

| | 4 μg | 10 μg | 25 μg | Placebo |
|---|---|---|---|---|
| Day 1 | 14.1 (9.37) | 11.9 (9.76) | 9.1 (7.43) | 12.1 (9.39) |
| Day 14 | 10.9 (9.03) | 10.4 (8.93) | 6.3 (6.9) | 12.2 (9.24) |

TABLE 112
Tₘₐₓ P-values Pairwise Test vs. 4 μg

| | 10 μg | 25 μg |
|---|---|---|
| Day 1 | 0.4862 | 0.0849 |
| Day 14 | 0.8711 | 0.0982 |

TABLE 113
Tₘₐₓ P-values Pairwise Test vs. Placebo

| | 4 μg | 10 μg | 25 μg |
|---|---|---|---|
| Day 1 | 0.5341 | 0.9449 | 0.2997 |
| Day 14 | 0.6824 | 0.5639 | 0.0391 |

### Estrone Conjugates

TABLE 114
Pharmacokinetics Estrone Conjugates Baseline (pg/mL)

| | 4 μg | 10 μg | 25 μg | Placebo |
|---|---|---|---|---|
| Baseline | 250.3(162.91) | 259.7(208.51) | 374.4(586.45) | 280.7(171.26) |

TABLE 115
Pharmacokinetics Estrone Conjugates Day 1 (pg/mL)

| | 4 μg | 10 μg | 25 μg | Placebo |
|---|---|---|---|---|
| Predose | 225.1(215.01) | 218.6(147.84) | 312.4(410.38) | 271.2(153.33) |
| 2 hour | 206.8(163.2) | 273.1(176.59) | 396.6(408.16) | 223.4(162.11) |
| 4 hour | 241.7(176.87) | 267.2(161.79) | 413.3(343.25) | 241.8(139.77) |
| 6 hour | 240.6(181.14) | 266(184.92) | 477.8(472.66) | 265(154.01) |
| 10 hour | 223(150.42) | 243.5(173.71) | 436.4(461) | 258(133.21) |
| 24 hour | 229.4(186.79) | 268.4(221.29) | 306.4(322.91) | 268.8(153.22) |

TABLE 116
Pharmacokinetics Estrone Conjugates Day 14 (pg/mL)

| | 4 μg | 10 μg | 25 μg | Placebo |
|---|---|---|---|---|
| Predose | 212.7(140.19) | 319.1(326.71) | 411.1(624.14) | 256.1(133.07) |
| 2 hour | 212.4(145.02) | 420.4(560.53) | 434.3(491.31) | 285.6(158.61) |
| 4 hour | 240.2(155.7) | 429.3(506.01) | 505.1(618.47) | 273.1(148.76) |
| 6 hour | 225.8(164.76) | 359.2(346.26) | 483.8(515.95) | 267.7(181.53) |
| 10 hour | 238.3(152.45) | 417.6(517.51) | 492.5(598.16) | 306.9(178.68) |
| 24 hour | 206.4(154.26) | 349(345.91) | 309.6(380.88) | 240.1(115.84) |

TABLE 117
Pharmacokinetics Estrone Conjugates End of Study (pg/mL)

| | 4 μg | 10 μg | 25 μg | Placebo |
|---|---|---|---|---|
| Post | 237.4(151.19) | 221.7(188.05) | 499.7(1089.67) | 250(148.72) |
| Dosing | | | | |

### Estrone Conjugates Area Under the Curve (0-24 Hours)

TABLE 118
Estrone Conjugates Area Under the Curve (0-24 hours) (h*pg/mL)

| | 4 μg | 10 μg | 25 μg | Placebo |
|---|---|---|---|---|
| Day 1 | 5077.5(3798.39) | 5931.9(4209.95) | 9126(9186.37) | 5637.9(3151.49) |
| Day 14 | 5172.9(3382.89) | 8978(9811.23) | 9930.2(11711.99) | 6275.2(3397.54) |

TABLE 119
Estrone Conjugates Area Under the Curve (0-24
hours) (Baseline Adjusted) (h*pg/mL)

| | 4 μg | 10 μg | 25 μg | Placebo |
|---|---|---|---|---|
| Day 1 | 375.5(843.98) | 422.4(473.83) | 2454.3(2600.25) | 83(229.06) |
| Day 14 | 660.5(1230.69) | 3767.2(7671.38) | 3059(4792.46) | 665.4(1552.19) |

TABLE 120
Estrone Conjugates Area Under the Curve (0-24
hours) P-values Pairwise Test vs. 4 μg

| | 10 μg | 25 μg |
|---|---|---|
| Day 1 | 0.5219 | 0.0931 |
| Day 14 | 0.1392 | 0.1166 |

TABLE 121
Estrone Conjugates Area Under the Curve (0-24
hours) P-values Pairwise Test vs. Placebo

| | 4 μg | 10 μg | 25 μg |
|---|---|---|---|
| Day 1 | 0.639 | 0.8157 | 0.1472 |
| Day 14 | 0.3503 | 0.2898 | 0.2246 |

TABLE 122
Estrone Conjugates Area Under the Curve (0-24 hours)
P-values Pairwise Test vs. 4 μg (Baseline Adjusted)

| | 10 μg | 25 μg |
|---|---|---|
| Day 1 | 0.8349 | 0.0028 |
| Day 14 | 0.1087 | 0.0537 |

TABLE 123
Estrone Conjugates Area Under the Curve (0-24 hours) P-
values Pairwise Test vs. Placebo (Baseline Adjusted)

| | 4 μg | 10 μg | 25 μg |
|---|---|---|---|
| Day 1 | 0.1894 | 0.0134 | 0.001 |
| Day 14 | 0.992 | 0.1225 | 0.0654 |

TABLE 124
Estrone Conjugates Area Under the Curve (0-24
hours) Ratio (Day 14) of Day 14 to Day 1

| | 4 μg | 10 μg | 25 μg | Placebo |
|---|---|---|---|---|
| AUC Ratio of | 1.115(0.4539) | 1.444(1.0121) | 1.107(0.3545) | 1.125(0.4522) |
| Day 14 to Day 1 | | | | |
| Pairwise test vs | — | 0.2279 | 0.9587 | — |
| Pairwise test vs | 0.9459 | 0.2427 | 0.8975 | — |
| Placebo | | | | |

### Estrone Conjugates Cₘₐₓ

TABLE 125
Cₘₐₓ (pg/mL)

| | 4 μg | 10 μg | 25 μg | Placebo |
|---|---|---|---|---|
| Day | 273.1(196.36) | 329.4(226.58) | 542.1(475.49) | 309.8(146.07) |
| 1 | | | | |
| Day | 289(183.79) | 511.7(568.75) | 579.5(610.1) | 343.6(182.2) |
| 14 | | | | |

TABLE 126
Cₘₐₓ (Baseline Adjusted) (pg/mL)

| | 4 μg | 10 μg | 25 μg | Placebo |
|---|---|---|---|---|
| Day | 35.4(89.09) | 90.2(65.2) | 198.6(301.53) | 27.1(49.69) |
| 1 | | | | |
| Day | 48.2(132.61) | 277.8(493.64) | 236.1(372.42) | 67(121.81) |
| 14 | | | | |

TABLE 127
Cₘₐₓ P-values Pairwise Test vs. 4 μg

| | 10 μg | 25 μg |
|---|---|---|
| Day 1 | 0.4261 | 0.0333 |
| Day 14 | 0.1332 | 0.0685 |

TABLE 128
Cₘₐₓ P-values Pairwise Test vs. Placebo

| | 4 μg | 10 μg | 25 μg |
|---|---|---|---|
| Day 1 | 0.5369 | 0.7629 | 0.0625 |
| Day 14 | 0.3902 | 0.2533 | 0.1356 |

TABLE 129
Cₘₐₓ P-values Pairwise Test vs. 4 μg (Baseline Adjusted)

| | 10 μg | 25 μg |
|---|---|---|
| Day 1 | 0.039 | 0.0345 |
| Day 14 | 0.0726 | 0.0579 |

TABLE 130
Cₘₐₓ P-values Pairwise Test vs. Placebo (Baseline Adjusted)

| | 4 μg | 10 μg | 25 μg |
|---|---|---|---|
| Day 1 | 0.7444 | 0.0033 | 0.0318 |
| Day 14 | 0.6735 | 0.1065 | 0.0928 |

TABLE 131
Cₘₐₓ Ratio (Day 14) of Day 14 to Day 1

| | 4 μg | 10 μg | 25 μg | Placebo |
|---|---|---|---|---|
| Cₘₐₓ Ratio of | 1.13(0.4068) | 1.524(1.1682) | 1.144(0.4569) | 1.11(0.5404) |
| Day 14 to Day 1 | | | | |
| Pairwise test vs | — | 0.1969 | 0.9226 | — |
| Pairwise test vs | 0.9043 | 0.1919 | 0.8406 | — |
| Placebo | | | | |

### Estrone Conjugates C_{avg}

TABLE 132
C_{avg} (pg/mL)

| | 4 μg | 10 μg | 25 μg | Placebo |
|---|---|---|---|---|
| Day | 215.9(154.77) | 247.2(175.41) | 380.3(382.77) | 244.6(128.1) |
| 1 | | | | |
| Day | 215.5(140.95) | 374.1(408.8) | 413.8(488) | 261.5(141.56) |
| 14 | | | | |

TABLE 133
C_{avg} (Baseline Adjusted) (pg/mL)

| | 4 μg | 10 μg | 25 μg | Placebo |
|---|---|---|---|---|
| Day | −21.8(88.41) | 8(34.21) | 36.8(291.72) | −33.7(46.95) |
| 1 | | | | |
| Day | −25.3(120.69) | 140.2(330.6) | 70.3(300.36) | −7.9(89.89) |
| 14 | | | | |

TABLE 134
C_{avg} P-values Pairwise Test vs. 4 μg

| | 10 μg | 25 μg |
|---|---|---|
| Day 1 | 0.5701 | 0.1004 |
| Day 14 | 0.1392 | 0.1166 |

TABLE 135
C_{avg} P-values Pairwise Test vs. Placebo

| | 4 μg | 10 μg | 25 μg |
|---|---|---|---|
| Day 1 | 0.5562 | 0.9602 | 0.1741 |
| Day 14 | 0.3503 | 0.2898 | 0.2246 |

TABLE 136
C_{avg} P-values Pairwise Test vs. 4 μg (Baseline Adjusted)

| | 10 μg | 25 μg |
|---|---|---|
| Day 1 | 0.1804 | 0.4201 |
| Day 14 | 0.0606 | 0.2305 |

TABLE 137
C_{avg} P-values Pairwise Test vs. Placebo (Baseline Adjusted)

| | 4 μg | 10 μg | 25 μg |
|---|---|---|---|
| Day 1 | 0.6353 | 0.0047 | 0.3473 |
| Day 14 | 0.6439 | 0.0928 | 0.3244 |

TABLE 138
C_{avg} Ratio (Day 14) of Day 14 to Day 1

| | 4 μg | 10 μg | 25 μg | Placebo |
|---|---|---|---|---|
| C_{avg} Ratio of | 1.13(0.4068) | 1.524(1.1682) | 1.144(0.4569) | 1.11(0.5404) |
| Day 14 to Day 1 | | | | |
| Pairwise test vs | — | 0.1969 | 0.9226 | — |
| Pairwise test vs | 0.9043 | 0.1919 | 0.8406 | — |
| Placebo | | | | |

### Estrone Conjugates Tₘₐₓ

TABLE 139
Tₘₐₓ (h)

| | 4 μg | 10 μg | 25 μg | Placebo |
|---|---|---|---|---|
| Day 1 | 10.9(8.66) | 9.2(9.25) | 5.4(2.64) | 13.1(9.7) |
| Day 14 | 8.4(7.79) | 9(8.6) | 5.9(2.87) | 8.1(6.76) |

TABLE 140
Tₘₐₓ P-values Pairwise Test vs. 4 μg

| | 10 μg | 25 μg |
|---|---|---|
| Day 1 | 0.5609 | 0.0154 |
| Day 14 | 0.8173 | 0.2178 |

TABLE 141
Tmax P-values Pairwise Test vs. Placebo

| | 4 μg | 10 μg | 25 μg |
|---|---|---|---|
| Day 1 | 0.4893 | 0.2253 | 0.003 |
| Day 14 | 0.9256 | 0.739 | 0.2087 |

In the phase 3 trial, all doses of TX-004HR compared with placebo (MITT n=747) significantly improved the 4 co-primary endpoints at week 2 through week 12, as well as the secondary endpoints of vaginal dryness by week 6 and vulvar and/or vaginal itching or irritation by week 12 (except 4 μg, p=0.0503), and was well-tolerated with no treatment-related serious AEs reported. The phase 3 PK study (n=72) showed no difference in systemic E2 levels for 4 μg and 10 μg TX-004HR vs placebo, as measured by AUC and Cavg. E2 AUC and Cavg with 25 TX-004HR was higher than placebo, but average concentrations remained within the normal postmenopausal range (Table 142). E2 levels at day 84 were similar to placebo indicating no systemic drug accumulation. SHBG concentrations did not change with treatment. The two phase 2 studies (n=36 for each) of TX-004HR 10 μg and 25 μg resulted in statistically significantly lower E2 absorption than an approved E2 tablet at identical doses, with 25 μg TX-004HR demonstrating AUC less than ⅓ that of the approved product (Table 143).

TABLE 142
Phase 3 study PK parameters for E2 (unadjusted mean ± SD).

| | Dose | AUC₀₋₂₄ (pg * hr/mL) | | | C_{avg} (pg/mL) | | |
|---|---|---|---|---|---|---|---|
| Day | (μg) | TX-004HR | Placebo | p-value | TX-004HR | Placebo | p-value |
| 1 | 4 | 91.7 ± 37.9 | 116.6 ± 77.3 | NS | 3.92 ± 1.46 | 4.86 ± 3.22 | NS |
| | 10 | 138.2 ± 75.2 | 116.6 ± 77.3 | NS | 5.76 ± 3.13 | 4.86 ± 3.22 | NS |
| | 25 | 217.4 ± 99.0 | 116.6 ± 77.3 | 0.0021 | 9.06 ± 4.13 | 4.86 ± 3.22 | 0.0021 |
| 14 | 4 | 87.2 ± 42.8 | 104.2 ± 66.4 | NS | 3.63 ± 1.78 | 4.34 ± 2.77 | NS |
| | 10 | 110.1 ± 54.6 | 104.2 ± 66.4 | NS | 4.59 ± 2.27 | 4.34 ± 2.77 | NS |
| | 25 | 171.6 ± 80.1 | 104.2 ± 66.4 | 0.0108 | 7.15 ± 3.34 | 4.34 ± 2.77 | 0.0108 |

TABLE 143
Phase 2 studies PK parameters for E2
(baseline adjusted geometric mean).

| | AUC₀₋₂₄ (pg * hr/mL) | | | C_{avg} (pg/mL) | | |
|---|---|---|---|---|---|---|
| Dose | TX- | Vaginal | | TX- | Vaginal | |
| (μg) | 004HR | Tablet | p-value | 004HR | Tablet | p-value |
| 10 | 49.62 | 132.92 | <0.0001 | 14.38 | 20.38 | 0.0194 |
| 25 | 89.21 | 292.06 | <0.0001 | 23.08 | 42.70 | <0.0001 |

With robust efficacy demonstrated as early as 2 weeks and up to 12 weeks at all 3 doses, TX-004HR 4 μg and 10 μg showed negligible systemic E2 absorption, while 25 μg resulted in very low systemic absorption of E2 in the phase 3 trial. TX-004HR 10 μg and 25 μg showed lower systemic E2 exposure than equivalent doses of an approved E2 tablet. The absence of clinically meaningful increases in E2 concentrations paired with data consistent with a lack of systemic effects (e.g., no increase in SHBG) shows that TX-004 HR delivers excellent efficacy with negligible to very low systemic exposure.

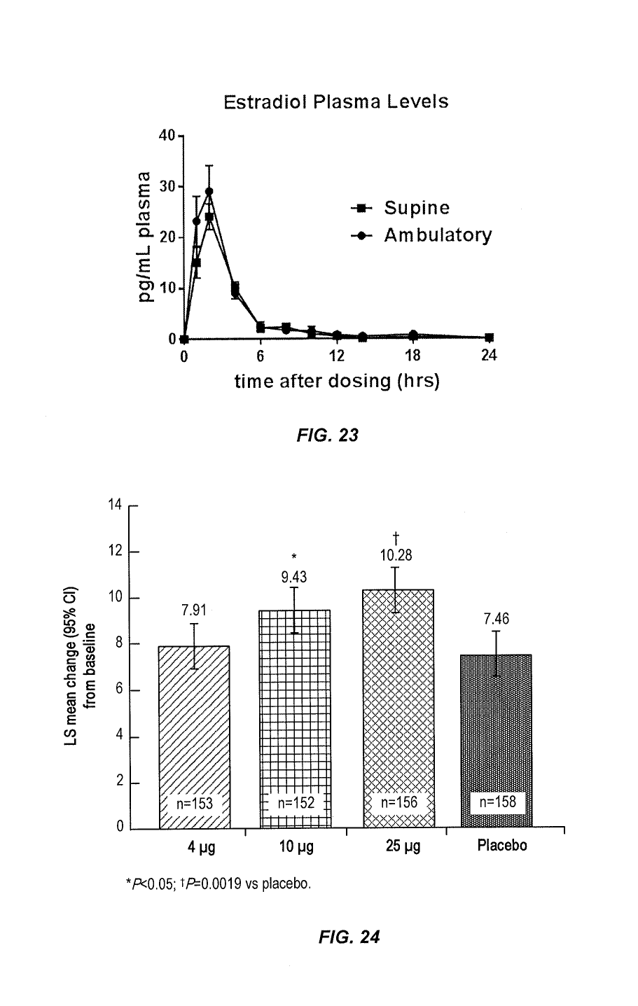

The impact of normal daily activities for 4 hours post dose was evaluated, in comparison with the impact of remaining in the supine position for 4 hours post dose on the pharmacokinetic (PK) profile of TX-004HR 25 mcg. In two studies, at the same site, the same sixteen healthy postmenopausal female subjects were fasted for at least 10 hours prior to dosing through 4 hours following dosing. Subjects received a 25 mcg dose of TX-004HR administered intravaginally by trained female study personnel. Following their first dose, the subjects were required to remain in a supine position for 4 hours following dosing. Following the second dose, after 5 minutes resting time, the subjects were instructed to be ambulatory in the clinic and refrain from reclining for the 4 hours following dosing. Blood samples were collected at pre-defined intervals up to 24 hours after dosing. Plasma samples were analyzed for estradiol using LC-MS/MS. See, e.g., FIG. 23. PK parameters were calculated on an individual and group mean basis with baseline correction.

The mean Cmax and AUC0-24 of estradiol was not significantly different with ambulation than with supination. On an individual subject basis, the majority showed similar Cmax and AUC0-24 levels with ambulation as with supination. There were no signs of posture having an effect on absorption rate as evidenced by the similarity in group average and individual subject Tmax. In addition, there was no difference between the group mean profiles when compared on an individual time point basis, further demonstrating that posture had no effect on absorption. The systemic exposure of estradiol in TX-004HR 25 mcg was generally low and occurred regardless of whether the subjects were ambulatory or supine for 4 hours after dosing. An important advantage of the formulation is that a woman can be ambulatory almost immediately after the formulation is administered, as opposed to other known formulations that require a subject to remain in a supine position after administration. Generally, other known formulations direct administration before bed at night because of the requirement to be supine, which requirement is unnecessary in the pharmaceutical compositions disclosed herein because the pharmaceutical compositions disclosed herein adhere to the vaginal tissue, the capsule dissolves rapidly, and the formulation is released into the vagina and rapidly absorbed by the vaginal tissue. Because activity level does not adversely affect the systemic absorption of estradiol, the formulation of the invention gives the patient more flexibility with her dosing regimen.

### Example 13: Safety Results in Randomized, Double-Blind, Placebo-Controlled Multicenter Study

Safety endpoints in the study included vital signs, clinical laboratory tests (blood chemistry, hematology, hormone levels, urine analysis), ECG, physical and gynecological examination findings, pap smears, endometrial biopsies, and adverse events (AEs). AEs included undesirable medical conditions occurring at any time during all study phases including the washout period, whether or not a study treatment had been administered. An AE was considered treatment emergent if it occurred after study drug administration, or if it was pre-existing and worsened during 120 days post-dose follow up. Participants were given a diary with instructions to record product use, sexual activity, symptoms/complaints, and other medications. AEs, concomitant medications, and vital signs were recorded and assessed at each study visit from screening to week 12.

TX-004HR had a favorable safety profile and was well tolerated. No clinically significant differences in AEs were observed between treatment and placebo groups (Table 144). Headache was the most commonly reported TEAE, followed by vaginal discharge, nasopharyngitis, and vulvovaginal pruritus (Table 144). Headache was the only treatment-related TEAE that was numerically more frequent in women receiving TX-004HR than those receiving placebo (3.7% for 4-μg dose vs 3.1% for placebo). Vaginal discharge was reported by numerically fewer women in any of the TX-004HR groups than by women in the placebo group. Most TEAEs were mild to moderate in severity. Few participants (1.8%) discontinued the study due to AEs.

TABLE 144
Number (%) of treatment emergent adverse events (TEAE) reported
for ≥3% in any treatment arm of the safety population.

| | TX- | | TX- | | TX- | | | |
|---|---|---|---|---|---|---|---|---|
| | 004HR | | 004HR | | 004HR | | | |
| | 4 μg | | 10 μg | | 25 μg | | Placebo | |
| Preferred Term | (n = 191) | | (n = 191) | | (n = 190) | | (n = 192) | |
| Any subject with | 97 | (50.8) | 94 | (49.2) | 93 | (48.9) | 111 | (57.8) |
| reported TEAE | | | | | | | | |
| Headache | 12 | (6.3) | 14 | (7.3) | 6 | (3.2) | 15 | (7.8) |
| Vaginal discharge | 5 | (2.6) | 6 | (3.1) | 4 | (2.1) | 13 | (6.8) |
| Nasopharyngitis | 5 | (2.6) | 6 | (3.1) | 7 | (3.7) | 10 | (5.2) |
| Vulvovaginal pruritus | 4 | (2.1) | 3 | (1.6) | 7 | (3.7) | 10 | (5.2) |
| Back pain | 9 | (4.7) | 1 | (0.5) | 4 | (2.1) | 8 | (4.2) |
| Urinary tract infection | 5 | (2.6) | 5 | (2.6) | 8 | (4.2) | 4 | (2.1) |
| Upper respiratory tract | 5 | (2.6) | 6 | (3.1) | 3 | (1.6) | 5 | (2.6) |
| infection | | | | | | | | |
| Oropharyngeal pain | 1 | (0.5) | 0 | (0) | 6 | (3.2) | 1 | (0.5) |

Nine serious TEAEs were reported in 8 subjects; however, none were considered related to treatment. Complete heart block, appendicitis, endophthalmitis, and chronic obstructive pulmonary disease were each reported by a different participant in the 25 μg group. Sinus node dysfunction and ankle fracture were both reported for one women, and arthralgia and malignant melanoma were each reported for one women in the 10 μg group. None of the women in the 4 μg group had reports of serious TEAEs. One woman in the placebo group was reported to have a cervical myelopathy. No deaths occurred during the study.

No diagnoses of endometrial hyperplasia or malignancy from endometrial biopsies were observed at week 12. Total cholesterol numerically decreased from baseline to week 12 by a mean of 0.024 mmol/L to 0.07 mmol/L in the treatment groups, and by 0.008 mmol/L in the placebo group. No clinically meaningful increases in triglycerides were observed in any active treatment groups compared with placebo. Sex hormone binding globulin (SHBG) concentrations (measured in a subset of 72 women) did not increase with treatment relative to placebo or baseline at week 12. No clinically significant changes in any laboratory parameters were found.

The phase 3 clinical trial demonstrated that TX-004HR at 4 μg, 10 μg, and 25 μg doses is safe and effective for treating vaginal changes and self-reported symptoms of VVA in postmenopausal women. Statistically significant and clinically meaningful improvements in all of the 4 pre-specified co-primary endpoints (increase in the percentage of vaginal superficial cells, decrease in the percentage of vaginal parabasal cells and vaginal pH, and decrease in severity of the MBS of dyspareunia) occurred as early as 2 weeks with all 3 doses of TX-004HR as compared with placebo, and were sustained throughout the 12-week trial. Additionally, improvements were found for the secondary endpoints of vaginal dryness and vulvar or vaginal irritation and itching. These improvements were achieved without increasing systemic estrogen concentrations (4 μg and 10 μg) or with negligible (25 μg) systemic estrogen exposure, as found in pharmacokinetic studies. TX-004HR was also well-tolerated with no clinically significant differences found between treatment and placebo groups in any AEs or treatment-related AEs, and no treatment-related serious AEs.

The results demonstrate early onset of action in the clinical signs of VVA with statistically significantly improved changes compared with placebo. The efficacy results here were somewhat numerically higher than data from a 12-week, randomized, controlled trial that compared a 10-μg vaginal estradiol tablet with placebo, which showed significant improvements in the percentages of superficial and parabasal cells, and in pH compared with placebo (see, Simon et al. Obstet Gynecol. 2008; 112:1053-1060). At 12 weeks, improvements were smaller with the 10-μg estradiol tablet (change of 13% in superficial cells, −37% in parabasal cells, and −1.3 in vaginal pH) than what was observed in this study with the 10-μg TX-004HR dose (change of 17% in superficial cells, −44% in parabasal cells, and −1.4 in vaginal pH). While improvements in some objective (cell and pH) endpoints were seen with the estradiol tablet within 2 weeks of treatment, the patient-reported improvements in a composite score of subjective symptoms were not observed until 8 weeks of therapy, which can be perceived as a disadvantage for many users. That clinical trial did not assess individual symptoms. A second randomized, controlled trial of 10-μg and 25-μg estradiol tablets similarly did not find significant improvements over placebo in the composite score of vaginal symptoms with either dose until 7 weeks of treatment (week 2, NS). Likewise, the SERM, ospemifene, was evaluated in a clinical trial for the treatment of dyspareunia, and statistically significant improvements were not observed until week 12. See, Bachmann et al. Obstet Gynecol. 2008; 111:67-76; Portman et al. Menopause. 2013; 20:623-630.

Importantly, the results reported here showed significant improvement in dyspareunia within 2 weeks with all 3 doses of TX-004HR, with reductions in severity scores from 1.5 to 1.7 points at week 12, which were comparable or superior to reductions of 1.2 to 1.6 points reported for other currently approved dyspareunia treatments. See, Vagifem® (estradiol vaginal tablets) Prescribing Information. Bagsvaerd, Denmark: Novo Nordisk Pharmaceuticals Inc.; 2012; Premarin® (conjugated estrogens tablets, USP) Prescribing Information. Philadelphia, Pa.: Wyeth Pharmaceuticals Inc.; 2010; Osphena® (ospemifene) tablets, for oral use. Prescribing Information. Shionogi, Inc. 2013.

Additionally, vaginal dryness improved from week 2 with 10 μg and 25 μg TX-004HR. None of the currently available products reported as early an onset of action for the symptom of vaginal dryness associated with VVA as did TX-004HR. Furthermore, TX-004HR 10 μg and 25 μg showed significant improvement in vaginal irritation and/or itching at week 12, while none of the currently available products on the market are reported to improve these symptoms. See, Portman et al. Maturitas. 2014; 78:91-98; Eriksen et al. Eur J Obstet Gynecol Reprod Biol. 1992; 44:137-144.

Based on a large survey of postmenopausal women in the United States, only a small proportion (7%) of women are thought to receive prescription vaginal estrogen therapy alone for their VVA, probably due to lack of information about available treatments, avoidance of discussion of the topic with health care practitioners, or dissatisfaction with currently available products (see, e.g., Kingsberg et al. J Sex Med. 2013; 10:1790-1799). Eliminating the need for an applicator or individually measuring doses is intended to give women a more positive user experience and thus potentially better compliance, resulting in overall better efficacy of treatment.

The results with TX-004HR in this study exemplify one of the advantages of local vaginal estrogen therapies: rapid symptom resolution without increasing systemic estrogen concentrations. The mean area under the concentration-time curve (AUC) and average concentration (Cavg) for estradiol were not significantly different from placebo with 4 μg and 10 μg TX-004HR. Although statistically higher AUC for estradiol was observed with the 25 μg dose, estradiol levels remained within the postmenopausal range with no evidence of accumulation by day 84. Although there was negligible systemic absorption, rapid efficacy was observed within 2 weeks of dosing with all doses of TX-004HR.

TX-004HR was well-tolerated. The 4 most commonly reported TEAEs, including vaginal discharge and vulvovaginal pruritus, were experienced by fewer women in any TX-004HR group than in the placebo group, and were mostly mild to moderate in severity. By comparison, in a 12-week study of the efficacy of ospemifene, vaginal discharge was reported more than 6-times more frequently in the ospemifene group than in the placebo group (see, Portman et al. Menopause. 2013; 20:623-630). Genital pruritus was also reported 4-times more frequently in women treated with Vagifem 10-μg tablets than with placebo in a 12-month randomized study (see, Vagifem® (estradiol vaginal tablets) Prescribing Information. Bagsvaerd, Denmark: Novo Nordisk Pharmaceuticals Inc.; 2012). Importantly, endometrial findings after TX-004HR were benign as no hyperplasia or malignancies were reported in biopsies at 12 weeks. Onset of effect was seen as early as 2 weeks and was maintained throughout the study. TX-004HR was well tolerated as reported here and systemic estrogen exposure was negligible to very low as demonstrated by the pharmacokinetic study.

### Example 14: Results of Female Sexual Function Index in Randomized, Double-Blind, Placebo-Controlled Multicenter Study

The trial was a randomized, double-blind, placebo controlled, multicenter, phase 3 study. Treatments were self-administered vaginally, once daily, for 2 weeks and then twice weekly, for 10 weeks. Female sexual dysfunction (FSD) was evaluated using the multidimensional Female Sexual Function Index (FSFI) at baseline and at week 12. The FSFI is a brief, validated, self-reporting questionnaire consisting of 19 questions designed to assess the areas of arousal, desire, orgasm, lubrication, and pain. The Index defines sexual dysfunction by a total FSFI score (the sum of the individual domain scores) of ≤26.55 out of a possible maximum score of 36.

Postmenopausal women (40-75 years; BMI ≤38 kg/m2) were included if they had ≤5% superficial cells on vaginal cytological smear; vaginal pH >5.0; self-identified most bothersome symptom (MBS) of moderate-to severe dyspareunia; and anticipated sexual activity (with vaginal penetration) during the trial period. Vulvar and vaginal atrophy (VVA) treatments, including vaginal lubricants and moisturizers, were discontinued within 7 days prior to screening. Use of oral estrogen-, progestin-, androgen-, or SERM-containing drug products were prohibited within 8 weeks of study start. Changes from baseline in total and individual domain FSFI scores for each dose were compared with placebo using ANCOVA with baseline as a covariate.

764 postmenopausal women were randomized to 4 μg (n=191), 10 μg (n=191), or 25 μg (n=190) vaginal estradiol softgel capsules or placebo (n=192). The majority of the women were white (87%) with a mean age of 59 years and a mean BMI of 26.7 kg/m2 (Table 145). The FSFI questionnaire was completed by those who were not in the PK sub-study (n=692; 90.6%). The average baseline total FSFI score of 14.8 for all women indicated FSD in the subjects.

TABLE 145
Summary of subjects enrolled in study

| | Com- | Com- | Com- | |
|---|---|---|---|---|
| | position | position | position | Com- |
| | 4 | 5 | 6 | position |
| | 4 μg | 10 μg | 25 μg | 7 |
| | (n = 186) | (n = 188) | (n = 186) | (n = 187) |
| Age, years | | | | |
| Mean ± SD | 59.8 ± 6.0 | 58.6 ± 6.3 | 58.8 ± 6.2 | 59.4 ± 6.0 |
| Race, n (%) | | | | |
| White | 162 (87.1) | 165 (87.8) | 161 (86.6) | 160 (85.6) |
| Black or | 20 (10.8) | 21 (11.2) | 24 (12.9) | 21 (11.2) |
| African | | | | |
| American | | | | |
| Asian | 3 (1.6) | 2 (1.1) | 1 (0.5) | 1 (0.5) |
| BMI, kg/m² | | | | |
| Mean ± SD | 26.6 ± 4.9 | 26.8 ± 4.7 | 26.9 ± 4.8 | 26.6 ± 4.6 |
| Baseline total | | | | |
| FSFI Score | | | | |
| Mean ± SD | 14.8 ± 6.13 | 15.8 ± 6.24 | 14.2 ± 6.21 | 14.4 ± 6.61 |
| Baseline FSFI | | | | |
| Pain Score | | | | |
| Mean ± SD | 1.6 ± 1.11 | 1.8 ± 1.22 | 1.7 ± 1.17 | 1.7 ± 1.20 |

The Female Sexual Function Index (FSFI) total summary score is a numerically continuous measure that was descriptively summarized at Visits 2 and 6 and the change in the total summary score (Visit 6 minus Visit 2) was also descriptively summarized. The domain sub-scores and the changes in the domain sub-scores were also descriptively summarized. Summaries were by treatment arm, and all active treatment arms combined.

In addition, the change in mean from baseline of each active treatment group from the placebo group for each numerically continuous endpoint was evaluated. The least square (LS) mean changes and the 95% CI for the difference in LS Mean changes between treated and placebo are provided. The FSFI Questionnaire consists of 19 questions divided among 6 domains, and has a minimum total score of 2.0 and a maximum score of 36.0 points. The FSFI questionnaire was administered to the randomized population except for those subjects in the PK sub-study. At Baseline, the overall mean Total Score was 14.8 (14.8 for the 4 μg group; 15.8 for the 10 μg group; 14.2 for the 25 μg group; and 14.4 for the placebo group). The LS mean change in the FSFI Total Score and domain scores from Baseline to Week 12 are summarized in Table 146.

Change from Baseline to Week 12 in FSFI total score and domains compared to placebo was assessed.

After 12 weeks, total FSFI scores numerically improved from baseline in all groups, including placebo. Total FSFI score significantly increased with the 10 μg group (P<0.05) and the 25 μg group (P=0.0019) versus placebo (FIG. 24).

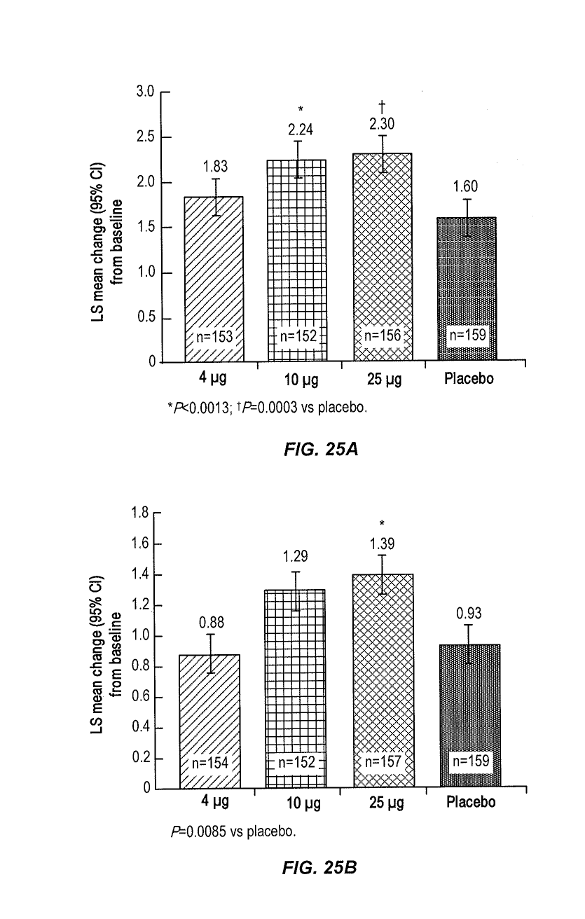

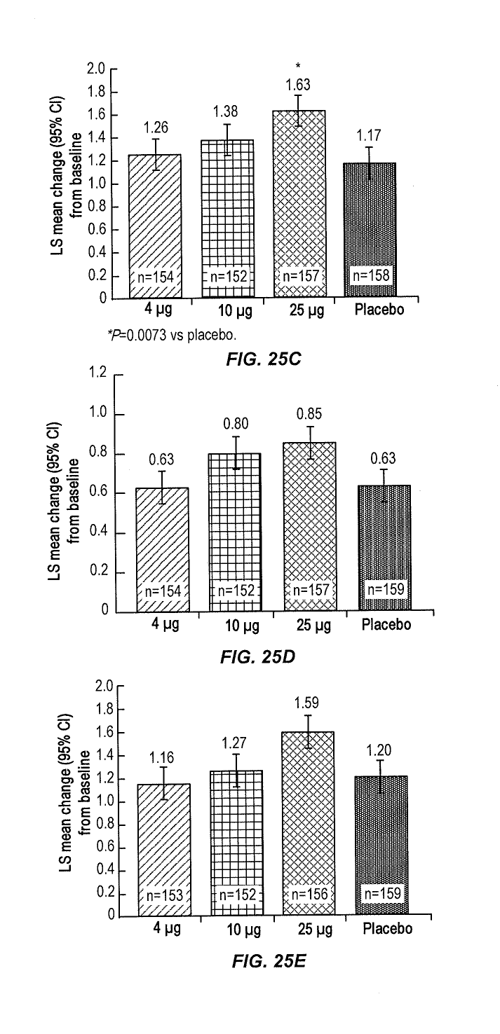

FSFI lubrication and pain domain scores improved numerically in all groups including placebo from baseline to 12 weeks; improvements for the 10 μg group and the 25 μg group were statistically significantly greater than with placebo (FIG. 25A). The 25 μg composition significantly improved FSFI arousal (P=0.0085) and satisfaction (P=0.0073) domain scores at 12 weeks (FIG. 25B, FIG. 25C). All three doses were comparable to placebo in their effect on the FSFI domains of desire and orgasm (FIG. 25D, FIG. 25E). The 4 μg composition and placebo provided similar levels of improvement. The compositions improved FSFI in a dose-dependent manner, with the 25 μg dose having the greatest improvement. All three doses were efficacious, and the numeric improvement in subjective symptoms was highest for subjects in the 10 and 25 μg groups. The observed placebo response could be attributed to the coconut oil (Miglyol) in the formulation for the placebo and the estradiol compositions, which may also contribute to the observed benefits.

TABLE 146
Female Sexual Function Index Total and Domain Scores:

| | | 4 μg | | 10 μg | | 25 μg | | Placebo | |
|---|---|---|---|---|---|---|---|---|---|
| Category | Score | Mean | SD | Mean | SD | Mean | SD | Mean | SD |
| Total | Baseline | 14.8 | 6.13 | 15.8 | 6.24 | 14.2 | 6.21 | 14.4 | 6.61 |
| | Week 12 | 22.6 | 8.4 | 24.8 | 7.59 | 24.8 | 7.59 | 22 | 8.54 |
| | Change | 7.98 | 7.551 | 8.85 | 7.361 | 10.49 | 8.176 | 7.74 | 8.41 |
| | LS | 7.909 | 0.9075 | 9.431 | 0.0492 | 10.283 | 0.0019 | 7.458 | — |
| | Mean | | | | | | | | |
| Arousal | Baseline | 2.8 | 1.44 | 2.9 | 1.43 | 2.7 | 1.5 | 2.7 | 1.41 |
| | Week 12 | 3.6 | 1.61 | 4.1 | 1.47 | 4.1 | 1.39 | 3.6 | 1.52 |
| | Change | 0.88 | 1.615 | 1.16 | 1.632 | 1.43 | 1.646 | 1.02 | 1.607 |
| | LS | 0.876 | 0.9777 | 1.288 | 0.0581 | 1.393 | 0.008 | 0.927 | — |
| | Mean | | | | | | | | |
| Desire | Baseline | 2.6 | 1.01 | 2.7 | 1.13 | 2.6 | 1.09 | 2.7 | 1.07 |
| | Week 12 | 3.3 | 1.11 | 3.5 | 1.13 | 3.5 | 1.06 | 3.3 | 1.21 |
| | Change | 0.64 | 1.065 | 0.78 | 1.113 | 0.87 | 1.105 | 0.62 | 1.102 |
| | LS | 0.626 | 1 | 0.801 | 0.2753 | 0.849 | 0.1139 | 0.628 | — |
| | Mean | | | | | | | | |
| Lubrication | Baseline | 2.1 | 1.25 | 2.3 | 1.25 | 2 | 1.19 | 2 | 1.29 |
| | Week 12 | 3.9 | 1.84 | 4.4 | 1.56 | 4.3 | 1.65 | 3.6 | 1.77 |
| | Change | 1.84 | 1.782 | 2.12 | 1.612 | 2.36 | 1.744 | 1.64 | 1.871 |
| | LS | 1.835 | 0.4023 | 2.243 | 0.0012 | 2.3 | 0.0003 | 1.591 | — |
| | Mean | | | | | | | | |
| Orgasm | Baseline | 2.7 | 1.74 | 2.9 | 1.74 | 2.4 | 1.68 | 2.4 | 1.73 |
| | Week 12 | 3.8 | 1.89 | 4.1 | 1.75 | 4.1 | 1.66 | 3.7 | 1.97 |
| | Change | 1.12 | 1.93 | 1.09 | 1.821 | 1.68 | 1.857 | 1.31 | 1.86 |
| | LS | 1.162 | 0.9978 | 1.273 | 0.9424 | 1.59 | 0.0763 | 1.189 | — |
| | Mean | | | | | | | | |
| Satisfaction | Baseline | 2.9 | 1.37 | 3.2 | 1.43 | 2.9 | 1.37 | 2.9 | 1.49 |
| | Week 12 | 4.2 | 1.54 | 4.4 | 1.37 | 4.6 | 1.35 | 4.1 | 1.55 |
| | Change | 1.31 | 1.512 | 1.24 | 1.534 | 1.64 | 1.613 | 1.23 | 1.661 |
| | LS | 1.256 | 0.8798 | 1.382 | 0.3484 | 1.628 | 0.0063 | 1.165 | — |
| | Mean | | | | | | | | |

While the pharmaceutical compositions and methods have been described in terms of what are presently considered to be practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar embodiments. This disclosure includes any and all embodiments of the following claims.

1. A method for treating moderate to severe dyspareunia in a subject, the method comprising:
intravaginally administering a pharmaceutical composition comprising estradiol to the subject, wherein the pharmaceutical composition is administered to the lower third of the vagina closest to the vaginal opening, and wherein the estradiol is not transported to the uterus of the subject.

2. The method of claim 1, wherein the pharmaceutical composition is a liquid pharmaceutical composition comprising 4 μg to 25 μg of estradiol, and wherein the liquid pharmaceutical composition is contained in a capsule.

3. The method of claim 1, wherein the administering is carried out by digitally inserting the capsule into the lower third of the vagina closest to the vaginal opening.

4. (canceled)

5. The method of claim 2, wherein the capsule is a bioadhesive capsule.

6. The method of claim 5, wherein the capsule is a gelatin capsule.

7. (canceled)

8. The method of claim 2, wherein the capsule adheres to the vaginal tissue of the subject and dissolves, ruptures, or disintegrates, thereby releasing the liquid pharmaceutical composition.

9. The method of claim 2, wherein the liquid pharmaceutical composition spreads over a surface area consisting of the vagina, the vulva and the labia.

10. The method of claim 6, wherein the soft gelatin capsule and the liquid pharmaceutical composition are fully absorbed by the vaginal tissue of the subject.

11. The method of claim 2, wherein the only discharge that occurs after intravaginally administering the liquid pharmaceutical composition is a natural discharge.

12. The method of claim 2, wherein the subject is ambulatory immediately after intravaginally administering the liquid pharmaceutical composition.

13. The method of claim 2, wherein the subject is ambulatory for a period of time beginning 5 minutes to 120 minutes after intravaginally administering the liquid pharmaceutical composition.

14. The method of any one of claims 2, 3, 5, 6, and 8-13, wherein the liquid pharmaceutical composition further comprises a solubilizing agent.

15. The method of any one of claim 14, wherein the solubilizing agent is an oil.

16. The method of any one of claim 15, wherein the oil comprises at least one C6-C12 fatty acid or a glycol, monoglyceride, diglyceride, or triglyceride ester thereof.

17. The method of any one of claims 2, 3, 5, 6, and 8-16, wherein the liquid pharmaceutical composition further comprises a thickener or a surfactant.

18. The method of any one of claims 2, 3, 5, 6, and 8-17, wherein the liquid pharmaceutical composition does not include a hydrophilic gel-forming bioadhesive agent.

19. The method of any one of claims 2, 3, 5, 6, and 8-18, wherein estradiol is the only active hormone in the liquid pharmaceutical composition.

20. The method of any one of claims 2, 3, 5, 6, and 8-19, wherein the liquid pharmaceutical composition includes 4 μg estradiol, 10 μg estradiol, or 25 μg estradiol.

21-22. (canceled)

23. The method of any one of claims 2, 3, 5, 6, and 8-20, wherein the intravaginal administration is conducted daily for two weeks, and twice weekly thereafter.

24. The method of claim 1, wherein the intravaginal administration is conducted at any time of day, but is conducted at about the same time each day.

25. The method of claim 1, wherein the treatment is effective within two weeks of the first administration.

26. The method of any one of claims 2, 3, 5, 6, and 8-20, and 23-25, comprising increasing the level of vaginal secretions in a subject, as assessed by visual examination, increasing the number of vaginal rugae in the subject, as assessed by visual examination, decreasing vaginal bleeding or petechiae in the subject, as assessed by visual examination, or changing the color of the vaginal mucosa in the subject from transparent to pink, or from pale pink to pink, as assessed by visual examination, and combinations thereof.

27-29. (canceled)

30. The method of any one of claims 2, 3, 5, 6, and 8-20, and 23-26, wherein the treatment decreases the severity of vaginal dryness within two weeks, wherein the treatment decreases the severity of vulvar or vaginal itching within two weeks, wherein the treatment decreases the severity of dyspareunia within two weeks, or wherein the treatment does not cause endometrial hyperplasia, and combinations thereof.

31-34. (canceled)

35. A method for avoiding transport of estradiol to the uterus of a subject in need of estradiol, the method comprising:
intravaginally administering a pharmaceutical composition comprising estradiol to the subject, wherein the pharmaceutical composition is administered to the lower third of the vagina closest to the vaginal opening.

36-39. (canceled)

40. A method for reestrogenizing the vagina, labia, or vulva without transporting estradiol to the uterus, the method comprising:
intravaginally administering a liquid pharmaceutical composition comprising 4 μg to 25 μg of estradiol to a subject in need thereof, wherein the liquid pharmaceutical composition is contained in a capsule, and wherein estradiol is not transported to the uterus of the subject.
